(19) United States
(12) Patent Application Publication
(10) Pub. No.: US 2012/0172333 A1
(43) Pub. Date: Jul. 5, 2012

(54) PYRROLO-PYRIDINE DERIVATIVES AS ACTIVATORS OF AMPK

(75) Inventor: Olivier Mirguet, Les Ulis (FR)

(73) Assignee: GLAXOSMITHKLINE LLC, Philadelphia, PA (US)

(21) Appl. No.: 13/394,927

(22) PCT Filed: Sep. 8, 2010
(86) PCT No.: PCT/EP10/63196

§ 371 Date: Mar. 8, 2012

(30) Foreign Application Priority Data
Sep. 10, 2009 (GB) 0915892.4

(51) Int. Cl. A61K 31/437; C07F 7/10; A61K 31/695; A61P 3/10; A61P 3/00; A61P 3/04; A61P 9/12; A61P 25/28; A61P 25/16; A61P 25/00; A61P 29/00; A61P 31/18; A61P 31/22; A61P 31/14; A61P 35/00; A61P 25/18; A61P 25/08; A61P 9/10; A61P 3/06; A61K 31/506; A61P 43/00; A61P 21/00; C07D 471/04

(52) U.S. Cl. 514/63; 546/113; 546/14; 514/300; 544/333; 514/256

(57) ABSTRACT

The present invention relates to pyrrolopyridone compounds of the formula (I),

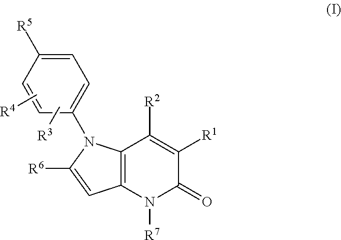

salts thereof, to pharmaceutical compositions containing them and their use in medicine. In particular, the invention relates to compounds as activators of AMPK.

### FIELD OF THE INVENTION

The present invention relates to a novel class of compounds which are activators of AMP-activated protein kinase (AMPK) (AMPK-activators), compositions comprising said compounds, methods of synthesis and uses for such compounds in treating various diseases mediated by AMPK, such as type 1 (Type I) diabetes, type 2 (Type II) diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

### BACKGROUND OF THE INVENTION

AMPK has been established as a sensor and regulator of cellular energy homeostasis (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003)). Allosteric activation of this kinase due to rising AMP levels occurs in states of cellular energy depletion. The resulting serine/threonine phosphorylation of target enzymes leads to an adaptation of cellular metabolism to the low energy state. The net effect of AMPK activation induced changes is inhibition of ATP consuming processes and activation of ATP generating pathways, and therefore regeneration of ATP stores. Examples of AMPK substrates include acetyl-CoA-carboxylase (ACC) and HMG-CoA-reductase (Carling, D. et. al. A common bicyclic protein kinase cascade inactivates the regulatory enzymes of fatty acid and cholesterol biosynthesis. FEBS Letters 223:217 (1987)). Phosphorylation and therefore inhibition of ACC leads to a decrease in fatty acid synthesis (ATP-consuming) and at the same time to an increase in fatty acid oxidation (ATP-generating). Phosphorylation and resulting inhibition of HMG-CoA reductase leads to a decrease in cholesterol synthesis. Other substrates of AMPK include hormone sensitive lipase (Garton, A. J. et. al. Phosphorylation of bovine hormone-sensitive lipase by the AMP-activated protein kinase. A possible antilipolytic mechanism. Eur. J. Biochem. 179:249 (1989)), glycerol-3-phosphate acyltransferase (Muoio, D. M. et. al. AMP-activated kinase reciprocally regulates triacylglycerol synthesis and fatty acid oxidation in liver and muscle: evidence that sn-glycerol-3-phosphate acyltransferase is a novel target. Biochem. J. 338:783 (1999)), malonyl-CoA decarboxylase (Saha, A. K. et. al. Activation of malonyl-CoA decarboxylase in rat skeletal muscle by contraction and the AMP-activated protein kinase activator 5-aminoimidazole-4-carboxamide-1-.beta.-D-ribofuranoside. J. Biol. Chem. 275:24279 (2000)), some of which are potential drug targets for components of metabolic syndrome. Additional processes that are believed to be regulated through AMPK activation, but for which the exact AMPK substrates have not been identified, include stimulation of glucose transport in skeletal muscle and expressional regulation of key genes in fatty acid and glucose metabolism in liver (Hardie, D. G. and Hawley, S. A. AMP-activated protein kinase: the energy charge hypothesis revisited. Bioessays 23: 1112 (2001), Kemp, B. E. et. al. AMP-activated protein kinase, super metabolic regulator. Biochem. Soc. Transactions 31:162 (2003), Musi, N. and Goodyear, L. J. Targeting the AMP-activated protein kinase for the treatment of Type 2 diabetes. Current Drug Targets-Immune, Endocrine and Metabolic Disorders 2:119 (2002)). For example, decreased expression of glucose-6-phosphatase (Lochhead, P. A. et. al. 5-aminoimidazole-4-carboxamide riboside mimics the effects of insulin on the expression of the 2 key gluconeogenic genes PEPCK and glucose-6-phosphatase. Diabetes 49:896 (2000)), a key enzyme in hepatic glucose production, and SREBP-1c (Zhou, G. et. al. Role of AMP-activated protein kinase in mechanism of metformin action. The J. of Clin. Invest. 108: 1167 (2001)), a key lipogenic transcription factor, has been found following AMPK stimulation.

More recently an involvement of AMPK in the regulation of not only cellular but also whole body energy metabolism has become apparent. It was shown that the adipocyte-derived hormone leptin leads to a stimulation of AMPK and therefore to an increase in fatty acid oxidation in skeletal muscle (Minokoshi, Y. et. al. Leptin stimulates fatty-acid oxidation by activating AMP-activated protein kinase. Nature 415: 339 (2002)). Adiponectin, another adipocyte derived hormone leading to improved carbohydrate and lipid metabolism, has been demonstrated to stimulate AMPK in liver and skeletal muscle (Yamauchi, T. et. al. Adiponectin stimulates glucose utilization and fatty acid oxidation by activating AMP-activated protein kinase. Nature Medicine 8: 1288 (2002), Tomas, E. et. al. Enhanced muscle fat oxidation and glucose transport by ACRP30 globular domain: Acetyl-CoA carboxylase inhibition and AMP-activated protein kinase activation. PNAS 99: 16309(2002)). The activation of AMPK in these circumstances seems to be independent of increasing cellular AMP levels but rather due to phosphorylation by one or more yet to be identified upstream kinases.

Based on the knowledge of the above-mentioned consequences of AMPK activation, certain beneficial effects could be expected from in vivo activation of AMPK. In liver, decreased expression of gluconeogenic enzymes could reduce hepatic glucose output and improve overall glucose homeostasis, and both direct inhibition and/or reduced expression of key enzymes in lipid metabolism could lead to decreased fatty acid and cholesterol synthesis and increased fatty acid oxidation. Stimulation of AMPK in skeletal muscle could increase glucose uptake and fatty acid oxidation with resulting improvement of glucose homeostasis and, due to a reduction in intra-myocyte triglyceride accumulation, to improved insulin action. Finally, the increase in energy expenditure could lead to a decrease in body weight. The combination of these effects in metabolic syndrome could be expected to reduce the risk for acquiring cardiovascular diseases.

Several studies in rodents support this hypothesis (Bergeron, R. et. al. Effect of 5-aminoimidazole-4-carboxamide-1(beta)-D-ribofuranoside infusion on in vivo glucose metabolism in lean and obese Zucker rats. Diabetes 50:1076 (2001), Song, S. M. et. al. 5-Aminoimidazole-4-darboxamide ribonucleoside treatment improves glucose homeostasis in insulin-resistant diabeted (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying feature of the insulin resistance syndrome. Diabetes 51: 2199 (2002)). Until recently most in vivo studies have relied on the AMPK activator AICAR, a cell permeable precursor of ZMP. ZMP acts as an intracellular AMP mimic, and, when accumulated to high enough levels, is able to stimulate AMPK activity (Corton, J. M. et. al. 5-Aminoimidazole-4-carboxamide ribonucleoside, a specific method for activating AMP-activated protein kinase in intact cells? Eur. J. Biochem. 229: 558 (1995)). However, ZMP also acts as an AMP mimic in the regulation of other enzymes, and is therefore not a specific AMPK activator (Musi, N. and Goodyear, L. J. Targeting the AMP-activated protein kinase for the treatment of Type 2 diabetes. Current Drug Targets-Immune, Endocrine and Metabolic Disorders 2:119 (2002)). Several in vivo studies have demonstrated beneficial effects of both acute and chronic AICAR administration in rodent models of obesity and Type 2 diabetes (Bergeron, R. et. al. Effect of 5-aminoimidazole-4-carboxamide-1(beta)-D-ribofuranoside infusion on in vivo glucose metabolism in lean and obese Zucker rats. Diabetes 50:1076 (2001), Song, S. M. et. al. 5-Aminoimidazole-4-darboxamide ribonucleoside treatment improves glucose homeostasis in insulin-resistant diabetic (ob/ob) mice. Diabetologia 45:56 (2002), Halseth, A. E. et. al. Acute and chronic treatment of ob/ob and db/db mice with AICAR decreases blood glucose concentrations. Biochem. and Biophys. Res. Comm. 294:798 (2002), Buhl, E. S. et. al. Long-term AICAR administration reduces metabolic disturbances and lowers blood pressure in rats displaying feature of the insulin resistance syndrome. Diabetes 51: 2199 (2002)). For example, 7 week AICAR administration in the obese Zucker (fa/fa) rat leads to a reduction in plasma triglycerides and free fatty acids, an increase in HDL cholesterol, and a normalization of glucose metabolism as assessed by an oral glucose tolerance test (Minokoshi, Y. et. al. Leptin stimulates fatty-acid oxidation by activating AMP-activated protein kinase. Nature 415: 339 (2002)). In both ob/ob and db/db mice, 8 day AICAR administration reduces blood glucose by 35% (Halseth, A. E. et. al. Acute and chronic treatment of ob/ob and db/db mice with AICAR decreases blood glucose concentrations. Biochem. and Biophys. Res. Comm. 294:798 (2002)). In addition to AICAR, more recently it was found that the diabetes drug metformin can activate AMPK in vivo at high concentrations (Zhou, G. et. al. Role of AMP-activated protein kinase in mechanism of metformin action. The J. of Clin. Invest. 108: 1167 (2001), Musi, N. et. al. Metformin increases AMP-activated protein kinase activity in skeletal muscle of subjects with Type 2 diabetes. Diabetes 51: 2074 (2002)), although it has to be determined to what extent its antidiabetic action relies on this activation. As with leptin and adiponectin, the stimulatory effect of metformin is indirect via a mild inhibition of mitochondrial respiratory chain complex 1 (Leverve X. M. et al. Mitochondrial metabolism and type-2 diabetes: a specific target of metformin. Diabetes Metab. 29: 6588 (2003)). In addition to pharmacologic intervention, several transgenic mouse models have been developed in the last years and initial results are becoming available. Expression of dominant negative AMPK in skeletal muscle of transgenic mice has demonstrated that the AICAR effect on stimulation of glucose transport is dependent on AMPK activation (Mu, J. et. al. A role for AMP-activated protein kinase in contraction and hypoxia-regulated glucose transport in skeletal muscle. Molecular Cell 7: 1085 (2001)), and therefore likely not caused by non-specific ZMP effects. Similar studies in other tissues will help to further define the consequences of AMPK activation. It is believed that pharmacologic activation of AMPK may have benefits in relation to metabolic syndrome with improved glucose and lipid metabolism and a reduction in body weight. To qualify a patient as having metabolic syndrome, three out of the five following criteria must be met: elevated blood pressure above 130/85 mmHg, fasting blood glucose above 110 mg/dl, abdominal obesity above 40″ (men) or 35″ (women) waist circumference, and blood lipid changes as defined by an increase in triglycerides above 150 mg/dl or decreased HDL cholesterol below 40 mg/dl (men) or 50 mg/dl (women). Therefore, the combined effects that may be achieved through activation of AMPK in a patient who qualifies as having metabolic syndrome would raise the interest of this target.

Lowering of blood pressure has been reported to be a consequence of AMPK activation (Buhl, E. S. et. al. Long-term AICAR administration reduces metabolic disturbances and lowers blood pressure in rats displaying feature of the insulin resistance syndrome. Diabetes 51: 2199 (2002)), therefore activation of AMPK might have beneficial effects in hypertension. Through combination of some or all of the above-mentioned effects stimulation of AMPK may to reduce the incidence of cardiovascular diseases (e.g. MI, stroke). Increased fatty acid synthesis is a characteristic of many tumor cells, therefore decreased synthesis of fatty acids through activation of AMPK could be useful as a cancer therapy (Huang X. et al. Important role of the LKB1-AMPK pathway in suppressing tumorigenesis in PTEN-deficient mice. Biochem J. 412: 211 (2008). AMPK can also be considered as a metabolic tumor suppressor and AMPK activators could be helpful in general cancer therapy (Luo Z. Et al. AMPK as a metabolic tumor suppressor: control of metabolism and cell growth. Future Oncol. 6: 457 (2010)). Stimulation of AMPK has been shown to stimulate production of ketone bodies from astrocytes (Blazquez, C. et. al. The AMP-activated protein kinase is involved in the regulation of ketone body production by astrocytes. J. Neurochem. 73: 1674 (1999)), and might therefore be a strategy to treat ischemic events in the brain. Stimulation of AMPK has been shown to improve cognition and neurodegenerative diseases in a mice model (Dagon Y. et al. Nutritional status, cognition, and survival: a new role for leptin and AMP kinase. J. Biol. Chem. 280:42142 (2005)). Stimulation of AMPK has been shown to stimulate expression of uncoupling protein 3 (UCP3) in skeletal muscle (Zhou, M. et. al. UCP-3 expression in skeletal muscle: effects of exercise, hypoxia, and AMP-activated protein kinase. Am. J. Physiol. Endocrinol. Metab. 279: E622 (2000)) and might therefore be a way to prevent damage from reactive oxygen species. Endothelial NO synthase (eNOS) has been shown to be activated through AMPK mediated phosphorylation (Chen, Z.-P., et. al. AMP-activated protein kinase phosphorylation of endothelial NO synthase. FEBS Letters 443: 285 (1999)), therefore AMPK activation may be used to improve local circulatory systems. AMPK has also been described to directly affect PGC-1alpha activity through phosphorylation and then regulate mitochondria biogenesis (Jager S, et al. AMP-activated protein kinase (AMPK) action in skeletal muscle via direct phosphorylation of PGC-1alpha. Proc Natl Acad Sci 104:12017 (2007)). AMPK activation can be then a way to treat mitochondrial disorders (e.g. sarcopenia and some mitochondrial rare diseases). Recently, several reports describe beneficial effect of AMPK activation on virus infection. While virus infection is found to reduce AMPK activity in infected cells or tissues, AMPK activation is proposed as a anti-viral therapy (Mankouri J. et al., Enhanced hepatitis C virus genome replication and lipid accumulation mediated by inhibition of AMP-activated protein kinase, Proc Natl Acad Sci 107: 11549 (2010)).

### SUMMARY OF THE INVENTION

The present invention provides compounds of formula (I):

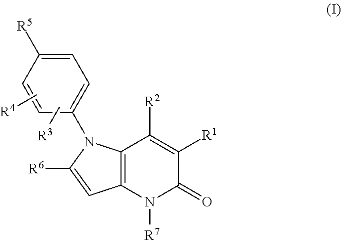

wherein
R¹ represents:
(i) —CN, H or —CO₂C₁₋₃alkyl (such as —CO₂C₂H₅); or
(ii) -phenyl optionally substituted by one or two groups independently selected from —C₁₋₄alkyl (such as —CH₃), —OC₁₋₄alkyl (such as —OCH₃ or —OC₂H₅), —OH, —C₁₋₄haloalkyl (such as —CF₃), —NO₂, —CN, —CO₂H, —C(═O)NH₂ or halogen (such as chloro, bromo or fluoro);
   R² represents —OH;
   R³ and R⁴ each independently represent H, —C₁₋₄alkyl (such as —CH₃), —OC₁₋₄alkyl (such as —OCH₃) or fluoro;
   R⁵ represents
(i) H, —C₁₋₄alkyl (such as —CH₃ or —C₂H₅), —OC₁₋₄alkyl (such as —OCH₃), —CO₂C₁₋₄alkyl (such as —CO₂C₂H₅), —CN, —C₁₋₄alkylCN (such as —CH₂CN), —C₁₋₄haloalkyl (such as —CF₃), —OC₁₋₄haloalkyl (such as —OCF₃), —C(═O)C₁₋₄alkyl (such as —C(═O)CH₃), —XC(═O)C₁₋₄alkyl (such as —XC(═O)CH₃) or halogen (such as chloro, bromo or fluoro); or
(ii) —C₆₋₁₀aryl (such as phenyl), -(5-10 membered heteroaryl), -(5-10 membered heterocyclyl) or —C₃₋₈cycloalkyl (such as cyclohexyl) wherein the —C₆₋₁₀aryl, -(5-10 membered heteroaryl), -(5-10 membered heterocyclyl) or —C₃₋₈cycloalkyl is optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro);
   X represents O or NR⁸;
   R⁶ represents H, —C₁₋₄alkyl (such as —CH₃), —CN or chloro;
   R⁷ represents H or —C₁₋₄alkyl (such as —CH₃);
   R⁸ represents H or —C₁₋₄alkyl (such as —CH₃); and
   R⁹ and R¹⁰ each independently represent H or —C₁₋₄alkyl (such as —CH₃);
   or a salt thereof.

The present invention also provides compounds of formula (IA) which are a subset of compounds of formula (I):

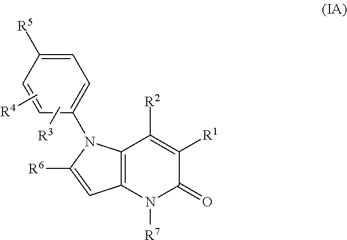

wherein
R¹ represents:
(i) —CN, H or —CO₂C₁₋₃alkyl (such as —CO₂C₂H₅);
(ii) -phenyl optionally substituted by one or two groups independently selected from —C₁₋₄alkyl (such as —CH₃), —OC₁₋₄alkyl (such as —OCH₃), —OH, —C₁₋₄haloalkyl (such as —CF₃), —CN or halogen (such as chloro, bromo or fluoro);
   R² represents —OH;
   R³ and R⁴ each independently represent H or fluoro;
   R⁵ represents
(i) H, —C₁₋₄alkyl (such as —CH₃), —OC₁₋₄alkyl (such as —OCH₃), —CO₂C₁₋₄alkyl (such as —CO₂C₂H₅) or halogen (such as chloro, bromo or fluoro); or
(ii) —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl is optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro);
   X represents O or NR⁸;
   R⁶ represents H, —C₁₋₄alkyl (such as —CH₃) or chloro;
   R⁷ represents H or —C₁₋₄alkyl (such as —CH₃); and
   R⁸ represents H or —C₁₋₄alkyl (such as —CH₃);
   or a salt thereof.

In another aspect, the present invention provides pharmaceutical compositions comprising a compound of formula (I) or a pharmaceutically acceptable salt thereof.

In another aspect, the present invention provides methods of treating type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer comprising administration of a therapeutically effective amount of a compound of formula (I) or a pharmaceutically acceptable salt thereof to a subject in need thereof.

In another aspect, the present invention provides methods of treating diabetes, metabolic syndrome, atherosclerosis, dyslipidaemia, obesity, hypertension, cerebral ischemia, cognitive defect and cancer comprising administration of a therapeutically effective amount of a compound of formula (I) or a pharmaceutically acceptable salt thereof to a subject in need thereof.

In another aspect, the present invention provides methods of treating type 2 diabetes, obesity or dyslipidaemia comprising administration of a therapeutically effective amount of a compound of formula (I) or a pharmaceutically acceptable salt thereof to a subject in need thereof.

In another aspect, the invention provides a compound of formula (I) or a pharmaceutically acceptable salt thereof for use in human or veterinary medical therapy.

In another aspect, the invention provides a compound of formula (I) or a pharmaceutically acceptable salt thereof, for use in the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

In another aspect, the invention provides a compound of formula (I) or a pharmaceutically acceptable salt thereof, for use in the treatment of diabetes, metabolic syndrome, atherosclerosis, dyslipidaemia, obesity, hypertension, cerebral ischemia, cognitive defect and cancer.

In another aspect, the invention provides a compound of formula (I) or a pharmaceutically acceptable salt thereof, for use in the treatment type 2 diabetes, obesity or dyslipidaemia.

In another aspect, the invention provides the use of a compound of formula (I) or a pharmaceutically acceptable salt thereof in the manufacture of a medicament for the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

In another aspect, the invention provides the use of a compound of formula (I) or a pharmaceutically acceptable salt thereof in the manufacture of a medicament for the treatment of diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof in the manufacture of a medicament for the treatment of type 2 diabetes, obesity or dyslipidaemia.

### DESCRIPTION OF THE EMBODIMENTS

All aspects and embodiments of the invention described herein are in respect of compounds of formula I and (IA) unless otherwise specified.

In one aspect of the invention, R¹ represents —CN, H or —CO₂C₁₋₃alkyl (such as —CO₂C₂H₅).

In another aspect of the invention, R¹ represents CN. In another aspect of the invention, R¹ represents H. In another aspect of the invention, R¹ represents —CO₂C₁₋₃alkyl. In another aspect of the invention R¹ represents —CO₂C₂H₅.

In another aspect of the invention, R¹ represents phenyl optionally substituted by one or two groups independently selected from —C₁₋₄alkyl (such as —CH₃), —OC₁₋₄alkyl (such as —OCH₃ or —OC₂H₅), —OH, —C₁₋₄haloalkyl (such as —CF₃), —NO₂, —CN, —CO₂H, —C(═O)NH₂ or halogen (such as chloro, bromo or fluoro).

In another aspect of the invention, R¹ represents phenyl optionally substituted by one or two groups independently selected from —C₁₋₄alkyl (such as —CH₃), —OC₁₋₄alkyl (such as —OCH₃), —OH, —C₁₋₄haloalkyl (such as —CF₃), —CN or halogen (such as chloro, bromo or fluoro).

In another aspect of the invention, R¹ represents phenyl substituted by one or two groups independently selected from —C₁₋₄alkyl (such as —CH₃), —OC₁₋₄alkyl (such as —OCH₃ or —OC₂H₅), —OH, —C₁₋₄haloalkyl (such as —CF₃), —NO₂, —CN, —CO₂H, —C(═O)NH₂ or halogen (such as chloro, bromo or fluoro).

In another aspect of the invention, R¹ represents phenyl substituted by one or two groups independently selected from —C₁₋₄alkyl (such as —CH₃), —OC₁₋₄alkyl (such as —OCH₃), —OH, —C₁₋₄haloalkyl (such as —CF₃), —CN or halogen (such as chloro, bromo or fluoro).

In another aspect of the invention, R¹ represents phenyl optionally substituted by a group independently selected from —C₁₋₄alkyl (such as —CH₃), —OC₁₋₄alkyl (such as —OCH₃ or —OC₂H₅), —OH, —C₁₋₄haloalkyl (such as —CF₃), —NO₂, —CN, —CO₂H, —C(═O)NH₂ or halogen (such as chloro, bromo or fluoro).

In another aspect of the invention, R¹ represents phenyl optionally substituted by a group selected from —C₁₋₄alkyl (such as —CH₃), —OC₁₋₄alkyl (such as —OCH₃), —OH, —C₁₋₄haloalkyl (such as —CF₃), —CN or halogen (such as chloro, bromo or fluoro).

In another aspect of the invention, R¹ represents phenyl substituted by a group independently selected from —C₁₋₄alkyl (such as —CH₃), —OC₁₋₄alkyl (such as —OCH₃ or —OC₂H₅), —OH, —C₁₋₄haloalkyl (such as —CF₃), —NO₂, —CN, —CO₂H, —C(═O)NH₂ or halogen (such as chloro, bromo or fluoro).

In another aspect of the invention, R¹ represents phenyl substituted by a group selected from —C₁₋₄alkyl (such as —CH₃), —OC₁₋₄alkyl (such as —OCH₃), —OH, —C₁₋₄haloalkyl (such as —CF₃), —CN or halogen (such as chloro, bromo or fluoro).

In another aspect of the invention, R¹ represents phenyl optionally substituted by one group selected from —OC₁₋₄alkyl (such as —OCH₃), —CN or halogen (such as chloro, bromo or fluoro).

In another aspect of the invention, R¹ represents phenyl substituted by a group selected from —OC₁₋₄alkyl (such as —OCH₃), —CN or halogen (such as chloro, bromo or fluoro).

In another aspect of the invention, R¹ represents phenyl substituted by a group independently selected from —CH₃, —OCH₃, —OC₂H₅, —OH, —CF₃, —NO₂, —CN, —CO₂H, —C(═O)NH₂ or halogen (such as chloro, bromo or fluoro).

In another aspect of the invention, R¹ represents phenyl optionally substituted by a group selected from —OCH₃, —CN or halogen (such as chloro, bromo or fluoro).

In another aspect of the invention, R¹ represents phenyl substituted by a group selected from —OCH₃, —CN or halogen (such as chloro, bromo or fluoro).

In another aspect of the invention, R¹ represents phenyl substituted by fluoro.

In another aspect of the invention, R¹ represents phenyl.

In one aspect of the invention, R³ and R⁴ each independently represent H. In another aspect of the invention, R³ and R⁴ each independently represent fluoro.

In another aspect of the invention, R³ and R⁴ each independently represent —C₁₋₄alkyl (such as —CH₃). In another aspect of the invention, R³ and R⁴ each independently represent —CH₃. In another aspect of the invention, R³ and R⁴ each independently represent —OC₁₋₄alkyl (such as —CH₃). In another aspect of the invention, R³ and R⁴ each independently represent —OCH₃.

In one aspect of the invention, R³ represents fluoro when R⁴ represents H. In another aspect of the invention, R³ represents H when R⁴ represents fluoro.

In one aspect of the invention, R³ represents —OCH₃ when R⁴ represents H. In another aspect of the invention, R³ represents H when R⁴ represents —OCH₃.

In one aspect of the invention, R³ represents —CH₃ when R⁴ represents H. In another aspect of the invention, R³ represents H when R⁴ represents —CH₃.

In one aspect of the invention, R⁵ represents H, —C₁₋₄alkyl (such as —CH₃ or —C₂H₅), —OC₁₋₄alkyl (such as —OCH₃), —CO₂C₁₋₄alkyl (such as —CO₂O₂H₅), —CN, —C₁₋₄alkylCN (such as —CH₂CN), —C₁₋₄haloalkyl (such as —CF₃), —OC₁₋₄haloalkyl (such as —OCF₃), —C(═O)C₁₋₄alkyl (such as —C(═O)CH₃), —XC(═O)C₁₋₄alkyl (such as —XC(═O)CH₃) or halogen (such as chloro, bromo or fluoro).

In another aspect of the invention R⁵ represents —C₁₋₄alkyl (such as —CH₃), —OC₁₋₄alkyl (such as —OCH₃), —CO₂C₁₋₄alkyl (such as —CO₂O₂H₅) or halogen (such as chloro, bromo or fluoro).

In another aspect of the invention, R⁵ represents H, —CH₃, —C₂H₅, —OCH₃, —CO₂O₂H₅, —CN, —CH₂CN, —CF₃, —OCF₃, —C(═O)CH₃, —XC(═O)CH₃ or halogen (such as chloro, bromo or fluoro).

In another aspect of the invention, R⁵ represents —CH₃, —OCH₃, —CO₂C₂H₅ or halogen (such as chloro, bromo or fluoro).

In another aspect of the invention, R⁵ represents —OCH₃. In another aspect of the invention, R⁵ represents —CO₂C₂H₅. In another aspect of the invention, R⁵ represents halogen (such as chloro, bromo or fluoro). In another aspect of the invention, R⁵ represents —CH₃. In another aspect of the invention, R⁵ represents —C₂H₅ (ethyl). In another aspect of the invention, R⁵ represents —CN. In another aspect of the invention, R⁵ represents —CH₂CN. In another aspect of the invention, R⁵ represents —CF₃. In another aspect of the invention, R⁵ represents —OCF₃. In another aspect of the invention, R⁵ represents H or bromo. In another aspect of the invention, R⁵ represents H. In another aspect of the invention, R⁵ represents halogen. In another aspect of the invention, R⁵ represents bromo. In another aspect of the invention, R⁵ represents fluoro.

In another aspect of the invention R⁵ represents —C₆₋₁₀aryl (such as phenyl), -(5-10 membered heteroaryl), -(5-10 membered heterocyclyl) or —C₃₋₈cycloalkyl (such as cyclohexyl) wherein the —C₆₋₁₀aryl), -(5-10 membered heteroaryl), -(5-10 membered heterocyclyl) or —C₃₋₈cycloalkyl is optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl is optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl), -(5-10 membered heteroaryl), -(5-10 membered heterocyclyl) or —C₃₋₈cycloalkyl (such as cyclohexyl) wherein the —C₆₋₁₀aryl, —C₅₋₁₀heteroaryl, —C₅₋₁₀heterocyclyl or —C₃₋₈cycloalkyl is substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl is substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl), -(5-10 membered heteroaryl), -(5-10 membered heterocyclyl) or —C₃₋₈cycloalkyl (such as cyclohexyl) wherein the —C₆₋₁₀aryl, —C₅₋₁₀heteroaryl, —C₅₋₁₀heterocyclyl or —C₃₋₈cycloalkyl is substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl is substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl), -(5-10 membered heteroaryl), -(5-10 membered heterocyclyl) or —C₃₋₈cycloalkyl (such as cyclohexyl) wherein the —C₆₋₁₀aryl, —C₅₋₁₀heteroaryl, —C₅₋₁₀heterocyclyl or —C₃₋₈cycloalkyl is substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl), —C₅₋₁₀heteroaryl or —C₅₋₁₀heterocyclyl wherein the —C₆₋₁₀aryl, —C₅₋₁₀heteroaryl or —C₅₋₁₀heterocyclyl is substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl) optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl) optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl) substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl) substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl) substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl) substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents —C₆₋₁₀aryl (such as phenyl) substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₆₋₁₀aryl (such as phenyl) substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention R⁵ represents phenyl substituted by one or two groups independently selected from —CH₃, —OCH₃, —CH₂CH═CHCH₂, —OH, —CH₂OH, —CF₃, —CN, —CO₂H, —CH₂OH, —NHC(═O)CH₃, —Si(CH₃)₃, —SO₂NH₂, —C(═O)NH₂, —NH₂ or halogen (such as chloro, bromo or fluoro).

In another aspect of the invention R⁵ represents phenyl substituted by a group independently selected from —CH₃, —OCH₃, —CH₂CH═CHCH₂, —OH, —CH₂OH, —CF₃, —CN, —CO₂H, —CH₂OH, —NHC(═O)CH₃, —Si(CH₃)₃, —SO₂NH₂, —C(═O)NH₂, —NH₂ or halogen (such as chloro, bromo or fluoro).

In another aspect of the invention, R⁵ represents —C₆₋₁₀aryl (such as phenyl) substituted by one or two groups independently selected from —CH₃, —OCH₃, —CH₂CH═CHCH₂, —OH, —CH₂OH, —CF₃, —CN, —CO₂H, —CH₂OH, —XC(═O)CH₃, —Si(CH₃)₃ or halogen (such as chloro, bromo or fluoro).

In another aspect of the invention R⁵ represents —C₆₋₁₀aryl (such as phenyl).

In another aspect of the invention, R⁵ represents phenyl.

In another aspect of the invention, R⁵ represents -(5-10 membered heteroaryl) optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₅₋₁₀heteroaryl optionally substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention, R⁵ represents -(5-10 membered heteroaryl) substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₅₋₁₀heteroaryl substituted by one, two or three groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention, R⁵ represents -(5-10 membered heteroaryl) substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁹R¹⁰, —C(═O)NR⁹R¹⁰, —NR⁹R¹⁰ or halogen (such as chloro, bromo or fluoro).

In another aspect of the invention, R⁵ represents —C₅₋₁₀heteroaryl substituted by one or two groups independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃) or halogen (such as chloro, bromo or fluoro).

In another aspect of the invention, R⁵ represents -(5-10 membered heteroaryl) substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃), —Si(C₁₋₃alkyl)₃ (such as —Si(CH₃)₃), —SO₂NR⁸R¹⁰, —C(═O)NR⁸R¹⁰, —NR⁸R¹⁰ or halogen (such as chloro, bromo or fluoro).

In another aspect of the invention, R⁵ represents —C₅₋₁₀heteroaryl substituted by a group independently selected from —C₁₋₄alkyl (such as —CH₃), —OC₁₋₄alkyl (such as —OCH₃), —C₂₋₄alkenyl (such as —CH₂CH═CHCH₂), —OH, —C₁₋₄alkyleneOH (such as —CH₂OH), —C₁₋₄haloalkyl (such as —CF₃), —CN, —CO₂H, —C₁₋₄alkyleneCO₂H (such as —CH₂CO₂H), —XC(═O)C₁₋₄alkyl (such as —XC(═O)CH₃)—Si(C₁₋₃alkyl)₃ (such as —Si(CH₃)₃) or halogen (such as chloro, bromo or fluoro).

In another aspect of the invention R⁵ represents -(5-10 membered heteroaryl) substituted by one or two groups independently selected from —CH₃, —OCH₃, —CH₂CH═CHCH₂, —OH, —CH₂OH, —CF₃, —CN, —CO₂H, —CH₂CO₂H, —NHC(═O)CH₃, —Si(CH₃)₃, —SO₂NH₂, —C(═O)NH₂, —NH₂ or halogen (such as chloro, bromo or fluoro).

In another aspect of the invention R⁵ represents -(5-10 membered heteroaryl) substituted by a group independently selected from —CH₃, —OCH₃, —CH₂CH═CHCH₂, —OH, —CH₂OH, —CF₃, —CN, —CO₂H, —CH₂CO₂H, —NHC(═O)CH₃, —Si(CH₃)₃, —SO₂NH₂, —C(═O)NH₂, —NH₂ or halogen (such as chloro, bromo or fluoro).

In another aspect of the invention, R⁵ represents —C₅₋₁₀heteroaryl substituted by one or two groups independently selected from —CH₃, —OCH₃, —CH₂CH═CHCH₂, —OH, —CH₂OH, —CF₃, —CN, —CO₂H, —CH₂CO₂H, —XC(═O)CH₃, —Si(CH₃)₃ or halogen (such as chloro, bromo or fluoro).

In another aspect of the invention, R⁵ represents —C₅₋₁₀heteroaryl.

In another aspect of the invention, R⁵ represents —C₅₋₁₀heterocyclyl.

In another aspect of the invention, R⁵ represents —C₃₋₈cycloalkyl (such as cyclohexyl).

In another aspect of the invention, R⁵ represents pyridinyl. In another aspect of the invention, R⁵ represents thiophene. In another aspect of the invention, R⁵ represents benzodioxolane. In another aspect of the invention, R⁵ represents benzodioxane. In another aspect of the invention, R⁵ represents oxazole. In another aspect of the invention, R⁵ represents pyrrole. In another aspect of the invention, R⁵ represents cyclohexane.

In one aspect of the invention, X represents O. In another aspect of the invention, X represents NR⁸.

In one aspect of the invention, R⁶ represents H. In another aspect of the invention, R⁶ represents —C₁₋₄alkyl. In another aspect of the invention, R⁶ represents —CH₃. In another aspect of the invention, R⁶ represents H or chloro. In another aspect of the invention, R⁶ represents chloro. In another aspect of the invention, R⁶ represents CN.

In one aspect of the invention, R⁷ represents H. In another aspect of the invention, R⁷ represents —C₁₋₄alkyl. In another aspect of the invention, R⁷ represents —CH₃.

In one aspect of the invention, R⁸ represents H. In another aspect of the invention, R⁸ represents —C₁₋₄alkyl. In another aspect of the invention, R⁸ represents —CH₃.

In one aspect of the invention, R⁹ represents H. In another aspect of the invention, R⁹ represents —C₁₋₄alkyl. In another aspect of the invention, R⁹ represents —CH₃.

In one aspect of the invention, R¹⁰ represents H. In another aspect of the invention, represents —C₁₋₄alkyl. In another aspect of the invention, R¹⁰ represents —CH₃.

Each of the aspects of the invention are independent unless stated otherwise. Nevertheless the skilled person will understand that all the permutations of the aspects herein described are within the scope of the invention. Thus it is to be understood that the present invention covers all combinations of suitable, convenient and exemplified aspects described herein.

As used herein, the term “alkyl” refers to straight or branched hydrocarbon chains containing the specified number of carbon atoms. For example, —C₁₋₄alkyl refers to a straight or branched “alkyl” containing at least 1, and at most 4, carbon atoms. Examples of “alkyl” as used herein include, but are not limited to, methyl, ethyl, n-propyl, n-butyl, isobutyl, isopropyl and t-butyl.

As used herein, the term “alkylene” refers to straight or branched chain saturated hydrocarbon linker groups containing the specified number of carbon atoms. For example, —C₁₋₄alkylene refers to a straight or branched “alkylene” containing at least 1, and at most 4, carbon atoms. Examples of “alkylene” as used herein include, but are not limited to, methylene (—CH₂—) and ethylene (—CH₂CH₂—).

As used herein, the term “alkenyl’ refers to straight or branched unsaturated hydrocarbon groups, wherein the unsaturation is present only as double bonds and containing the specified number of carbon atoms. For example —C₂₋₄alkenyl refers to straight or branched chain unsaturated hydrocarbon groups containing one or more double bond(s) and having from 2 to 4 carbon atoms. Examples of “alkenyl” as used herein include, but are not limited to, ethenyl (—CH═CH₂) and propenyl (—CH═CHCH₃ or —CH₂CH═CH₂).

As used herein, the term “—C₆₋₁₀aryl” refers to an aromatic carbocyclic moiety containing 6 to 10 carbon ring-atoms. The definition includes both monocyclic and bicyclic ring systems and bicyclic structures at least a portion of which is aromatic and the other part is saturated, partially or fully unsaturated. Examples of aryl groups as used herein include, but are not limited to, naphthyl, anthryl, phenanthryl, indanyl, indenyl, azulenyl, azulanyl, fluorenyl, phenyl and naphthyl; and more specifically phenyl.

As used herein, the term “—C₃₋₈cycloalkyl” as used herein refers to a saturated monocyclic hydrocarbon ring of 3 to 8 carbon atoms. Examples of such groups as used herein include, but are not limited to, cyclopropyl, cyclobutyl, cyclopentyl, cyclohexyl, cycloheptyl or cyclooctyl; and more specifically cyclohexyl.

As used herein, the term “halogen” or “halo” refers to a fluorine (fluoro), chlorine (chloro), bromine (bromo) or iodine (iodo) atom.

As used herein, the term “haloalkyl” refers to an alkyl group having one or more carbon atoms and wherein at least one hydrogen atom is replaced with a halogen atom, for example a fluoro atom. For example, —C₁₋₄haloalkyl refers to an alkyl group containing at least 1, and at most 4, carbon atoms and at least one halogen atom, for example a fluoro atom. Examples of “haloalkyl” groups as used herein include, but are not limited to, trifluoromethyl (—CF₃).

As used herein, the term “-(5-10 membered heteroaryl)” or “—C₅₋₁₀heteroaryl” refers to an aromatic cyclic group containing 5 to 9 ring-atoms 1, 2, 3 or 4 of which are hetero-atoms independently selected from nitrogen, oxygen and sulphur and the remaining ring-atoms are carbon, e.g. benzothiophene. This definition includes both monocyclic and bicyclic ring systems and bicyclic structures at least a portion of which is aromatic and the other part is saturated, partially or fully unsaturated.

As used herein, the term “-(5-10 membered heterocyclyl)” or “—C₅₋₁₀heterocyclyl” refers to a cyclic group containing 5 to 9 ring-atoms 1, 2, 3 or 4 of which are hetero-atoms independently selected from nitrogen, oxygen and sulphur and the remaining ring-atoms are carbon, wherein said cyclic group is saturated, partially or fully unsaturated but, which is not aromatic. This definition includes bicyclic structures provided the moiety is non-aromatic.

As used herein, the term “substituted” refers to substitution with the named substituent or substituents, multiple degrees of substitution being allowed unless otherwise stated.

For the avoidance of doubt, the term “independently” means that where more than one substituent is selected from a number of possible substituents, those substituents may be the same or different.

Also included in the present invention are pharmaceutically acceptable salt complexes. In certain embodiments of the invention, pharmaceutically acceptable salts of the compounds according to formula I may be preferred over the respective free base or free acid because such salts impart greater stability or solubility to the molecule thereby facilitating formulation into a dosage form. Therefore, the present invention also covers the pharmaceutically acceptable salts of the compounds of formula (I).

Therefore, in one aspect of the invention there is provided a compound of formula (I) or a salt thereof wherein the salt is a pharmaceutically acceptable salt.

As used herein, the term “pharmaceutically acceptable”, refers to salts, molecular entities and other ingredients of compositions that are generally physiologically tolerable and do not typically produce untoward reactions when administered to a subject (e.g. human). The term “pharmaceutically acceptable” also means approved by a regulatory agency of the Federal or a state government or listed in the U.S. Pharmacopoeia or other generally recognized pharmacopoeia for use in a subject, and more particularly in humans.

As used herein, the term “subject” refers to an animal, in particular a mammal and more particularly to a human or a domestic animal or an animal serving as a model for a disease (e.g., mouse, monkey, etc.). In one aspect, the subject is a human.

Salts of compounds of formula (I) which are suitable for use in medicine are those wherein the counterion is pharmaceutically acceptable. However, salts having non-pharmaceutically acceptable counterions are within the scope of the present invention, for example, for use as intermediates in the preparation of other compounds of formula (I) and their pharmaceutically acceptable salts.

Suitable pharmaceutically acceptable salts will be apparent to those skilled in the art and include for example base addition salts e.g. ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine or for example acid addition salts formed from acids which form non-toxic salts e.g. hydrochloride, hydrobromide, hydroiodide, sulphate, bisulphate, nitrate, phosphate, hydrogen phosphate, acetate, maleate, malate, fumarate, lactate, tartrate, citrate, formate, gluconate, succinate, piruvate, oxalate, oxaloacetate, trifluoroacetate, saccharate, benzoate, methansulphonate, ethanesulphonate, benzenesulphonate, p-toluensulphonate, methanesulphonic, ethanesulphonic, p-toluenesulphonic, and isethionate. For a review on suitable salts see Berge et al. J. Pharm. Sci., 1977, 66, 1-19. The invention includes within its scope all possible stoichiometric and non-stoichiometric forms of the salts of the compounds of formula (I).

Those skilled in the art of organic chemistry will appreciate that many organic compounds can form complexes with solvents in which they are reacted or from which they are precipitated or crystallized. These complexes are known as “solvates”.

Solvates of the compounds of formula (I) and solvates of the salts of the compounds of formula (I) are included within the scope of the present invention.

As used herein, the term “solvate” refers to a complex of variable stoichiometry formed by a solute (in this invention, a compound of formula (I) or a salt thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Most preferably the solvent used is water and the solvate may also be referred to as a hydrate.

Solvates of compounds of formula (I) which are suitable for use in medicine are those wherein the solvent is pharmaceutically acceptable. However, solvates having non-pharmaceutically acceptable solvents are within the scope of the present invention, for example, for use as intermediates in the preparation of other compounds of formula (I) and their pharmaceutically acceptable salts.

In one aspect, the compounds of formula (I) may be in the form of pharmaceutically acceptable salts, solvates or solvates of salts. In a further aspect, the compounds of formula (I) may be in the form of pharmaceutically acceptable salts.

As used herein, the term “compounds of the invention” means the compounds according to formula (I) and pharmaceutically acceptable salts thereof. The term “a compound of the invention” means any one of the compounds of the invention as defined below.

Prodrugs of the compounds of formula (I) are included within the scope of the present invention.

As used herein, the term “prodrug” means a compound which is converted within the body, e.g. by hydrolysis in the blood, into its active form that has medical effects. Pharmaceutically acceptable prodrugs are described in T. Higuchi and V. Stella, Prodrugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987 and in D. Fleishner, S. Ramon and H. Barba “Improved oral drug delivery: solubility limitations overcome by the use of prodrugs”, Advanced Drug Delivery Reviews (1996) 19(2) 115-130. Prodrugs are any covalently bonded carriers that release a compound of structure (I) in vivo when such prodrug is administered to a patient. Prodrugs are generally prepared by modifying functional groups in a way such that the modification is cleaved in vivo yielding the parent compound. Prodrugs may include, for example, compounds of this invention wherein hydroxy or amine groups are bonded to any group that, when administered to a patient, cleaves to form the hydroxy or amine groups. Thus, representative examples of prodrugs include (but are not limited to) phosphonate, carbamate, acetate, formate and benzoate derivatives of hydroxy and amine functional groups of the compounds of formula (I).

Certain compounds of formula (I) may exist in stereoisomeric forms (e.g. they may contain one or more asymmetric carbon atoms). The individual stereoisomers (enantiomers and diastereomers) and mixtures or racemic mixtures thereof are included within the scope of the present invention.

Likewise, it is understood that compounds of formula (I) may exist in tautomeric forms other than that shown in the formula and these are also included within the scope of the present invention. In particular, compounds of formula (I) may exist in the following tautomeric forms when R⁷ is H.

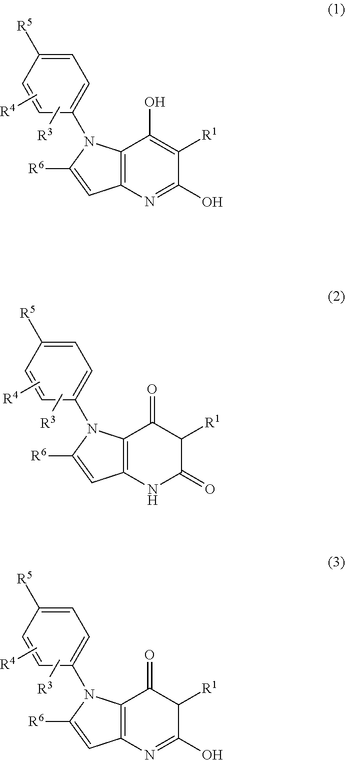

All possible tautomeric forms of the compounds of formula (I) are contemplated to be within the scope of the present invention.

It will be appreciated that racemic compounds of formula (I) may be optionally resolved into their individual enantiomers. Such resolutions may conveniently be accomplished by standard methods known in the art. For example, a racemic compound of formula (I) may be resolved by chiral preparative HPLC. An individual stereoisomer may also be prepared from a corresponding optically pure intermediate or by resolution, such as H.P.L.C. of the corresponding mixture using a suitable chiral support or by fractional crystallisation of the diastereoisomeric salts formed by reaction of the corresponding mixture with a suitable optically active acid or base, as appropriate.

It will be appreciated that compounds of the invention may exist as geometric isomers (cis/trans or (E)/(Z)). The present invention includes the individual geometric isomers of the compounds of the invention and, where appropriate, mixtures thereof. The compounds of formula (I) may be in crystalline or amorphous form. Furthermore, some of the crystalline forms of the compounds of formula (I) may exist as polymorphs, which are included in the present invention.

In one aspect of the invention there is provided a compound of formula (I) selected from the group consisting of:
1-(4-Bromophenyl)-7-hydroxy-5-oxo-4,5-dihydro-1H-pyrrolo[3,2-b]pyridine-6-carbonitrile,
7-Hydroxy-1-(2′-hydroxy-4-biphenylyl)-5-oxo-4,5-dihydro-1H-pyrrolo[3,2-b]pyridine-6-carbonitrile,
7-Hydroxy-1-[4′-(methyloxy)-4-biphenylyl]-5-oxo-4,5-dihydro-1H-pyrrolo[3,2-b]pyridine-6-carbonitrile,
7-Hydroxy-1-[2′-(methyloxy)-4-biphenylyl]-5-oxo-4,5-dihydro-1H-pyrrolo[3,2-b]pyridine-6-carbonitrile,
7-Hydroxy-1-[3′-(methyloxy)-4-biphenylyl]-5-oxo-4,5-dihydro-1H-pyrrolo[3,2-b]pyridine-6-carbonitrile,
1-(4′-Fluoro-4-biphenylyl)-7-hydroxy-5-oxo-4,5-dihydro-1H-pyrrolo[3,2-b]pyridine-6-carbonitrile,
1-(2′-Cyano-4-biphenylyl)-7-hydroxy-5-oxo-4,5-dihydro-1H-pyrrolo[3,2-b]pyridine-6-carbonitrile,
1-(3′-Fluoro-4-biphenylyl)-7-hydroxy-5-oxo-4,5-dihydro-1H-pyrrolo[3,2-b]pyridine-6-carbonitrile,
7-Hydroxy-5-oxo-1-phenyl-4,5-dihydro-1H-pyrrolo[3,2-b]pyridine-6-carbonitrile,
7-Hydroxy-1-[2′-hydroxy-3′-(methyloxy)-4-biphenylyl]-5-oxo-4,5-dihydro-1H-pyrrolo[3,2-b]pyridine-6-carbonitrile,
1-(2′-Fluoro-4-biphenylyl)-7-hydroxy-5-oxo-4,5-dihydro-1H-pyrrolo[3,2-b]pyridine-6-carbonitrile,
1-(4′-Chloro-4-biphenylyl)-7-hydroxy-5-oxo-4,5-dihydro-1H-pyrrolo[3,2-b]pyridine-6-carbonitrile,
7-Hydroxy-1-(4′-hydroxy-4-biphenylyl)-5-oxo-4,5-dihydro-1H-pyrrolo[3,2-b]pyridine-6-carbonitrile,
7-Hydroxy-5-oxo-1-[4′-(trifluoromethyl)-4-biphenylyl]-4,5-dihydro-1H-pyrrolo[3,2-b]pyridine-6-carbonitrile,
7-Hydroxy-1-(4′-methyl-4-biphenylyl)-5-oxo-4,5-dihydro-1H-pyrrolo[3,2-b]pyridine-6-carbonitrile,
1-(4′-Cyano-4-biphenylyl)-7-hydroxy-5-oxo-4,5-dihydro-1H-pyrrolo[3,2-b]pyridine-6-carbonitrile,
N-[4′-(6-Cyano-7-hydroxy-5-oxo-4,5-dihydro-1H-pyrrolo[3,2-b]pyridin-1-yl)-4-biphenylyl]acetamide,
1-(2′-Chloro-4-biphenylyl)-7-hydroxy-5-oxo-4,5-dihydro-1H-pyrrolo[3,2-b]pyridine-6-carbonitrile,
7-Hydroxy-1-{4-[6-(methyloxy)-3-pyridinyl]phenyl}-5-oxo-4,5-dihydro-1H-pyrrolo[3,2-b]pyridine-6-carbonitrile,
1-(4′-Fluoro-4-biphenylyl)-7-hydroxy-4-methyl-5-oxo-4,5-dihydro-1H-pyrrolo[3,2-b]pyridine-6-carbonitrile,
7-Hydroxy-1-(2′-hydroxy-4-biphenylyl)-1,4-dihydro-5H-pyrrolo[3,2-b]pyridin-5-one,
1-[4-(6-Fluoro-3-pyridinyl)phenyl]-7-hydroxy-5-oxo-4,5-dihydro-1H-pyrrolo[3,2-b]pyridine-6-carbonitrile,
1-(4-Bromophenyl)-2-chloro-7-hydroxy-5-oxo-4,5-dihydro-1H-pyrrolo[3,2-b]pyridine-6-carbonitrile,
2-Chloro-7-hydroxy-1-[2′-hydroxy-3′-(methyloxy)-4-biphenylyl]-5-oxo-4,5-dihydro-1H-pyrrolo[3,2-b]pyridine-6-carbonitrile,
2-Chloro-1-(2′-fluoro-4-biphenylyl)-7-hydroxy-5-oxo-4,5-dihydro-1H-pyrrolo[3,2-b]pyridine-6-carbonitrile,
2-Chloro-7-hydroxy-1-(2′-hydroxy-4-biphenylyl)-5-oxo-4,5-dihydro-1H-pyrrolo[3,2-b]pyridine-6-carbonitrile,
2-Chloro-7-hydroxy-1-(4′-methyl-4-biphenylyl)-5-oxo-4,5-dihydro-1H-pyrrolo[3,2-b]pyridine-6-carbonitrile,
2-Chloro-1-(4′-fluoro-4-biphenylyl)-7-hydroxy-5-oxo-4,5-dihydro-1H-pyrrolo[3,2-b]pyridine-6-carbonitrile,
2-Chloro-7-hydroxy-1-[4′-(methyloxy)-4-biphenylyl]-5-oxo-4,5-dihydro-1H-pyrrolo[3,2-b]pyridine-6-carbonitrile,
Ethyl 7-hydroxy-1-(2′-hydroxy-4-biphenylyl)-5-oxo-4,5-dihydro-1H-pyrrolo[3,2-b]pyridine-6-carboxylate,
4′-(6-Cyano-7-hydroxy-5-oxo-4,5-dihydro-1H-pyrrolo[3,2-b]pyridin-1-yl)-2-hydroxy-3-biphenylcarboxylic acid,
1-(2′,4′-Difluoro-4-biphenylyl)-7-hydroxy-5-oxo-4,5-dihydro-1H-pyrrolo[3,2-b]pyridine-6-carbonitrile,
7-Hydroxy-5-oxo-1-[2′-(trifluoromethyl)-4-biphenylyl]-4,5-dihydro-1H-pyrrolo[3,2-b]pyridine-6-carbonitrile,
1-(2′,4′-Dichloro-4-biphenylyl)-7-hydroxy-5-oxo-4,5-dihydro-1H-pyrrolo[3,2-b]pyridine-6-carbonitrile,
1-[4-(5-Chloro-2-thienyl)phenyl]-7-hydroxy-5-oxo-4,5-dihydro-1H-pyrrolo[3,2-b]pyridine-6-carbonitrile,
1-(4-Bromophenyl)-6-(4-fluorophenyl)-7-hydroxy-1,4-dihydro-5H-pyrrolo[3,2-b]pyridin-5-one,
6-(4-Fluorophenyl)-7-hydroxy-1-(2′-hydroxy-4-biphenylyl)-1,4-dihydro-5H-pyrrolo[3,2-b]pyridin-5-one,
1-(4-Bromophenyl)-7-hydroxy-6-[4-(methyloxy)phenyl]-1,4-dihydro-5H-pyrrolo[3,2-b]pyridin-5-one,
4-[7-Hydroxy-1-(2′-hydroxy-4-biphenylyl)-5-oxo-4,5-dihydro-1H-pyrrolo[3,2-b]pyridin-6-yl]benzonitrile,
7-Hydroxy-1-(2′-hydroxy-4-biphenylyl)-6-phenyl-1,4-dihydro-5H-pyrrolo[3,2-b]pyridin-5-one,
Ethyl 4-(6-cyano-7-hydroxy-5-oxo-4,5-dihydro-1H-pyrrolo[3,2-b]pyridin-1-yl)benzoate,
7-Hydroxy-1-[4-(methyloxy)phenyl]-5-oxo-4,5-dihydro-1H-pyrrolo[3,2-b]pyridine-6-carbonitrile,
1-(4-Bromophenyl)-7-hydroxy-1,4-dihydro-5H-pyrrolo[3,2-b]pyridin-5-one,
1-(2′-Fluoro-4′-methyl-4-biphenylyl)-7-hydroxy-5-oxo-4,5-dihydro-1H-pyrrolo[3,2-b]pyridine-6-carbonitrile,
7-Hydroxy-5-oxo-1-[4′-(trimethylsilyl)-4-biphenylyl]-4,5-dihydro-1H-pyrrolo[3,2-b]pyridine-6-carbonitrile,
1-[4-(1,3-Benzodioxol-5-yl)phenyl]-7-hydroxy-5-oxo-4,5-dihydro-1H-pyrrolo[3,2-b]pyridine-6-carbonitrile,
1-(2′,4′-Dimethyl-4-biphenylyl)-7-hydroxy-5-oxo-4,5-dihydro-1H-pyrrolo[3,2-b]pyridine-6-carbonitrile,
7-Hydroxy-5-oxo-1-[4-(3-thienyl)phenyl]-4,5-dihydro-1H-pyrrolo[3,2-b]pyridine-6-carbonitrile,
7-Hydroxy-1-[4-(6-methyl-2-pyridinyl)phenyl]-5-oxo-4,5-dihydro-1H-pyrrolo[3,2-b]pyridine-6-carbonitrile,
7-Hydroxy-5-oxo-1-[4-(4-pyridinyl)phenyl]-4,5-dihydro-1H-pyrrolo[3,2-b]pyridine-6-carbonitrile,
1-[4-(5-Cyano-2-pyridinyl)phenyl]-7-hydroxy-5-oxo-4,5-dihydro-1H-pyrrolo[3,2-b]pyridine-6-carbonitrile,
1-[4-(3,5-Dimethyl-4-isoxazolyl)phenyl]-7-hydroxy-5-oxo-4,5-dihydro-1H-pyrrolo[3,2-b]pyridine-6-carbonitrile,
7-Hydroxy-5-oxo-1-[4-(3-pyridinyl)phenyl]-4,5-dihydro-1H-pyrrolo[3,2-b]pyridine-6-carbonitrile,
7-Hydroxy-1-[4-(5-methyl-2-pyridinyl)phenyl]-5-oxo-4,5-dihydro-1H-pyrrolo[3,2-b]pyridine-6-carbonitrile,
7-Hydroxy-1-{4-[5-(methyloxy)-2-pyridinyl]phenyl}-5-oxo-4,5-dihydro-1H-pyrrolo[3,2-b]pyridine-6-carbonitrile,
7-Hydroxy-5-oxo-1-[4-(1H-pyrrol-2-yl)phenyl]-4,5-dihydro-1H-pyrrolo[3,2-b]pyridine-6-carbonitrile,
1-(3′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-3′-methyl-4-biphenylyl)-5-oxo-4,5-dihydro-1H-pyrrolo[3,2-b]pyridine-6-carbonitrile,
7-Hydroxy-5-oxo-1-[4-(2-thienyl)phenyl]-4,5-dihydro-1H-pyrrolo[3,2-b]pyridine-6-carbonitrile,
1-(3′-Chloro-2′-hydroxy-4-biphenylyl)-7-hydroxy-5-oxo-4,5-dihydro-1H-pyrrolo[3,2-b]pyridine-6-carbonitrile,
1-(5′-Fluoro-2′-hydroxy-4-biphenylyl)-7-hydroxy-5-oxo-4,5-dihydro-1H-pyrrolo[3,2-b]pyridine-6-carbonitrile,
1-(2′-Fluoro-6′-hydroxy-4-biphenylyl)-7-hydroxy-5-oxo-4,5-dihydro-1H-pyrrolo[3,2-b]pyridine-6-carbonitrile,
1-(4′-Chloro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-5′-methyl-4-biphenylyl)-5-oxo-4,5-dihydro-1H-pyrrolo[3,2-b]pyridine-6-carbonitrile,
4′-(6-Cyano-7-hydroxy-5-oxo-4,5-dihydro-1H-pyrrolo[3,2-b]pyridine-1-yl)-6-hydroxy-3-biphenylcarboxylic acid,
2-Chloro-1-(5′-fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[2′-hydroxy-3′-(methyloxy)-5′-(2-propen-1-yl)-4-biphenylyl]-5-oxo-4,5-dihydro-1H-pyrrolo[3,2-b]pyridine-6-carbonitrile,
1-[4-(2,3-Dihydro-1,4-benzodioxin-6-yl)phenyl]-7-hydroxy-5-oxo-4,5-dihydro-1H-pyrrolo[3,2-b]pyridine-6-carbonitrile,
2-Chloro-7-hydroxy-1-(2′-hydroxy-3′-methyl-4-biphenylyl)-5-oxo-4,5-dihydro-1H-pyrrolo[3,2-b]pyridine-6-carbonitrile,
4′-(2-Chloro-6-cyano-7-hydroxy-5-oxo-4,5-dihydro-1H-pyrrolo[3,2-b]pyridine-1-yl)-6-hydroxy-3-biphenylcarboxylic acid,
1-(2′-Chloro-6′-hydroxy-4-biphenylyl)-7-hydroxy-5-oxo-4,5-dihydro-1H-pyrrolo[3,2-b]pyridine-6-carbonitrile,
7-Hydroxy-1-{4-[2-(methyloxy)-3-pyridinyl]phenyl}-5-oxo-4,5-dihydro-1H-pyrrolo[3,2-b]pyridine-6-carbonitrile,
2-Chloro-7-hydroxy-5-oxo-1-[4-(3-thienyl)phenyl]-4,5-dihydro-1H-pyrrolo[3,2-b]pyridine-6-carbonitrile,
1-(4′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[4-(2-hydroxy-3-pyridinyl)phenyl]-5-oxo-4,5-dihydro-1H-pyrrolo[3,2-b]pyridine-6-carbonitrile,
2-Chloro-1-(4′-chloro-2′-hydroxy-4-biphenylyl)-7-hydroxy-5-oxo-4,5-dihydro-1H-pyrrolo[3,2-b]pyridine-6-carbonitrile,
7-Hydroxy-1-{4-[4-(methyloxy)-3-pyridinyl]phenyl}-5-oxo-4,5-dihydro-1H-pyrrolo[3,2-b]pyridine-6-carbonitrile,
1-[4-(2-Furanyl)phenyl]-7-hydroxy-5-oxo-4,5-dihydro-1H-pyrrolo[3,2-b]pyridine-6-carbonitrile,
7-Hydroxy-2-methyl-5-oxo-1-[4-(3-thienyl)phenyl]-4,5-dihydro-1H-pyrrolo[3,2-b]pyridine-6-carbonitrile,
4-[1-(5′-Fluoro-2′-hydroxy-4-biphenylyl)-7-hydroxy-5-oxo-4,5-dihydro-1H-pyrrolo[3,2-b]pyridin-6-yl]benzonitrile,
4-[7-Hydroxy-1-(2′-hydroxy-5′-methyl-4-biphenylyl)-5-oxo-4,5-dihydro-1H-pyrrolo[3,2-b]pyridin-6-yl]benzonitrile,
4-[7-Hydroxy-1-(2′-hydroxy-3′-methyl-4-biphenylyl)-5-oxo-4,5-dihydro-1H-pyrrolo[3,2-b]pyridin-6-yl]benzonitrile,
2-Chloro-1-(6′-fluoro-2′-hydroxy-4-biphenylyl)-7-hydroxy-5-oxo-4,5-dihydro-1H-pyrrolo[3,2-b]pyridine-6-carbonitrile,
2-Chloro-1-(4′-fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[4-(4-methyl-2-thienyl)phenyl]-5-oxo-4,5-dihydro-1H-pyrrolo[3,2-b]pyridine-6-carbonitrile,
7-Hydroxy-5-oxo-1-[4-(1H-pyrrol-3-yl)phenyl]-4,5-dihydro-1H-pyrrolo[3,2-b]pyridine-6-carbonitrile,
7-Hydroxy-5-oxo-1-[4-(1,3-thiazol-2-yl)phenyl]-4,5-dihydro-1H-pyrrolo[3,2-b]pyridine-6-carbonitrile,
7-Hydroxy-5-oxo-1-[4-(1,3-thiazol-4-yl)phenyl]-4,5-dihydro-1H-pyrrolo[3,2-b]pyridine-6-carbonitrile,
7-Hydroxy-1-[4-(3-methyl-2-thienyl)phenyl]-5-oxo-4,5-dihydro-1H-pyrrolo[3,2-b]pyridine-6-carbonitrile,
5-[4-(6-Cyano-7-hydroxy-5-oxo-4,5-dihydro-1H-pyrrolo[3,2-b]pyridin-1-yl)phenyl]-2-thiophenesulfonamide,
7-Hydroxy-1-[4-(1-methyl-1H-pyrazol-4-yl)phenyl]-5-oxo-4,5-dihydro-1H-pyrrolo[3,2-b]pyridine-6-carbonitrile,
7-Hydroxy-1-[4-(2-methyl-1,3-thiazol-4-yl)phenyl]-5-oxo-4,5-dihydro-1H-pyrrolo[3,2-b]pyridine-6-carbonitrile,
2-Chloro-1-[2′-fluoro-6′-(methyloxy)-4-biphenylyl]-7-hydroxy-5-oxo-4,5-dihydro-1H-pyrrolo[3,2-b]pyridine-6-carbonitrile,
7-Hydroxy-1-[4-(5-methyl-2-thienyl)phenyl]-5-oxo-4,5-dihydro-1H-pyrrolo[3,2-b]pyridine-6-carbonitrile,
5-[4-(2-Chloro-6-cyano-7-hydroxy-5-oxo-4,5-dihydro-1H-pyrrolo[3,2-b]pyridin-1-yl)phenyl]-2-thiophenesulfonamide,
2-Chloro-1-[4-(5-chloro-2-thienyl)phenyl]-7-hydroxy-5-oxo-4,5-dihydro-1H-pyrrolo[3,2-b]pyridine-6-carbonitrile,
4-[4-(2-Chloro-6-cyano-7-hydroxy-5-oxo-4,5-dihydro-1H-pyrrolo[3,2-b]pyridin-1-yl)phenyl]-2-thiophenecarboxamide,
1-[4-(1-Benzothien-3-yl)phenyl]-2-chloro-7-hydroxy-5-oxo-4,5-dihydro-1H-pyrrolo[3,2-b]pyridine-6-carbonitrile,
2-Chloro-7-hydroxy-1-{4-[5-(methyloxy)-2-pyridinyl]phenyl}-5-oxo-4,5-dihydro-1H-pyrrolo[3,2-b]pyridine-6-carbonitrile,
2-Chloro-7-hydroxy-5-oxo-1-[4-(1,3-thiazol-4-yl)phenyl]-4,5-dihydro-1H-pyrrolo[3,2-b]pyridine-6-carbonitrile,
2-Chloro-1-[4-(4-cyano-3-thienyl)phenyl]-7-hydroxy-5-oxo-4,5-dihydro-1H-pyrrolo[3,2-b]pyridine-6-carbonitrile,
4-[4-(2-Chloro-6-cyano-7-hydroxy-5-oxo-4,5-dihydro-1H-pyrrolo[3,2-b]pyridin-1-yl)phenyl]-3-thiophenecarboxamide,
2-Chloro-7-hydroxy-5-oxo-1-[4-(3-pyridinyl)phenyl]-4,5-dihydro-1H-pyrrolo[3,2-b]pyridine-6-carbonitrile,
2-Chloro-1-[2′-chloro-6′-(methyloxy)-4-biphenylyl]-7-hydroxy-5-oxo-4,5-dihydro-1H-pyrrolo[3,2-b]pyridine-6-carbonitrile,
2-Chloro-7-hydroxy-1-[4-(3-methyl-2-thienyl)phenyl]-5-oxo-4,5-dihydro-1H-pyrrolo[3,2-b]pyridine-6-carbonitrile,
2-Chloro-7-hydroxy-1-[4-(1-methyl-1H-pyrazol-4-yl)phenyl]-5-oxo-4,5-dihydro-1H-pyrrolo[3,2-b]pyridine-6-carbonitrile,
2-Chloro-7-hydroxy-1-[4-(2-methyl-1,3-thiazol-4-yl)phenyl]-5-oxo-4,5-dihydro-1H-pyrrolo[3,2-b]pyridine-6-carbonitrile,
2-Chloro-7-hydroxy-1-(3′-hydroxy-4-biphenylyl)-5-oxo-4,5-dihydro-1H-pyrrolo[3,2-b]pyridine-6-carbonitrile,
1-[4-(6-Amino-2-pyridinyl)phenyl]-2-chloro-7-hydroxy-5-oxo-4,5-dihydro-1H-pyrrolo[3,2-b]pyridine-6-carbonitrile,
2-Chloro-7-hydroxy-1-[3′-(hydroxymethyl)-4-biphenylyl]-5-oxo-4,5-dihydro-1H-pyrrolo[3,2-b]pyridine-6-carbonitrile,
7-Hydroxy-5-oxo-1-[4-(3-thienyl)phenyl]-4,5-dihydro-1H-pyrrolo[3,2-b]pyridine-2,6-dicarbonitrile,
2-Chloro-7-hydroxy-1-[4-(5-methyl-2-thienyl)phenyl]-5-oxo-4,5-dihydro-1H-pyrrolo[3,2-b]pyridine-6-carbonitrile,
2-Chloro-7-hydroxy-5-oxo-1-[4-(2-thienyl)phenyl]-4,5-dihydro-1H-pyrrolo[3,2-b]pyridine-6-carbonitrile,
2-Chloro-1-(3′-fluoro-2′-hydroxy-4-biphenylyl)-7-hydroxy-5-oxo-4,5-dihydro-1H-pyrrolo[3,2-b]pyridine-6-carbonitrile,
2-Chloro-1-(3′-chloro-2′-hydroxy-4-biphenylyl)-7-hydroxy-5-oxo-4,5-dihydro-1H-pyrrolo[3,2-b]pyridine-6-carbonitrile,
2-Chloro-1-(2′-fluoro-4′-methyl-4-biphenylyl)-7-hydroxy-5-oxo-4,5-dihydro-1H-pyrrolo[3,2-b]pyridine-6-carbonitrile,
2-Chloro-1-[2′-chloro-4′-(methyloxy)-4-biphenylyl]-7-hydroxy-5-oxo-4,5-dihydro-1H-pyrrolo[3,2-b]pyridine-6-carbonitrile,
1-(4-Ethylphenyl)-7-hydroxy-5-oxo-4,5-dihydro-1H-pyrrolo[3,2-b]pyridine-2,6-dicarbonitrile,
2-Chloro-1-(2′-chloro-6′-hydroxy-4-biphenylyl)-7-hydroxy-5-oxo-4,5-dihydro-1H-pyrrolo[3,2-b]pyridine-6-carbonitrile,
2-Chloro-7-hydroxy-1-(2′-hydroxy-5′-methyl-4-biphenylyl)-5-oxo-4,5-dihydro-1H-pyrrolo[3,2-b]pyridine-6-carbonitrile,
2-Chloro-6-(2-fluorophenyl)-7-hydroxy-1-[4-(methyloxy)phenyl]-1,4-dihydro-5H-pyrrolo[3,2-b]pyridin-5-one,
2-Chloro-6-(3-fluorophenyl)-7-hydroxy-1-[4-(methyloxy)phenyl]-1,4-dihydro-5H-pyrrolo[3,2-b]pyridine-5-one,
2-Chloro-1-(4-ethylphenyl)-7-hydroxy-6-phenyl-1,4-dihydro-5H-pyrrolo[3,2-b]pyridine-5-one,
2-Chloro-6-(4-fluorophenyl)-7-hydroxy-1-[4-(methyloxy)phenyl]-1,4-dihydro-5H-pyrrolo[3,2-b]pyridin-5-one,
2-Chloro-7-hydroxy-1-[4-(methyloxy)phenyl]-6-phenyl-1,4-dihydro-5H-pyrrolo[3,2-b]pyridin-5-one,
2-Chloro-1-(4-fluorophenyl)-7-hydroxy-6-phenyl-1,4-dihydro-5H-pyrrolo[3,2-b]pyridin-5-one,
2-Chloro-6-(2-fluorophenyl)-7-hydroxy-1-[3-(methyloxy)phenyl]-1,4-dihydro-5H-pyrrolo[3,2-b]pyridin-5-one,
2-Chloro-6-(3-fluorophenyl)-7-hydroxy-1-[3-(methyloxy)phenyl]-1,4-dihydro-5H-pyrrolo[3,2-b]pyridin-5-one,
2-Chloro-6-(4-fluorophenyl)-7-hydroxy-1-[3-(methyloxy)phenyl]-1,4-dihydro-5H-pyrrolo[3,2-b]pyridin-5-one,
2-Chloro-7-hydroxy-1-[3-(methyloxy)phenyl]-6-phenyl-1,4-dihydro-5H-pyrrolo[3,2-b]pyridin-5-one,
2-Chloro-6-(4-fluorophenyl)-7-hydroxy-1-(4-methylphenyl)-1,4-dihydro-5H-pyrrolo[3,2-b]pyridin-5-one,
2-Chloro-7-hydroxy-6-phenyl-1-[4-(trifluoromethyl)phenyl]-1,4-dihydro-5H-pyrrolo[3,2-b]pyridin-5-one,
2-Chloro-7-hydroxy-1-(4-methylphenyl)-6-phenyl-1,4-dihydro-5H-pyrrolo[3,2-b]pyridin-5-one,
2-Chloro-6-(2-fluorophenyl)-7-hydroxy-1-(4-methylphenyl)-1,4-dihydro-5H-pyrrolo[3,2-b]pyridin-5-one,
2-Chloro-6-(3-fluorophenyl)-7-hydroxy-1-(4-methylphenyl)-1,4-dihydro-5H-pyrrolo[3,2-b]pyridin-5-one,
2-Chloro-6-(2-fluorophenyl)-7-hydroxy-1-[4-(trifluoromethyl)phenyl]-1,4-dihydro-5H-pyrrolo[3,2-b]pyridin-5-one,
2-Chloro-6-(3-fluorophenyl)-7-hydroxy-1-[4-(trifluoromethyl)phenyl]-1,4-dihydro-5H-pyrrolo[3,2-b]pyridin-5-one,
2-Chloro-6-(2-fluorophenyl)-7-hydroxy-1-{4-[(trifluoromethyl)oxy]phenyl}-1,4-dihydro-5H-pyrrolo[3,2-b]pyridin-5-one,
2-Chloro-6-[3-(ethyloxy)phenyl]-7-hydroxy-1-(4-methylphenyl)-1,4-dihydro-5H-pyrrolo[3,2-b]pyridin-5-one,
4-[2-Chloro-7-hydroxy-1-(4-methylphenyl)-5-oxo-4,5-dihydro-1H-pyrrolo[3,2-b]pyridin-6-yl]benzonitrile,
2-Chloro-1-(3,4-dimethylphenyl)-7-hydroxy-6-phenyl-1,4-dihydro-5H-pyrrolo[3,2-b]pyridin-5-one,
2-Chloro-7-hydroxy-1-(4-methylphenyl)-6-(3-nitrophenyl)-1,4-dihydro-5H-pyrrolo[3,2-b]pyridin-5-one,
3-[2-Chloro-7-hydroxy-1-(4-methylphenyl)-5-oxo-4,5-dihydro-1H-pyrrolo[3,2-b]pyridin-6-yl]benzonitrile,
[4-(2-Chloro-7-hydroxy-5-oxo-6-phenyl-4,5-dihydro-1H-pyrrolo[3,2-b]pyridin-1-yl)phenyl]acetonitrile,
6-(3-Bromophenyl)-2-chloro-7-hydroxy-1-(4-methylphenyl)-1,4-dihydro-5H-pyrrolo[3,2-b]pyridin-5-one,
3-[2-Chloro-7-hydroxy-1-(4-methylphenyl)-5-oxo-4,5-dihydro-1H-pyrrolo[3,2-b]pyridin-6-yl]benzoic acid,
1-(4-Ethylphenyl)-7-hydroxy-5-oxo-6-phenyl-4,5-dihydro-1H-pyrrolo[3,2-b]pyridine-2-carbonitrile,
6-(2-Fluorophenyl)-7-hydroxy-1-[4-(methyloxy)phenyl]-1,4-dihydro-5H-pyrrolo[3,2-b]pyridin-5-one,
6-(3-Fluorophenyl)-7-hydroxy-1-[4-(methyloxy)phenyl]-1,4-dihydro-5H-pyrrolo[3,2-b]pyridin-5-one,
2-Chloro-7-hydroxy-6-[3-(methyloxy)phenyl]-1-[4-(3-thienyl)phenyl]-1,4-dihydro-5H-pyrrolo[3,2-b]pyridin-5-one,
3-{2-Chloro-7-hydroxy-5-oxo-1-[4-(3-thienyl)phenyl]-4,5-dihydro-1H-pyrrolo[3,2-b]pyridin-6-yl}benzoic acid,
2-Chloro-7-hydroxy-6-phenyl-1-[4-(3-thienyl)phenyl]-1,4-dihydro-5H-pyrrolo[3,2-b]pyridin-5-one,
2-{2-Chloro-7-hydroxy-5-oxo-1-[4-(3-thienyl)phenyl]-4,5-dihydro-1H-pyrrolo[3,2-b]pyridin-6-yl}benzoic acid,
4-{2-Chloro-7-hydroxy-5-oxo-1-[4-(3-thienyl)phenyl]-4,5-dihydro-1H-pyrrolo[3,2-b]pyridin-6-yl}benzonitrile,
3-{2-Chloro-7-hydroxy-5-oxo-1-[4-(3-thienyl)phenyl]-4,5-dihydro-1H-pyrrolo[3,2-b]pyridin-6-yl}benzonitrile,
1-(4-Acetylphenyl)-2-chloro-7-hydroxy-5-oxo-4,5-dihydro-1H-pyrrolo[3,2-b]pyridine-6-carbonitrile,
N-[4-(2-Chloro-6-cyano-7-hydroxy-5-oxo-4,5-dihydro-1H-pyrrolo[3,2-b]pyridin-1-yl)phenyl]acetamide,
2-Chloro-7-hydroxy-1-(4′-methyl-4-biphenylyl)-6-phenyl-1,4-dihydro-5H-pyrrolo[3,2-b]pyridin-5-one,
2-Chloro-1-(4-cyclohexylphenyl)-7-hydroxy-6-phenyl-1,4-dihydro-5H-pyrrolo[3,2-b]pyridin-5-one,
4-{2-Chloro-7-hydroxy-5-oxo-1-[4-(3-thienyl)phenyl]-4,5-dihydro-1H-pyrrolo[3,2-b]pyridin-6-yl}benzamide,
3-{2-Chloro-7-hydroxy-5-oxo-1-[4-(3-thienyl)phenyl]-4,5-dihydro-1H-pyrrolo[3,2-b]pyridin-6-yl}benzamide,
4-{2-Chloro-7-hydroxy-5-oxo-1-[4-(3-thienyl)phenyl]-4,5-dihydro-1H-pyrrolo[3,2-b]pyridin-6-yl}benzoic acid,
2-Chloro-7-hydroxy-142′-hydroxy-3′-(methyloxy)-4-biphenylyl]-6-phenyl-1,4-dihydro-5H-pyrrolo[3,2-b]pyridin-5-one,
2-Chloro-6-(2-fluorophenyl)-7-hydroxy-1-[2′-hydroxy-3′-(methyloxy)-4-biphenylyl]-1,4-dihydro-5H-pyrrolo[3,2-b]pyridin-5-one,
2-Chloro-6-(3-chlorophenyl)-7-hydroxy-1-[2′-hydroxy-3′-(methyloxy)-4-biphenylyl]-1,4-dihydro-5H-pyrrolo[3,2-b]pyridin-5-one,
3-{2-Chloro-7-hydroxy-1-[2′-hydroxy-3′-(methyloxy)-4-biphenylyl]-5-oxo-4,5-dihydro-1H-pyrrolo[3,2-b]pyridin-6-yl}benzoic acid,
4-{2-Chloro-7-hydroxy-142′-hydroxy-3′-(methyloxy)-4-biphenylyl]-5-oxo-4,5-dihydro-1H-pyrrolo[3,2-b]pyridin-6-yl}benzonitrile,
2-Chloro-7-hydroxy-1-[2′-hydroxy-3′-(methyloxy)-4-biphenylyl]-6-[3-(methyloxy)phenyl]-1,4-dihydro-5H-pyrrolo[3,2-b]pyridin-5-one, and
4-{2-Chloro-7-hydroxy-1-[2′-hydroxy-3′-(methyloxy)-4-biphenylyl]-5-oxo-4,5-dihydro-1H-pyrrolo[3,2-b]pyridin-6-yl}benzoic acid,
   or a salt thereof.

In another aspect, the present invention comprises a compound of formula (I) selected from the group consisting of:
1-(4-Bromophenyl)-7-hydroxy-5-oxo-4,5-dihydro-1H-pyrrolo[3,2-b]pyridine-6-carbonitrile,
7-Hydroxy-1-(2′-hydroxy-4-biphenylyl)-5-oxo-4,5-dihydro-1H-pyrrolo[3,2-b]pyridine-6-carbonitrile,
7-Hydroxy-1-[4′-(methyloxy)-4-biphenylyl]-5-oxo-4,5-dihydro-1H-pyrrolo[3,2-b]pyridine-6-carbonitrile,
7-Hydroxy-1-[2′-(methyloxy)-4-biphenylyl]-5-oxo-4,5-dihydro-1H-pyrrolo[3,2-b]pyridine-6-carbonitrile,
7-Hydroxy-1-[3′-(methyloxy)-4-biphenylyl]-5-oxo-4,5-dihydro-1H-pyrrolo[3,2-b]pyridine-6-carbonitrile,
1-(4′-Fluoro-4-biphenylyl)-7-hydroxy-5-oxo-4,5-dihydro-1H-pyrrolo[3,2-b]pyridine-6-carbonitrile,
1-(2′-Cyano-4-biphenylyl)-7-hydroxy-5-oxo-4,5-dihydro-1H-pyrrolo[3,2-b]pyridine-6-carbonitrile,
1-(3′-Fluoro-4-biphenylyl)-7-hydroxy-5-oxo-4,5-dihydro-1H-pyrrolo[3,2-b]pyridine-6-carbonitrile,
7-Hydroxy-5-oxo-1-phenyl-4,5-dihydro-1H-pyrrolo[3,2-b]pyridine-6-carbonitrile,
7-Hydroxy-1-[2′-hydroxy-3′-(methyloxy)-4-biphenylyl]-5-oxo-4,5-dihydro-1H-pyrrolo[3,2-b]pyridine-6-carbonitrile,
1-(2′-Fluoro-4-biphenylyl)-7-hydroxy-5-oxo-4,5-dihydro-1H-pyrrolo[3,2-b]pyridine-6-carbonitrile,
1-(4′-Chloro-4-biphenylyl)-7-hydroxy-5-oxo-4,5-dihydro-1H-pyrrolo[3,2-b]pyridine-6-carbonitrile,
7-Hydroxy-1-(4′-hydroxy-4-biphenylyl)-5-oxo-4,5-dihydro-1H-pyrrolo[3,2-b]pyridine-6-carbonitrile,
7-Hydroxy-5-oxo-1-[4′-(trifluoromethyl)-4-biphenylyl]-4,5-dihydro-1H-pyrrolo[3,2-b]pyridine-6-carbonitrile,
7-Hydroxy-1-(4′-methyl-4-biphenylyl)-5-oxo-4,5-dihydro-1H-pyrrolo[3,2-b]pyridine-6-carbonitrile,
1-(4′-Cyano-4-biphenylyl)-7-hydroxy-5-oxo-4,5-dihydro-1H-pyrrolo[3,2-b]pyridine-6-carbonitrile,
N-[4′-(6-Cyano-7-hydroxy-5-oxo-4,5-dihydro-1H-pyrrolo[3,2-b]pyridin-1-yl)-4-biphenylyl]acetamide,
1-(2′-Chloro-4-biphenylyl)-7-hydroxy-5-oxo-4,5-dihydro-1H-pyrrolo[3,2-b]pyridine-6-carbonitrile,
7-Hydroxy-1-{4-[6-(methyloxy)-3-pyridinyl]phenyl}-5-oxo-4,5-dihydro-1H-pyrrolo[3,2-b]pyridine-6-carbonitrile,
1-(4′-Fluoro-4-biphenylyl)-7-hydroxy-4-methyl-5-oxo-4,5-dihydro-1H-pyrrolo[3,2-b]pyridine-6-carbonitrile,
7-Hydroxy-1-(2′-hydroxy-4-biphenylyl)-1,4-dihydro-5H-pyrrolo[3,2-b]pyridin-5-one,
1-[4-(6-Fluoro-3-pyridinyl)phenyl]-7-hydroxy-5-oxo-4,5-dihydro-1H-pyrrolo[3,2-b]pyridine-6-carbonitrile,
1-(4-Bromophenyl)-2-chloro-7-hydroxy-5-oxo-4,5-dihydro-1H-pyrrolo[3,2-b]pyridine-6-carbonitrile,
2-Chloro-7-hydroxy-1-[2′-hydroxy-3′-(methyloxy)-4-biphenylyl]-5-oxo-4,5-dihydro-1H-pyrrolo[3,2-b]pyridine-6-carbonitrile,
2-Chloro-1-(2′-fluoro-4-biphenylyl)-7-hydroxy-5-oxo-4,5-dihydro-1H-pyrrolo[3,2-b]pyridine-6-carbonitrile,
2-Chloro-7-hydroxy-1-(2′-hydroxy-4-biphenylyl)-5-oxo-4,5-dihydro-1H-pyrrolo[3,2-b]pyridine-6-carbonitrile,
2-Chloro-7-hydroxy-1-(4′-methyl-4-biphenylyl)-5-oxo-4,5-dihydro-1H-pyrrolo[3,2-b]pyridine-6-carbonitrile,
2-Chloro-1-(4′-fluoro-4-biphenylyl)-7-hydroxy-5-oxo-4,5-dihydro-1H-pyrrolo[3,2-b]pyridine-6-carbonitrile,
2-Chloro-7-hydroxy-1-[4′-(methyloxy)-4-biphenylyl]-5-oxo-4,5-dihydro-1H-pyrrolo[3,2-b]pyridine-6-carbonitrile,
Ethyl 7-hydroxy-1-(2′-hydroxy-4-biphenylyl)-5-oxo-4,5-dihydro-1H-pyrrolo[3,2-b]pyridine-6-carboxylate,
4′-(6-Cyano-7-hydroxy-5-oxo-4,5-dihydro-1H-pyrrolo[3,2-b]pyridin-1-yl)-2-hydroxy-3-biphenylcarboxylic acid,
1-(2′,4′-Difluoro-4-biphenylyl)-7-hydroxy-5-oxo-4,5-dihydro-1H-pyrrolo[3,2-b]pyridine-6-carbonitrile,
7-Hydroxy-5-oxo-1-[2′-(trifluoromethyl)-4-biphenylyl]-4,5-dihydro-1H-pyrrolo[3,2-b]pyridine-6-carbonitrile,
1-(2′,4′-Dichloro-4-biphenylyl)-7-hydroxy-5-oxo-4,5-dihydro-1H-pyrrolo[3,2-b]pyridine-6-carbonitrile,
1-(4-Bromophenyl)-6-(4-fluorophenyl)-7-hydroxy-1,4-dihydro-5H-pyrrolo[3,2-b]pyridin-5-one,
6-(4-Fluorophenyl)-7-hydroxy-1-(2′-hydroxy-4-biphenylyl)-1,4-dihydro-5H-pyrrolo[3,2-b]pyridin-5-one,
1-(4-Bromophenyl)-7-hydroxy-6-[4-(methyloxy)phenyl]-1,4-dihydro-5H-pyrrolo[3,2-b]pyridin-5-one,
4-[1-(4-Bromophenyl)-7-hydroxy-5-oxo-4,5-dihydro-1H-pyrrolo[3,2-b]pyridin-6-yl]benzonitrile,
7-Hydroxy-1-(2′-hydroxy-4-biphenylyl)-6-phenyl-1,4-dihydro-5H-pyrrolo[3,2-b]pyridin-5-one,
Ethyl 4-(6-cyano-7-hydroxy-5-oxo-4,5-dihydro-1H-pyrrolo[3,2-b]pyridin-1-yl)benzoate,
7-Hydroxy-1-[4-(methyloxy)phenyl]-5-oxo-4,5-dihydro-1H-pyrrolo[3,2-b]pyridine-6-carbonitrile,
1-(4-Bromophenyl)-7-hydroxy-1,4-dihydro-5H-pyrrolo[3,2-b]pyridin-5-one,
1-(2′-Fluoro-4′-methyl-4-biphenylyl)-7-hydroxy-5-oxo-4,5-dihydro-1H-pyrrolo[3,2-b]pyridine-6-carbonitrile,
7-Hydroxy-5-oxo-1-[4′-(trimethylsilyl)-4-biphenylyl]-4,5-dihydro-1H-pyrrolo[3,2-b]pyridine-6-carbonitrile,
1-[4-(1,3-Benzodioxol-5-yl)phenyl]-7-hydroxy-5-oxo-4,5-dihydro-1H-pyrrolo[3,2-b]pyridine-6-carbonitrile,
1-(2′,4′-Dimethyl-4-biphenylyl)-7-hydroxy-5-oxo-4,5-dihydro-1H-pyrrolo[3,2-b]pyridine-6-carbonitrile,
7-Hydroxy-5-oxo-1-[4-(3-thienyl)phenyl]-4,5-dihydro-1H-pyrrolo[3,2-b]pyridine-6-carbonitrile,
7-Hydroxy-1-[4-(6-methyl-2-pyridinyl)phenyl]-5-oxo-4,5-dihydro-1H-pyrrolo[3,2-b]pyridine-6-carbonitrile,
7-Hydroxy-5-oxo-1-[4-(4-pyridinyl)phenyl]-4,5-dihydro-1H-pyrrolo[3,2-b]pyridine-6-carbonitrile,
1-[4-(5-Cyano-2-pyridinyl)phenyl]-7-hydroxy-5-oxo-4,5-dihydro-1H-pyrrolo[3,2-b]pyridine-6-carbonitrile,
1-[4-(3,5-Dimethyl-4-isoxazolyl)phenyl]-7-hydroxy-5-oxo-4,5-dihydro-1H-pyrrolo[3,2-b]pyridine-6-carbonitrile,
7-Hydroxy-5-oxo-1-[4-(3-pyridinyl)phenyl]-4,5-dihydro-1H-pyrrolo[3,2-b]pyridine-6-carbonitrile,
7-Hydroxy-1-[4-(5-methyl-2-pyridinyl)phenyl]-5-oxo-4,5-dihydro-1H-pyrrolo[3,2-b]pyridine-6-carbonitrile,
7-Hydroxy-1-{4-[5-(methyloxy)-2-pyridinyl]phenyl}-5-oxo-4,5-dihydro-1H-pyrrolo[3,2-b]pyridine-6-carbonitrile,
7-Hydroxy-5-oxo-1-[4-(1H-pyrrol-2-yl)phenyl]-4,5-dihydro-1H-pyrrolo[3,2-b]pyridine-6-carbonitrile,
1-(3′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-3′-methyl-4-biphenylyl)-5-oxo-4,5-dihydro-1H-pyrrolo[3,2-b]pyridine-6-carbonitrile,
7-Hydroxy-5-oxo-1-[4-(2-thienyl)phenyl]-4,5-dihydro-1H-pyrrolo[3,2-b]pyridine-6-carbonitrile,
1-(3′-Chloro-2′-hydroxy-4-biphenylyl)-7-hydroxy-5-oxo-4,5-dihydro-1H-pyrrolo[3,2-b]pyridine-6-carbonitrile,
1-(5′-Fluoro-2′-hydroxy-4-biphenylyl)-7-hydroxy-5-oxo-4,5-dihydro-1H-pyrrolo[3,2-b]pyridine-6-carbonitrile,
1-(2′-Fluoro-6′-hydroxy-4-biphenylyl)-7-hydroxy-5-oxo-4,5-dihydro-1H-pyrrolo[3,2-b]pyridine-6-carbonitrile,
1-(4′-Chloro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-5′-methyl-4-biphenylyl)-5-oxo-4,5-dihydro-1H-pyrrolo[3,2-b]pyridine-6-carbonitrile,
4′-(6-Cyano-7-hydroxy-5-oxo-4,5-dihydro-1H-pyrrolo[3,2-b]pyridin-1-yl)-6-hydroxy-3-biphenylcarboxylic acid,
2-Chloro-1-(5′-fluoro-2′-hydroxy-4-biphenylyl)-7-hydroxy-5-oxo-4,5-dihydro-1H-pyrrolo[3,2-b]pyridine-6-carbonitrile,
1-(3′-Chloro-2′-hydroxy-4-biphenylyl)-7-hydroxy-5-oxo-4,5-dihydro-1H-pyrrolo[3,2-b]pyridine-6-carbonitrile,
1-[4-(2,3-Dihydro-1,4-benzodioxin-6-yl)phenyl]-7-hydroxy-5-oxo-4,5-dihydro-1H-pyrrolo[3,2-b]pyridine-6-carbonitrile,
2-Chloro-7-hydroxy-1-(2′-hydroxy-3′-methyl-4-biphenylyl)-5-oxo-4,5-dihydro-1H-pyrrolo[3,2-b]pyridine-6-carbonitrile,
4′-(2-Chloro-6-cyano-7-hydroxy-5-oxo-4,5-dihydro-1H-pyrrolo[3,2-b]pyridin-1-yl)-6-hydroxy-3-biphenylcarboxylic acid,
1-(2′-Chloro-6′-hydroxy-4-biphenylyl)-7-hydroxy-5-oxo-4,5-dihydro-1H-pyrrolo[3,2-b]pyridine-6-carbonitrile,
7-Hydroxy-1-{4-[2-(methyloxy)-3-pyridinyl]phenyl}-5-oxo-4,5-dihydro-1H-pyrrolo[3,2-b]pyridine-6-carbonitrile,
2-Chloro-7-hydroxy-5-oxo-1-[4-(3-thienyl)phenyl]-4,5-dihydro-1H-pyrrolo[3,2-b]pyridine-6-carbonitrile,
1-(4′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[4-(2-hydroxy-3-pyridinyl)phenyl]-5-oxo-4,5-dihydro-1H-pyrrolo[3,2-b]pyridine-6-carbonitrile, and
2-Chloro-1-(4′-chloro-2′-hydroxy-4-biphenylyl)-7-hydroxy-5-oxo-4,5-dihydro-1H-pyrrolo[3,2-b]pyridine-6-carbonitrile,
   or a salt thereof.

Compounds of the invention have been found to activate AMPK and may therefore be useful in the treatment of type 1 diabetes, type 2 diabetes, metabolic syndrome, atherosclerosis, dyslipidaemia, mitochondrial disorders, sarcopenia, obesity, hypertension, cerebral ischemia, cognitive defect, Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

Compounds of the invention have been found to activate AMPK and may therefore be useful in the treatment of diabetes, metabolic syndrome, atherosclerosis, dyslipidaemia, obesity, hypertension, cerebral ischemia, cognitive defect and cancer.

Within the context of the present invention, the terms describing the indications used herein are classified in the Merck Manual of Diagnosis and Therapy, 17^{th} Edition and/or the International Classification of Diseases 10^{th} Edition (ICD-10). The various subtypes of the disorders mentioned herein are contemplated as part of the present invention.

In one aspect, the invention provides a compound of formula (I) or a pharmaceutically acceptable salt thereof for use in medical therapy.

In one aspect, the invention provides the use of a compound of formula (I) or a pharmaceutically acceptable salt thereof for the manufacture of a medicament for treating a disease or a condition mediated by AMPK activation.

In another aspect, the invention provides the use of a compound of formula (I) or a pharmaceutically acceptable salt thereof for the manufacture of a medicament for treating type 1 diabetes, type 2 diabetes, metabolic syndrome, atherosclerosis, dyslipidaemia, mitochondrial disorders, sarcopenia, obesity, hypertension, cerebral ischemia, cognitive defect, Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

In another aspect, the invention provides the use of a compound of formula (I) or a pharmaceutically acceptable salt thereof for the manufacture of a medicament for treating diabetes, metabolic syndrome, atherosclerosis, dyslipidaemia, obesity, hypertension, cerebral ischemia, cognitive defect and cancer.

In another aspect, the invention provides the use of a compound of formula (I) or a pharmaceutically acceptable salt thereof for the manufacture of a medicament for treating type 2 diabetes, obesity or dyslipidaemia.

In another aspect, the invention provides the use of a compound of formula (I) or a pharmaceutically acceptable salt thereof for the manufacture of a medicament for treating type 2 diabetes, dyslipidaemia and cancer.

In one aspect, the invention provides a compound of formula (I) or a pharmaceutically acceptable salt thereof for use in treating a disease or a condition mediated by AMPK activation.

In another aspect, the invention provides a compound of formula (I) or a pharmaceutically acceptable salt thereof for use in treating type 1 diabetes, type 2 diabetes, metabolic syndrome, atherosclerosis, dyslipidaemia, mitochondrial disorders, sarcopenia, obesity, hypertension, cerebral ischemia, cognitive defect, Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer.

In another aspect, the invention provides a compound of formula (I) or a pharmaceutically acceptable salt thereof for use in treating diabetes, metabolic syndrome, atherosclerosis, dyslipidaemia, obesity, hypertension, cerebral ischemia, cognitive defect and cancer.

In another aspect, the invention provides a compound of formula (I) or a pharmaceutically acceptable salt thereof for use in treating type 2 diabetes, obesity or dyslipidaemia.

In another aspect, the invention provides a compound of formula (I) or a pharmaceutically acceptable salt thereof for use in treating type 2 diabetes, dyslipidaemia and cancer.

In one aspect, the invention provides a method of treating a disease or a condition mediated by AMPK activation, which method comprises administering to a subject, for example a mammal, including human, a therapeutically effective amount of a compound of formula (I) or a pharmaceutically acceptable salt thereof.

In another aspect, the invention provides a method of treating type 1 diabetes, type 2 diabetes, metabolic syndrome, atherosclerosis, dyslipidaemia, mitochondrial disorders, sarcopenia, obesity, hypertension, cerebral ischemia, cognitive defect, Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer, which method comprises administering to a subject, for example a mammal, including human, a therapeutically effective amount of a compound of formula (I) or a pharmaceutically acceptable salt thereof.

In another aspect, the invention provides a method of treating diabetes, metabolic syndrome, atherosclerosis, dyslipidaemia, obesity, hypertension, cerebral ischemia, cognitive defect and cancer, which method comprises administering to a subject, for example a mammal, including human, a therapeutically effective amount of a compound of formula (I) or a pharmaceutically acceptable salt thereof.

In another aspect, the invention provides a method of treating type 2 diabetes, obesity or dyslipidaemia, which method comprises administering to a subject, for example a mammal, including human, a therapeutically effective amount of a compound of formula (I) or a pharmaceutically acceptable salt thereof.

In another aspect, the invention provides a method of treating type 2 diabetes, dyslipidaemia and cancer, which method comprises administering to a subject, for example a mammal, including human, a therapeutically effective amount of a compound of formula (I) or a pharmaceutically acceptable salt thereof.

It will be appreciated that reference to “treatment” and “therapy” includes acute treatment or prophylaxis as well as the alleviation of established symptoms and/or retardation of progression of the disease, and may include the suppression of symptom recurrence in an asymptomatic patient.

It will be appreciated that reference to “treatment” and “therapy” includes acute treatment as well as the alleviation of established symptoms and/or retardation of progression of the disease, and may include the suppression of symptom recurrence in an asymptomatic patient.

### Pharmaceutical Compositions

While it is possible that, for use in the methods of the invention, a compound of formula (I) or a pharmaceutically acceptable salt thereof may be administered as the bulk substance, it is preferable to present the active ingredient in a pharmaceutical formulation, for example, wherein the agent is in admixture with at least one pharmaceutically acceptable carrier selected with regard to the intended route of administration and standard pharmaceutical practice.

Accordingly, the present invention also includes a pharmaceutical composition comprising a) a compound of formula (I) or a pharmaceutically acceptable salt thereof and b) one or more pharmaceutically acceptable carriers.

The term “pharmaceutically acceptable carrier” refers to a diluent, excipient, and/or vehicle with which an active compound is administered. The pharmaceutical compositions of the invention may contain combinations of more than one carrier. Such pharmaceutical carriers can be sterile liquids, such as water, saline solutions, aqueous dextrose solutions, aqueous glycerol solutions, and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers or diluents are well known in the pharmaceutical art, and are described, for example, in “Remington's Pharmaceutical Sciences” by E. W. Martin, 18th Edition. The choice of pharmaceutical carrier can be selected with regard to the intended route of administration and standard pharmaceutical practice. The pharmaceutical compositions may comprise as, in addition to, the carrier any suitable binder(s), lubricant(s), suspending agent(s) and/or coating agent(s).

The carrier, diluent and/or excipient must be “pharmaceutically acceptable” in the sense of being compatible with the other ingredients of the composition and not deleterious to the recipient thereof.

An “pharmaceutically acceptable excipient” means an excipient that is useful in preparing a pharmaceutical composition that is generally safe, non-toxic and neither biologically nor otherwise undesirable, and includes an excipient that is acceptable for veterinary use as well as human pharmaceutical use.

Examples of pharmaceutically acceptable diluent(s) useful in the compositions of the invention include, but are not limited to water, ethanol, propylene glycol and glycerine.

Examples of pharmaceutically acceptable binders for oral compositions useful herein include, but are not limited to, acacia; cellulose derivatives, such as methylcellulose, carboxymethylcellulose, hydroxypropylmethylcellulose, hydroxypropylcellulose or hydroxyethylcellulose; gelatin, glucose, dextrose, xylitol, polymethacrylates, polyvinylpyrrolidone, sorbitol, starch, pre-gelatinized starch, tragacanth, xanthane resin, alginates, magnesium-aluminum silicate, polyethylene glycol or bentonite.

Examples of pharmaceutically acceptable lubricants useful in the compositions of the invention include, but are not limited to, magnesium stearate, talc, polyethylene glycol, polymers of ethylene oxide, sodium lauryl sulfate, magnesium lauryl sulfate, sodium oleate, sodium stearyl fumarate, and colloidal silicon dioxide.

Examples of pharmaceutically acceptable suspending agents useful in the compositions of the invention include, but are not limited tosorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non aqueous vehicles (which may include edible oils), for example almond oil, oily esters such as glycerine, propylene glycol, or ethyl alcohol; preservatives, for example methyl or propyl p-hydroxybenzoate or sorbic acid.

Examples of pharmaceutically acceptable coating materials useful in the compositions of the invention include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof.

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

The present invention relates to a pharmaceutical composition for the treatment of type 2 diabetes, dyslipidaemia or cancer comprising a compound of formula (I) or a pharmaceutically acceptable salt thereof.

The present invention relates to a pharmaceutical composition for the treatment of type 2 diabetes, obesity or dyslipidaemia comprising a compound of formula (I) or a pharmaceutically acceptable salt thereof.

The present invention further relates to a pharmaceutical composition comprising a) 10 to 2000 mg of a compound of formula (I) or a pharmaceutically acceptable salt thereof, and b) 0.1 to 2 g of one or more pharmaceutically acceptable carriers.

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any suitable route, and include those in a form adapted for oral, parenteral, transdermal, inhalation, sublingual, topical, implant, nasal, enterally (or other mucosally) administration to mammals including humans. The pharmaceutical compositions may be formulated in conventional manner using one or more pharmaceutically acceptable carriers or excipients. In one aspect, the pharmaceutical composition is formulated for oral administration

The compositions may be in the form of tablets, capsules, powders, granules, lozenges, such as oral or sterile parenteral solutions or suspensions.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice.

Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

The compounds of the invention may also, for example, be formulated as suppositories containing conventional suppository bases e.g. cocoa butter or other glyceride for use in human or veterinary medicine or as pessaries e.g., containing conventional pessary bases.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

As indicated, the compound of the present invention can be administered intranasally or by inhalation and is conveniently delivered in the form of a dry powder inhaler or an aerosol spray presentation from a pressurized container, pump, spray or nebulizer with the use of a suitable propellant, e.g., a hydrofluoroalkane such as 1,1,1,2-tetrafluoroethane (HFA 134AT) or 1,1,1,2,3,3,3-heptafluoropropane (HFA 227EA), or a mixture thereof. In the case of a pressurized aerosol, the dosage unit may be determined by providing a valve to deliver a metered amount. The pressurized container, pump, spray or nebulizer may contain a solution or suspension of the active compound, e.g., using a mixture of ethanol and the propellant as the solvent, which may additionally contain a lubricant e.g. sorbitan trioleate.

Capsules and cartridges (made, for example, from gelatin) for use in an inhaler or insufflator may be formulated to contain a powder mix of the compound and a suitable powder base such as lactose or starch.

Advantageously, agents such as a local anaesthetic, preservative and buffering agent can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compounds of the invention may be administered for immediate-, delayed-, modified-, sustained-, pulsed- or controlled-release applications.

In one aspect, oral compositions are slow, delayed or positioned release (e.g., enteric especially colonic release) tablets or capsules. This release profile can be achieved, for example, by use of a coating resistant to conditions within the stomach but releasing the contents in the colon or other portion of the GI tract wherein a lesion or inflammation site has been identified. Or a delayed release can be achieved by a coating that is simply slow to disintegrate. Or the two (delayed and positioned release) profiles can be combined in a single formulation by choice of one or more appropriate coatings and other excipients. Such formulations constitute a further feature of the present invention.

Suitable compositions for delayed or positioned release and/or enteric coated oral formulations include tablet formulations film coated with materials that are water resistant, pH sensitive, digested or emulsified by intestinal juices or sloughed off at a slow but regular rate when moistened. Suitable coating materials include, but are not limited to, hydroxypropyl methylcellulose, ethyl cellulose, cellulose acetate phthalate, polyvinyl acetate phthalate, hydroxypropyl methylcellulose phthalate, polymers of metacrylic acid and its esters, and combinations thereof. Plasticizers such as, but not limited to polyethylene glycol, dibutylphthalate, triacetin and castor oil may be used. A pigment may also be used to color the film. Suppositories are be prepared by using carriers like cocoa butter, suppository bases such as Suppocire C, and Suppocire NA50 (supplied by Gattefossé Deutschland GmbH, D-Weil am Rhein, Germany) and other Suppocire type excipients obtained by interesterification of hydrogenated palm oil and palm kernel oil (C₈-C₁₈ triglycerides), esterification of glycerol and specific fatty acids, or polyglycosylated glycerides, and whitepsol (hydrogenated plant oils derivatives with additives). Enemas are formulated by using the appropriate active compound according to the present invention and solvents or excipients for suspensions. Suspensions are produced by using micronized compounds, and appropriate vehicle containing suspension stabilizing agents, thickeners and emulsifiers like carboxymethylcellulose and salts thereof, polyacrylic acid and salts thereof, carboxyvinyl polymers and salts thereof, alginic acid and salts thereof, propylene glycol alginate, chitosan, hydroxypropylcellulose, hydroxypropylmethylcellulose, hydroxyethylcellulose, ethylcellulose, methylcellulose, polyvinyl alcohol, polyvinyl pyrrolidone, N-vinylacetamide polymer, polyvinyl methacrylate, polyethylene glycol, pluronic, gelatin, methyl vinyl ether-maleic anhydride copolymer, soluble starch, pullulan and a copolymer of methyl acrylate and 2-ethylhexyl acrylate lecithin, lecithin derivatives, propylene glycol fatty acid esters, glycerin fatty acid esters, sorbitan fatty acid esters, polyoxyethylene sorbitan fatty acid esters, polyethylene glycol fatty acid esters, polyoxyethylene hydrated caster oil, polyoxyethylene alkyl ethers, and pluronic and appropriate buffer system in pH range of 6.5 to 8. The use of preservatives, masking agents is suitable. The average diameter of micronized particles can be between 1 and 20 micrometers, or can be less than 1 micrometer. Compounds can also be incorporated in the formulation by using their water-soluble salt forms.

Alternatively, materials may be incorporated into the matrix of the tablet e.g. hydroxypropyl methylcellulose, ethyl cellulose or polymers of acrylic and metacrylic acid esters. These latter materials may also be applied to tablets by compression coating.

The compositions may contain from 0.1% by weight, preferably from 10-60% by weight, of the active ingredient, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e., the number of doses of a compound of the invention given per day for a defined number of days, can be ascertained by those skilled in the art using conventional course of treatment determination tests.

The compounds of formula (I) or pharmaceutically acceptable salt(s) thereof may also be used in combination with other therapeutic agents. The invention thus provides, in a further aspect, a combination comprising a) a compound of formula (I) or pharmaceutically acceptable salt thereof and b) one or more further therapeutically active agent(s).

The combinations referred to above may conveniently be presented for use in the form of a pharmaceutical composition and thus pharmaceutical compositions comprising a combination as defined above together with one or more pharmaceutically acceptable carriers thereof represent a further aspect of the invention.

Compounds of the invention may be administered in combination with other therapeutically active agents. Preferred therapeutic agents are selected from the list consisting of: insulin, bisguanidine, metformin, a DPP-IV inhibitor, sitagliptin, an inhibitor of cholesteryl ester transferase (CETP inhibitors), a HMG-CoA reductase inhibitor, a microsomal triglyceride transfer protein, a peroxisome proliferator-activated receptor activator (PPAR), a bile acid reuptake inhibitor, a cholesterol absorption inhibitor, a cholesterol synthesis inhibitor, a fibrate, niacin, an ion-exchange resin, an antioxidant, an inhibitor of AcylCoA: cholesterol acyltransferase (ACAT inhibitor), a cannabinoid 1 antagonist, a bile acid sequestrant, a corticosteroid, a vitamin D3 derivative, a retinoid, an immunomodulator, an anti androgen, a keratolytic agent, an anti-microbial, a platinum chemotherapeutic, an antimetabolite, hydroxyurea, a taxane, a mitotic disrupter, an anthracycline, dactinomycin, an alkylating agent and a cholinesterase inhibitor.

When the compound of formula (I) or pharmaceutically acceptable salt thereof is used in combination with a second therapeutically active agent the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian.

The combinations referred to above may conveniently be presented for use in the form of a pharmaceutical formulation and thus pharmaceutical formulations comprising a combination as defined above together with at least one pharmaceutically acceptable carrier and/or excipient comprise a further aspect of the invention.

The individual components of such combinations may be administered either sequentially or simultaneously in separate or combined pharmaceutical formulations by any convenient route.

When administration is sequential, either the AMPK activator or the second therapeutically active agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art.

### Methods of Preparation

Compounds of formula (I) and salts thereof may be prepared by the general methods outlined hereinafter or any method known in the art, said methods constituting a further aspect of the invention. R¹ to R⁷ are as defined above unless otherwise specified. Throughout the specification, general formulae are designated by Roman numerals (I), (II), (III), (IV) etc.

In a general process, compounds of formula (I), wherein R⁵ is as defined above other than H or bromo (formula (Ia)), may be prepared according to reaction scheme 1 by reacting compounds of formula (I), wherein R⁵ is bromo (formula (Ib)), with the appropriate boronic acid (IIa) in the presence of an inorganic base such as cesium carbonate and a catalyst (such as Pd(PPh₃)₄) in a suitable solvent such as a 1,4-dioxane/water mixture (suitably at 100 to 160° C.).

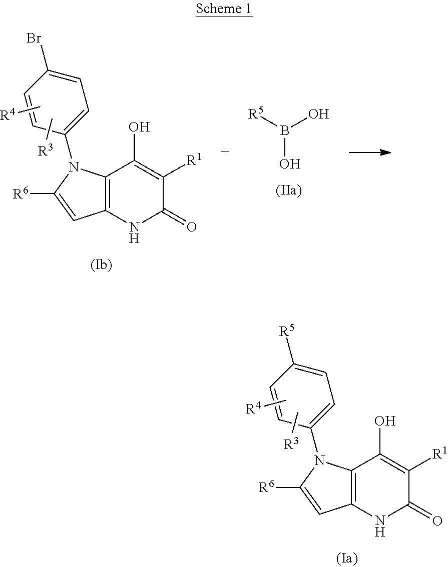

Compounds of formula (IIa) are commercially available or may be prepared by methods known in the literature or processes known to those skilled in the art.

Compounds of formula (Ib) may be prepared according to Scheme 12.

Compounds of formula (I), wherein R⁵ is as defined above other than H or bromo (formula (Ia)), may be alternatively prepared according to reaction scheme 2 by reacting compounds of formula (Ic), with the appropriate R⁵-halide (III) in the presence of an inorganic base such as cesium carbonate and a catalyst (such as Pd(PPh₃)₄) in a suitable solvent such as as a 1,4-dioxane/water mixture or a 1,4-dioxane/ethanol/water mixture (suitably at 100 to 160° C.).

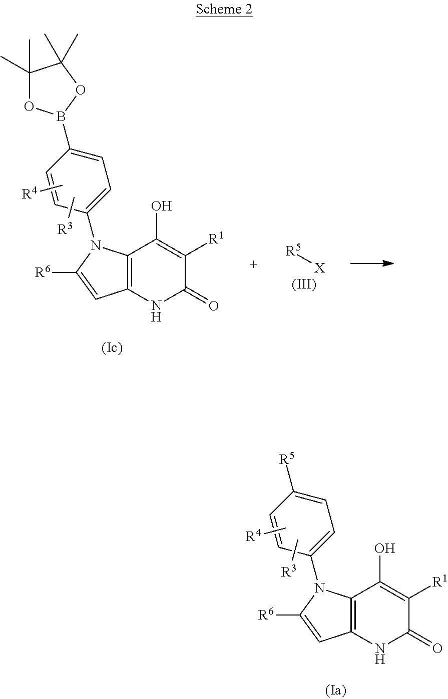

Compounds of formula (Ic) may be prepared according to Scheme 25.

Compounds of formula (III) are commercially available or may be prepared by methods known in the literature or processes known to those skilled in the art.

Compounds of formula (I), wherein R⁵ is as defined above other than H or bromo (formula (Ia)), may be alternatively prepared according to reaction scheme 3 by reacting compounds of formula (IVb), with the appropriate R⁵-halide (III) in the presence of an inorganic base such as cesium carbonate and a catalyst (such as Pd(PPh₃)₄) in a suitable solvent such as a 1,4-dioxane/water mixture or a 1,4-dioxane/ethanol/water mixture (suitably at 100 to 160° C.).

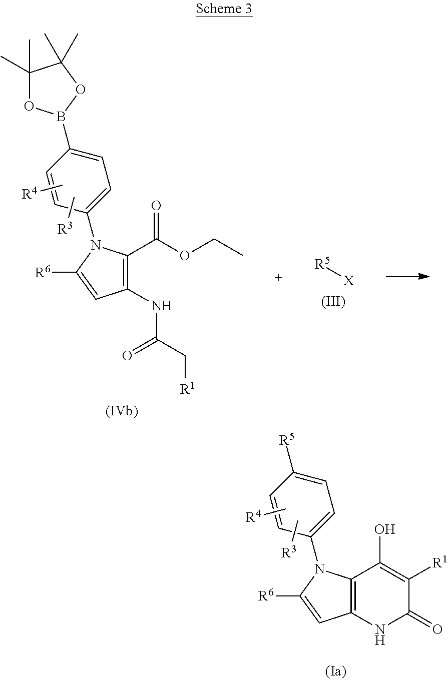

Compounds of formula (IVb) may be prepared according to Scheme 25. Compounds of formula (I), wherein

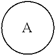

represents a —C₆₋₁₀aryl, —C₅₋₁₀heteroaryl, —C₅₋₁₀heterocyclyl or a —C₅₋₁₀cycloalkyl group (formula (Id)), may be alternatively prepared according to reaction scheme 4 by reacting compounds of formula (Ie) in the presence of BBr₃ in a suitable solvent such as DCM (suitably at RT).

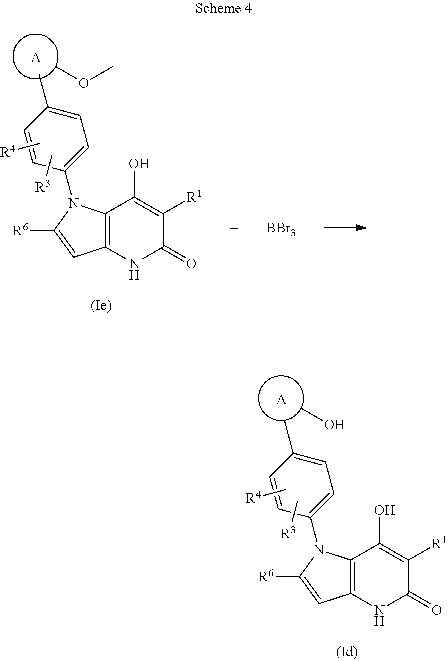

Compounds of formula (Ie) may be prepared according to Scheme 1 and/or Scheme 2 and/or Scheme 3.

Compounds of formula (I), wherein R¹ is phenyl substituted by a carboxylic acid (formula (Ig)) or a carboxamide (formula (If)), may be prepared according to reaction scheme 5 by reacting compounds of formula (Ih) in the presence of KOH in a suitable solvent such as an ethanol/water mixture.

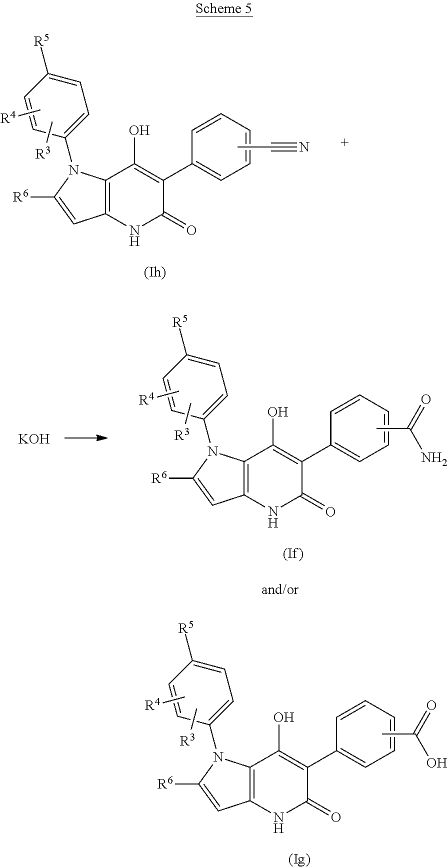

Compounds of formula (Ih) may be prepared according to Scheme 1 and/or Scheme 2 and/or Scheme 3.

Compounds of formula (I), wherein R⁵ is H (formula (Ii)), may be prepared according to reaction scheme 6 by hydrogenating compounds of formula (Ib) in the presence of a catalyst (such as Pd/C) in a suitable solvent such as methanol.

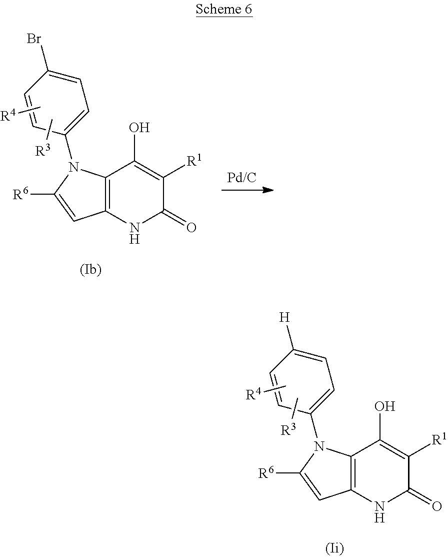

Compounds of formula (I), wherein R⁷ is —C₁₋₄alkyl (formula (Ij)) may be prepared according to reaction scheme 7 by reacting compounds of formula (Ia) with a base such as sodium hydride in the presence of a —C₁₋₄alkylating agent (R⁷—X), such as methyl iodide, in a suitable solvent such as DMF (suitably at 110° C. under microwave irradiation).

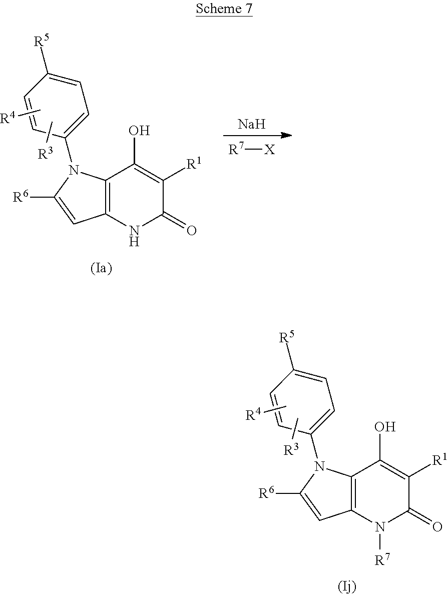

Compounds of formula R⁷—X are commercially available or may be prepared by methods known in the literature or processes known to those skilled in the art.

Compounds of formula (I), wherein R⁵ is —NHCOCH₃ (formula (Ik)) may be prepared according to reaction scheme 8 by reacting compounds of (I), wherein R⁵ is —NH₂ (formula (Im)) with acetyl chloride in a suitable solvent such as a THF/DMF mixture. Compounds of formula (Im) may be prepared by reacting compounds of formula (Ib) with ammonium hydroxide in the presence of a catalyst such as copper iodide and a ligand such as 2,4-pentanedione in a suitable solvent such as DMF (suitably at 100° C.).

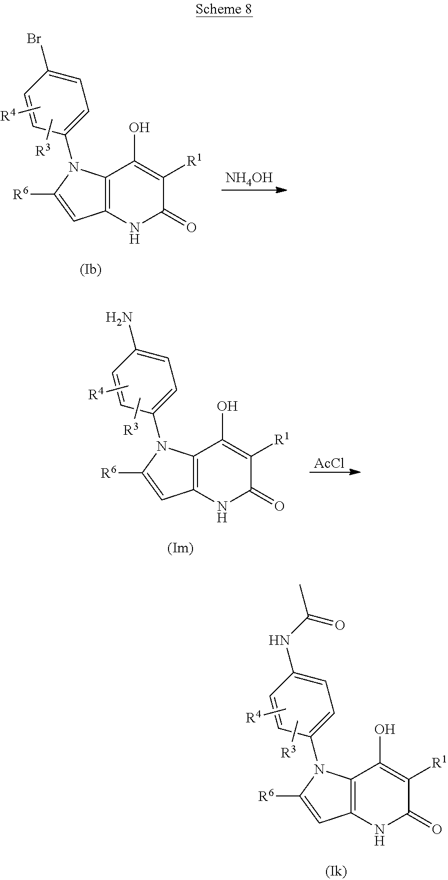

Compounds of formula (I), wherein R¹ is H (formula (In)), may be prepared according to reaction scheme 9 by reacting compounds of formula (I), wherein R¹ is —CO₂C₂H₅ and R⁵ is bromo (formula (Io)), with the appropriate R⁵-boron derivative (IIb) such as the appropriate boronic acid in the presence of an inorganic base such as cesium carbonate and a catalyst (such as Pd(PPh₃)₄) in a suitable solvent such as 1,4-dioxane (suitably at 100 to 160° C.).

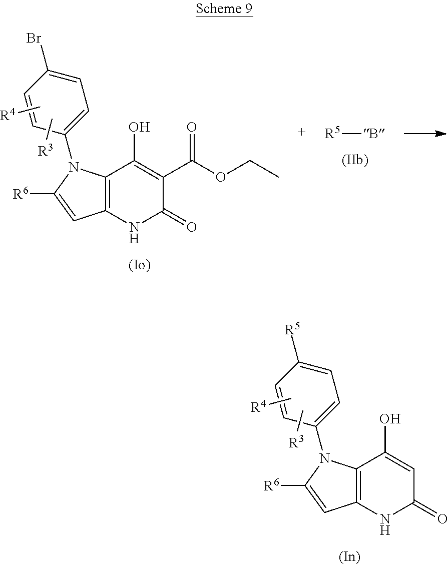

Compounds of formula (Io) may be prepared according to Scheme 12.

Compounds of formula (IIb) are commercially available or may be prepared by methods known in the literature or processes known to those skilled in the art.

Compounds of formula (I), wherein R¹ is H and R⁵ is bromo (formula (Ip)), may be prepared according to reaction scheme 10 by reacting compounds of formula (I), wherein R¹ is —CO₂C₂H₅ and R⁵ is bromo (formula (Io)), with KOH in a suitable solvent such as water (suitably at 120° C.).

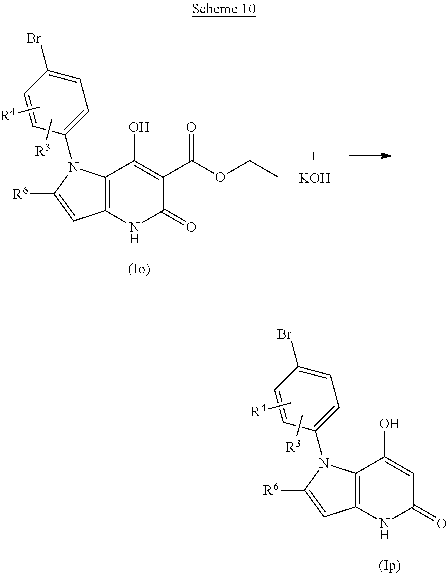

Compounds of formula (I), wherein R⁵ is

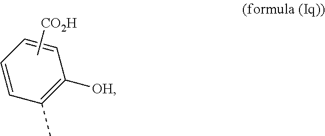

may be prepared according to reaction scheme 11 by reacting compounds of formula (I), wherein R⁵ is bromo (formula (Ib)), with the appropriate boronic acid (IIc) in the presence of an inorganic base such as cesium carbonate and a catalyst (such as Pd(PPh₃)₄) in a suitable solvent such as a 1,4-dioxane/water mixture (suitably at 100 to 160° C.).

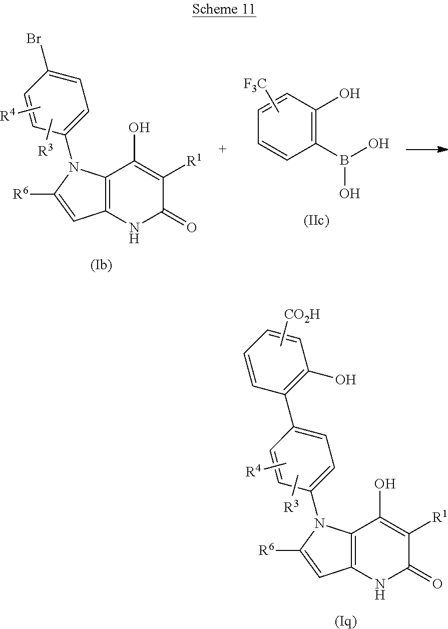

Compounds of formula (IIc) are commercially available or may be prepared by methods known in the literature or processes known to those skilled in the art.

Compounds of formula (I) may be prepared according to reaction scheme 12 by reacting compounds of formula (IV) in the presence of a base such as sodium hydride or potassium tertbutoxide or potassium hexamethyldisilazane in a suitable solvent such as THF or DMSO (suitably at room temperature or reflux).

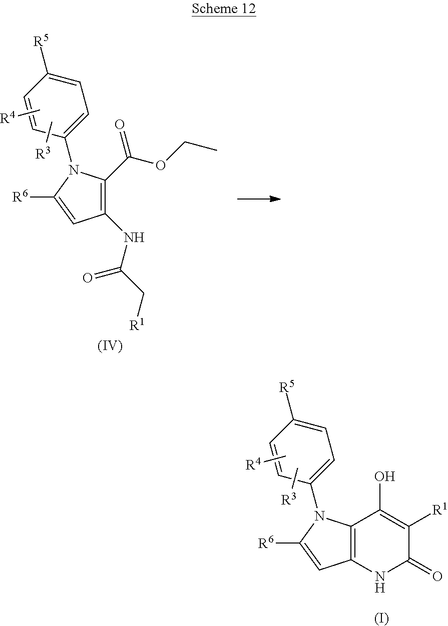

Compounds of formula (IV) may be prepared according to Scheme 13 and/or Scheme 14 and/or Scheme 15.

Compounds of formula (IV) may be prepared according to reaction scheme 13 by reacting compounds of formula (IV), wherein R⁵ is bromo (formula (IVa)), with the appropriate R⁵-boron derivative (IIb) such as an appropriate boronic acid or potassium trifluoroborate derivative in the presence of an inorganic base such as cesium carbonate and a catalyst (such as Pd(PPh₃)₄) in a suitable solvent such as 1,4-dioxane (suitably at 100 to 160° C.).

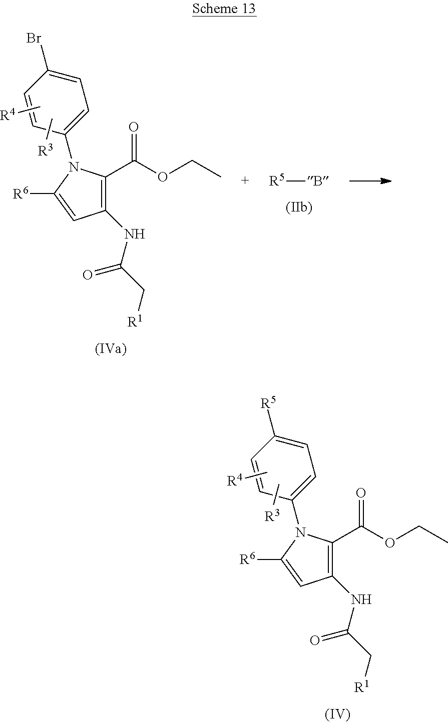

Compounds of formula (IVa) may be prepared according to Scheme 15.

Compounds of formula (IV) may be also prepared according to reaction scheme 14 by reacting compounds of formula (IVb), with the appropriate R⁵-halide derivative (III) in the presence of an inorganic base such as cesium carbonate and a catalyst (such as Pd(PPh₃)₄) in a suitable solvent such as 1,4-dioxane (suitably at 100 to 160° C.).

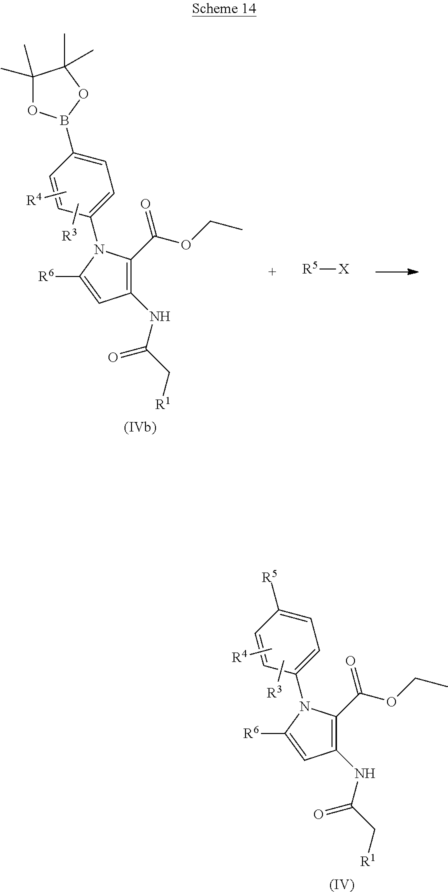

Compounds of formula (IVb) may be prepared according to Scheme 25.

Compounds of formula (IV) may also be prepared according to reaction scheme 15 by reacting compounds of formula (V) with acetic acid derivatives (VI) in the presence of a coupling reagent such as DCC or EDC/HOBT or COMU and a base such as triethylamine in a suitable solvent such as acetonitrile (suitably at room temperature) or with acetyl chloride derivatives (VII) in the presence of a base such as triethylamine in a suitable solvent such as DCM (suitably at room temperature).

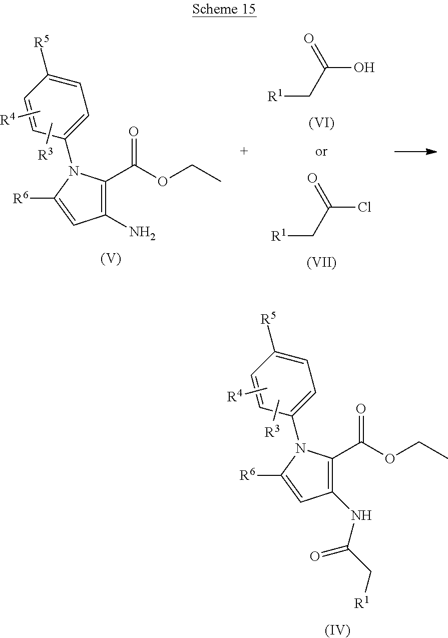

Compounds of formula (V) may be prepared according to Scheme 16, Scheme 18, Scheme 19 and/or Scheme 20.

Compounds of formula (VI) or (VII) are commercially available or may be prepared by methods known in the literature or processes known to those skilled in the art.

Compounds of formula (V), wherein R⁶ is methyl (formula (Va) may be prepared according to reaction scheme 16 by reacting compounds of formula (V), wherein R⁶ is chlorine (formula (Vb)), in the presence of trimethylboroxin, an inorganic base such as cesium carbonate and a catalyst (such as palladium acetate and {2′,6′-bis[(1-methylethyl)oxy]-2-biphenylyl}(dicyclohexyl)phosphane as a ligand) in a suitable solvent such as ethanol.

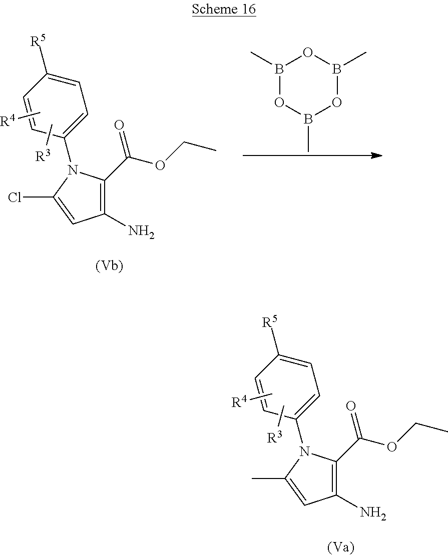

Compounds of formula (Vb) may be prepared according to Scheme 18, Scheme 19 and/or Scheme 20.

Compounds of formula (IV), wherein R⁶ is cyano (formula (IVc) may be prepared according to reaction scheme 17 by reacting compounds of formula (IV), wherein R⁶ is chlorine (formula (IVd)), in the presence of zinc cyanide and a catalyst (such as palladium trifluoroacetate and 2-(di-t-butylphosphino)-1,1′-binapthyl as a ligand) in a suitable solvent such as N,N-dimethylacetamide.

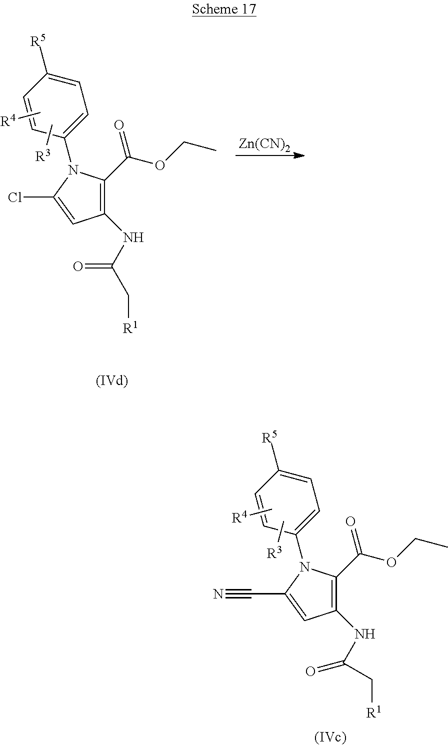

Compounds of formula (IVd) may be prepared according to Scheme 15.

Compounds of formula (V) may be prepared according to reaction scheme 18 by reacting compounds of formula (V), wherein R⁵ is bromo (formula (Vc)), with the appropriate R⁵-boron derivative (IIb) such as the appropriate boronic acid or potassium trifluoroborate derivative in the presence of an inorganic base such as cesium carbonate and a catalyst (such as Pd(PPh₃)₄) in a suitable solvent such as 1,4-dioxane (suitably at 100 to 160° C.).

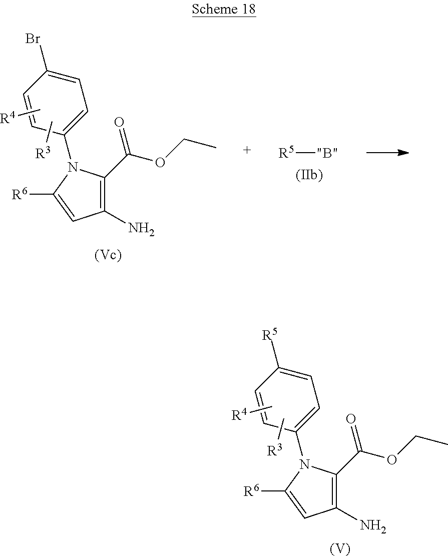

Compounds of formula (Vc) may be prepared according to Scheme to 16, Scheme 19 and/or Scheme 20.

Compounds of formula (V) may be prepared according to reaction scheme 19 by reacting compounds of formula (Vd), with the appropriate R⁵-halide derivative (III) in the presence of an inorganic base such as cesium carbonate and a catalyst (such as Pd(PPh₃)₄) in a suitable solvent such as 1,4-dioxane (suitably at 100 to 160° C.).

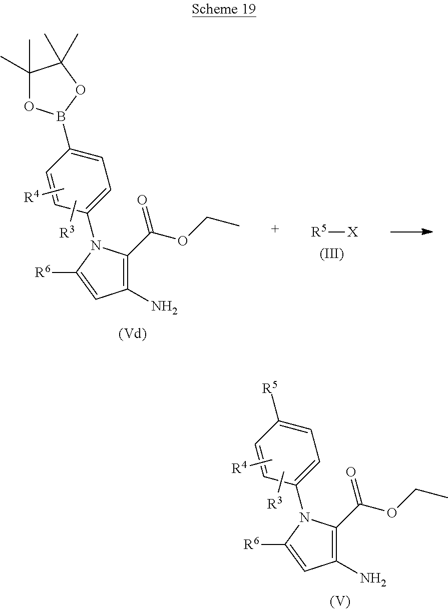

Compounds of formula (Vd) may be prepared according to Scheme 25.

Compounds of formula (V) may be prepared according to reaction scheme 20 by reacting compounds of formula (VIII) in the presence of an acid such as HCl in a suitable solvent such as ethanol (suitably at reflux).

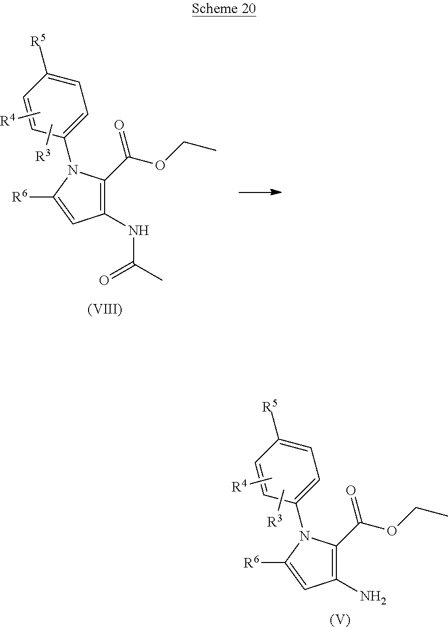

Compounds of formula (VIII) may be prepared according to Scheme 21 and/or Scheme 22.

Compounds of formula (VIII), wherein R⁶ is chlorine (formula (VIIIa)), may be prepared according to reaction scheme 21 by reacting compounds of formula (IX), wherein R⁶ is H, with N-Chlorosuccinimide (NCS) (commercially available) in a suitable solvent such as chloroform (suitably at room temperature).

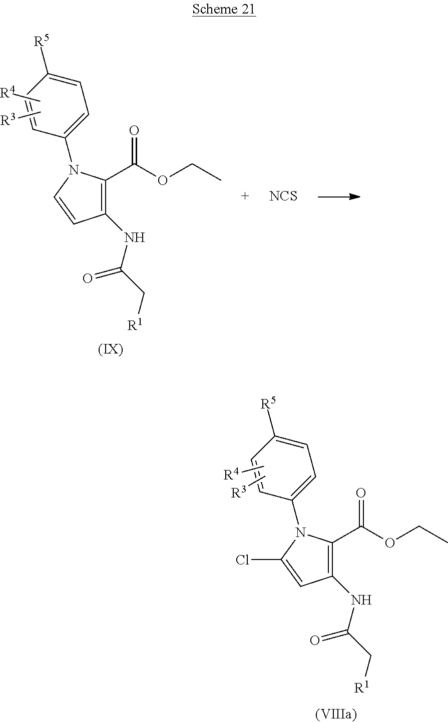

Compounds of formula (IX) may be prepared according to Scheme 22.

Compounds of formula (VIII) may be alternatively prepared according to reaction scheme 22 by reacting compounds of formula (X) with the appropriate phenylboronic acid derivative (XI) or halide derivative (XII) in the presence of a copper catalyst such as copper acetate or copper iodide and a base such as pyridine or tripotassium phosphate and N,N′-dimethyl-1,2-ethanediamine in a suitable solvent such as DCM (suitably at room temperature) or toluene (suitably at reflux).

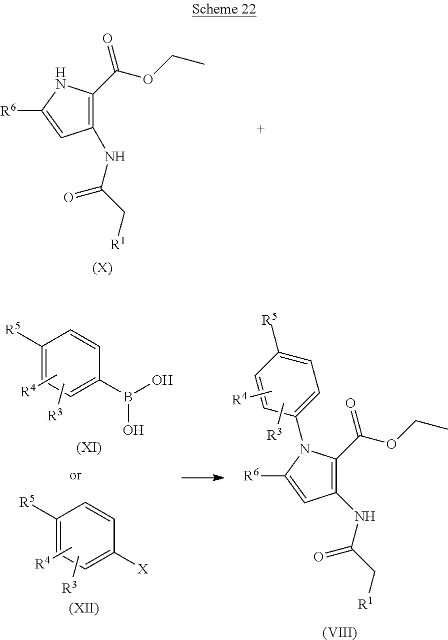

Compounds of formula (X) may be prepared according to Scheme 23 and/or Scheme 24.

Compounds of formula (XI) and (XII) are commercially available or may be prepared by methods known in the literature or processes known to those skilled in the art.

Compounds of formula (X), wherein R⁶ is Cl (formula (Xa)), may be prepared according to reaction scheme 23 by reacting compounds of formula (Xb) with N-Chlorosuccinimide (NCS) (commercially available) in a suitable solvent such as chloroform (suitably at room temperature).

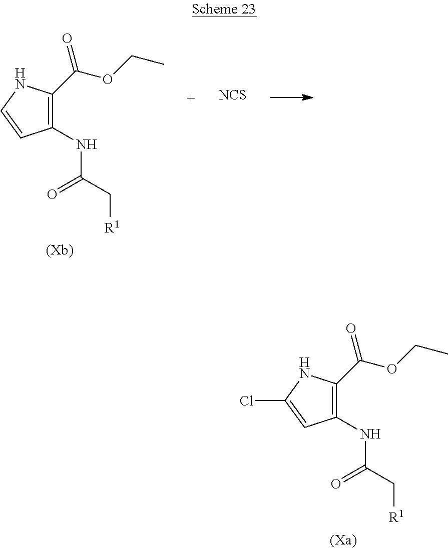

Compounds of formula (Xb) may be made according to Scheme 24.

Compounds of formula (X), wherein R⁶ is H (formula (Xb)), may be prepared according to reaction scheme 24 by reacting a compound of formula (XIV) with an acylchloride derivative of formula (VII) in the presence of a base such as triethylamine in a suitable solvent such as DCM (suitably at room temperature).

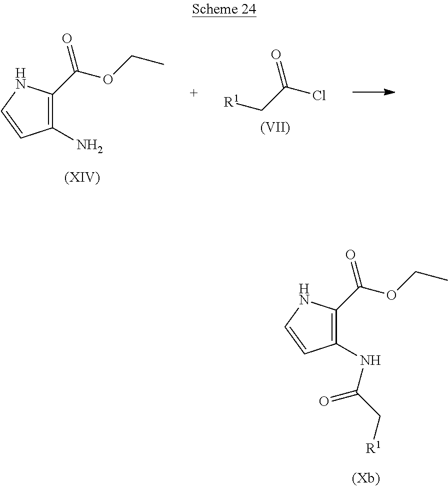

Compounds of formula (XIV) are commercially available or may be prepared by methods known in the literature or processes known to those skilled in the art.

Compounds of formula (Ic) may be prepared according to reaction scheme 25 by reacting compounds of formula (IVb) with a base such as sodium hydride or potassium tertbutoxide or potassium hexamethyldisilazane in a suitable solvent such as THF or DMSO (suitably at room temperature or reflux). Compounds of formula (IVb) may be prepared by reacting compounds of formula (Vd) with acetic acid derivatives (VI) in the presence of a coupling reagent such as DCC or EDC/HOBT or COMU and a base such as triethylamine in a suitable solvent such as acetonitrile (suitably at room temperature). Compounds of formula (Vd) may be prepared by reacting compounds of formula (V) with 4,4,4′,4′,5,5,5′,5′-octamethyl-2,2′-bi-1,3,2-dioxaborolane in the presence of an inorganic base such as potassium acetate and a catalyst (such as Pd(dppf)Cl₂) in a suitable solvent such as 1,4-dioxane (suitably at 100° C.).

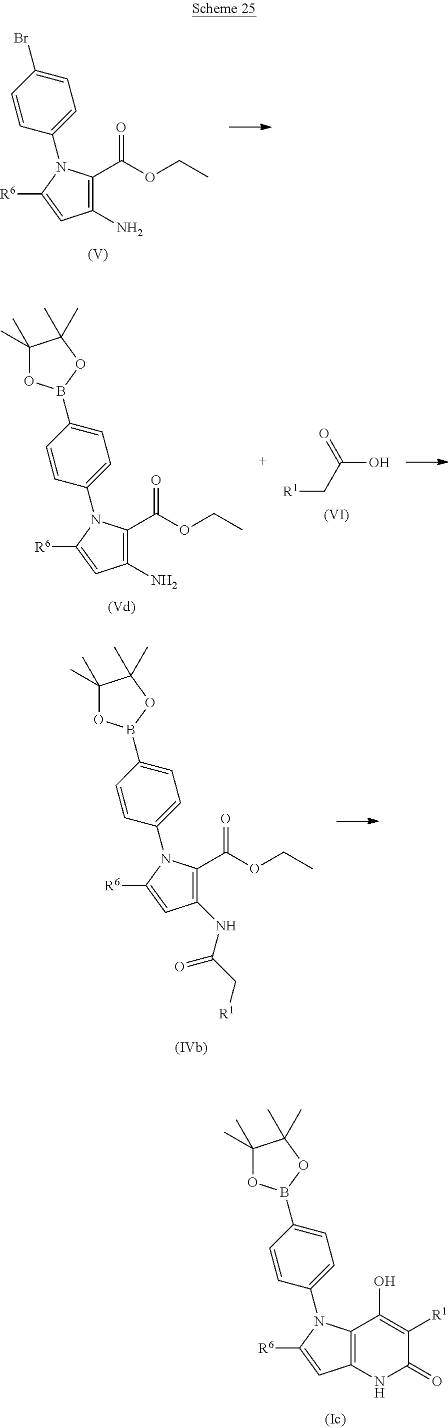

Alternatively, compounds of formula (IV), wherein R⁶ is defined as chloro (formula (IVe)), may be prepared according to reaction scheme 26 by reacting compounds of formula (IVf), wherein R⁶ is H, with N-chlorosuccinimide (NCS) (commercially available) in a suitable solvent such as chloroform (suitably at room temperature).

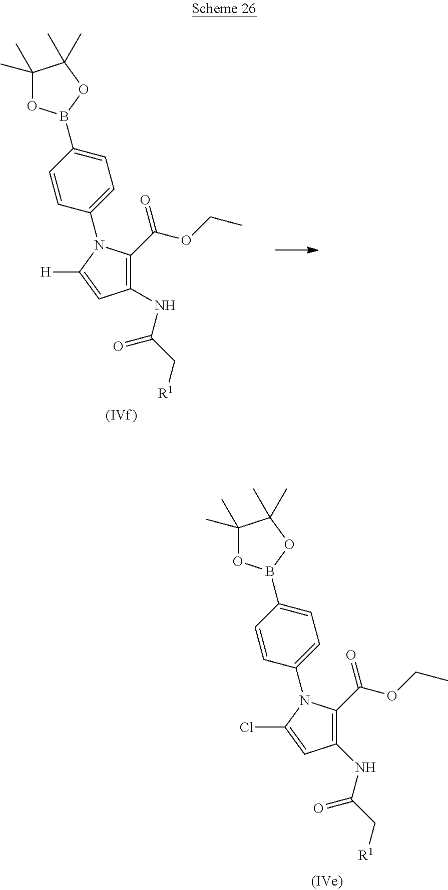

Compounds of formula (IVf) may be prepared according Scheme 25.

Further details for the preparation of compounds of formula (I) are found in the Examples section hereinafter.

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Those skilled in the art will appreciate that in the preparation of compounds of formula (I) and/or solvates thereof it may be necessary and/or desirable to protect one or more sensitive groups in the molecule or the appropriate intermediate to prevent undesirable side reactions. Suitable protecting groups for use according to the present invention are well known to those skilled in the art and may be used in a conventional manner. See, for example, “Protective groups in organic synthesis” by T. W. Greene and P. G. M. Wuts (John Wiley & sons 1991) or “Protecting Groups” by P. J. Kocienski (Georg Thieme Verlag 1994). Examples of suitable amino protecting groups include acyl type protecting groups (e.g. formyl, trifluoroacetyl, acetyl), aromatic urethane type protecting groups (e.g. benzyloxycarbonyl (Cbz) and substituted Cbz), aliphatic urethane protecting groups (e.g. 9-fluorenylmethoxycarbonyl (Fmoc), t-butyloxycarbonyl (Boc), isopropyloxycarbonyl, cyclohexyloxycarbonyl) and alkyl or aralkyl type protecting groups (e.g. benzyl, trityl, chlorotrityl).

The synthesis of the target compound is completed by removing any protecting groups, which are present in the penultimate intermediate using standard techniques, which are well-known to those skilled in the art. The final product is then purified, as necessary, using standard techniques such as silica gel chromatography, HPLC on silica gel, and the like or by recrystallization.

Various intermediate compounds used in the above-mentioned process, including but not limited to certain compounds of formulae (IV), (V) and (VIII) constitute a further aspect of the present invention.

### DEFINITIONS

AcOH Acetic acid
COMU (1-Cyano-2-ethoxy-2-oxoethylidenaminooxy)dimethylamino-morpholino-carbenium hexafluorophosphate

### DCC N,N′-Dicyclohexylcarbodiimide

### DCM Dichloromethane

### DMF N,N-dimethylformamide

DMSO d6 Deuterated dimethylsulfoxide

### DMSO Dimethylsulfoxide

EDC/(EDCl) 1-Ethyl-3-(3-Dimethylaminopropyl)carbodiimide (hydrochloride)
EtOAc/AcOEt Ethyl acetate

### h Hours

### HOBT N-Hydroxybenzotriazole

HRMS High resolution mass spectroscopy
Int. Intermediate
LC Liquid chromatography
LCMS Liquid chromatography mass spectroscopy

### MDAP Mass-Directed Auto Prep

Min. Minutes

### NCS N-chlorosuccinimide

Pd/C Palladium on carbon
RT Room temperature
Rt Retention time
Sat. Saturated
SM Starting material
SPE Solid phase extraction

### THF Tetrahydrofuran

TLC Thin-layer chromatography

The compounds and processes of the present invention will be better understood in connection with the following examples, which are intended as an illustration only and not limiting the scope of the invention. Various changes and modifications to the disclosed embodiments will be apparent to those skilled in the art and such changes and modifications including, without limitation, those relating to the chemical structures, substituents, derivatives, formulations and/or methods of the invention may be made without departing from the spirit of the invention and the scope of the appended claims.

Regardless of how the preparation of compounds are represented in the present specification no inference can be drawn that particular batches (or mixtures of two or more batches) of intermediates were used in the next stage of the preparation. The examples and intermediates are intended to illustrate the synthetic routes suitable for preparation of the same, to assist the skilled persons understanding of the present invention.

Where reference is made to the use of a “similar” procedure, as will be appreciated by those skilled in the art, such a procedure may involve minor variation, for example reaction temperature, reagent/solvent amount, reaction time, work-up conditions or chromatographic purification conditions.

Compounds are named using ACD/Name PRO 6.02 chemical naming software (Advanced Chemistry Development Inc., Toronto, Ontario, M5H2L3, Canada).

1H NMR spectra were acquired on a 300 MHz Brucker spectrometer [Bruker AV300] (AV300) or a 400 MHz Bruker [Bruker AV400] spectrometer. Sample was dissolved in DMSO-d6 or CDCl₃ and chemical shifts were reported in ppm relative to the solvent residual peak. Coupling constants (J) are in units of hertz (Hz). Splitting patterns describe apparent multiplicities and are designated as s (singlet), d (doublet), t (triplet), q (quartet), dd (double doublet), dt (double triplet), m (multiplet), br (broad).

### Analytical Method LC-MS

### Methods:

(a) Analytical HPLC was conducted on a X-Terra MS C18 column (2.5 μm 30×3 mm id) eluting with 0.01M ammonium acetate in water (solvent A) and 100% acetonitrile (solvent B), using the following elution gradient 0 to 4 minutes. 0 to 100% B, 4 to 5 minutes 100% B at a flow rate of 1.1 ml/minute. The mass spectra (MS) were recorded on a Waters ZQ mass spectrometer using electrospray positive ionisation [ES+ to give (M+H)⁺ molecular ions] or electrospray negative ionisation [ES− to give (M−H)⁻ molecular ions] modes.
(b) Analytical HPLC was conducted on a X-Terra MS C18 column (3.5 μm 30×4.6 mm id) eluting with 0.01M ammonium acetate in water (solvent A) and 100% methanol (solvent B), using the following elution gradient 0 to 7.5 minutes, 10 to 100% B, 7.5 to 10 minutes 100% B, 10.5 to 12 min 10% B at a flow rate of 1.4 ml/minute. The mass spectra (MS) were recorded on a Waters ZQ mass spectrometer using electrospray positive ionisation [ES+ to give (M+H)⁺ molecular ions] or electrospray negative ionisation [ES− to give (M−H)⁻ molecular ions] modes.

### Analytical LC-HRMS

Analytical HPLC was conducted on a Waters XBridge column (2.5 μm 30×3 mm id) eluting with 0.01M ammonium acetate in water (solvent A) and 100% acetonitrile (solvent B) using the following elution gradient: 0 to 0.5 minutes, 5% B; 0.5 to 3.75 minutes, 5% B to 100% B; 3.75 to 4.5 minutes, 100% B; 4.5 to 5 minutes, 100% B to 5% B; 5 to 5.5 minutes, 5% B at a flowrate of 1.3 mL/min with a temperature of 40° C. The mass spectra (MS) were recorded on a Waters LCT mass spectrometer using electrospray positive ionisation [ES+ve to give (M+H)+ molecular ion] or electrospray negative ionisation [ES-ve to give (M−H)⁻ molecular ion] modes.

The following non-limiting Examples illustrate the present invention.

### Intermediate 1

### Ethyl 3-(acetylamino)-1H-pyrrole-2-carboxylate

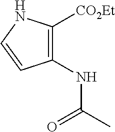

Method A: To a suspension of ethyl 3-amino-1H-pyrrole-2-carboxylate hydrochloride (1 g, 4.98 mmol, commercially available from Combi-Blocks) in DCM (50 mL) at 0° C. was added drop-wise triethylamine (2 mL, 14.43 mmol) and acetyl chloride (0.45 mL, 6.31 mmol). The reaction mixture was then stirred from 0° C. to RT for 12 hours before being quenched with 1N HCl. The organic layer was separated and washed successively with sat. NaHCO₃ and brine, dried over Na₂SO₄, filtered and concentrated under reduced pressure. The product was purified by chromatography on an Isco Companion RF. The sample was loaded on 100 g Biotage silica column and then the purification was carried out using DCM/MeOH 100/0 to 90/10. The appropriate fractions were combined and evaporated in vacuo to give the required product ethyl 3-(acetylamino)-1H-pyrrole-2-carboxylate (0.99 g, 5.05 mmol, 100% yield) as a yellow solid. ¹H NMR: (CDCl₃, 400 Hz) δ 9.12 (br s, 1H), 8.78 (br s, 1H), 7.05 (s, 1H), 6.81 (s, 1H), 4.34 (q, J=7.0 Hz, 2H), 2.18 (s, 3H), 1.37 (t, J=7.0 Hz, 3H). LCMS: (M+H)⁺: 197; Rt: 1.93 min.

Method B: To a suspension of ethyl 3-amino-1H-pyrrole-2-carboxylate (commercially available from Combi-Blocks, 25 g, 131 mmol) in dichloromethane (DCM) (150 mL) at 0° C. was added triethylamine (40.1 mL, 289 mmol). After stirring for 10 minutes, a solution of acetyl chloride (10.26 mL, 144 mmol) in dichloromethane (DCM) (50 mL) was added dropwise. The reaction mixture was then stirred from 0° C. to RT for 3 h before being quenched with sat NaHCO₃. More DCM was added to solubilise a precipitate. The organic layer was separated and washed with brine, dried over Na₂SO₄, filtered and concentrated under reduced pressure. The product was purified by recrystallisation in diisopropyl oxide to give ethyl 3-(acetylamino)-1H-pyrrole-2-carboxylate (22 g, 107 mmol, 81% yield) as a white powder. LCMS: (M+H)⁺: 197; Rt: 1.92 min

### Intermediate 2

### Ethyl 3-(acetylamino)-1-(4-bromophenyl)-1H-pyrrole-2-carboxylate

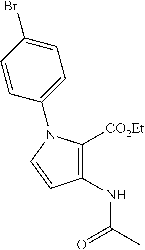

Method A: Copper(II) acetate (1.37 g, 7.57 mmol) was added to a solution of 4-bromophenylboronic acid (2.03 g, 10.09 mmol), ethyl 3-(acetylamino)-1H-pyrrole-2-carboxylate (Intermediate 1) (0.99 g, 5.05 mmol) and pyridine (0.81 mL, 10.04 mmol) in DCM (20 mL) at RT. The reaction mixture was stirred for 24 hours and monitoring of the reaction by LCMS showed the reaction was incomplete with SM remaining. 4-bromophenylboronic acid (2.027 g, 10.09 mmol), copper(II) acetate (1.375 g, 7.57 mmol) and pyridine (0.81 mL, 10.04 mmol) were added again in the same order and the mixture was stirred for another 72 hours. The reaction mixture was then concentrated under reduced pressure. The crude extract was then purified by chromatography on an Isco Companion RF. The sample was loaded on 100 g Biotage silica column then the purification was carried out using cyclohexane/EtOAc 100/0 to 50/50. The appropriate fractions were combined and concentrated in vacuo to give the required product ethyl 3-(acetylamino)-1-(4-bromophenyl)-1H-pyrrole-2-carboxylate (1.36 g, 3.87 mmol, 77% yield) as a colorless oil which solidified. ¹H NMR: (CDCl₃, 400 Hz) δ 9.54 (s, 1H), 7.54 (d, J=7.5 Hz, 2H), 7.18 (s, 1H), 7.14 (d, J=7.5 Hz, 2H), 4.11 (q, J=7.0 Hz, 2H), 2.21 (s, 3H), 1.03 (t, J=7.0 Hz, 3H). LCMS: (M+H)⁺: 351, 353; Rt: 3.28 min.

### Method B:

Copper(II) acetate (6.57 g, 36.2 mmol) was added to a solution of 4-bromophenylboronic acid (9.68 g, 48.2 mmol), ethyl 3-(acetylamino)-1H-pyrrole-2-carboxylate (Intermediate 1) (4.73 g, 24.11 mmol) and pyridine (3.9 mL, 48.3 mmol) in DCM (100 mL) at RT. The reaction mixture was stirred for 48 h before 4-bromophenylboronic acid (9.68 g, 48.2 mmol), copper(II) acetate (6.57 g, 36.2 mmol) and pyridine (3.9 mL, 48.3 mmol) were added again in the same order. The mixture was stirred for another 24 h. All the reactants were added again and the mixture stirred for another 15 h. The reaction mixture was then washed respectively with 1N HCl, water and brine. The combined organic layers were dried over Na₂SO₄, filtered and concentrated under reduced pressure. The crude was then purified by chromatography on an Isco Companion RF. The sample was loaded on 340 g Biotage SNAP silica (Si) column then the purification was carried out using eluting with cyclohexane/EtOAc 100/0 to 60/40. The appropriate fractions were combined and concentrated in vacuo to give the required product ethyl 3-(acetylamino)-1-(4-bromophenyl)-1H-pyrrole-2-carboxylate (7.91 g, 22.52 mmol, 93% yield) as a colourless oil which solidified. LCMS: (M+H)⁺: 351, 353; Rt: 3.26 min.

### Intermediate 3

### Ethyl 3-amino-1-(4-bromophenyl)-1H-pyrrole-2-carboxylate

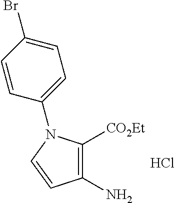

Method A: A solution of ethyl 3-(acetylamino)-1-(4-bromophenyl)-1H-pyrrole-2-carboxylate (Intermediate 2) (1.15 g, 3.27 mmol) and concentrated HCl (4 mL, 48.7 mmol) in ethanol (50 mL) was refluxed for 2 hours before being concentrated under reduced pressure. The crude solid was triturated in hot CH₃CN and the solid filtered and dried to give the desired compound ethyl 3-amino-1-(4-bromophenyl)-1H-pyrrole-2-carboxylate (0.62 g, 1.794 mmol, 54.8% yield) as a white solid. ¹H NMR: (DMSO) δ 8.34 (br s, 2H), 7.64 (d, J=8.0 Hz, 2H), 7.30 (d, J=8.0 Hz, 2H), 7.22 (s, 1H), 6.35 (s, 1H), 4.09 (q, J=7.1 Hz, 2H), 1.12 (t, J=7.1 Hz, 3H). LCMS: (M+H)⁺: 309, 311; Rt: 3.19 min.

Method B: A solution of ethyl 3-(acetylamino)-1-(4-bromophenyl)-1H-pyrrole-2-carboxylate (Intermediate 2) (7.91 g, 22.52 mmol) and concentrated HCl (9 mL, 110 mmol) in ethanol (300 mL) was refluxed for 18 h before being concentrated under reduced pressure. The crude solid was triturated in CH₃CN and the solid filtrated and dried to give the desired compound ethyl 3-amino-1-(4-bromophenyl)-1H-pyrrole-2-carboxylate (5.45 g, 15.77 mmol, 70.0% yield) as a white solid. LCMS: (M+H)⁺: 309, 311; Rt: 3.17 min.

### Intermediate 4

### Ethyl 1-(4-bromophenyl)-3-[(cyanoacetyl)amino]-1H-pyrrole-2-carboxylate

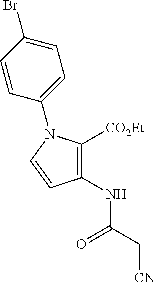

Method A: To a solution of ethyl 3-amino-1-(4-bromophenyl)-1H-pyrrole-2-carboxylate (Intermediate 3) (0.62 g, 1.794 mmol), cyanoacetic acid (0.23 g, 2.70 mmol) and triethylamine (550 μL, 3.95 mmol) in acetonitrile (20 mL) at RT was added drop-wise a solution of DCC (0.61 g, 2.96 mmol) in acetonitrile (5 mL). The reaction mixture was stirred for 48 hours and monitoring of the reaction by LCMS showed the reaction was incomplete with SM remaining. Cyanoacetic acid (100 mg), triethylamine (200 μL) and DCC (100 mg) were added and the mixture was stirred for another 48 hours before being filtered and washed with acetonitrile. The filtrate was concentrated under reduced pressure and the crude solid triturated in MeOH, filtered and dried to give the desired product ethyl 1-(4-bromophenyl)-3-[(cyanoacetyl)amino]-1H-pyrrole-2-carboxylate (520 mg, 1.382 mmol, 77% yield) as a off-white solid. ¹H NMR: (DMSO-d6, 400 Hz) δ 10.15 (s, 1H), 7.56 (d, J=7.7 Hz, 2H), 7.16 (d, J=7.7 Hz, 2H), 7.13 (s, 1H), 6.82 (s, 1H), 4.20 (q, J=7.0 Hz, 2H), 3.59 (s, 2H), 1.09 (t, J=7.0 Hz, 3H). LCMS: (M+H)⁺: 374, 376; Rt: 3.31 min.

Method B: To a solution of ethyl 3-amino-1-(4-bromophenyl)-1H-pyrrole-2-carboxylate (Intermediate 3) (5.45 g, 15.77 mmol), cyanoacetic acid (2.012 g, 23.65 mmol) and triethylamine (6 mL, 43.0 mmol) in acetonitrile (180 mL) at RT was added dropwise a solution of DCC (5.21 g, 25.2 mmol) in acetonitrile (45 mL). The reaction mixture was stirred for 72 h before being filtered. The filtrate was concentrated under reduced pressure and the crude solid triturated in MeOH, filtered and dried to give the desired product ethyl 1-(4-bromophenyl)-3-[(cyanoacetyl)amino]-1H-pyrrole-2-carboxylate (4.5 g, 11.96 mmol, 76% yield) as a white solid. LCMS: (M+H)⁺: 376, 378; Rt: 3.25 min.

### Intermediate 5

### Ethyl 1-(4-bromophenyl)-3-{[3-(ethyloxy)-3-oxopropanoyl]amino}-1H-pyrrole-2-carboxylate

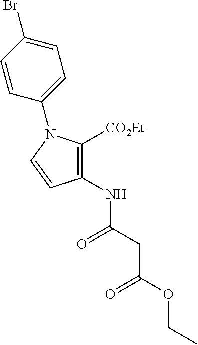

Method A: To a solution of ethyl 3-amino-1-(4-bromophenyl)-1H-pyrrole-2-carboxylate (Intermediate 3) (5 g, 14.47 mmol), and triethylamine (4.44 mL, 31.8 mmol) in DCM (60 mL) at RT was added drop-wise a solution of ethyl 3-chloro-3-oxopropanoate (2.037 mL, 15.91 mmol) in DCM (10 mL). The reaction mixture was stirred overnight and monitoring of the reaction by LCMS showed the reaction was complete. The reaction mixture was evaporated to dryness, the residue extracted with EtOAc/water and then washed with sat. NaHCO₃ and brine. The organic layer was dried over Na₂SO₄ and evaporated to dryness. The desired product ethyl 1-(4-bromophenyl)-3-{[3-(ethyloxy)-3-oxopropanoyl]amino}-1H-pyrrole-2-carboxylate (6.1 g, 14.41 mmol, 100% yield) was obtained as a cream solid. ¹H NMR: (DMSO-d6, 300 Hz) δ 9.99 (br s, 1H), 7.64 (d, J=8.6 Hz, 2H), 7.31 (d, J=8.6 Hz, 2H), 7.17 (d, J=3.0 Hz, 1H), 6.97 (d, J=3.0 Hz, 1H), 4.15 (q, J=7.1 Hz, 2H), 4.09 (q, J=7.0 Hz, 2H), 3.63 (s, 2H), 1.22 (t, J=7.0 Hz, 3H), 1.03 (t, J=7.1 Hz, 2H). LCMS: (M+H)⁺: 425; Rt: 3.68 min.

Method B: To a solution of ethyl 3-amino-1-(4-bromophenyl)-1H-pyrrole-2-carboxylate (Intermediate 3) (5 g, 14.47 mmol) and triethylamine (4.44 mL, 31.8 mmol) in DCM (60 mL) at RT was added dropwise a solution of ethyl 3-chloro-3-oxopropanoate (2.037 mL, 15.91 mmol) in DCM (10 mL). The reaction mixture was stirred overnight. The reaction mixture was evaporated to dryness, the residue extracted with EtOAc/water and then washed with sat. NaHCO₃ and brine. The organic layer was dried over Na₂SO₄ and evaporated to dryness. The desired product ethyl 1-(4-bromophenyl)-3-{[3-(ethyloxy)-3-oxopropanoyl]amino}-1H-pyrrole-2-carboxylate (6.1 g, 14.41 mmol, 100% yield) was obtained as a cream solid. LCMS: (M+H)⁺: 423, 425; Rt: 3.68 min.

### Intermediate 6

### Ethyl 1-(4-bromophenyl)-7-hydroxy-5-oxo-4,5-dihydro-1H-pyrrolo[3,2-b]pyridine-6-carboxylate

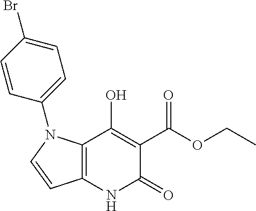

Method A: To a solution of ethyl 1-(4-bromophenyl)-3-{[3-(ethyloxy)-3-oxopropanoyl]amino}-1H-pyrrole-2-carboxylate (Intermediate 5) (5.07 g, 11.98 mmol) in DMF (50 mL) at 0° C. was added portionwise sodium hydride (60% in oil, 2.87 g, 71.9 mmol). After hydrogen evolution stopped, the reaction mixture was left to attain RT overnight. Water (85 ml) was added to the reaction mixture, then acidified till pH=1, the precipitate was filtered and dried. The solid was triturated in hot AcCN and then left to cool down, it was then filtered to give the desired product ethyl 1-(4-bromophenyl)-7-hydroxy-5-oxo-4,5-dihydro-1H-pyrrolo[3,2-b]pyridine-6-carboxylate (4.4 g, 11.67 mmol, 97% yield) as a cream powder. ¹H NMR: (DMSO-d6, 400 Hz) δ 14.18 (br s, 1H), 11.51 (br s, 1H), 7.66 (d, J=8.1 Hz, 2H), 7.54 (m, 1H), 7.50 (m, 1H), 7.43 (d, J=8.1 Hz, 2H), 6.16 (m, 1H), 4.30 (q, J=7.0 Hz, 2H), 1.27 (t, J=7.0 Hz, 3H). LCMS: (M+H)⁺: 379; Rt: 3.04 min.

Method B: To a solution of ethyl 1-(4-bromophenyl)-3-{[3-(ethyloxy)-3-oxopropanoyl]amino}-1H-pyrrole-2-carboxylate (Intermediate 5) (5.07 g, 11.98 mmol) in DMF (50 mL) at 0° C. was added portionwise sodium hydride (60% in oil, 2.87 g, 71.9 mmol). After hydrogen evolution stopped, the reaction mixture was left to attain RT overnight. Water (85 mL) was added to the reaction mixture, then acidified to pH 1, the precipitate was filtered and dried. The solid was triturated in hot acetonitrile and then left to cool down, it was then filtered to give the desired product ethyl 1-(4-bromophenyl)-7-hydroxy-5-oxo-4,5-dihydro-1H-pyrrolo[3,2-b]pyridine-6-carboxylate (4.4 g, 11.67 mmol, 97% yield) as a cream powder. LCMS: (M+H)⁺: 377, 379; Rt: 3.04 min.

### Intermediate 7

### Ethyl 3-(acetylamino)-5-chloro-1H-pyrrole-2-carboxylate

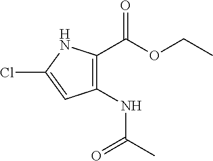

Method A: Ethyl 3-(acetylamino)-1H-pyrrole-2-carboxylate (Intermediate 1) (40 g, 204 mmol) was dissolved in chloroform (250 mL) and N-chlorosuccinimide (NCS) (28.6 g, 214 mmol) was added portion-wise. The mixture was stirred at RT for 1 h, and after TLC, was warmed to 35° C. during 2 hours. The mixture was then poured into water and extracted with DCM, dried over Na₂SO₄, and concentrated in vacuo. The mixture was triturated in DCM and the precipitate was filtered, washed with a small of DCM and washed with Et₂O to give ethyl 3-(acetylamino)-5-chloro-1H-pyrrole-2-carboxylate (20 g, 42% yield) as a white solid. ¹H NMR: (DMSO-d6, 300 Hz) δ 12.45 (br s, 1H), 9.23 (s, 1H), 6.68 (s, 1H), 4.27 (q, J=7.1 Hz, 2H), 2.08 (s, 3H), 1.30 (t, J=7.1 Hz, 3H). LCMS: (M+H)⁺: 231; Rt: 2.32 min.

Method B: To a solution of ethyl 3-(acetylamino)-1H-pyrrole-2-carboxylate (Intermediate 1) (10 g, 51.0 mmol) in chloroform (150 mL) was added slowly N-chlorosuccinimide (NCS) (7.49 g, 56.1 mmol) and the reaction mixture was stirred at RT for 48 h. Water was added and the product was extracted with DCM. The organic layer was dried over Na₂SO₄, filtered and evaporated off. The residue was purified by chromatography on silica gel eluting with DCM/MeOH 100/0 to 90/10 to give the product ethyl 3-(acetylamino)-5-chloro-1H-pyrrole-2-carboxylate (5.8 g, 25.1 mmol, 49.3% yield) as white powder. LCMS: (M+H)⁺: 231; Rt: 2.30 min.

### Intermediate 8

### Ethyl 3-(acetylamino)-1-(4-bromophenyl)-5-chloro-1H-pyrrole-2-carboxylate

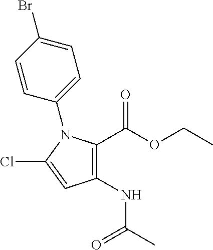

Method A: To a suspension of ethyl 3-(acetylamino)-5-chloro-1H-pyrrole-2-carboxylate (Intermediate 7) (200 mg, 0.867 mmol) and molecular sieves 4A (500 mg, 0.867 mmol) in DCM (5 mL) was added (4-bromophenyl)boronic acid (192 mg, 0.954 mmol), copper(II) acetate (173 mg, 0.954 mmol) and Et₃N (0.181 mL, 1.301 mmol). The reaction mixture was stirred at room temperature overnight. TLC and LCMS showed the reaction was incomplete. More (4-bromophenyl)boronic acid (192 mg, 0.954 mmol) was added every 2 hours until the reaction was complete (4 equiv. In total). The mixture was filtered on silica pad (DCM and MeOH) and the brown filtrate was concentrated. The residue was purified by chromatography on silica gel (interchim 12 g) (DCM/MeOH 100/0 to 99/1) to give the product ethyl 3-(acetylamino)-1-(4-bromophenyl)-5-chloro-1H-pyrrole-2-carboxylate (350 mg, 0.862 mmol, 99% yield) as a light yellow oil. ¹H NMR: (DMSO-d6, 300 Hz) δ 9.50 (s, 1H), 7.70 (m, 2H), 7.30 (m, 2H), 7.01 (s, 1H), 3.98 (q, J=7.1 Hz, 2H), 2.13 (s, 3H), 0.91 (t, J=7.1 Hz, 3H). LCMS: (M+H)⁺: 385, 387; Rt: 3.83 min.

Method B: To a suspension of ethyl 3-(acetylamino)-5-chloro-1H-pyrrole-2-carboxylate (Intermediate 7) (5.8 g, 25.1 mmol) in DCM (250 mL) was added (4-bromophenyl)boronic acid (5.56 g, 27.7 mmol), copper(II) acetate (5.0 g, 27.7 mmol) and pyridine (3.05 mL, 37.7 mmol). The reaction mixture was stirred at room temperature for two days. (4-Bromophenyl)boronic acid (5.6 g, 27.7 mmol), copper(II) acetate (5.0 g, 27.7 mmol) and pyridine (3.05 mL, 37.7 mmol) were added again every 2 days (4.4 equiv. in total). Water was added and the product was extracted with DCM. The organic layer was dried over Na₂SO₄ filtered and evaporated off. The residue was purified by chromatography on silica gel eluting with DCM/MeOH 100/0 to 90/10 to give the product ethyl 3-(acetylamino)-1-(4-bromophenyl)-5-chloro-1H-pyrrole-2-carboxylate (2.8 g, 6.90 mmol, 27.4% yield) as a yellow oil. LCMS: (M+H)⁺: 387; Rt: 3.48 min.

### Intermediate 9

### Ethyl 3-amino-1-(4-bromophenyl)-5-chloro-1H-pyrrole-2-carboxylate

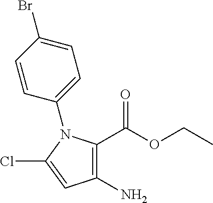

Method A: To a solution of ethyl 3-(acetylamino)-1-(4-bromophenyl)-5-chloro-1H-pyrrole-2-carboxylate (Intermediate 8) (9 g, 23.34 mmol) in ethanol (200 mL) was added concentrated HCl (9.58 mL, 117 mmol). The mixture was refluxed for 2 hours before being concentrated in vacuo and the precipitate was triturated in diethyl ether and filtered to give ethyl 3-amino-1-(4-bromophenyl)-5-chloro-1H-pyrrole-2-carboxylate (8.53 g, 96% yield) as a white solid. ¹H NMR: (DMSO-d6, 300 Hz) δ 7.67 (d, J=8.6 Hz, 2H), 7.24 (d, J=8.6 Hz, 2H), 6.17 (s, 1H), 3.97 (q, J=7.1 Hz, 2H), 0.98 (t, J=7.1 Hz, 3H). LCMS: (M+H)⁺: 343, 345; Rt: 3.74 min.

Method B: To a solution of ethyl 3-(acetylamino)-1-(4-bromophenyl)-5-chloro-1H-pyrrole-2-carboxylate (Intermediate 8) (1.3 g, 3.37 mmol) in ethanol (20 mL) was added concentrated HCl (1.38 mL, 16.9 mmol). The mixture was refluxed for 2 hours before being concentrated in vacuo and the precipitate was triturated in diethyl ether and filtered to give ethyl 3-amino-1-(4-bromophenyl)-5-chloro-1H-pyrrole-2-carboxylate (820 mg, 64% yield) as a white solid. LCMS: (M+H)⁺: 345; Rt: 3.42 min.

### Intermediate 10

### Ethyl 1-(4-bromophenyl)-5-chloro-3-[(cyanoacetyl)amino]-1H-pyrrole-2-carboxylate

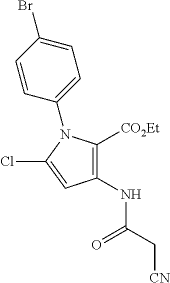

Method A: To a solution of ethyl 3-amino-1-(4-bromophenyl)-5-chloro-1H-pyrrole-2-carboxylate (Intermediate 9) (7.5 g, 19.73 mmol), and triethylamine (8.25 mL, 59.2 mmol) in DCM (100 mL) at RT was added cyanoacetic acid (3.36 g, 39.5 mmol), EDCl (4.54 g, 23.68 mmol) and HOBt (3.63 g, 23.68 mmol). The reaction mixture was stirred at RT overnight. Cyanoacetic acid (1 equiv.), HOBt (1 equiv.) and EDCl (1 equiv.) were added again and the reaction mixture was stirred for another 2 hours at RT. The reaction mixture was washed with 1N HCl, 1N NaOH, and sat NaCl. The organic layer was dried over anhydrous Na₂SO₄, filtered and concentrated in vacuo. The mixture was triturated in MeOH to give ethyl 1-(4-bromophenyl)-5-chloro-3-[(cyanoacetyl)amino]-1H-pyrrole-2-carboxylate (5.66 g, 66% yield) as an off-white solid. ¹H NMR: (DMSO-d6, 300 Hz) δ 9.84 (s, 1H), 7.72 (d, J=8.7 Hz, 2H), 7.33 (d, J=8.7 Hz, 2H), 6.99 (s, 1H), 4.14 (s, 2H), 4.01 (q, J=7.1 Hz, 2H), 0.92 (t, J=7.1 Hz, 3H). LCMS: (M+H)⁺: 411, 413; Rt: 3.82 min.

Method B: To a solution of ethyl 3-amino-1-(4-bromophenyl)-5-chloro-1H-pyrrole-2-carboxylate (Intermediate 9) (2.5 g, 6.58 mmol) and triethylamine (1.0 mL, 7.24 mmol) in acetonitrile (150 mL) were added cyanoacetic acid (0.839 g, 9.87 mmol) and DCC (2.239 g, 10.85 mmol). The mixture was stirred at 50° C. for 24 h before being filtered. The filtrate was evaporated off to give after purification by chromatography on silica gel eluting with DCM/MeOH 100/0 to 95/5 the desired product ethyl 1-(4-bromophenyl)-5-chloro-3-[(cyanoacetyl)amino]-1H-pyrrole-2-carboxylate (2.6 g, 6.33 mmol, 96% yield) as a yellow oil. LCMS: (M−H)⁻: 410; Rt: 3.63 min.

### Intermediate 11

### Ethyl 3-{[(4-fluorophenyl)acetyl]amino}-1H-pyrrole-2-carboxylate

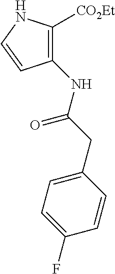

To a suspension of ethyl 3-amino-1H-pyrrole-2-carboxylate (2 g, 10.49 mmol) and triethylamine (3.21 mL, 23.08 mmol) in DCM (20 mL) at 0° C. was added drop-wise (4-fluorophenyl)acetyl chloride (1.811 g, 10.49 mmol). The reaction mixture was then stirred from 0° C. to RT for 1 h before being quenched with 1N HCl. The organic layer was separated and washed successively with sat. NaHCO₃ and brine, dried over Na₂SO₄, filtered and concentrated under reduced pressure to give ethyl 3-{[(4-fluorophenyl)acetyl]amino}-1H-pyrrole-2-carboxylate (3.52 g, 12.13 mmol, 116% yield) as an orange solid. LCMS: (M+H)⁺: 291; Rt: 2.83 min.

### Intermediate 12

### Ethyl 1-(4-bromophenyl)-3-{[(4-fluorophenyl)acetyl]amino}-1H-pyrrole-2-carboxylate

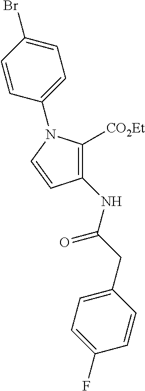

Copper(II) acetate (3.34 g, 18.40 mmol) was added to a solution of 4-bromophenylboronic acid (3.69 g, 18.40 mmol), ethyl 3-{[(4-fluorophenyl)acetyl]amino}-1H-pyrrole-2-carboxylate (Intermediate 11) (3.56 g, 12.26 mmol) and pyridine (2.97 mL, 36.8 mmol) in DCM (100 mL) at RT. The reaction mixture was stirred for 24 h before being quenched with 1N HCl (50 ml). The precipitate was filtered through a pad of Celite and washed with ethyl acetate. The organic layer was then dried over Na₂SO₄, concentrated under reduced pressure and purified by chromatography on a Biotage. The sample was loaded on 100 g Biotage silica (Si) column then the purification was carried out using cyclohexane/AcOEt 100/0 to 80/20. The appropriate fractions were combined and concentrated in vacuo to give the required product ethyl 1-(4-bromophenyl)-3-{[(4-fluorophenyl)acetyl]amino}-1H-pyrrole-2-carboxylate (2.9 g, 6.51 mmol, 53.1% yield) as a cream solid. LCMS: (M+H)⁺: 445, 447; Rt: 3.81 min.

### Intermediate 13

### Ethyl 3-({[4-(methyloxy)phenyl]acetyl}amino)-1H-pyrrole-2-carboxylate

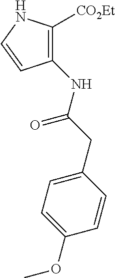

To a solution of [4-(methyloxy)phenyl]acetic acid (2.156 g, 12.97 mmol), ethyl 3-amino-1H-pyrrole-2-carboxylate (2 g, 12.97 mmol) and triethylamine (1.989 mL, 14.27 mmol) in acetonitrile (60 mL) at RT was added drop-wise a solution of DCC (4.28 g, 20.76 mmol) in acetonitrile (20 mL). The reaction mixture was stirred for 2 h before being filtered. The filtrate was concentrated under reduced pressure and extracted with EtOAc/water then washed successively with sat. NaHCO₃ and brine. The organic layer was dried over Na₂SO₄ and evaporated. The crude solid was dissolved in DCM and precipitated with cyclohexane, filtered and dried to give the desired product ethyl 3-({[4-(methyloxy)phenyl]acetyl}amino)-1H-pyrrole-2-carboxylate (1.5 g, 4.96 mmol, 38.2% yield) as a cream powder. LCMS: (M+H)⁺: 303; Rt: 2.83 min.

### Intermediate 14

### Ethyl 1-(4-bromophenyl)-3-({[4-(methyloxy)phenyl]acetyl}amino)-1H-pyrrole-2-carboxylate

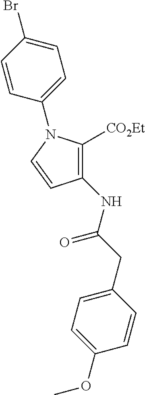

Copper(II) acetate (1.352 g, 7.44 mmol) was added to a solution of 4-bromophenylboronic acid (1.495 g, 7.44 mmol), ethyl 3-({[4-(methyloxy)phenyl]acetyl}amino)-1H-pyrrole-2-carboxylate (Intermediate 13) (1.5 g, 4.96 mmol) and pyridine (1.201 mL, 14.88 mmol) in DCM (100 mL) at RT. The reaction mixture was stirred for 24 h before being quenched with 15 ml of HCl 1N. The resulting solid was filtered through a pad of Celite and washed with DCM (110 mL). The organic layer was dried over Na₂SO₄, concentrated under reduced pressure and then purified by chromatography on a Biotage SP4. The sample was loaded on 100 g Biotage silica (Si) column and the purification carried out using cyclohexane/AcOEt 100/0 to 80/20. The appropriate fractions were combined and concentrated in vacuo to give the required product ethyl 1-(4-bromophenyl)-3-({[4-(methyloxy)phenyl]acetyl}amino)-1H-pyrrole-2-carboxylate (1 g, 2.187 mmol, 44.1% yield) as a cream solid. LCMS: (M+H)⁺: 457, 459; Rt: 3.72 min.

### Intermediate 15

### Ethyl 1-(4-bromophenyl)-3-{[4-cyanophenyl)acetyl]amino}-1H-pyrrole-2-carboxylate

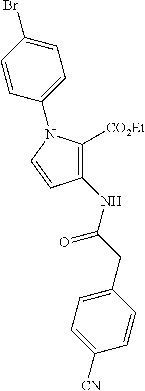

To a solution of (4-cyanophenyl)acetic acid (1.043 g, 6.47 mmol), ethyl 3-amino-1-(4-bromophenyl)-1H-pyrrole-2-carboxylate (Intermediate 3) (2 g, 6.47 mmol) and triethylamine (0.992 mL, 7.12 mmol) in acetonitrile (30 mL) at RT was added drop-wise a solution of DCC (2.002 g, 9.70 mmol) in acetonitrile (5 mL). The reaction mixture was stirred for 2 h before being filtered off. The filtrate was concentrated under reduced pressure and extracted with EtOAc/water then washed successively with sat. NaHCO₃ and brine. The organic layer was dried over Na₂SO₄ and evaporated. The product was dissolved in DCM and precipitated in cyclohexane before being filtered and dried to give ethyl 1-(4-bromophenyl)-3-{[(4-cyanophenyl)acetyl]amino}-1H-pyrrole-2-carboxylate (1.46 g, 3.23 mmol, 49.9% yield) as a white solid. LCMS: (M+H)⁺: 452, 454; Rt: 3.74 min.

### Intermediate 16

### 4-[1-(4-Bromophenyl)-7-hydroxy-5-oxo-4,5-dihydro-1H-pyrrolo[3,2-b]pyridin-6-yl]benzonitrile

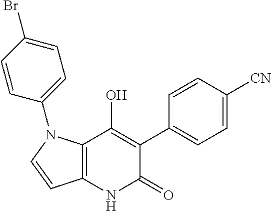

DMSO (1.5 mL) was added to a mixture of ethyl 1-(4-bromophenyl)-3-{[(4-cyanophenyl)acetyl]amino}-1H-pyrrole-2-carboxylate (Intermediate 15) (800 mg, 1.769 mmol) and potassium tert-butoxide (397 mg, 3.54 mmol). The reaction mixture was then stirred at 100° C. overnight before water was added. The resulting solid was filtered and dried to give 4-[1-(4-bromophenyl)-7-hydroxy-5-oxo-4,5-dihydro-1H-pyrrolo[3,2-b]pyridin-6-yl]benzonitrile (461 mg, 1.135 mmol, 64.2% yield) as a yellow solid. LCMS: (M+H)⁺: 406, 408; Rt: 2.37 min.

### Intermediate 17

### Ethyl 1-(4-bromophenyl)-3-[(phenylacetyl)amino]-1H-pyrrole-2-carboxylate

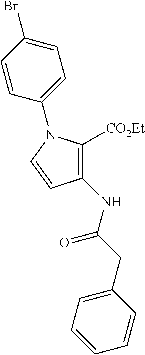

To a suspension of ethyl 3-amino-1-(4-bromophenyl)-1H-pyrrole-2-carboxylate (Intermediate 3) (2.5 g, 8.09 mmol) and triethylamine (2.473 mL, 17.79 mmol) in DCM (50 mL) at 0° C. was added drop-wise phenylacetyl chloride (1.283 mL, 9.70 mmol). The reaction mixture was then stirred from 0° C. to RT for 1 h before being quenched with 1N HCl. The organic layer was separated and washed successively with sat. NaHCO₃ and brine, dried over Na₂SO₄. The solvent was removed under reduced pressure and the product was purified using Biotage (cyclohexane/ethyl acetate 100/0 to 70/30 as eluant) The appropriate fractions were combined and concentrated in vacuo to give ethyl 1-(4-bromophenyl)-3-[(phenylacetyl)amino]-1H-pyrrole-2-carboxylate (2.6 g, 6.08 mmol, 75% yield) as a yellow oil. LCMS: (M+H)⁺: 427, 429; Rt: 3.76 min.

### Intermediate 18

### 1-(4-Bromophenyl)-7-hydroxy-6-phenyl-1,4-dihydro-5H-pyrrolo[3,2-b]pyridin-5-one

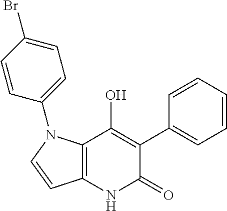

Dimethyl Sulfoxide (DMSO) (3.750 mL) was added to a mixture of ethyl 1-(4-bromophenyl)-3-[(phenylacetyl)amino]-1H-pyrrole-2-carboxylate (Intermediate 17) (1.1 g, 2.57 mmol) and potassium tert-butoxide (0.578 g, 5.15 mmol). The reaction mixture was then stirred at 100° C. overnight before being quenched with 1N HCl (2 mL). Water was added and the resulting solid was filtered and dried to give 1-(4-bromophenyl)-7-hydroxy-6-phenyl-1,4-dihydro-5H-pyrrolo[3,2-b]pyridin-5-one (890 mg, 2.335 mmol, 91% yield) as a brown solid. LCMS: (M+H)⁺: 381, 383; Rt: 2.60 min.

### Intermediate 19

### Ethyl 3-(acetylamino)-1-(4-acetylphenyl)-1H-pyrrole-2-carboxylate

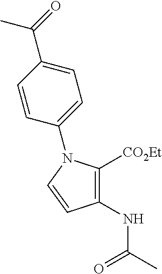

A suspension of ethyl 3-(acetylamino)-1H-pyrrole-2-carboxylate (Intermediate 1) (5 g, 25.5 mmol), 1-(4-bromophenyl)ethanone (10.14 g, 51.0 mmol), tripotassium phosphate (16.23 g, 76 mmol) and N,N′-dimethyl-1,2-ethanediamine (1.646 mL, 15.29 mmol) in toluene (130 mL) was degassed with argon for 20 min before copper(I) iodide (2.427 g, 12.74 mmol) was added. The reaction mixture was then stirred at reflux overnight under atmosphere of argon before being filtered through Celite and concentrated to dryness. The crude mixture was taken up in EtOAc and washed successively with 1N HCl and brine. The organic layers was dried over Na₂SO₄, filtered and concentrated under reduced pressure. The crude brown oil was then purified by chromatography on silica gel eluting with cyclohexane/EtOAc 100/0 to 60/40. The appropriate fractions were combined and concentrated in vacuo to give the required product ethyl 3-(acetylamino)-1-(4-acetylphenyl)-1H-pyrrole-2-carboxylate (5.75 g, 18.29 mmol, 71.8% yield) as a light yellow solid. LCMS: (M+H)⁺: 315; Rt: 2.81 min.

### Intermediate 20

### Ethyl 3-(acetylamino)-1-[4-(methyloxy)phenyl]-1H-pyrrole-2-carboxylate

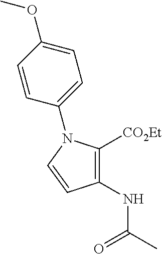

Method A: Copper(II) acetate (1.018 g, 5.61 mmol) was added to a solution of [4-(methyloxy)phenyl]boronic acid (0.852 g, 5.61 mmol), pyrrole (1 g, 5.10 mmol) and pyridine (0.617 mL, 7.65 mmol) in DCM (25 mL) at RT. The reaction mixture was stirred for 18 h and monitoring of the reaction by LCMS showed the reaction was incomplete with SM remaining. [4-(methyloxy)phenyl]boronic acid (0.852 g, 5.61 mmol), copper(II) acetate (1.018 g, 5.61 mmol) and pyridine (0.617 mL, 7.65 mmol) were added again in the same order and the mixture was stirred another 24 h. Monitoring of the reaction by LCMS showed the reaction was incomplete with SM remaining and all the reactants were added again every day for 7 days. The reaction mixture was concentrated to dryness and the resulting solid was taken up in EtOAc and filtered through Celite. The filtrate was then washed respectively with 1N HCl and brine. The organic layers was dried over Na₂SO₄, filtered and concentrated under reduced pressure. The crude was then purified by chromatography on a Isco Companion RF. The sample was loaded on 50 g Biotage SNAP silica column then the purification was carried out using a cyclohexane/AcOEt 100/0 to 50/50. The appropriate fractions were combined and concentrated in vacuo to give the required product ethyl 3-(acetylamino)-1-[4-(methyloxy)phenyl]-1H-pyrrole-2-carboxylate (1.05 g, 3.47 mmol, 68.1% yield) as an orange solid. LCMS: (M+H)⁺: 303; Rt: 3.13 min.

Method B: Copper(II) acetate (1.018 g, 5.61 mmol) was added to a solution of [4-(methyloxy)phenyl]boronic acid (0.852 g, 5.61 mmol), ethyl 3-(acetylamino)-1H-pyrrole-2-carboxylate (Intermediate 1) (1 g, 5.10 mmol) and pyridine (0.617 mL, 7.65 mmol) in DCM (25 mL) at RT. The reaction mixture was stirred for 18 h and [4-(methyloxy)phenyl]boronic acid (0.852 g, 5.61 mmol), copper(II) acetate (1.018 g, 5.61 mmol) and pyridine (0.617 mL, 7.65 mmol) were added again in the same order. The mixture was stirred another 24 h and all the reactants were added again every day for 7 days (7.7 equiv. in total). The reaction mixture was concentrated to dryness and the resulting solid was taken up in EtOAc and filtered through Celite. The filtrate was then washed respectively with 1N HCl and brine. The organic layers was dried over Na₂SO₄, filtered and concentrated under reduced pressure. The crude was then purified by chromatography on silica gel eluting with cyclohexane/EtOAc 100/0 to 50/50. The appropriate fractions were combined and concentrated in vacuo to give the required product ethyl 3-(acetylamino)-1-[4-(methyloxy)phenyl]-1H-pyrrole-2-carboxylate (1.05 g, 3.47 mmol, 68.1% yield) as an orange solid. LCMS: (M+H)⁺: 303; Rt: 3.13 min.

Intermediates 21 to 29 of formula (VIII), wherein R³ is H, were prepared by methods analogous to that described for Intermediate 20 from Intermediate 1 using the appropriate boronic acid.

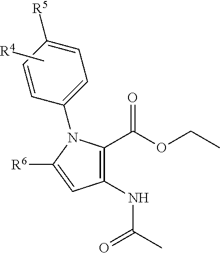

TABLE 1

| Intermediate | Name | R⁴ | R⁵ | R⁶ | Physical data |
|---|---|---|---|---|---|
| 21 | Ethyl 3-(acetylamino)-1-(4- | H | Et | H | ¹H NMR: (CDCl₃, |
| | ethylphenyl)-1H-pyrrole-2- | | | | 400 MHz) δ 9.55 (br s, |
| | carboxylate | | | | 1H), 7.22 (m, 2H), 7.18- |
| | | | | | 7.14 (m, 3H), 6.80 (m, |
| | | | | | 1H), 4.08 (q, J = 7.2 Hz, |
| | | | | | 2H), 2.71 (q, J = 7.6 Hz, |
| | | | | | 2H), 2.21 (s, 3H), 1.28 (t, |
| | | | | | J = 7.6 Hz, 3H), 0.97 (t, |
| | | | | | J = 7.2 Hz, 3H) |
| 22 | Ethyl 3-(acetylamino)-1-(4- | H | F | H | LCMS: (M + H)⁺: 291; |
| | fluorophenyl)-1H-pyrrole-2- | | | | Rt: 3.11 min. |
| | carboxylate | | | | |
| 23 | Ethyl 3-(acetylamino)-1-[3- | 3-OMe | H | H | LCMS: (M + H)⁺: 303; |
| | (methyloxy)phenyl]-1H- | | | | Rt: 3.12 min |
| | pyrrole-2-carboxylate | | | | |
| 24 | Ethyl 3-(acetylamino)-1-(4- | H | Me | H | LCMS: (M + H)⁺: 287; |
| | methylphenyl)-1H-pyrrole-2- | | | | Rt: 3.33 min |
| | carboxylate | | | | |
| 25 | Ethyl 3-(acetylamino)-1-[4- | H | CF₃ | H | LCMS: (M + H)⁺: 341; |
| | (trifluoromethyl)phenyl]-1H- | | | | Rt: 3.17 min |
| | pyrrole-2-carboxylate | | | | |
| 26 | Ethyl 3-(acetylamino)-1-{4- | H | OCF₃ | H | LCMS: (M + H)⁺: 357; |
| | [(trifluoromethyl)oxy]phenyl}- | | | | Rt: 3.53 min |
| | 1H-pyrrole-2-carboxylate | | | | |
| 27 | Ethyl 3-(acetylamino)-5- | 3-Me | Me | Cl | LCMS: (M + H)⁺: 335; |
| | chloro-1-(3,4- | | | | Rt: 3.82 min |
| | dimethylphenyl)-1H-pyrrole- | | | | |
| | 2-carboxylate | | | | |
| 28 | Ethyl 3-(acetylamino)-1-[4- | H | CH₂CN | H | LCMS: (M + H)⁺: 312; |
| | (cyanomethyl)phenyl]-1H- | | | | Rt: 2.89 min |
| | pyrrole-2-carboxylate | | | | |
| 29 | Ethyl 3-(acetylamino)-1-(4- cyclohexylphenyl)-1H- pyrrole-2-carboxylate | H | 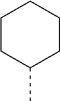 | H | LCMS: (M + H)⁺: 355; Rt: 4.23 min. |

### Intermediate 30

### Ethyl 3-(acetylamino)-5-chloro-1-[4-(methyloxy)phenyl]-1H-pyrrole-2-carboxylate

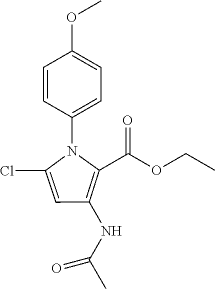

To a solution of ethyl 3-(acetylamino)-1-[4-(methyloxy)phenyl]-1H-pyrrole-2-carboxylate (Intermediate 20) (3.26 g, 10.78 mmol) in tetrahydrofuran (THF) (100 mL) at RT was added NCS (1.512 g, 11.32 mmol). The mixture was stirred at RT for 18 h before being concentrated to dryness. The mixture was taken up in DCM, washed with water, dried over Na₂SO₄, filtered and concentrated under reduced pressure. The crude product was then purified by chromatography on an Isco Companion RF. The sample was loaded on 100 g Biotage SNAP silica column then the purification was carried out using a cyclohexane/EtOAc 95/5 to 70/30. The appropriate fractions were combined and concentrated in vacuo to give the required product ethyl 3-(acetylamino)-5-chloro-1-[4-(methyloxy)phenyl]-1H-pyrrole-2-carboxylate (3.12 g, 9.26 mmol, 86% yield) as a yellow oil. LCMS: (M+H)⁺: 337; Rt: 3.41 min.

Intermediates 31 to 39 of formula (VIII), wherein R³ is H and R⁶ is Cl, were prepared by methods analogous to that described for Intermediate 30 using NCS.

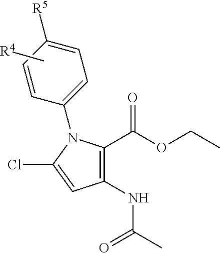

TABLE 2

| | | From | | | |
|---|---|---|---|---|---|
| Intermediate | Name | Int. | R⁴ | R⁵ | Physical data |
| 31 | Ethyl 3-(acetylamino)-1- | 19 | H | Ac | LCMS: (M + H)⁺: 349; |
| | (4-acetylphenyl)-5- | | | | Rt: 3.13 min. |
| | chloro-1H-pyrrole-2- | | | | |
| | carboxylate | | | | |
| 32 | Ethyl 3-(acetylamino)-5- | 21 | H | Et | ¹H NMR: (CDCl₃, 400 MHz) |
| | chloro-1-(4-ethylphenyl)- | | | | δ 9.62 (br s, 1H), 7.27 (m, |
| | 1H-pyrrole-2-carboxylate | | | | 2H), 7.16 (s, 1H), 7.11 (m, |
| | | | | | 2H), 3.99 (q, J = 7.1 Hz, |
| | | | | | 2H), 2.73 (q, J = 7.6 Hz, |
| | | | | | 2H), 2.20 (s, 3H), 1.29 (t, |
| | | | | | J = 7.6 Hz, 3H), 0.86 (t, J = |
| | | | | | 7.1 Hz, 3H). |
| 33 | Ethyl 3-(acetylamino)-5- | 22 | H | F | LCMS: (M + H)⁺: 325; |
| | chloro-1-(4- | | | | Rt: 3.47 min. |
| | fluorophenyl)-1H- | | | | |
| | pyrrole-2-carboxylate | | | | |
| 34 | Ethyl 3-(acetylamino)-5- | 23 | 3-OMe | H | LCMS: (M + H)⁺: 337; |
| | chloro-1-[3- | | | | Rt: 3.40 min. |
| | (methyloxy)phenyl]-1H- | | | | |
| | pyrrole-2-carboxylate | | | | |
| 35 | Ethyl 3-(acetylamino)-5- | 24 | H | Me | LCMS: (M + H)⁺: 321; |
| | chloro-1-(4- | | | | Rt: 3.64 min. |
| | methylphenyl)-1H- | | | | |
| | pyrrole-2-carboxylate | | | | |
| 36 | Ethyl 3-(acetylamino)-5- | 25 | H | CF₃ | LCMS: (M + H)⁺: 375; |
| | chloro-1-[4- | | | | Rt: 3.75 min. |
| | (trifluoromethyl)phenyl]- | | | | |
| | 1H-pyrrole-2-carboxylate | | | | |
| 37 | Ethyl 3-(acetylamino)-5- | 26 | H | OCF₃ | LCMS: (M + H)⁺: 391; |
| | chloro-1-{4- | | | | Rt: 3.80 min. |
| | [(trifluoromethyl)oxy] | | | | |
| | phenyl}-1H-pyrrole-2- | | | | |
| | carboxylate | | | | |
| 38 | Ethyl 3-(acetylamino)-5- | 28 | H | CH₂CN | LCMS: (M + H)⁺: 346; |
| | chloro-1-[4- | | | | Rt: 3.08 min. |
| | (cyanomethyl)phenyl]- | | | | |
| | 1H-pyrrole-2-carboxylate | | | | |
| 39 | Ethyl 3-(acetylamino)-5- chloro-1-(4- cyclohexylphenyl)-1H- pyrrole-2-carboxylate | 29 | H | 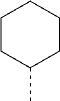 | LCMS: (M + H)⁺: 389; Rt: 4.31 min. |

### Intermediate 40

### Ethyl 3-(acetylamino)-5-cyano-1-(4-ethylphenyl)-1H-pyrrole-2-carboxylate

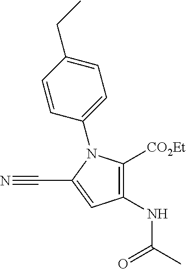

To a suspension of ethyl 3-(acetylamino)-5-chloro-1-(4-ethylphenyl)-1H-pyrrole-2-carboxylate (Intermediate 32) (500 mg, 1.49 mmol), zinc cyanide (105 mg, 0.89 mmol), zinc powder (19 mg, 0.298 mmol) and 2-(di-t-butylphosphino)-1,1′-binapthyl (178 mg, 0.447 mmol) in N,N-dimethylacetamide (7.5 mL) was added trifluoroacetate palladium (II) (49 mg, 0.149 mmol) under argon atmosphere. The reaction mixture was stirred for 16 h at 95° C. before being quenched with sat. NaHCO₃. The aqueous layer was extracted with EtOAc and washed with brine. The product was purified by chromatography on silica gel eluting with petroleum ether/EtOAc 80/20 to give ethyl 3-(acetylamino)-5-cyano-1-(4-ethylphenyl)-1H-pyrrole-2-carboxylate (170 mg, 30% yield) as a white solid. ¹H NMR: (CDCl₃, 400 MHz) δ 9.45 (br s, 1H), 7.75 (s, 1H), 7.29 (m, 2H), 7.18 (m, 2H), 4.06 (q, J=7.1 Hz, 2H), 2.74 (q, J=7.6 Hz, 2H), 2.22 (s, 3H), 1.28 (t, J=7.6 Hz, 3H), 0.90 (t, J=7.1 Hz, 3H).

### Intermediate 41

### Ethyl 3-amino-1-[4-(methyloxy)phenyl]-1H-pyrrole-2-carboxylate

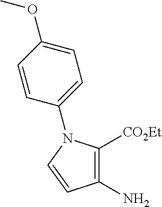

A solution of ethyl 3-(acetylamino)-1-[4-(methyloxy)phenyl]-1H-pyrrole-2-carboxylate (Intermediate 20) (1.05 g, 3.47 mmol) and concentrated HCl (1.426 mL, 17.37 mmol) in ethanol (30 mL) was refluxed for 18 h before being concentrated under reduced pressure. The crude solid was triturated in CH₃CN and the solid filtrated and dried to give the desired compound ethyl 3-amino-1-[4-(methyloxy)phenyl]-1H-pyrrole-2-carboxylate hydrochloride (870 mg, 2.93 mmol, 84% yield) as an off-white solid. LCMS: (M+H)⁺: 261; Rt: 2.82 min.

Intermediates 42 to 53 of formula (V), wherein R³ is H, were prepared by methods analogous to that described for Intermediate 41.

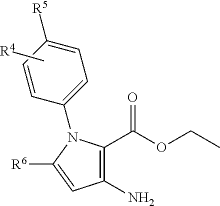

TABLE 3

| | | From | | | | |
|---|---|---|---|---|---|---|
| Intermediate | Name | Int. | R⁴ | R⁵ | R⁶ | Physical data |
| 42 | Ethyl 3-amino-5-chloro-1-[4- | 30 | H | OMe | Cl | LCMS: (M + H)⁺: 295; |
| | (methyloxy)phenyl]-1H- | | | | | Rt: 3.28 min. |
| | pyrrole-2-carboxylate | | | | | |
| | hydrochloride | | | | | |
| 43 | Ethyl 1-(4-acetylphenyl)-3- | 31 | H | Ac | Cl | LCMS: (M + H)⁺: 307; |
| | amino-5-chloro-1H-pyrrole- | | | | | Rt: 3.01 min. |
| | 2-carboxylate hydrochloride | | | | | |
| 44 | Ethyl 3-amino-5-chloro-1-(4- | 32 | H | Et | Cl | ¹H NMR: (CDCl₃, |
| | ethylphenyl)-1H-pyrrole-2- | | | | | 400 MHz) δ 7.31 (m, |
| | carboxylate hydrochloride | | | | | 2H), 7.14 (m, 2H), |
| | | | | | | 6.87 (m, 1H), 4.22 |
| | | | | | | (q, J = 7.1 Hz, 2H), |
| | | | | | | 2.75 (q, J = 7.6 Hz, |
| | | | | | | 2H), 1.30 (t, J = 7.6 |
| | | | | | | Hz, 3H), 1.09 (t, J = |
| | | | | | | 7.1 Hz, 3H). |
| 45 | Ethyl 3-amino-5-chloro-1-(4- | 33 | H | F | Cl | LCMS: (M + H)⁺: 283; |
| | fluorophenyl)-1H-pyrrole-2- | | | | | Rt: 3.36 min. |
| | carboxylate hydrochloride | | | | | |
| 46 | Ethyl 3-amino-5-chloro-1-[3- | 34 | 3-OMe | H | Cl | LCMS: (M + H)⁺: 295; |
| | (methyloxy)phenyl]-1H- | | | | | Rt: 3.30 min. |
| | pyrrole-2-carboxylate | | | | | |
| | hydrochloride | | | | | |
| 47 | Ethyl 3-amino-5-chloro-1-(4- | 35 | H | Me | Cl | LCMS: (M + H)⁺: 279; |
| | methylphenyl)-1H-pyrrole-2- | | | | | Rt: 3.48 min. |
| | carboxylate hydrochloride | | | | | |
| 48 | Ethyl 3-amino-5-chloro-1-[4- | 36 | H | CF₃ | Cl | LCMS: (M + H)⁺: 333; |
| | (trifluoromethyl)phenyl]-1H- | | | | | Rt: 3.68 min. |
| | pyrrole-2-carboxylate | | | | | |
| | hydrochloride | | | | | |
| 49 | Ethyl 3-amino-5-chloro-1-{4- | 37 | H | OCF₃ | Cl | LCMS: (M + H)⁺: 349; |
| | [(trifluoromethyl)oxy]phenyl}- | | | | | Rt: 3.73 min. |
| | 1H-pyrrole-2-carboxylate | | | | | |
| | hydrochloride | | | | | |
| 50 | Ethyl 3-amino-5-chloro-1- | 27 | 3-Me | Me | Cl | LCMS: (M + H)⁺: 293; |
| | (3,4-dimethylphenyl)-1H- | | | | | Rt: 3.69 min. |
| | pyrrole-2-carboxylate | | | | | |
| | hydrochloride | | | | | |
| 51 | Ethyl 3-amino-5-chloro-1-[4- | 38 | H | CH₂CN | Cl | LCMS: (M + H)⁺: 304; |
| | (cyanomethyl)phenyl]-1H- | | | | | Rt: 3.00 min. |
| | pyrrole-2-carboxylate | | | | | |
| | hydrochloride | | | | | |
| 52 | Ethyl 3-amino-5-chloro-1-(4- cyclohexylphenyl)-1H- pyrrole-2-carboxylate hydrochloride | 39 | H | 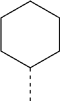 | Cl | LCMS: (M + H)⁺: 347; Rt: 4.18 min. |
| 53 | Ethyl 3-amino-5-cyano-1-(4- | 40 | H | Et | CN | ¹H NMR (400 MHz, |
| | ethylphenyl)-1H-pyrrole-2- | | | | | CDCl₃) δ ppm 7.41 |
| | carboxylate hydrochloride | | | | | (s, 1 H), 7.34 (m, 2 |
| | | | | | | H), 7.22 (m, 2 H), |
| | | | | | | 4.25 (q, J = 7.1 Hz, |
| | | | | | | 2 H), 2.76 (q, J = 7.6 |
| | | | | | | Hz, 2 H), 1.30 (t, J = |
| | | | | | | 7.6 Hz, 3 H), 1.08 (t, |
| | | | | | | J = 7.1 Hz, 3 H). |

### Intermediate 54

### Ethyl 3-amino-1-[4-(4,4,5,5-tetramethyl-1,3,2-dioxaborolan-2-yl)phenyl]-1H-pyrrole-2-carboxylate

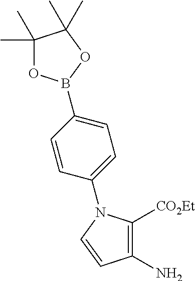

To a solution of ethyl 3-amino-1-(4-bromophenyl)-1H-pyrrole-2-carboxylate (Intermediate 3) (70 g, 226 mmol) in dioxane (800 mL) was added successively 4,4,4′,4′,5,5,5′,5′-octamethyl-2,2′-bi-1,3,2-dioxaborolane (86.3 g, 340 mmol), potassium acetate (66.6 g, 680 mmol) and Pd(dppf)Cl₂ (16.6 g, 73 mmol). The reaction mixture was stirred overnight at 100° C. before being cooled to RT and filtered. The filtrate was then concentrated to dryness and the residue was taken up in EtOAc, washed with brine, dried over Na₂SO₄, filtered and concentrated under reduced pressure. Purification by chromatography afforded ethyl 3-amino-1-[4-(4,4,5,5-tetramethyl-1,3,2-dioxaborolan-2-yl)phenyl]-1H-pyrrole-2-carboxylate (61.3 g, 76% yield). LCMS: (M+H)⁺: 357; Rt: 2.16 min.

### Intermediate 55

### Ethyl 1-(4-acetylphenyl)-5-chloro-3-[(cyanoacetyl)amino]-1H-pyrrole-2-carboxylate

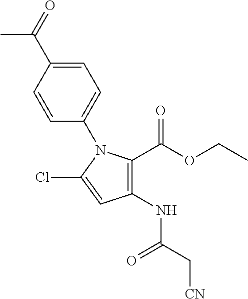

To a suspension of ethyl 1-(4-acetylphenyl)-3-amino-5-chloro-1H-pyrrole-2-carboxylate (Intermediate 43) (0.69 g, 2.010 mmol), cyanoacetic acid (0.342 g, 4.02 mmol) and triethylamine (0.560 mL, 4.02 mmol) in DCM (20 mL) at RT was added EDC (0.771 g, 4.02 mmol) and HOBT (0.616 g, 4.02 mmol). The reaction mixture was stirred from 0° C. to RT for 2 h before being washed successively with 1N HCl, sat. NaHCO₃ and brine. The organic layer was dried over anhydrous Na₂SO₄, filtered and concentrated in vacuo. The product was purified by chromatography on a Isco Companion RF. The sample was loaded on 100 g Biotage SNAP silica column then the purification was carried out using cyclohexane/EtOAc 100/0 to 60/40. The appropriate fractions were combined and concentrated in vacuo to give the required product ethyl 1-(4-acetylphenyl)-5-chloro-3-[(cyanoacetyl)amino]-1H-pyrrole-2-carboxylate (0.7 g, 1.873 mmol, 93% yield) as a yellow oil. LCMS: (M+H)⁺: 374; Rt: 3.12 min.

### Intermediate 56

### Ethyl 3-[(cyanoacetyl)amino]-1-[4-(methyloxy)phenyl]-1H-pyrrole-2-carboxylate

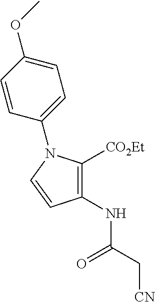

To a solution of ethyl 3-amino-1-[4-(methyloxy)phenyl]-1H-pyrrole-2-carboxylate (Intermediate 41) (200 mg, 0.674 mmol), cyanoacetic acid (86 mg, 1.011 mmol) and triethylamine (0.24 mL, 1.722 mmol) in acetonitrile (7 mL) at RT was added dropwise a solution of DCC (209 mg, 1.011 mmol) in acetonitrile (2 mL). The reaction mixture was stirred for 72 h and monitoring of the reaction by LCMS showed the reaction was complete. The filtrate was concentrated under reduced pressure. The crude solid was triturated in EtOH, filtered and dried to give the desired product ethyl 3-[(cyanoacetyl)amino]-1-[4-(methyloxy)phenyl]-1H-pyrrole-2-carboxylate (180 mg, 0.550 mmol, 82% yield) as an off-white solid. LCMS: (M+H)⁺: 328; Rt: 3.21 min.

### Intermediate 57

### Ethyl 3-[(cyanoacetyl)amino]-1-[4-(4,4,5,5-tetramethyl-1,3,2-dioxaborolan-2-yl)phenyl]-1H-pyrrole-2-carboxylate

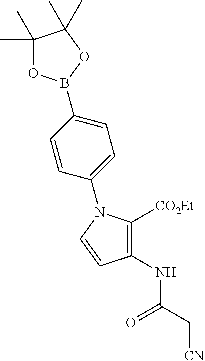

Ethyl 3-amino-1-[4-(4,4,5,5-tetramethyl-1,3,2-dioxaborolan-2-yl)phenyl]-1H-pyrrole-2-carboxylate (Intermediate 54) (10 g, 28.1 mmol) was dissolved in DMF (100 mL) and cyanoacetic acid (4.78 g, 56.1 mmol), HOBT (4.73 g, 30.9 mmol), EDC (5.92 g, 30.9 mmol) and triethylamine (9.78 mL, 70.2 mmol) were added. The mixture was stirred at RT for 2 hours before being concentrated to dryness and poured into water. The precipitate was filtered off, dried under vacuum to give ethyl 3-[(cyanoacetyl)amino]-1-[4-(4,4,5,5-tetramethyl-1,3,2-dioxaborolan-2-yl)phenyl]-1H-pyrrole-2-carboxylate (10 g, 23.63 mmol, 84% yield) as a white powder. LCMS: (M+H)⁺: 424; Rt: 3.86 min.

### Intermediate 58

### 7-Hydroxy-5-oxo-1-[4-(4,4,5,5-tetramethyl-1,3,2-dioxaborolan-2-yl)phenyl]-4,5-dihydro-1H-pyrrolo[3,2-b]pyridine-6-carbonitrile

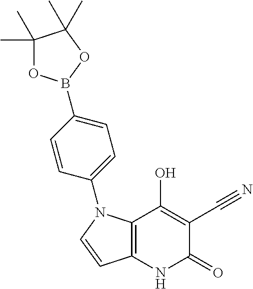

To a solution of ethyl 3-[(cyanoacetyl)amino]-1-[4-(4,4,5,5-tetramethyl-1,3,2-dioxaborolan-2-yl)phenyl]-1H-pyrrole-2-carboxylate (Intermediate 57) (10 g, 23.63 mmol) in THF (150 ml) was added sodium hydride (2.362 g, 60% suspension in oil, 59.1 mmol). The reaction mixture was stirred at 45° C. for 54 hours and to complete the reaction sodium hydride (2.362 g, 60% suspension in oil, 59.1 mmol) was added again and the reaction mixture was stirred at 45° C. for another 16 hours. The reaction was concentrated to dryness after quenching with MeOH. After acidification with 1N HCl, the resulting precipitate was filtered off and dried under vacuum to give a mixture of pinacol boronate and boronic acid (1:1 mixture) (8 g, 80% yield). The solid was used without further purification. LCMS: (M+H)⁺: 378; Rt: 2.69 min.

### Intermediate 59

### Ethyl 3-amino-5-chloro-1-[4-(3-thienyl)phenyl]-1H-pyrrole-2-carboxylate

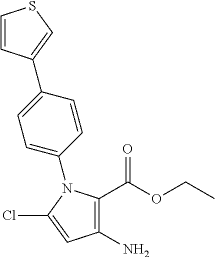

Method A: Ethyl 3-amino-1-(4-bromophenyl)-5-chloro-1H-pyrrole-2-carboxylate (Intermediate 9) (300 mg, 0.873 mmol), potassium 3-thiophenetrifluoroborate (149 mg, 0.786 mmol), palladium(II) acetate (9.80 mg, 0.044 mmol) and cesium carbonate (853 mg, 2.62 mmol) were mixed in ethanol (20 mL) to give a grey mixture. The reaction mixture was stirred at 70° C. for 7 minutes in a Biotage microwave. Purification by silica gel chromatography using DCM as an eluent gave ethyl 3-amino-5-chloro-1-[4-(3-thienyl)phenyl]-1H-pyrrole-2-carboxylate (140 mg, 0.404 mmol, 46.2% yield) as a pale yellow oil. LCMS: (M+H)⁺: 347; Rt: 6.47 min.

Method B: Ethyl 3-amino-1-(4-bromophenyl)-5-chloro-1H-pyrrole-2-carboxylate (Intermediate 9) (1 g, 2.91 mmol), potassium 3-thiophenetrifluoroborate (664 mg, 3.49 mmol), tetrakis(triphenylphosphine)palladium (5 mg, 0.004 mmol) and cesium carbonate (3.03 g, 9.31 mmol) were mixed in a 1,4-dioxane/ethanol 2/1 mixture (6 mL) and stirred at 120° C. for 5 minutes in a microwave reactor. Purification by chromatography on silica gel eluting with cyclohexane/EtOAc 95/5 to 70/30 gave ethyl 3-amino-5-chloro-1-[4-(3-thienyl)phenyl]-1H-pyrrole-2-carboxylate (700 mg, 69% yield) as a solid. LCMS: (M+H)⁺: 347; Rt: 3.69 min.

Intermediates 60 and 61 of formula (V), wherein R³ and R⁴ are both H and R⁶ is Cl, were prepared by methods analogous to that described for Intermediate 59 from ethyl 3-amino-1-(4-bromophenyl)-5-chloro-1H-pyrrole-2-carboxylate (Intermediate 9) using the appropriate potassium trifluoroborate.

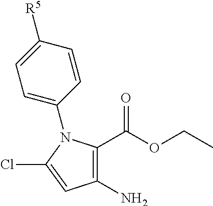

TABLE 4

| Intermediate | Name | R⁵ | Physical data |
|---|---|---|---|
| 60 | Ethyl 3-amino-5-chloro- 1-[4-(5-methyl-2- thienyl)phenyl]-1H- pyrrole-2-carboxylate | 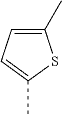 | LCMS: (M + H)⁺: 361; Rt: 4.01 min. |
| 61 | Ethyl 3-amino-5-chloro- 1-[4-(2-thienyl)phenyl]- 1H-pyrrole-2- carboxylate | 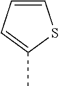 | LCMS: (M + H)⁺: 347; Rt: 3.81 min. |

### Intermediate 62

### Ethyl 3-amino-1-[4-(2-furanyl)phenyl]-1H-pyrrole-2-carboxylate

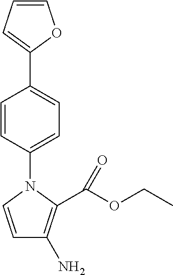

Ethyl 3-amino-1-(4-bromophenyl)-1H-pyrrole-2-carboxylate, (Intermediate 3) (398 mg, 1.286 mmol), potassium 2-furantrifluoroborate (291 mg, 1.672 mmol), palladium(II) acetate (14.43 mg, 0.064 mmol), {2′,6′-bis[(1-methylethyl)oxy]-2-biphenylyl}(dicyclohexyl)phosphane (60 mg, 0.129 mmol) and cesium carbonate (1257 mg, 3.86 mmol) were mixed in ethanol (8 mL) to give a grey mixture. The reaction mixture was stirred at 100° C. for 7 minutes in a Biotage microwave. The reaction mixture was concentrated in vacuo, taken in DCM and washed with 1N HCl. The organic layer was dried over anhydrous Na₂SO₄, filtered and concentrated in vacuo. Purification by chromatography on silica gel eluting with DCM gave ethyl 3-amino-1-[4-(2-furanyl)phenyl]-1H-pyrrole-2-carboxylate (350 mg, 1.181 mmol, 92% yield) as a pale yellow oil. LCMS: (M+H)⁺: 297; Rt: 5.75 min.

### Intermediate 63

### Ethyl 3-amino-5-methyl-1-[4-(3-thienyl)phenyl]-1H-pyrrole-2-carboxylate

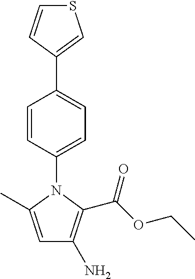

Trimethylboroxin (101 mg, 0.807 mmol), ethyl 3-amino-5-chloro-1-[4-(3-thienyl)phenyl]-1H-pyrrole-2-carboxylate (Intermediate 59) (140 mg, 0.404 mmol), {2′,6′-bis[(1-methylethyl)oxy]-2-biphenylyl}(dicyclohexyl)phosphane (18.84 mg, 0.040 mmol), palladium(II) acetate (4.53 mg, 0.020 mmol) and cesium carbonate (395 mg, 1.211 mmol) were mixed in ethanol (8 mL) and stirred at 130° C. for 7 minutes in a Biotage microwave. The reaction mixture was concentrated in vacuo, taken in DCM and washed with 1N HCl. The organic layer was dried over anhydrous Na₂SO₄, filtered on a small pad of silica gel and concentrated in vacuo to give ethyl 3-amino-5-methyl-1-[4-(3-thienyl)phenyl]-1H-pyrrole-2-carboxylate (120 mg, 0.368 mmol, 91% yield) as a pale brown oil. LCMS: (M+H)⁺: 327; Rt: 6.08 min.

### Intermediate 64

### Ethyl 3-amino-5-chloro-1-[2′-fluoro-6′-(methyloxy)-4-biphenylyl]-1H-pyrrole-2-carboxylate

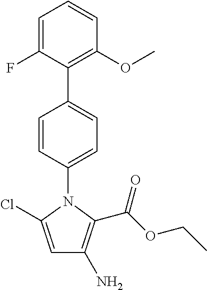

To a solution of ethyl 3-amino-1-(4-bromophenyl)-5-chloro-1H-pyrrole-2-carboxylate (Intermediate 9) (1.2 g, 3.16 mmol) in a 1,4-dioxane (3 mL)/ethanol (0.3 mL) mixture were added [2-fluoro-6-(methyloxy)phenyl]boronic acid (805 mg, 4.74 mmol), cesium carbonate (3.09 g, 9.47 mmol) and tetrakis(triphenylphosphine) palladium (50 mg, 0.043 mmol). The reaction mixture was stirred at 140° C. for 5 minutes in a Biotage microwave before being filtered and concentrated to dryness. Purification by chromatography on silica gel eluting with cyclohexane/EtOAc 95/5 to 60/40 gave ethyl 3-amino-5-chloro-142′-fluoro-6′-(methyloxy)-4-biphenylyl]-1H-pyrrole-2-carboxylate (850 mg, 2.186 mmol, 69.2% yield) as a pale yellow oil. LCMS: (M+H)⁺: 389; Rt: 3.72 min.

Intermediates 65 and 66 of formula (V), wherein R³ and R⁴ are both H and R⁶ is chlorine, were prepared by methods analogous to that described for Intermediate 64 from ethyl 3-amino-1-(4-bromophenyl)-5-chloro-1H-pyrrole-2-carboxylate (Intermediate 9) using the appropriate boronic acid.

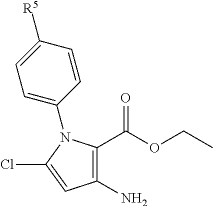

TABLE 5

| Intermediate | Name | R⁵ | Physical data |
|---|---|---|---|
| 65 | Ethyl 3-amino-5-chloro- 1-(4′-methyl-4- biphenylyl)-1H-pyrrole- 2-carboxylate | 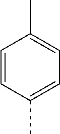 | LCMS: (M + H)⁺: 355; Rt: 3.98 min. |
| 66 | Ethyl 3-amino-5-chloro- 1-[2′-hydroxy-3′- (methyloxy)-4- biphenylyl]-1H-pyrrole- 2-carboxylate | 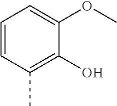 | ¹H NMR: (DMSO-d6, 400 MHz) δ 8.71 (s, 1H), 7.59 (m, 2H), 7.21 (m, 2H), 7.00-6.80 (m, 3H), 5.87 (s, 1H), 5.56 (br s, 2H), 3.91 (q, 2H), 3.86 (s, 3H), 0.91 (t, 3H). |

### Intermediate 67

### Ethyl 3-amino-5-cyano-1-(4-(thiophen-3-yl)phenyl)-1H-pyrrole-2-carboxylate

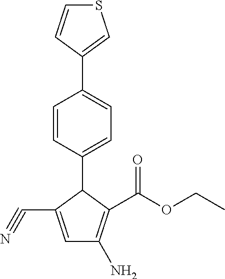

To a suspension of ethyl 3-amino-5-chloro-1-(4-(thiophen-3-yl)phenyl)-1H-pyrrole-2-carboxylate (Intermediate 59) (500 mg, 1.44 mmol), zinc cyanide (102 mg, 0.864 mmol), zinc powder (10 mg, 0.29 mmol) and 2-(di-t-butylphosphino)-1,1′-binapthyl (173 mg, 0.432 mmol) in N,N-dimethylacetamide was added trifluoroacetate palladium (II) (48 mg, 0.145 mmol). The reaction was run in four batches under argon atmosphere and the reaction mixture was stirred for 16 h at 95° C. before being quenched with sat. NaHCO₃. The aqueous layer was extracted with DCM and the product was purified by chromatography on silica gel eluting with petroleum ether/EtOAc 75/25. The resulting solid was triturated in pentane and dried to give ethyl 3-amino-5-cyano-1-(4-(thiophen-3-yl)phenyl)-1H-pyrrole-2-carboxylate (805 mg, 41% yield) as a pale yellow solid. ¹H NMR: (CDCl₃, 400 MHz) δ 7.68 (m, 2H), 7.53 (m, 1H), 7.43-7.42 (m, 2H), 7.31 (m, 2H), 6.45 (s, 1H), 4.13 (q, J=7.2 Hz, 2H), 1.04 (t, J=7.2 Hz, 3H).

### Intermediate 68

### Ethyl 5-chloro-3-[(cyanoacetyl)amino]-1-[4-(3-thienyl)phenyl]-1H-pyrrole-2-carboxylate

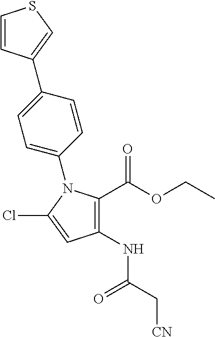

Method A: To a solution of ethyl 3-amino-5-chloro-1-[4-(3-thienyl)phenyl]-1H-pyrrole-2-carboxylate (Intermediate 59) (140 mg, 0.404 mmol) in DMF (20 mL) were added successively cyanoacetic acid (68.7 mg, 0.807 mmol), HOBT (74.2 mg, 0.484 mmol), EDC (93 mg, 0.484 mmol) and triethylamine (0.141 mL, 1.009 mmol). The reaction mixture was stirred at RT for 24 hours before being evaporated to dryness and poured into water. The precipitate was filtered off, washed with water and dried under vacuum to give ethyl 5-chloro-3-[(cyanoacetyl)amino]-1-[4-(3-thienyl)phenyl]-1H-pyrrole-2-carboxylate (120 mg, 0.290 mmol, 71.8% yield) as a yellow powder which was used without further purification in the next step. LCMS: (M+H)⁺: 414, 416; Rt: 6.45 min.

Method B: To a suspension of ethyl 3-amino-5-chloro-1-[4-(3-thienyl)phenyl]-1H-pyrrole-2-carboxylate (Intermediate 59) (1.63 g, 4.25 mmol), cyanoacetic acid (0.796 g, 9.36 mmol) and triethylamine (2.371 mL, 17.01 mmol) in DCM (30 mL) at RT was added EDC (1.794 g, 9.36 mmol) and HOBT (1.433 g, 9.36 mmol). The reaction mixture was stirred from 0° C. to RT for 2 h before being washed successively with 1N HCl, sat. NaHCO₃ and brine. The organic layer was dried over anhydrous Na₂SO₄, filtered and concentrated in vacuo. The product was purified by chromatography on an Isco Companion RF. The sample was loaded on 100 g Biotage SNAP silica column then the purification was carried out using cyclohexane/EtOAc 100/0 to 60/40. The appropriate fractions were combined and concentrated in vacuo to give the required product ethyl 5-chloro-3-[(cyanoacetyl)amino]-1-[4-(3-thienyl)phenyl]-1H-pyrrole-2-carboxylate (1.26 g, 3.04 mmol, 71.6% yield) as a white foam. LCMS: (M+H)⁺: 414; Rt: 3.74 min.

Intermediates 69 to 75 of formula (IV), wherein R³ and R⁴ are both H and R¹ is cyano, were prepared by methods analogous to that described for Intermediate 68 using the appropriate Intermediate. HATU was used for Intermediate 74 instead of EDC/HOBT

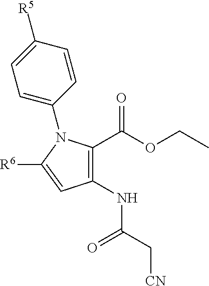

TABLE 6

| | | From | | | |
|---|---|---|---|---|---|
| Intermediate | Name | Int. | R⁶ | R⁵ | Physical data |
| 69 | Ethyl 3- [(cyanoacetyl)amino]- 1-[4-(2-furanyl)phenyl]- 1H-pyrrole-2- carboxylate | 62 | H | 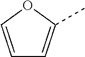 | LCMS: (M + H)⁺: 364; Rt: 5.88 min. |
| 70 | Ethyl 3- [(cyanoacetyl)amino]- 5-methyl-1-[4-(3- thienyl)phenyl]-1H- pyrrole-2-carboxylate | 63 | Me | 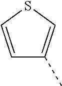 | LCMS: (M − H)⁻: 392; Rt: 6.22 min. |
| 71 | Ethyl 5-chloro-3- [(cyanoacetyl)amino]- 1-[4-(5-methyl-2- thienyl)phenyl]-1H- pyrrole-2-carboxylate | 60 | Cl | 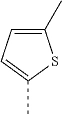 | LCMS: (M + H)⁺: 428; Rt: 4.05 min. |
| 72 | Ethyl 5-chloro-3- [(cyanoacetyl)amino]- 1H-pyrrole-2- carboxylate | 61 | Cl | 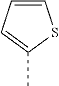 | LCMS: (M + H)⁺: 414; Rt: 3.85 min |
| 73 | Ethyl 5-chloro-3- [(cyanoacetyl)amino]- 1-[2′-fluoro-6′- (methyloxy)-4- biphenylyl]-1H-pyrrole- 2-carboxylate | 64 | Cl | 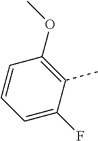 | LCMS: (M + H)⁺: 456; Rt: 3.79 min. |
| 74 | Ethyl 5-cyano-3- [(cyanoacetyl)amino]- 1-[4-(3-thienyl)phenyl]- 1H-pyrrole-2- carboxylate | 67 | CN | 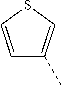 | LCMS: (M − H)⁻ : 403; Rt: 3.58 min. |
| 75 | Ethyl 5-cyano-3- [(cyanoacetyl)amino]- 1-(4-ethylphenyl)-1H- pyrrole-2-carboxylate | 53 | CN | Et | LCMS: (M − H)⁻: 349; Rt: 3.48 min. |

### Intermediate 76

### Ethyl 5-chloro-3-[(cyanoacetyl)amino]-1-[4-(4,4,5,5-tetramethyl-1,3,2-dioxaborolan-2-yl)phenyl]-1H-pyrrole-2-carboxylate

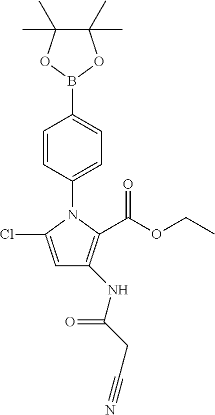

To a solution of ethyl 3-[(cyanoacetyl)amino]-1-[4-(4,4,5,5-tetramethyl-1,3,2-dioxaborolan-2-yl)phenyl]-1H-pyrrole-2-carboxylate (Intermediate 57) (3.45 g, 8.15 mmol) in THF (50 mL) was added NCS (1.197 g, 8.97 mmol). The reaction mixture was stirred 2 h at RT then overnight at 50° C. The reaction mixture was concentrated in vacuo, taken in DCM (200 mL) and washed with water then brine. The organic layer was dried over anhydrous Na₂SO₄, filtered and concentrated in vacuo. The product was purified by chromatography on an Isco Companion. The sample was loaded on 50 g Biotage silica (Si) column then the purification was carried out using Cyclohexane/EtOAc 100/0 to 60/40. The appropriate fractions were combined and concentrated in vacuo to give ethyl 5-chloro-3-[(cyanoacetyl)amino]-1-[4-(4,4,5,5-tetramethyl-1,3,2-dioxaborolan-2-yl)phenyl]-1H-pyrrole-2-carboxylate (2.45 g, 5.35 mmol, 65.7% yield) as a white foam. LCMS: (M−H)⁻: 456; Rt: 3.98 min.

### Intermediate 77

### Ethyl 5-chloro-3-(2-cyanoacetamido)-1-(4-(5-cyanothiophen-3-yl)phenyl)-1H-pyrrole-2-carboxylate

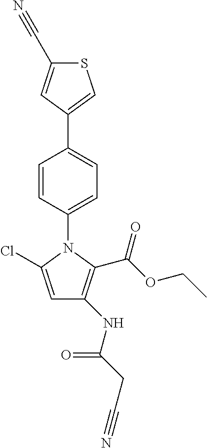

Ethyl 5-chloro-3-[(cyanoacetyl)amino]-1-[4-(4,4,5,5-tetramethyl-1,3,2-dioxaborolan-2-yl)phenyl]-1H-pyrrole-2-carboxylate (Intermediate 76) (300 mg, 0.655 mmol), 4-bromothiophene-2-carbonitrile (160 mg, 0.85 mmol), 1.0M solution of Na₂CO₃ (1.3 mL, 1.3 mmol) in water and Pd(PPh₃)₄ (7.57 mg, 6.55 μmol) were mixed together in 1,4-dioxane (7 mL). The reaction vessel was sealed and heated in a Biotage Initiator to 130° C. for 10 min. Monitoring of the reaction by LC/MS showed the reaction was incomplete with SM remaining. The reaction mixture was then heated to 130° C. for another 5 min before being concentrated in vacuo after addition of EtOH. DCM was added to the residue which was filtered then directly purified by chromatography on silica gel eluting with cyclohexane/EtOAc 100/0 to 70/30. The appropriate fractions were collected and concentrated in vacuo to give ethyl 5-chloro-3-(2-cyanoacetamido)-1-(4-(5-cyanothiophen-3-yl)phenyl)-1H-pyrrole-2-carboxylate (120 mg, 42% yield). LCMS: (M−H)⁻: 437; Rt: 3.59 min.

Intermediates 78 to 87 of formula (IV), wherein R³ and R⁴ are both H, R⁶ is Cl and R¹ is cyano, were prepared by methods analogous to that described for Intermediate 77 from Intermediate 76 using the appropriate bromo derivative.

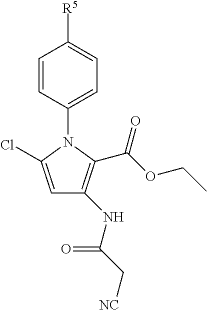

TABLE 7

| Intermediate | Name | R⁵ | Physical data |
|---|---|---|---|
| 78 | Ethyl 1-(4- (benzo[b]thiophen-3- yl)phenyl)-5-chloro-3-(2- cyanoacetamido)-1H- pyrrole-2-carboxylate | 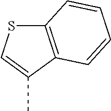 | LCMS: (M − H)⁻: 462; Rt: 4.09 min. |
| 79 | Ethyl 5-chloro-3- [(cyanoacetyl)amino]-1-{4- [5-(methyloxy)-2- pyridinyl]phenyl}-1H- pyrrole-2-carboxylate | 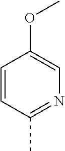 | LCMS: (M + H)⁺: 439; Rt: 3.50 min. |
| 80 | Ethyl 5-chloro-3- [(cyanoacetyl)amino]-1-[4- (1,3-thiazol-4-yl)phenyl]-1H- pyrrole-2-carboxylate | 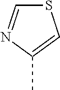 | LCMS: (M + H)⁺: 415; Rt: 3.38 min. |
| 81 | Ethyl 5-chloro-3-(2- cyanoacetamido)-1-(4-(4- cyanothiophen-3-yl)phenyl)- 1H-pyrrole-2-carboxylate | 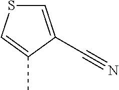 | LCMS: (M − H)⁻: 437; Rt: 3.49 min. |
| 82 | Ethyl 5-chloro-3- [(cyanoacetyl)amino]-1-[4- (3-pyridinyl)phenyl]-1H- pyrrole-2-carboxylate | 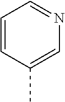 | LCMS: (M + H)⁺: 409; Rt: 3.20 min. |
| 83 | Ethyl 5-chloro-3- [(cyanoacetyl)amino]-1-[4- (3-methyl-2-thienyl)phenyl]- 1H-pyrrole-2-carboxylate | 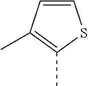 | LCMS: (M + H)⁺: 428; Rt: 4.00 min. |
| 84 | Ethyl 5-chloro-3- [(cyanoacetyl)amino]-1-[4- (1-methyl-1H-pyrazol-4- yl)phenyl]-1H-pyrrole-2- carboxylate | 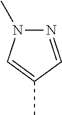 | LCMS: (M + H)⁺: 412; Rt: 3.11 min. |
| 85 | Ethyl 5-chloro-3- [(cyanoacetyl)amino]-1-[4- (2-methyl-1,3-thiazol-4- yl)phenyl]-1H-pyrrole-2- carboxylate | 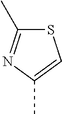 | LCMS: ( M + H )⁺: 429; Rt: 3.54 min. |
| 86 | Ethyl 1-[4-(6-amino-2- pyridinyl)phenyl]-5-chloro-3- [(cyanoacetyl)amino]-1H- pyrrole-2-carboxylate | 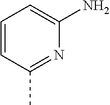 | LCMS: (M + H)⁺: 424; Rt: 3.14 min. |
| 87 | Ethyl 5-chloro-3- [(cyanoacetyl)amino]-1-[3′- (hydroxymethyl)-4- biphenylyl]-1H-pyrrole-2- carboxylate | 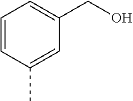 | LCMS: (M − H)⁻: 436; Rt: 3.20 min. |

### Intermediate 88

### Ethyl 5-chloro-1-[2′-chloro-6′-(methyloxy)-4-biphenylyl]-3-[(cyanoacetyl)amino]-1H-pyrrole-2-carboxylate

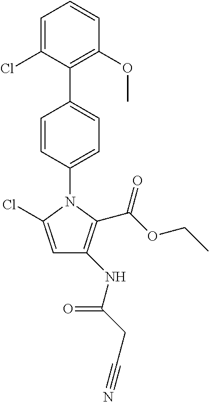

To a solution of ethyl 1-(4-bromophenyl)-5-chloro-3-[(cyanoacetyl)amino]-1H-pyrrole-2-carboxylate (Intermediate 10) (500 mg, 1.218 mmol) and 2-chloro-6-methoxyphenylboronic acid (681 mg, 3.65 mmol) were added Pd(PPh₃)₄ (28.1 mg, 0.024 mmol) and Na₂CO₃ (3.65 mL, 3.65 mmol) 1M in water in 1,4-dioxane (5 mL). The reaction vessel was sealed and heated in Biotage Initiator to 120° C. for 5 min. The reaction mixture was concentrated in vacuo, taken in DCM (25 mL) and washed with water. The organic layer was dried over anhydrous Na₂SO₄, filtered and concentrated in vacuo. The residue was loaded on 10 g Biotage silica (Si) column then the purification was carried out using a cyclohexane/EtOAc 100/0 to 80/20 gradient. The appropriate fractions were combined and concentrated in vacuo to give ethyl 5-chloro-1-[2′-chloro-6′-(methyloxy)-4-biphenylyl]-3-[(cyanoacetyl)amino]-1H-pyrrole-2-carboxylate (170 mg, 30% yield) as a yellow amorphous solid. LCMS: (M+H)⁺: 472; Rt: 3.97 min.

Intermediates 89 to 94 of formula (IV), wherein R³ and R⁴ are both H, R⁶ is Cl and R¹ is cyano were prepared by methods analogous to that described for Intermediate 88 from Intermediate 10 using the appropriate boronic acid derivative.

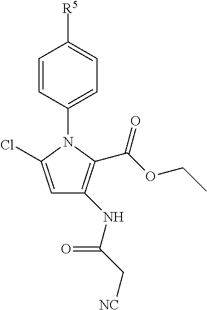

TABLE 8

| Intermediate | Name | R⁵ | Physical data |
|---|---|---|---|
| 89 | Ethyl 5-chloro-3- [(cyanoacetyl)amino]-1-(3′- hydroxy-4-biphenylyl)-1H- pyrrole-2-carboxylate | 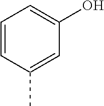 | LCMS: (M + H)⁺: 424; Rt: 3.27 min. |
| 90 | Ethyl 5-chloro-3- [(cyanoacetyl)amino]-1-(3′- fluoro-2′-hydroxy-4- biphenylyl)-1H-pyrrole-2- carboxylate | 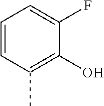 | LCMS: (M + H)⁺: 442; Rt: 3.48 min. |
| 91 | Ethyl 5-chloro-1-(3′-chloro- 2′-hydroxy-4-biphenylyl)-3- [(cyanoacetyl)amino]-1H- pyrrole-2-carboxylate | 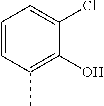 | LCMS: (M + H)⁺: 458; Rt: 3.68 min. |
| 92 | Ethyl 5-chloro-3- [(cyanoacetyl)amino]-1-(2′- fluoro-4′-methyl-4- biphenylyl)-1H-pyrrole-2- carboxylate | 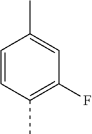 | LCMS: (M + H)⁺: 440; Rt: 4.04 min. |
| 93 | Ethyl 5-chloro-1-[2′-chloro- 4′-(methyloxy)-4- biphenylyl]-3- [(cyanoacetyl)amino]-1H- pyrrole-2-carboxylate | 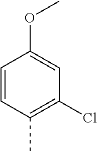 | LCMS: (M + H)⁺: 472; Rt: 4.01 min |
| 94 | Ethyl 5-chloro-3- [(cyanoacetyl)amino]-1-[5′- methyl-2′-(methyloxy)-4- biphenylyl]-1H-pyrrole-2- carboxylate | 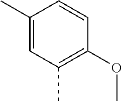 | LCMS: (M + H)⁺: 452; Rt: 3.99 min |

### Intermediate 95

### 2-Chloro-7-hydroxy-1-[5′-methyl-2′-(methyloxy)-4-biphenylyl]-5-oxo-4,5-dihydro-1H-pyrrolo[3,2-b]pyridine-6-carbonitrile

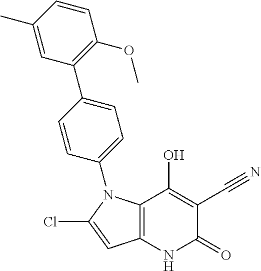

To a solution of ethyl 5-chloro-3-[(cyanoacetyl)amino]-1-[5′-methyl-2′-(methyloxy)-4-biphenylyl]-1H-pyrrole-2-carboxylate (Intermediate 94) (340 mg, 0.752 mmol) in DMSO (1 mL) was added dropwise potassium tert-butoxide (1.505 mL, 1M in THF, 1.505 mmol). The yellow reaction mixture was stirred at room temperature for 30 min. Water was added before being acidified with 1N HCl to pH 1. The precipitate was filtered, washed successively with water (5 mL), CH₃CN (5 mL) and Et₂O (5 mL) then dried. The product 2-chloro-7-hydroxy-1-[5′-methyl-2′-(methyloxy)-4-biphenylyl]-5-oxo-4,5-dihydro-1H-pyrrolo[3,2-b]pyridine-6-carbonitrile (220 mg, 0.504 mmol) was obtained as a beige powder and was used in the next step without further purification. LCMS: (M+H)⁺: 406; Rt: 3.02 min

### Intermediate 96

### Ethyl 5-chloro-3-{[(2-fluorophenyl)acetyl]amino}-1-[4-(methyloxy)phenyl]-1H-pyrrole-2-carboxylate

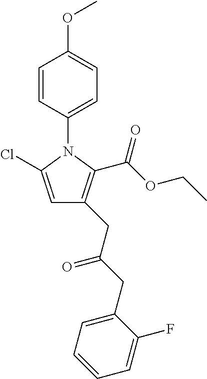

To a solution of (2-fluorophenyl)acetic acid (168 mg, 1.087 mmol) and ethyl 3-amino-5-chloro-1-[4-(methyloxy)phenyl]-1H-pyrrole-2-carboxylate (Intermediate 42) (300 mg, 0.906 mmol) in DCM (5 mL) at RT were added EDC (260 mg, 1.359 mmol), HOBT (208 mg, 1.359 mmol) and triethylamine (0.126 mL, 0.906 mmol). The reaction mixture was stirred at RT for 18 h before being filtered through a PL-MIXED MP SPE column (200 mg) using MeOH as eluent and concentrated in vacuo. The crude was purified by chromatography on an Isco Companion RF. The sample was loaded on 12 g Redisep Gold Rf silica column then the purification was carried out using cyclohexane/EtOAc 100/0 to 70/30. The appropriate fractions were combined and concentrated in vacuo to give ethyl 5-chloro-3-{[(2-fluorophenyl)acetyl]amino}-1-[4-(methyloxy)phenyl]-1H-pyrrole-2-carboxylate (300 mg, 0.696 mmol, 77% yield) as a colorless oil. LCMS: (M+H)⁺: 431; Rt: 3.93 min.

Intermediates 97 to 127 of formula (IV), wherein R³ is H, were prepared by methods analogous to that described for Intermediate 96 using the appropriate carboxylic acid. For Intermediates 102 and 120, (1-cyano-2-ethoxy-2-oxoethylidenaminooxy)dimethylamino-morpholino-carbenium hexafluorophosphate COMU) was used instead of EDC/HOBT as a coupling reagent.

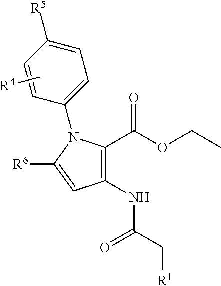

TABLE 9

| | | From | | | | | |
|---|---|---|---|---|---|---|---|
| Int. | Name | Int. | R¹ | R⁴ | R⁵ | R⁶ | Physical data |
| 97 | Ethyl 3-{[(3- fluorophenyl)acetyl] amino}-1-[4- (methyloxy)phenyl]-1H- pyrrole-2-carboxylate | 41 | 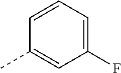 | H | OMe | H | LCMS: (M + H)⁺: 397; Rt : 3.68 min |
| 98 | Ethyl 3-{[(2- fluorophenyl)acetyl] amino}-1-[4- (methyloxy)phenyl]-1H- pyrrole-2-carboxylate | 41 | 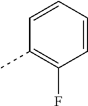 | H | OMe | H | LCMS: (M + H)⁺: 397; Rt: 3.66 min |
| 99 | Ethyl 5-chloro-3-{[(3- fluorophenyl)acetyl] amino}-1-[4- (methyloxy)phenyl]-1H- pyrrole-2-carboxylate | 42 | 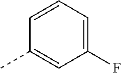 | H | OMe | Cl | LCMS: (M + H)⁺: 431; Rt: 3.95 min. |
| 100 | Ethyl 5-chloro-3-{[(4- fluorophenyl)acetyl] amino}-1-[4- (methyloxy)phenyl]-1H- pyrrole-2-carboxylate | 42 | 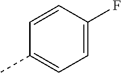 | H | OMe | Cl | LCMS: (M + H)⁺: 431; Rt: 3.93 min. |
| 101 | Ethyl 5-chloro-1-[4- (methyloxy)phenyl]-3- [(phenylacetyl)amino]- 1H-pyrrole-2-carboxylate | 42 | 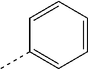 | H | OMe | Cl | LCMS: (M + H)⁺: 413; Rt: 3.91 min. |
| 102 | Ethyl 5-chloro-1-(4- fluorophenyl)-3- [(phenylacetyl)amino]- 1H-pyrrole-2-carboxylate | 45 | 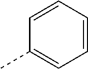 | H | F | Cl | LCMS: (M + H)⁺: 401; Rt: 3.93 min. |
| 103 | Ethyl 5-chloro-3-{[(2- fluorophenyl)acetyl] amino}-1-[3- (methyloxy)phenyl]-1H- pyrrole-2-carboxylate | 46 | 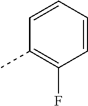 | 3-OMe | H | Cl | LCMS: (M + H)⁺: 431; Rt: 3.94 min. |
| 104 | Ethyl 5-chloro-3-{[(3- fluorophenyl)acetyl] amino}-1-[3- (methyloxy)phenyl]-1H- pyrrole-2-carboxylate | 46 | 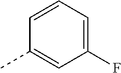 | 3-OMe | H | Cl | LCMS: (M + H)⁺: 431; Rt: 3.96 min. |
| 105 | Ethyl 5-chloro-3-{[(4- fluorophenyl)acetyl] amino}-1-[3- (methyloxy)phenyl]-1H- pyrrole-2-carboxylate | 46 | 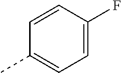 | 3-OMe | H | Cl | LCMS: (M + H)⁺: 431 Rt: 3.96 min. |
| 106 | Ethyl 5-chloro-1-[3- (methyloxy)phenyl]-3- [(phenylacetyl)amino]- 1H-pyrrole-2-carboxylate | 46 | 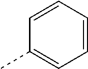 | 3-OMe | H | Cl | LCMS: (M + H)⁺: 413; Rt: 3.95 min. |
| 107 | Ethyl 5-chloro-3-{[(4- fluorophenyl)acetyl] amino}-1-(4- methylphenyl)-1H- pyrrole-2-carboxylate | 47 | 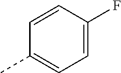 | H | Me | Cl | LCMS: (M + H)⁺: 415; Rt: 4.12 min. |
| 108 | Ethyl 5-chloro-3- [(phenylacetyl)amino]- 1-[4- (trifluoromethyl)phenyl]- 1H-pyrrole-2-carboxylate | 48 | 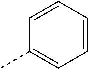 | H | CF₃ | Cl | LCMS: (M + H)⁺: 451; Rt: 4.14 min. |
| 109 | Ethyl 5-chloro-1-(4- methylphenyl)-3- [(phenylacetyl)amino]- 1H-pyrrole-2-carboxylate | 47 | 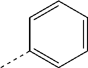 | H | Me | Cl | LCMS: (M + H)⁺: 397 Rt: 4.11 min. |
| 110 | Ethyl 5-chloro-3-{[(2- fluorophenyl)acetyl] amino}-1-(4- methylphenyl)-1H- pyrrole-2-carboxylate | 47 | 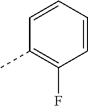 | H | Me | Cl | LCMS: (M + H)⁺: 415 Rt: 4.12 min. |
| 111 | Ethyl 5-chloro-3-{[(3- fluorophenyl)acetyl] amino}-1-(4- methylphenyl)-1H- pyrrole-2-carboxylate | 47 | 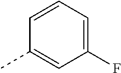 | H | Me | Cl | LCMS: (M + H)⁺: 415 Rt: 4.14 min. |
| 112 | Ethyl 5-chloro-3-{[(2- fluorophenyl)acetyl] amino}-1-[4- (trifluoromethyl)phenyl]- 1H-pyrrole-2-carboxylate | 48 | 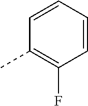 | H | CF₃ | Cl | LCMS: (M + H)⁺: 469 Rt: 4.13 min. |
| 113 | Ethyl 5-chloro-3-{[(3- fluorophenyl)acetyl] amino}-1-[4- (trifluoromethyl)phenyl]- 1H-pyrrole-2-carboxylate | 48 | 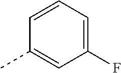 | H | CF₃ | Cl | LCMS: (M + H)⁺: 469 Rt: 4.16 min. |
| 114 | Ethyl 5-chloro-3-{[(2- fluorophenyl)acetyl] amino}-1-{4- [(trifluoromethyl)oxy] phenyl}-1H-pyrrole-2- carboxylate | 49 | 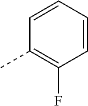 | H | OCF₃ | Cl | LCMS: (M + H)⁺: 485; Rt: 4.24 min |
| 115 | Ethyl 5-chloro-3-({[3- (ethyloxy)phenyl]acetyl} amino)-1-(4- methylphenyl)-1H- pyrrole-2-carboxylate | 47 | 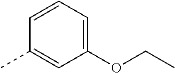 | H | Me | Cl | LCMS: (M + H)⁺: 441 Rt: 4.28 min |
| 116 | Ethyl 5-chloro-3-{[(4- cyanophenyl)acetyl] amino}-1-(4- methylphenyl)-1H- pyrrole-2-carboxylate | 47 | 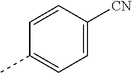 | H | Me | Cl | LCMS: (M + H)⁺: 422 Rt: 4.01 min |
| 117 | Ethyl 5-chloro-1-(4- methylphenyl)-3-{[(3- nitrophenyl)acetyl]amino}- 1H-pyrrole-2-carboxylate | 47 | 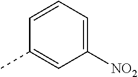 | H | Me | Cl | LCMS: (M + H)⁺: 442 Rt: 3.99 min |
| 118 | Ethyl 5-chloro-3-{[(3- cyanophenyl)acetyl]amino}- 1-(4-methylphenyl)- 1H-pyrrole-2-carboxylate | 47 | 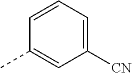 | H | Me | Cl | LCMS: (M + H)⁺: 422 Rt: 3.89 min |
| 119 | Ethyl 3-{[(3- bromophenyl)acetyl]amino}- 5-chloro-1-(4- methylphenyl)-1H- pyrrole-2-carboxylate | 47 | 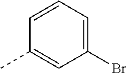 | H | Me | Cl | LCMS: (M + H)⁺: 477 Rt: 4.37 min |
| 120 | Ethyl 5-chloro-3- [(phenylacetyl)amino]- 1-[4-(3-thienyl)phenyl]- 1H-pyrrole-2-carboxylate | 59 | 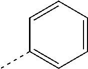 | H | 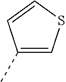 | Cl | LCMS: (M + H)⁺: 465; Rt: 4.28 min. |
| 121 | Ethyl 5-chloro-3-{[(4- cyanophenyl)acetyl] amino}-1-[4-(3- thienyl)phenyl]-1H- pyrrole-2-carboxylate | 59 | 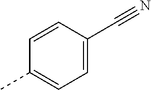 | H | 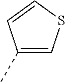 | Cl | LCMS: (M + H)⁺: 490; Rt: 4.15 min. |
| 122 | Ethyl 5-chloro-3-{[(3- cyanophenyl)acetyl] amino}-1-[4-(3- thienyl)phenyl]-1H- pyrrole-2-carboxylate | 59 | 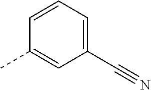 | H | 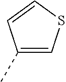 | Cl | LCMS: (M + H)⁺: 490; Rt: 3.98 min. |
| 123 | Ethyl 5-chloro-3-(2-(2- fluorophenyl)acetamido)- 1-(2′-hydroxy-3′- methoxy-[1,1′-biphenyl]- 4-yl)-1H-pyrrole-2- carboxylate | 66 | 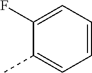 | H | 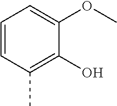 | Cl | LCMS: (M + H)⁺: 523; Rt: 4.14 min. |
| 124 | Ethyl 5-chloro-3-(2-(3- chlorophenyl)acetamido)- 1-(2′-hydroxy-3′- methoxy-[1,1′-biphenyl]- 4-yl)-1H-pyrrole-2- carboxylate | 66 | 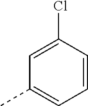 | H | 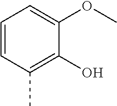 | Cl | LCMS: (M + H)⁺: 539; Rt: 4.15 min. |
| 125 | Ethyl 5-chloro-1-(2′- hydroxy-3′-methoxy- [1,1′-biphenyl]-4-yl)-3- (2-(3-(methoxycarbonyl) phenyl)acetamido)-1H- pyrrole-2-carboxylate | 66 | 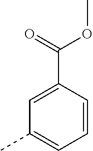 | H | 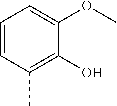 | Cl | LCMS: (M + H)⁺: 563; Rt: 3.91 min. |
| 126 | Ethyl 5-chloro-3-(2-(4- cyanophenyl)acetamido)- 1-(2′-hydroxy-3′- methoxy-[1,1′-biphenyl]- 4-yl)-1H-pyrrole-2- carboxylate | 66 | 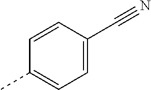 | H | 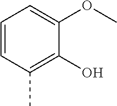 | Cl | LCMS: (M + H)⁺: 530; Rt: 3.81 min. |
| 127 | Ethyl 5-chloro-1-(2′- hydroxy-3′-methoxy- [1,1′-biphenyl]-4-yl)-3- (2-(3-methoxyphenyl) acetamido)-1H-pyrrole- 2-carboxylate | 66 | 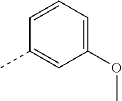 | H | 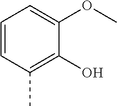 | Cl | LCMS: (M + H)⁺: 535; Rt: 3.15 min. |

### Intermediate 128

### Ethyl 5-chloro-1-(4-ethylphenyl)-3-[(phenylacetyl)amino]-1H-pyrrole-2-carboxylate

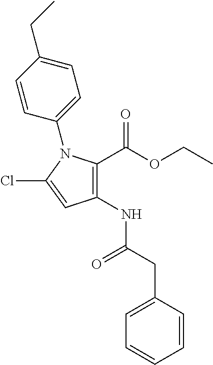

To a solution of ethyl 3-amino-5-chloro-1-(4-ethylphenyl)-1H-pyrrole-2-carboxylate (Intermediate 44) (500 mg, 1.519 mmol) in DCM (5 mL) was added successively pyridine (0.306 mL, 3.80 mmol) and phenylacetyl chloride (0.241 mL, 1.822 mmol). The reaction mixture was stirred for 2 h at RT before being washed with 1N HCl, sat. NaHCO₃ and brine. The organic layer was dried over Na₂SO₄, filtered and concentrated to dryness. The product was purified by chromatography on an Isco Companion RF. The sample was loaded on 25 g Biotage SNAP silica (Si) column then the purification was carried out using cyclohexane/EtOAc 100/0 to 80/20. The appropriate fractions were combined and concentrated in vacuo to give ethyl 5-chloro-1-(4-ethylphenyl)-3-[(phenylacetyl)amino]-1H-pyrrole-2-carboxylate (580 mg, 1.412 mmol, 93% yield) as a colourless oil. LCMS: (M+H)⁺: 411; Rt: 4.31 min.

Intermediates 129 to 134 of formula (IV), wherein R³ is H and R¹ is phenyl, were prepared by methods analogous to that described for Intermediate 128 using the appropriate intermediate.

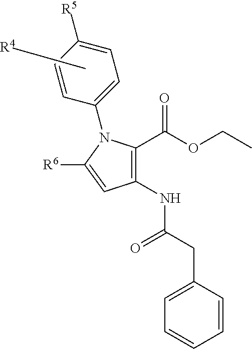

TABLE 10

| | | From | | | | |
|---|---|---|---|---|---|---|
| Intermediate | Name | Int. | R⁴ | R⁵ | R⁶ | Physical data |
| 129 | Ethyl 5-chloro-1-(3,4- | 50 | 3- | Me | Cl | LCMS: (M + H)⁺: |
| | dimethylphenyl)-3- | | Me | | | 411; Rt: 4.26 min. |
| | [(phenylacetyl)amino]-1H- | | | | | |
| | pyrrole-2-carboxylate | | | | | |
| 130 | Ethyl 5-chloro-1-[4- | 51 | H | CH₂CN | Cl | LCMS: (M + H)⁺: |
| | (cyanomethyl)phenyl]-3- | | | | | 422; Rt: 3.57 min. |
| | [(phenylacetyl)amino]-1H- | | | | | |
| | pyrrole-2-carboxylate | | | | | |
| 131 | Ethyl 5-chloro-1-(4- cyclohexylphenyl)-3- [(phenylacetyl)amino]-1H- pyrrole-2-carboxylate | 52 | H | 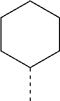 | Cl | LCMS: (M + H)⁺: 465; Rt: 4.59 min. |
| 132 | Ethyl 5-chloro-1-(4′- methyl-4-biphenylyl)-3- [(phenylacetyl)amino]-1H- pyrrole-2-carboxylate | 65 | H | 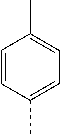 | Cl | LCMS: (M + H)⁺: 473; Rt: 4.51 min. |
| 133 | Ethyl 5-cyano-1-(4- | 53 | H | Et | CN | LCMS: (M + H)⁺: |
| | ethylphenyl)-3- | | | | | 402; Rt: 3.99 min. |
| | [(phenylacetyl)amino]-1H- | | | | | |
| | pyrrole-2-carboxylate | | | | | |
| 134 | Ethyl 5-chloro-1-[2′- hydroxy-3′-(methyloxy)-4- biphenylyl]-3- [(phenylacetyl)amino]-1H- pyrrole-2-carboxylate | 66 | H | 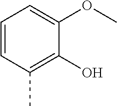 | Cl | ¹H NMR: (DMSO-d6, 400 MHz) δ 9.49 (s, 1H), 8.74 (s, 1H), 7.62 (m, 2H), 7.40- 7.35 (m, 5H), 7.28 (m, 2H), 7.04 (s, 1H), 7.00-6.80 (m, 3H), 3.90 (q, J = 6.81 Hz, 2H), 3.86 (s, 3H), 3.80 (s, 2H), 0.83 (t, J = 6.8 Hz, 3H). |

### Intermediate 135

### Methyl 3-(2-chloro-2-oxoethyl)benzoate

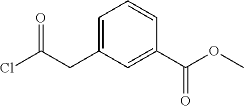

To a solution of {3-[(methyloxy)carbonyl]phenyl}acetic acid (prepared by methods analogous to those described in PCT International Application WO2005080367, 1 Sep. 2005) (400 mg, 2.060 mmol), in DCM (15 mL) at RT were added pyridine (17 μL, 0.206 mmol) and oxalyl chloride (2M in DCM, 1.236 mL, 2.472 mmol). The reaction mixture was left stirring overnight before being evaporated to dryness. Methyl 3-(2-chloro-2-oxoethyl)benzoate (410 mg, 1.928 mmol, 94% yield) was obtained as a yellow oil which was used in the next step without further purification.

### Intermediate 136

### Ethyl 2-(2-chloro-2-oxoethyl)benzoate

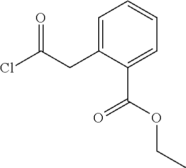

To a solution of {2-[(ethyloxy)carbonyl]phenyl}acetic acid (prepared by methods analogous to those described in PCT International Application WO2005080367, 1 Sep. 2005) (416 mg, 1.998 mmol), in DCM (15 mL) at RT were added pyridine (16 μL, 0.200 mmol) and oxalyl chloride (2M in DCM, 1.199 mL, 2.398 mmol). The reaction mixture was left stirring overnight before being evaporated to dryness. Ethyl 2-(2-chloro-2-oxoethyl)benzoate (460 mg, 2.030 mmol, 102% yield) was obtained as a yellow oil, which was used in the next step without further purification.

### Intermediate 137

### Ethyl 5-chloro-3-({[3-(methyloxy)phenyl]acetyl}amino)-1-[4-(3-thienyl)phenyl]-1H-pyrrole-2-carboxylate

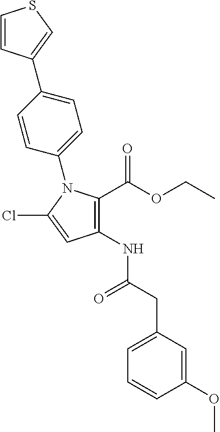

To a solution of ethyl 3-amino-5-chloro-1-[4-(3-thienyl)phenyl]-1H-pyrrole-2-carboxylate (Intermediate 59) (347 mg, 1.0 mmol) in DCM (15 mL) was added pyridine (0.405 mL, 5.00 mmol) at RT then was added 3-methoxyphenylacetyl chloride (0.203 mL, 1.301 mmol). The reaction mixture was stirred for 3 h before being extracted successively between DCM/water, 1N HCl, sat. NaHCO₃ and finally brine. The organic layer was dried over Na₂SO₄, filtered and evaporated to dryness. Ethyl 5-chloro-3-({[3-(methyloxy)phenyl]acetyl}amino)-1-[4-(3-thienyl)phenyl]-1H-pyrrole-2-carboxylate (490 mg, 0.990 mmol, 99% yield) was obtained as a yellow oil which was used in the next step without further purification. LCMS: (M+H)⁺: 495; Rt: 4.12 min.

Intermediates 138 and 139 of formula (IV), wherein R³ and R⁴ are both H, were prepared by methods analogous to that described for Intermediate 137 using the appropriate acyl chloride.

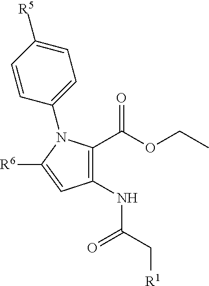

TABLE 11

| | | From | | | | |
|---|---|---|---|---|---|---|
| Int. | Name | Int. | R¹ | R⁵ | R⁶ | Physical data |
| 138 | Ethyl 5-chloro-3-[({3- [(methyloxy)carbonyl] phenyl}acetyl)amino]-1- [4-(3-thienyl)phenyl]-1H- pyrrole-2-carboxylate | 59 and 135 | 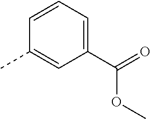 | 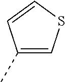 | Cl | LCMS: (M + H)⁺: 523; Rt: 4.09 min. |
| 139 | Ethyl 5-chloro-3-[({2- [(ethyloxy)carbonyl] phenyl}acetyl)amino]-1- [4-(3-thienyl)phenyl]-1H- pyrrole-2-carboxylate | 59 and 136 | 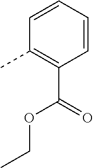 | 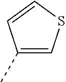 | Cl | LCMS: (M + H)⁺: 537; Rt: 4.20 min. |

### Intermediate 140

### 1-(4-Aminophenyl)-2-chloro-7-hydroxy-5-oxo-4,5-dihydro-1H-pyrrolo[3,2-b]pyridine-6-carbonitrile

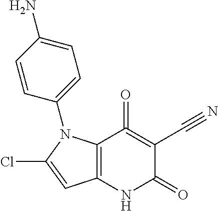

A solution of 1-(4-bromophenyl)-2-chloro-7-hydroxy-5-oxo-4,5-dihydro-1H-pyrrolo[3,2-b]pyridine-6-carbonitrile (Example 23) (3.1 g, 8.50 mmol), 2,4-pentadienone (0.085 g, 0.850 mmol), copper(I) iodide (0.162 g, 0.850 mmol) and ammonium hydroxide (9.93 mL, 255 mmol) in DMF (50 mL) was heated at 100° C. in a schlenk tube for 24 h. The reaction mixture was filtered through a bed of celite. The filtrate was neutralised with 1N HCl then extracted with EtOAc. The organic layer was dried over Na₂SO₄ and concentrated under reduced pressure. The residue was triturated with iPr₂O to give the product 1-(4-aminophenyl)-2-chloro-7-hydroxy-5-oxo-4,5-dihydro-1H-pyrrolo[3,2-b]pyridine-6-carbonitrile (1.75 g, 5.82 mmol, 68% yield) as a yellow powder. LCMS: (M+H)⁺: 301; Rt: 1.47 min.

### Intermediate 141

### Ethyl 3-(acetylamino)-1-{4-[(ethyloxy)carbonyl]phenyl}-1H-pyrrole-2-carboxylate

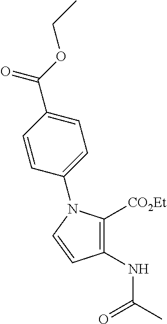

To a solution of ethyl 3-(acetylamino)-1H-pyrrole-2-carboxylate (Intermediate 1) (1 g, 5.10 mmol) in DCM (50 mL) was added {4-[(ethyloxy)carbonyl]phenyl}boronic acid (1.088 g, 5.61 mmol), pyridine (0.618 mL, 7.65 mmol) and copper(II) acetate (1.018 g, 5.61 mmol). The reaction mixture was stirred at room temperature for 2 days. {4-[(Ethyloxy)carbonyl]phenyl}boronic acid (1.088 g, 5.61 mmol) and pyridine (0.618 mL, 7.65 mmol) and copper(II) acetate (1.018 g, 5.61 mmol) were added again. The reaction was stirred at RT for another day before water was added and the product was extracted with DCM. The organic layer was dried over Na₂SO₄ filtered and evaporated off. The residue was purified by chromatography on silica gel eluting with (DCM/EtOAc 100/0 to 90/10) to give the product ethyl 3-(acetylamino)-1-{4-[(ethyloxy)carbonyl]phenyl}-1H-pyrrole-2-carboxylate (600 mg, 1.394 mmol, 27.3% yield) as a yellow oil. LCMS: (M+H)⁺: 345; Rt: 3.12 min.

### Intermediate 142

### Ethyl 3-amino-1-{4-[(ethyloxy)carbonyl]phenyl}-1H-pyrrole-2-carboxylate

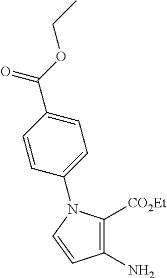

To a solution of ethyl 3-(acetylamino)-1-{4-[(ethyloxy)carbonyl]phenyl}-1H-pyrrole-2-carboxylate (Intermediate 141) (620 mg, 1.800 mmol) in ethanol (50 mL) was added concentrated HCl (0.191 mL, 6.30 mmol) and the reaction mixture was stirred at reflux for 16 h. After evaporation the residue was taken up in water and extracted with DCM. The organic layer was washed with a solution of NaHCO₃, dried over Na₂SO₄, filtered and evaporated to give ethyl 3-amino-1-{4-[(ethyloxy)carbonyl]phenyl}-1H-pyrrole-2-carboxylate (470 mg, 1.244 mmol, 69.1% yield) as yellow oil. LCMS: (M+H)⁺: 303; Rt: 3.38 min.

### Intermediate 143

### Ethyl 3-[(cyanoacetyl)amino]-1-{4-[(ethyloxy)carbonyl]phenyl}-1H-pyrrole-2-carboxylate

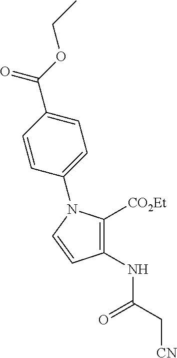

To a solution of ethyl 3-amino-1-{4-[(ethyloxy)carbonyl]phenyl}-1H-pyrrole-2-carboxylate (Intermediate 142) (470 mg, 1.555 mmol) in acetonitrile (10 mL) were added cyanoacetic acid (159 mg, 1.866 mmol), and a solution of DCC (529 mg, 2.57 mmol) in acetonitrile (10 mL). The mixture was stirred at 45° C. for 2 days. The resulting solid was filtered and discarded and the filtrate was evaporated off. The residue was purified by chromatography on silica gel eluting with (cyclohexane/EtOAc 100/0 to 0/100) to give the product ethyl 3-[(cyanoacetyl)amino]-1-{4-[(ethyloxy)carbonyl]phenyl}-1H-pyrrole-2-carboxylate (250 mg, 0.677 mmol, 43.5% yield) as yellow oil. LCMS: (M+H)⁺: 370; Rt: 3.11 min.

### Example 1

### 1-(4-Bromophenyl)-7-hydroxy-5-oxo-4,5-dihydro-1H-pyrrolo[3,2-b]pyridine-6-carbonitrile

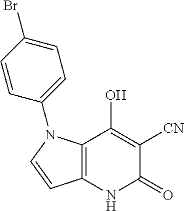

Method A: To a solution of ethyl 1-(4-bromophenyl)-3-[(cyanoacetyl)amino]-1H-pyrrole-2-carboxylate (Intermediate 4) (510 mg, 1.36 mmol) in tetrahydrofuran (5 mL) at RT was added portion-wise sodium hydride (65 mg, 1.625 mmol). After hydrogen evolution stopped, the reaction mixture was stirred at RT for 18 hours and at reflux for 24 hours before being cooled down and quenched with MeOH. The mixture was concentrated to dryness and taken up in MeOH with a few drops of water and triturated at reflux. After cooling down to RT, the solid was filtered and the resulting filtrate was concentrated under reduced pressure to give the desired compound 1-(4-bromophenyl)-7-hydroxy-5-oxo-4,5-dihydro-1H-pyrrolo[3,2-b]pyridine-6-carbonitrile (330 mg, 1.000 mmol, 74% yield) as an orange solid. ¹H NMR: (DMSO-d6, 400 MHz) δ 9.85 (s, 1H), 7.53 (m, 2H), 7.34 (m, 2H), 7.04 (d, J=3.0 Hz, 1H), 5.92 (d, J=3.0 Hz, 1H). LCMS: (M+H)⁺: 330, 332; Rt: 2.10 min.

Method B: To a solution of ethyl 1-(4-bromophenyl)-3-[(cyanoacetyl)amino]-1H-pyrrole-2-carboxylate (Intermediate 4) (4.5 g, 11.96 mmol) in THF (45 mL) at RT was added portionwise sodium hydride (60% suspension in oil, 574 mg, 14.35 mmol). After hydrogen evolution stopped, the reaction mixture was stirred at reflux for 48 h before being cooled down and quenched with MeOH. The mixture was concentrated to dryness and taken up in MeOH with a few drops of water and triturated at reflux. After cooling down to RT, the solid was filtered to give the desired compound 1-(4-bromophenyl)-7-hydroxy-5-oxo-4,5-dihydro-1H-pyrrolo[3,2-b]pyridine-6-carbonitrile (3.4 g, 10.30 mmol, 86% yield) as a light yellow solid. HRMS: calculated for C₁₄H₇BrN₃O₂(M−H)⁻: 329.9878; found: 329.9843; Rt: 1.96 min.

### Example 2

### 7-Hydroxy-1-(2′-hydroxy-4-biphenylyl)-5-oxo-4,5-dihydro-1H-pyrrolo[3,2-b]pyridine-6-carbonitrile

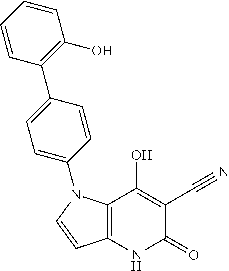

To a mixture of 1-(4-bromophenyl)-7-hydroxy-5-oxo-4,5-dihydro-1H-pyrrolo[3,2-b]pyridine-6-carbonitrile (Example 1) (330 mg, 1.0 mmol), (2-hydroxyphenyl)boronic acid (276 mg, 2.0 mmol) and cesium carbonate (651 mg, 2.0 mmol) in a 1,4-dioxane (8 mL)/water (2 mL) mixture was added Pd(PPh₃)₄ (4 mg, 3.46 μmol). The reaction vessel was sealed and heated in Biotage Initiator using initial high to 160° C. for 15 min. After cooling the mixture was filtered off and the filtrate was concentrated under reduced pressure. The residue was dissolved in an acetonitrile/AcOH (1:1) mixture (4 mL) and treated with charcoal then hot filtered, the filtrate was evaporated to dryness. The residue was triturated in methanol to give 7-hydroxy-1-(2′-hydroxy-4-biphenylyl)-5-oxo-4,5-dihydro-1H-pyrrolo[3,2-b]pyridine-6-carbonitrile (12 mg, 0.035 mmol, 3.50% yield) as a white solid. ¹H NMR: (DMSO-d6, 400 Hz) δ 9.75 (s, 1H), 9.59 (s, 1H), 7.51 (d, J=8.5 Hz, 2H), 7.38 (d, J=8.5 Hz, 2H), 7.28 (m, 1H), 7.16 (m, 1H), 7.04 (d, J=2.9 Hz, 1H), 6.96 (d, J=7.2 Hz, 1H), 6.89 (dd, J=7.5 and 7.5 Hz, 1H), 5.90 (d, J=2.9 Hz, 1H). LCMS: (M+H)⁺: 344; Rt: 2.28 min. HRMS: calculated for C₂₀H₁₄N₃O₃(M+H)⁺: 344.1035; found: 344.1003; Rt: 2.20 min.

Examples 3 to 8 of formula (I), wherein R¹ is cyano and R³, R⁴, R⁶ and R⁷ are all H were prepared by methods analogous to that described for Example 2 from Example 1 using the appropriate boronic acid.

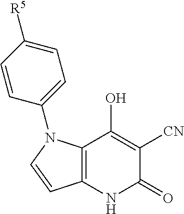

TABLE 12

| Example | Name | R⁵ | Physical data |
|---|---|---|---|
| 3 | 7-Hydroxy-1-[4′- (methyloxy)-4- biphenylyl]-5-oxo-4,5- dihydro-1H-pyrrolo[3,2- b]pyridine-6-carbonitrile | 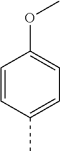 | ¹H NMR: (DMSO-d6, 300 Hz) δ 9.77 (s, 1H), 7.70-7.53 (m, 4H), 7.42 (d, J = 8.3 Hz, 2H), 7.11-6.99 (m, 3H), 5.91 (m, 1H), 3.80 (s, 3H). LCMS: (M + H)⁺: 358; Rt: 2.46 min. HRMS: calculated for C₂₁H₁₆N₃O₃ (M + H)⁺: 358.1191; found: 358.1191; Rt: 2.26 min. |
| 4 | 7-Hydroxy-1-[2′- (methyloxy)-4- biphenylyl]-5-oxo-4,5- dihydro-1H-pyrrolo[3,2- b]pyridine-6-carbonitrile | 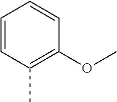 | ¹H NMR: (DMSO-d6, 300 Hz) δ 9.76 (s, 1H), 7.47-7.29 (m, 6H), 7.13 (d, J = 8.2 Hz, 1H), 7.09-7.00 (m, 2H), 5.91 (m, 1H), 3.80 (s, 3H). LCMS: (M + H)⁺: 358; Rt: 2.46 min. HRMS: calculated for C₂₁H₁₆N₃O₃ (M + H)⁺: 358.1191; found: 358.1176; Rt: 2.26 min. |
| 5 | 7-Hydroxy-1-[3′- (methyloxy)-4- biphenylyl]-5-oxo-4,5- dihydro-1H-pyrrolo[3,2- b]pyridine-6-carbonitrile | 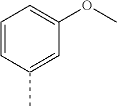 | ¹H NMR: (DMSO-d6, 400 Hz) δ 9.78 (s, 1H), 7.64 (d, J = 8.5 Hz, 2H), 7.45 (d, J = 8.5 Hz, 2H), 7.38 (d, J = 7.7 Hz, 1H), 7.26 (d, J = 7.7 Hz, 1H), 7.21 (s, 1H), 7.06 (d, J = 3.0 Hz, 1H), 6.94 (m, 1H), 5.91 (d, J = 3.0 Hz, 1H), 3.84 (s, 3H). LCMS: (M + H)⁺: 358; Rt: 2.47 min. HRMS: calculated for C₂₁H₁₆N₃O₃ (M + H)⁺: 358.1191; found: 358.1190; Rt: 2.28 min. |
| 6 | 1-(4′-Fluoro-4- biphenylyl)-7-hydroxy-5- oxo-4,5-dihydro-1H- pyrrolo[3,2-b]pyridine-6- carbonitrile | 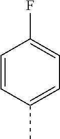 | ¹H NMR: (DMSO-d6, 300 Hz) δ 9.77 (s, 1H), 7.75 (d, J = 5.5 Hz, 1H), 7.72 (d, J = 5.5 Hz, 1H), 7.62 (d, J = 8.5 Hz, 2H), 7.45 (d, J = 8.5 Hz, 2H), 7.31 (m, 2H), 7.06 (d, J = 2.9 Hz, 1H), 5.91 (d, J = 2.9 Hz, 1H). LCMS: (M + H)⁺: 346; Rt: 2.49 min. HRMS: calculated for C₂₀H₁₃FN₃O₂ (M + H)⁺: 346.0992; found: 346.0980; Rt: 2.33 min. |
| 7 | 1-(2′-Cyano-4- biphenylyl)-7-hydroxy-5- oxo-4,5-dihydro-1H- pyrrolo[3,2-b]pyridine-6- carbonitrile | 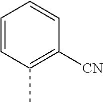 | ¹H NMR: (DMSO-d6, 300 Hz) δ 9.80 (s, 1H), 7.97 (d, J = 7.6 Hz, 1H), 7.82 (m, 1H), 7.67 (d, J = 7.6 Hz, 1H), 7.63-7.49 (m, 5H), 7.12 (m, 1H), 5.93 (m, 1H). LCMS: (M + H)⁺: 353; Rt: 2.32 min. HRMS: calculated for C₂₁H₁₃N₄O₂ (M + H)⁺: 353.1039; found: 353.1019; Rt: 2.13 min. |
| 8 | 1-(3′-Fluoro-4- biphenylyl)-7-hydroxy-5- oxo-4,5-dihydro-1H- pyrrolo[3,2-b]pyridine-6- carbonitrile | 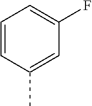 | ¹H NMR: (DMSO-d6, 300 Hz) δ 9.78 (s, 1H), 7.69 (d, J = 8.5 Hz, 2H), 7.61-7.43 (m, 3H), 7.47 (d, J = 8.5 Hz, 2H), 7.20 (m, 1H), 7.08 (d, J = 2.9 Hz, 1H), 5.92 (d, J = 2.9 Hz, 1H). LCMS: (M + H)⁺: 346; Rt: 2.50 min. HRMS: calculated or C₂₀H₁₃FN₃O₂ (M + H)⁺: 346.0992; found: 346.0989; Rt: 2.30 min. |

### Example 9

### 7-Hydroxy-5-oxo-1-phenyl-4,5-dihydro-1H-pyrrolo[3,2-b]pyridine-6-carbonitrile

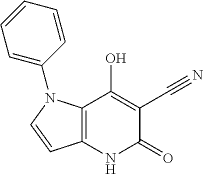

A solution of 1-(4-bromophenyl)-7-hydroxy-5-oxo-4,5-dihydro-1H-pyrrolo[3,2-b]pyridine-6-carbonitrile (Example 1) (200 mg, 0.606 mmol) in methanol (12 mL) was hydrogenated using the H-cube (settings: 40° C., 10 bar, 1 mL/min) and a 10% Pd/C cartridge as the catalyst. The solution was then concentrated under reduced pressure. The residue was triturated in refluxing acetonitrile to give 7-hydroxy-5-oxo-1-phenyl-4,5-dihydro-1H-pyrrolo[3,2-b]pyridine-6-carbonitrile (90 mg, 0.358 mmol, 59.1% yield) as a white powder. ¹H NMR: (DMSO-d6, 300 Hz) δ 11.39 (br s, 1H), 7.53-7.29 (m, 6H), 6.15 (d, j=2.9 Hz, 1H). LCMS: (M+H)⁺: 252; Rt: 1.80 min. HRMS: calculated for C14H10N3O2 (M+H)+: 252.0773; found: 252.0763; Rt: 1.95 min.

Examples 10 to 19 of formula (I), wherein R¹ is cyano and R³, R⁴, R⁶ and R⁷ are all H, were prepared by methods analogous to that described for Example 2 from Example 1 using the appropriate boronic acid.

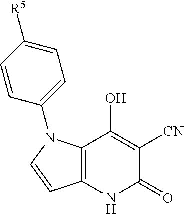

TABLE 13

| Example | Name | R⁵ | Physical data |
|---|---|---|---|
| 10 | 7-Hydroxy-1-[2′- hydroxy-3′-(methyloxy)- 4-biphenylyl]-5-oxo-4,5- dihydro-1H-pyrrolo[3,2- b]pyridine-6-carbonitrile | 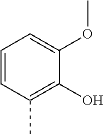 | ¹H NMR: (DMSO-d6, 300 MHz) δ 11.96 (br s, 1H), 8.75 (br s, 1H), 7.61 (d, J = 8.5 Hz, 2H), 7.51 (d, J = 3.0 Hz, 1H), 7.46 (d, J = 8.5 Hz, 2H), 7.05-6.83 (m, 3H), 6.20 (d, J = 3.0 Hz, 1H), 3.86 (s, 3H). LCMS: (M + H)⁺: 374; Rt: 2.60 min. HRMS: calculated for C₂₀H₁₆N₃O₄ (M + H)⁺: 374.1141; found: 374.1110; Rt: 2.10 min. |
| 11 | 1-(2′-Fluoro-4- biphenylyl)-7-hydroxy-5- oxo-4,5-dihydro-1H- pyrrolo[3,2-b]pyridine-6- carbonitrile | 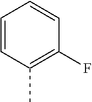 | ¹H NMR: (DMSO-d6, 300 Hz) δ 9.80 (s, 1H), 7.61-7.38 (m, 6H), 7.36- 7.28 (m, 2H), 7.09 (d, J = 3.1 Hz, 1H), 5.90 (d, J = 3.1 Hz, 1H). LCMS: (M + H)⁺: 346; Rt: 2.48 min. HRMS: calculated for C₂₀H₁₃FN₃O₂ (M + H)⁺: 346.0992; found; 346; Rt: 2.51 min. |
| 12 | 1-(4′-Chloro-4- biphenylyl)-7-hydroxy-5- oxo-4,5-dihydro-1H- pyrrolo[3,2-b]pyridine-6- carbonitrile | 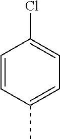 | ¹H NMR: (DMSO-d6, 300 Hz) δ 9.78 (s, 1H), 7.74 (d, J = 8.5 Hz, 2H), 7.65 (d, J = 8.5 Hz, 2H), 7.53 (d, J = 8.5 Hz, 2H), 7.47 (d, J = 8.5 Hz, 2H), 7.07 (d, J = 2.9 Hz, 1H), 5.92 (d, J = 2.9 Hz, 1H). LCMS: (M + H)⁺: 362; Rt: 2.65 min. HRMS: calculated for C₂₀H₁₃ClN₃O₂ (M + H)⁺: 362.0696; found; 362.0689; Rt: 2.63 min. |
| 13 | 7-Hydroxy-1-(4′- hydroxy-4-biphenylyl)-5- oxo-4,5-dihydro-1H- pyrrolo[3,2-b]pyridine-6- carbonitrile | 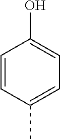 | ¹H NMR: (DMSO-d6, 300 Hz) δ 9.75 (s, 1H), 9.55 (s, 1H), 7.57-7.47 (m, 4H), 7.39 (d, J = 8.4 Hz, 2H), 7.03 (d, J = 3.0 Hz, 1H), 6.86 (d, J = 7.2 Hz, 2H), 5.90 (d, J = 3.09 Hz, 1H). LCMS: (M + H)⁺: 344; Rt: 2.13 min. HRMS: calculated for C₂₀H₁₄N₃O₃ (M + H)⁺: 344.1035; found: 344.1052; Rt: 2.22 min. |
| 14 | 7-Hydroxy-5-oxo-1-[4′- (trifluoromethyl)-4- biphenylyl]-4,5-dihydro- 1H-pyrrolo[3,2- b]pyridine-6-carbonitrile | 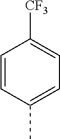 | ¹H NMR: (DMSO-d6, 300 Hz) δ 9.79 (s, 1H), 7.94 (d, J = 8.4 Hz, 2H), 7.83 (d, J = 8.4 Hz, 2H), 7.73 (d, J = 8.5 Hz, 2H), 7.52 (d, J = 8.5 Hz, 2H), 7.10 (d, J = 3.0 Hz, 1H), 5.93 (d, J = 3.0 Hz, 1H). LCMS: (M + H)⁺: 396; Rt: 2.74 min. HRMS: calculated for C₂₁H₁₃F₃N₃O₂ (M + H)⁺: 396.0960; found: 396.0971; Rt: 2.80 min. |
| 15 | 7-Hydroxy-1-(4′-methyl- 4-biphenylyl)-5-oxo-4,5- dihydro-1H-pyrrolo[3,2- b]pyridine-6-carbonitrile | 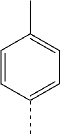 | ¹H NMR: (DMSO-d6, 300 Hz) δ 9.89 (s, 1H), 7.65-7.55 (m, 4H), 7.44 (d, J = 8.6 Hz, 2H), 7.29 (d, J = 8.0 Hz, 2H), 7.08 (d, J = 2.9 Hz, 1H), 5.93 (d, J = 2.9 Hz, 1H), 2.35 (s, 3H). LCMS: (M + H)⁺: 342; Rt: 2.59 min. HRMS: calculated for C₂₁H₁₆N₃O₂ (M + H)⁺: 342.1242; found: 342.1233; Rt; 2.63 min. |
| 16 | 1-(4′-Cyano-4- biphenylyl)-7-hydroxy-5- oxo-4,5-dihydro-1H- pyrrolo[3,2-b]pyridine-6- carbonitrile | 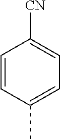 | ¹H NMR: (DMSO-d6, 300 Hz) δ 10.08 (br s, 1H), 7.99-7.90 (m, 4H), 7.98 (s, 1H), 7.77 (d, J = 8.6 Hz, 2H), 7.53 (d, J = 8.6 Hz, 2H), 7.16 (d, J = 2.7 Hz, 1H), 5.97 (d, J = 2.7 Hz, 1H). LCMS: (M + H)⁺: 353; Rt: 2.34 min. HRMS: calculated for C₂₁H₁₃N₄O₂ (M + H)⁺: 353.1039; found: 353.1032; Rt: 2.47 min. |
| 17 | N-[4′-(6-Cyano-7- hydroxy-5-oxo-4,5- dihydro-1H-pyrrolo[3,2- b]pyridin-1-yl)-4- biphenylyl]acetamide | 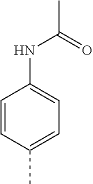 | ¹H NMR: (DMSO-d6, 300 Hz) δ 10.04 (s, 1H), 9.77 (s, 1H), 7.73- 7.56 (m, 6H), 7.43 (d, J = 8.6 Hz, 2H), 7.05 (d, J = 2.9 Hz, 1H), 5.91 (d, J = 2.9 Hz, 1H), 2.07 (s, 3H). LCMS: (M + H)⁺: 385; Rt: 2.08 min. HRMS: calculated for C₂₂H₁₇N₄O₃ (M + H)⁺: 385.1301; found: 385.1326; Rt: 2.19 min. |
| 18 | 1-(2′-Chloro-4- biphenylyl)-7-hydroxy-5- oxo-4,5-dihydro-1H- pyrrolo[3,2-b]pyridine-6- carbonitrile | 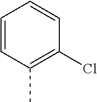 | ¹H NMR: (DMSO-d6, 300 Hz) δ 11.64 (s, 1H), 7.64-7.39 (m, 9H), 6.21 (d, J = 2.8 Hz, 1H). LCMS: (M + H)⁺: 362; Rt: 2.54 min. HRMS: calculated for C₂₀H₁₃ClN₃O₂ (M + H)⁺: 362.0696; found: 362.0701; Rt: 2.66 min. |
| 19 | 7-Hydroxy-1-{4-[6- (methyloxy)-3- pyridinyl]phenyl}-5-oxo- 4,5-dihydro-1H- pyrrolo[3,2-b]pyridine-6- carbonitrile | 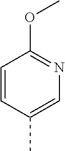 | ¹H NMR: (DMSO-d6, 300 Hz) δ 11.63 (br s, 1H), 8.55 (s, 1H), 8.08 (d, J = 8.9 Hz, 1H), 7.74 (d, J = 8.3 Hz, 1H), 7.52 (d, J = 8.3 Hz, 1H), 7.50 (m, 1H), 6.94 (d, J = 8.9 Hz, 1H), 6.20 (m, 1H), 3.91 (s, 3H). LCMS: (M + H)⁺: 359; Rt: 2.26 min. HRMS: calculated for C₂₀H₁₅N₄O₃ (M + H)⁺: 359.1144; found: 359.1110; Rt: 2.09 min. |

### Example 20

### 1-(4′-Fluoro-4-biphenylyl)-7-hydroxy-4-methyl-5-oxo-4,5-dihydro-1H-pyrrolo[3,2-b]pyridine-6-carbonitrile

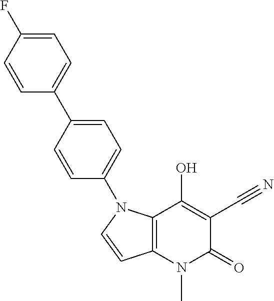

To a solution of 1-(4′-fluoro-4-biphenylyl)-7-hydroxy-5-oxo-4,5-dihydro-1H-pyrrolo[3,2-b]pyridine-6-carbonitrile (Example 6) (100 mg, 0.290 mmol) in DMF (5 mL) and was added portion-wise sodium hydride (60% suspension in oil, 12.74 mg, 0.319 mmol). After hydrogen evolution stopped, methyl iodide (0.018 μL, 0.290 mmol) was added, the tube was sealed and heated under Biotage initiator 8 (power: very High) at 110° C. for 15 min. After cooling water was added and the precipitate was filtered off and the resulting solid was recrystallised from acetonitrile to give 1-(4′-fluoro-4-biphenylyl)-7-hydroxy-4-methyl-5-oxo-4,5-dihydro-1H-pyrrolo[3,2-b]pyridine-6-carbonitrile (20 mg, 0.056 mmol, 19.22% yield) as a yellow crystals. LCMS: (M+H)⁺: 360; Rt: 2.81 min. HRMS: calculated for C₂₁H₁₅FN₃O₂ (M+H)⁺: 360.1148; found: 360.1119 Rt: 2.40 min.

### Example 21

### 7-Hydroxy-1-(2′-hydroxy-4-biphenylyl)-1,4-dihydro-5H-pyrrolo[3,2-b]pyridin-5-one

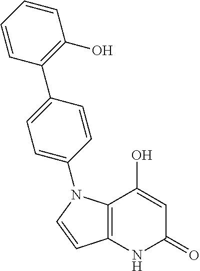

To a mixture of ethyl 1-(4-bromophenyl)-7-hydroxy-5-oxo-4,5-dihydro-1H-pyrrolo[3,2-b]pyridine-6-carboxylate (Intermediate 6) (942 mg, 2.497 mmol), (2-hydroxyphenyl)boronic acid (689 mg, 4.99 mmol) and cesium carbonate (1.63 mg, 4.99 mmol) in a 1,4-dioxane (8 mL)/water (2 mL) mixture was added Pd(PPh₃)₄ (115 mg, 0.100 mmol). The reaction vessel was sealed and heated in Biotage Initiator using initial high to 150° C. for 15 min. After cooling water was added and the precipitate was filtered off and the cake was hot triturated in acetic acid then treated with charcoal and filtered. The filtrate was evaporated and the residue was triturated in water. The residue was filtered and washed successively with acetonitrile and diethyl ether to give 7-hydroxy-1-(2′-hydroxy-4-biphenylyl)-1,4-dihydro-5H-pyrrolo[3,2-b]pyridin-5-one (130 mg, 0.408 mmol, 16.35% yield) as a cream powder. ¹H NMR: (DMSO-d6, 300 Hz) δ 10.56 (br s, 1H), 7.57 (d, J=8.4 Hz, 2H), 7.43 (d, J=8.4 Hz, 2H), 7.31 (d, J=6.6 Hz, 1H), 7.17 (d, J=3.2 Hz, 1H), 7.15 (m, 1H), 6.95 (d, J=8.1 Hz, 1H), 6.87 (dd, J=7.4 and 7.4 Hz, 1H), 6.07 (d, J=3.2 Hz, 1H), 5.19 (br s, 1H). LCMS: (M+H)⁺: 319; Rt: 2.44 min. HRMS: calculated for C₁₉H₁₅N₂O₃ (M+H)⁺: 319.1082; found: 319.1094; Rt: 2.22 min.

### Example 22

### 1-[4-(6-Fluoro-3-pyridinyl)phenyl]-7-hydroxy-5-oxo-4,5-dihydro-1H-pyrrolo[3,2-b]pyridine-6-carbonitrile

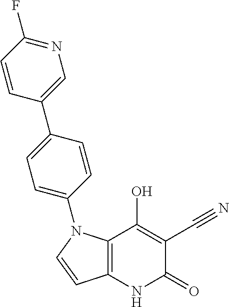

To a mixture of 1-(4-bromophenyl)-7-hydroxy-5-oxo-4,5-dihydro-1H-pyrrolo[3,2-b]pyridine-6-carbonitrile (Example 1) (330 mg, 1.000 mmol), (6-fluoro-3-pyridinyl)boronic acid (282 mg, 2.0 mmol) and cesium carbonate (651 mg, 2.0 mmol) in a 1,4-dioxane (4 mL)/water (1 mL) mixture was added Pd(PPh₃)₄ (46.0 mg, 0.040 mmol). The reaction vessel was sealed and heated in Biotage Initiator using initial high to 150° C. for 15 min. After cooling, water was added to the mixture and the resulting solid was filtered off and washed with sat. NH₄Cl. The solid was dissolved in hot ethanol and treated with charcoal, filtered and evaporated in vacuo. The residue was recrystallised in DMF and the white powder was rinsed with water and acetonitrile. 1-[4-(6-fluoro-3-pyridinyl)phenyl]-7-hydroxy-5-oxo-4,5-dihydro-1H-pyrrolo[3,2-b]pyridine-6-carbonitrile (5 mg, 0.014 mmol, 1.444% yield) was obtained as a white powder. ¹H NMR: (DMSO-d6, 300 Hz) δ 11.50 (br s, 1H), 8.62 (d, J=2.1 Hz, 1H), 8.35 (ddd, J=8.3, 8.3 and 2.6 Hz, 1H), 7.81 (d, J=8.5 Hz, 2H), 7.56 (d, J=8.5 Hz, 2H), 7.8 (d, J=3.0 Hz, 1H), 7.32 (dd, J=8.3 and 2.6 Hz, 1H), 6.20 (d, J=3.0 Hz, 1H). LCMS: (M+H)⁺: 347; Rt: 2.20 min. HRMS: calculated for C₁₉H₁₂FN₄O₂ (M+H)⁺: 347.0944; found: 347.0912; Rt: 2.01 min.

### Example 23

### 1-(4-Bromophenyl)-2-chloro-7-hydroxy-5-oxo-4,5-dihydro-1H-pyrrolo[3,2-b]pyridine-6-carbonitrile

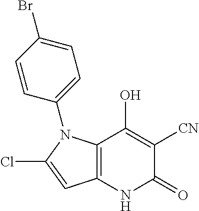

Method A: To a solution of ethyl 1-(4-bromophenyl)-5-chloro-3-[(cyanoacetyl)amino]-1H-pyrrole-2-carboxylate (Intermediate 10) (5.66 g, 13.78 mmol) in THF (50 mL) was added sodium hydride (0.662 g, 16.54 mmol). After hydrogen evolution stopped, the reaction mixture was stirred at 50° C. during 2 hours. Sodium hydride (1.2 eq) was added again twice and the mixture was stirred at 50° C. overnight. The mixture was then stirred at RT for two days before being quenched with 1N HCl. The reaction mixture was dissolved in EtOAc and the organic layer was concentrated in vacuo. The solid was triturated in EtOAc and filtered. The product was then purified on a Isco Companion with the filtrate. The sample was loaded on 120 g AIT silica (Si) column then the purification was carried out using a DCM/MeOH 100/0 to 80/20 gradient. The appropriate fractions were combined and concentrated in vacuo to give the required product (2.7 g, 70% yield) as a orange solid. ¹H NMR: (DMSO-d6, 300 Hz) δ 11.60 (br s, 1H), 7.70 (d, J=8.5 Hz, 2H), 7.38 (d, J=8.5 Hz, 2H), 6.30 (s, 1H). LCMS: (M+H)⁺: 364, 366; Rt: 2.55 min.

Method B: To a solution of ethyl 1-(4-bromophenyl)-5-chloro-3-[(cyanoacetyl)amino]-1H-pyrrole-2-carboxylate (Intermediate 10) (2.6 g, 6.33 mmol) in THF (50 ml) was added sodium hydride (60% suspension in oil, 0.304 g, 7.60 mmol). The reaction mixture was stirred at 45° C. for 54 h and after cooling water was added and acidified with 1N HCl to pH 1. The product was extracted with DCM and the organic layer was dried over Na₂SO₄, filtered and evaporated off. Recrystallisation in CH₃CN gave the product 1-(4-bromophenyl)-2-chloro-7-hydroxy-5-oxo-4,5-dihydro-1H-pyrrolo[3,2-b]pyridine-6-carbonitrile (880 mg, 2.293 mmol, 36.2% yield) as cream powder. LCMS: (M+H)⁺: 364, 366; Rt: 2.37 min. HRMS: calculated for C₁₄H₈BrClN₃O₂(M+H)⁺: 363.9488; found: 363.9469; Rt: 2.19 min.

### Example 24

### 2-Chloro-7-hydroxy-1-[2′-hydroxy-3′-(methyloxy)-4-biphenylyl]-5-oxo-4,5-dihydro-1H-pyrrolo[3,2-b]pyridine-6-carbonitrile

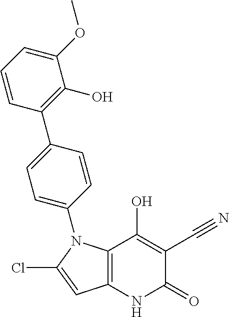

To a solution of 1-(4-bromophenyl)-2-chloro-7-hydroxy-5-oxo-4,5-dihydro-1H-pyrrolo[3,2-b]pyridine-6-carbonitrile (Example 23) (150 mg, 0.411 mmol) in 1,4-dioxane (5 mL)/water (3 mL) were added [2-hydroxy-3-(methyloxy)phenyl]boronic acid (83 mg, 0.494 mmol), cesium carbonate (402 mg, 1.234 mmol) and Pd(PPh₃)₄ (1.0 mg, 1.234 μmol). The reaction vessel was sealed and heated to 160° C. for 20 min in a microwave reactor. To complete the reaction, [2-hydroxy-3-(methyloxy)phenyl]boronic acid (83 mg, 0.494 mmol) and Pd(PPh₃)₄ (1.0 mg, 1.234 μmol) were added again and the reaction vessel was sealed and heated to 160° C. for 20 min in microwave reactor. After cooling, the reaction mixture was extracted, and the organic layer was washed with 1N HCl solution and evaporated off. The residue was dissolved with a hot MeOH/CH₃CN (80/20) mixture and treated with charcoal filtered. The filtrate was crystallined to give the product 2-chloro-7-hydroxy-1-[2′-hydroxy-3′-(methyloxy)-4-biphenylyl]-5-oxo-4,5-dihydro-1H-pyrrolo[3,2-b]pyridine-6-carbonitrile (50 mg, 0.116 mmol, 28.3% yield) as a white powder. ¹H NMR: (DMSO-d6, 300 MHz) δ 9.74 (br s, 1H), 8.71 (s, 1H), 7.56 (d, J=8.4 Hz, 2H), 7.23 (d, J=8.4 Hz, 2H), 7.02-6.84 (m, 3H), 5.94 (s, 1H), 3.86 (s, 3H). LCMS: (M+H)⁺: 408; Rt: 2.63 min. HRMS: calculated for C₂₁H₁₅ClN₃O₄ (M+H)⁺: 408.0751; found: 408.0765; Rt: 2.34 min.

Examples 25 to 29 of formula (I), wherein R³, R⁴ and R⁷ are all H and R⁶ is Cl, were prepared by methods analogous to that described for Example 24 from Example 23 using the appropriate boronic acid.

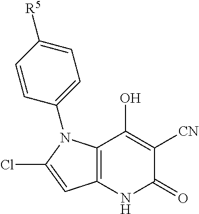

TABLE 14

| Example | Name | R⁵ | Physical data |
|---|---|---|---|
| 25 | 2-Chloro-1-(2′-fluoro-4- biphenylyl)-7-hydroxy-5- oxo-4,5-dihydro-1H- pyrrolo[3,2-b]pyridine-6- carbonitrile | 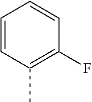 | ¹H NMR: (DMSO-d6, 300 Hz) δ 9.78 (br s, 1H), 7.65-7.55 (m, 3H), 7.44 (m, 1H), 7.40-7.25 (m, 4H), 5.97 (s, 1H). LCMS: (M + H)⁺: 380; Rt: 2.73 min. HRMS: calculated for C₂₀H₁₂ClFN₃O₂ (M + H)⁺: 380.0602; found: 380.0608; Rt: 2.45 min. |
| 26 | 2-Chloro-7-hydroxy-1- (2′-hydroxy-4- biphenylyl)-5-oxo-4,5- dihydro-1H-pyrrolo[3,2- b]pyridine-6-carbonitrile | 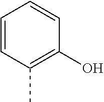 | ¹H NMR: (DMSO-d6, 300 Hz) δ 9.75 (br s, 1H), 9.66 (br s, 1H), 7.58 (m, 2H), 7.32 (dd, J = 7.7 and 1.7 Hz, 1H), 7.24 (m, 2H), 7.18 (m, 1H), 6.98 (m, 1H), 6.91 (m, 1H), 5.95 (s, 1H). LCMS: (M + H)⁺: 378; Rt: 2.59 min. HRMS: calculated for C₂₀H₁₃ClN₃O₃ (M + H)⁺: 378.0645; found : 378.0648; Rt: 2.33 min. |
| 27 | 2-Chloro-7-hydroxy-1- (4′-methyl-4-biphenylyl)- 5-oxo-4,5-dihydro-1H- pyrrolo[3,2-b]pyridine-6- carbonitrile | 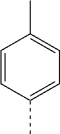 | ¹H NMR: (DMSO-d6, 300 Hz) δ 9.76 (s, 1H), 7.70-7.60 (m, 4H), 7.40-7.25 (m, 4H), 5.95 (, 1H), 2.36 (s, 3H). LCMS: (M + H)⁺: 376; Rt: 2.86 min. HRMS: calculated for C₂₁H₁₅ClN₃O₂ (M + H)⁺: 376.0853; found: 376.0861; Rt: 2.52 min. |
| 28 | 2-Chloro-1-(4′-fluoro-4- biphenylyl)-7-hydroxy-5- oxo-4,5-dihydro-1H- pyrrolo[3,2-b]pyridine-6- carbonitrile | 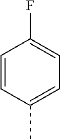 | ¹H NMR: (DMSO-d6, 300 Hz) δ 11.29 (br s, 1H), 7.85-7.70 (m, 2H), 7.76 (d, J = 8.7 Hz, 2H), 7.45 (d, J = 8.7 Hz, 2H), 7.33 (m, 2H), 6.25 (s, 1H). LCMS: (M + H)⁺: 380; Rt: 2.76 min. HRMS: calculated for C₂₀H₁₂ClFN₃O₂ (M + H)⁺: 380.0602; found: 380.0619; Rt: 2.42 min. |
| 29 | 2-Chloro-7-hydroxy-1- [4′-(methyloxy)-4- biphenylyl]-5-oxo-4,5- dihydro-1H-pyrrolo[3,2- b]pyridine-6-carbonitrile | 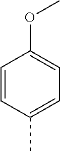 | ¹H NMR: (DMSO-d6, 300 Hz) δ 11.36 (br s, 1H), 7.75-7.66 (m, 2H), 7.41 (d, J = 8.3 Hz, 1H), 7.07 (d, J = 9.0 Hz, 1H), 6.25 (s, 1H), 3.82 (s, 3H). LCMS: (M + H)⁺: 392; Rt: 2.73 min. HRMS: calculated for C₂₁H₁₅ClN₃O₃ (M + H)⁺: 392.0802; found: 392.0819; Rt: 2.39 min. |

### Example 30

### Ethyl 7-hydroxy-1-(2′-hydroxy-4-biphenylyl)-5-oxo-4,5-dihydro-1H-pyrrolo[3,2-b]pyridine-6-carboxylate

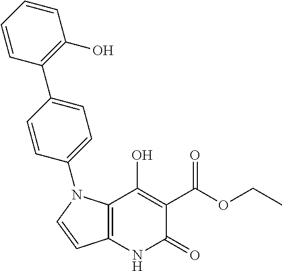

A mixture of ethyl 1-(4-bromophenyl)-7-hydroxy-5-oxo-4,5-dihydro-1H-pyrrolo[3,2-b]pyridine-6-carboxylate (Intermediate 6) (754 mg, 2.0 mmol), (2-hydroxyphenyl)boronic acid (414 mg, 3.00 mmol), tripotassium phosphate (424 mg, 2.0 mmol), tri-o-tolylphosphine (36.5 mg, 0.120 mmol) and palladium(II) acetate (17.95 mg, 0.080 mmol) in an ethanol (4 mL)/water (2 mL) mixture was heated in a microwave reactor. The reaction vessel was sealed and heated in Biotage Initiator using initial high to 100° C. for 15 min. After cooling, water was added and the precipitate was filtered off and the resulting solid was triturated in sat. NH₄Cl. The product was purified by chromatography on an Isco Companion. The sample was loaded on 10 g Biotage silica (Si) column then the purification was carried out using DCM/MeOH 100/0 to 90/10. The appropriate fractions were combined and concentrated in vacuo. The residue was triturated in acetonitrile to give ethyl 7-hydroxy-1-(2′-hydroxy-4-biphenylyl)-5-oxo-4,5-dihydro-1H-pyrrolo[3,2-b]pyridine-6-carboxylate (10 mg, 0.026 mmol, 1.281% yield) as a cream powder. ¹H NMR: (DMSO-d6, 300 Hz) δ 14.20 (br s, 1H), 11.38 (br s, 1H), 9.65 (s, 1H), 7.64 (d, J=8.4 Hz, 2H), 7.55 (m, 1H), 7.47 (d, J=8.4 Hz, 2H), 7.32 (d, J=7.4 Hz, 1H), 7.49 (m, 1H), 6.98 (d, J=8.0 Hz, 1H), 6.90 (m, 1H), 6.15 (d, J=2.4 Hz, 1H), 4.30 (q, J=7.0 Hz, 2H), 1.28 (t, J=7.0 Hz, 3H). LCMS: (M+H)⁺: 391; Rt: 2.78 min. HRMS: calculated for C₂₂H₁₉N₂O₅(M+H)⁺: 391.1294; found: 391.1283; Rt: 2.54 min.

Examples 31 to 35 of formula (I), wherein R¹ is cyano and R³, R⁴, R⁶ and R⁷ are all H were prepared by methods analogous to that described for Example 2 from Example 1 using the appropriate boronic acid.

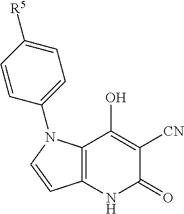

TABLE 15

| Example | Name | R⁵ | Physical data |
|---|---|---|---|
| 31 | 4′-(6-Cyano-7- hydroxy-5-oxo-4,5- dihydro-1H- pyrrolo[3,2-b]pyridin- 1-yl)-2-hydroxy-3- biphenylcarboxylic acid | 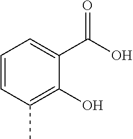 | ¹H NMR: (DMSO-d6, 300 Hz) δ 12.01 (br s, 1H), 11.64 (br s, 1H), 7.86 (dd, J = 7.8 and 1.6 Hz, 1H), 7.65 (m, 1H), 7.64 (d, J = 8.5 Hz, 2H), 7.52 (d, J = 3.1 Hz, 1H), 7.50 (d, J = 8.5 Hz, 2H), 7.05 (dd, J = 7.8 and 7.8 Hz, 1H), 6.21 (d, J = 3.0 Hz, 1H). LCMS: (M + H)⁺: 388; Rt: 1.97 min. HRMS: calculated for C₂₂H₁₄N₃O₅ (M + H)⁺: 388.0933; found: 388.0929; Rt: 2.42 min. |
| 32 | 1-(2′,4′-Difluoro-4- biphenylyl)-7-hydroxy- 5-oxo-4,5-dihydro-1H- pyrrolo[3,2-b]pyridine- 6-carbonitrile | 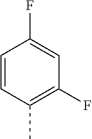 | ¹H NMR: (DMSO-d6, 300 Hz) δ 9.78 (s, 1H), 7.62 (m, 1H), 7.54- 7.45 (m, 4H), 7.38 (m, 1H), 7.22 (m, 1H), 7.08 (d, J = 3.0 Hz, 1H), 5.93 (d, J = 3.0 Hz, 1H). LCMS: (M + H)⁺: 364; Rt: 2.64 min. HRMS: calculated for C₂₀H₁₂F₂N₃O₂ (M + H)⁺: 362.0741; found: 362.0736; Rt: 2.31 min. |
| 33 | 7-Hydroxy-5-oxo-1-[2′- (trifluoromethyl)-4- biphenylyl]-4,5- dihydro-1H- pyrrolo[3,2-b]pyridine- 6-carbonitrile | 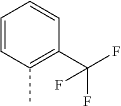 | ¹H NMR: (DMSO-d6, 300 Hz) δ 9.78 (s, 1H), 7.85 (d, J = 7.6 Hz, 1H), 7.75 (m, 1H), 7.62 (m, 1H), 7.54-7.40 (m, 3H), 7.29 (d, J = 7.4 Hz, 2H), 7.11 (d, J = 3.0 Hz, 1H), 5.93 (d, J = 3.0 Hz, 1H). LCMS: (M + H)⁺: 396; Rt: 2.74 min. HRMS: calculated for C₂₁H₁₃F₃N₃O₂ (M + H)⁺: 394.0804; found: 394.0782; Rt: 2.38 min. |
| 34 | 1-(2′,4′-Dichloro-4- biphenylyl)-7-hydroxy- 5-oxo-4,5-dihydro-1H- pyrrolo[3,2-b]pyridine- 6-carbonitrile | 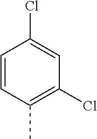 | LCMS: (M + H)⁺: 396; Rt: 2.90 min. HRMS: calculated for C₂₀H₁₂Cl₂N₃O₂ (M + H)⁺: 396.0306; found: 396.0302. Rt: 2.53 min |
| 35 | 1-[4-(5-Chloro-2- thienyl)phenyl]-7- hydroxy-5-oxo-4,5- dihydro-1H- pyrrolo[3,2-b]pyridine- 6-carbonitrile | 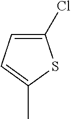 | LCMS: (M + H)⁺: 368; Rt: 2.66 min. HRMS: calculated for C₁₈H₉ClN₃O₂S (M − H)⁻: 366.0104; found: 366.0110; Rt: 2.54 min |

### Example 36

### 1-(4-Bromophenyl)-6-(4-fluorophenyl)-7-hydroxy-1,4-dihydro-5H-pyrrolo[3,2-b]pyridin-5-one

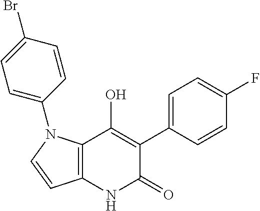

Ethyl 1-(4-bromophenyl)-3-{[(4-fluorophenyl)acetyl]amino}-1H-pyrrole-2-carboxylate (Intermediate 12) (1 g, 2.246 mmol) was dissolved in DMSO (3 mL) and potassium tert-butoxide (0.504 g, 4.49 mmol) was added. The reaction mixture was stirred at room temperature for 30 min then was heated at 110° C. for 4 h. 1N HCl was added and the resulting solid was filtered and purified with Biotage using DCM/MeOH 100/0 to 90/10 as eluent to give 1-(4-bromophenyl)-6-(4-fluorophenyl)-7-hydroxy-1,4-dihydro-5H-pyrrolo[3,2-b]pyridin-5-one (250 mg, 0.614 mmol, 27.3% yield) as a yellow solid. LCMS: (M+H)⁺: 399, 401; Rt: 2.72 min. HRMS: calculated for C₁₉H₁₃BrFN₂O₂ (M+H)⁺: 399.0144; found: 399.0160; Rt: 2.40 min.

### Example 37

### 6-(4-Fluorophenyl)-7-hydroxy-1-(2′-hydroxy-4-biphenylyl)-1,4-dihydro-5H-pyrrolo[3,2-b]pyridin-5-one

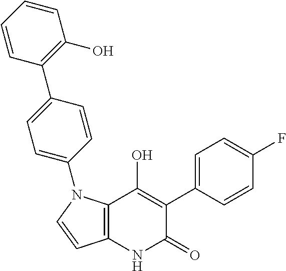

To a solution of 1-(4-bromophenyl)-6-(4-fluorophenyl)-7-hydroxy-1,4-dihydro-5H-pyrrolo[3,2-b]pyridin-5-one (Example 36) (150 mg, 0.376 mmol) in a 1,4-dioxane (5 mL) and water (5 mL) mixture were added (2-hydroxyphenyl)boronic acid (104 mg, 0.751 mmol), cesium carbonate (245 mg, 0.751 mmol) and Pd(PPh₃)₄ (2 mg, 1.879 μmol) and the reaction mixture was stirred at 100° C. for 15 h before being evaporated off. The product was filtered through a guanidine column (SPE) and then purified using biotage (DCM 100% to DCM/MeOH 100/0 to 90/10. as eluent). The appropriate fractions were combined and concentrated under reduced pressure to give 6-(4-fluorophenyl)-7-hydroxy-1-(2′-hydroxy-4-biphenylyl)-1,4-dihydro-5H-pyrrolo[3,2-b]pyridin-5-one (10 mg, 0.023 mmol, 6.13% yield) as a brown solid. LCMS: (M+H)⁺: 413; Rt: 2.68 min. HRMS: calculated for C₂₅H₁₆FN₂O₃ (M−H)⁻: 411.1145; found: 411.1175; Rt: 2.40 min.

### Example 38

### 1-(4-Bromophenyl)-7-hydroxy-6-[4-(methyloxy)phenyl]-1,4-dihydro-5H-pyrrolo[3,2-b]pyridin-5-one

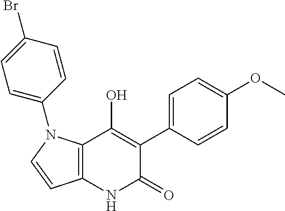

Ethyl 1-(4-bromophenyl)-3-({[4-(methyloxy)phenyl]acetyl}amino)-1H-pyrrole-2-carboxylate (Intermediate 14) (730 mg, 1.596 mmol) was dissolved in DMSO (2 mL) and potassium tert-butoxide (358 mg, 3.19 mmol) was added at room temperature. The mixture was then stirred at 100° C. overnight before being quenched with 1N HCl. The resulting solid was filtered and purified by chromatography on a Biotage SP4. The sample was loaded on 50 g Biotage silica (Si) column then the purification was carried out using DCM/MeOH 100/0 to 90/10. The appropriate fractions were combined and concentrated in vacuo to give 1-(4-bromophenyl)-7-hydroxy-6-[4-(methyloxy)phenyl]-1,4-dihydro-5H-pyrrolo[3,2-b]pyridin-5-one (50 mg, 7% yield) as an off-white solid. LCMS: (M+H)⁺: 411, 413; Rt: 2.71 min. HRMS: calculated for C₂₀H₁₆BrN₂O₃ (M+H)⁺: 411.0344; found: 411.0379; Rt: 2.51 min.

### Example 39

### 4-[7-hydroxy-1-(2′-hydroxy-4-biphenylyl)-5-oxo-4,5-dihydro-1H-Pyrrolo[3,2-b]pyridin-6-yl]benzonitrile

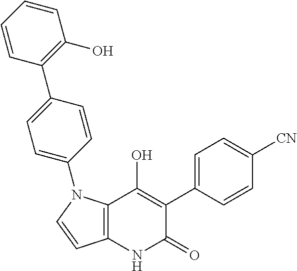

To a solution of 4-[1-(4-bromophenyl)-7-hydroxy-5-oxo-4,5-dihydro-1H-pyrrolo[3,2-b]pyridin-6-yl]benzonitrile (Intermediate 16) (200 mg, 0.492 mmol) and (2-hydroxyphenyl)boronic acid (81 mg, 0.591 mmol) in 1,4-dioxane (5 mL) was added a solution of Cs₂CO₃ (481 mg, 1.477 mmol) in water (1 mL). The mixture was purged with nitrogen and Pd(PPh₃)₄ (11.38 mg, 9.85 μmol) was added. The reaction vessel was sealed and heated in Biotage Initiator at 130° C. for 20 min. The reaction mixture was concentrated and the resulting oil was triturated in water then in MeOH. After filtration, the solid was dried under reduced pressure to give 4-[7-hydroxy-1-(2′-hydroxy-4-biphenylyl)-5-oxo-4,5-dihydro-1H-pyrrolo[3,2-b]pyridin-6-yl]benzonitrile (10 mg, 0.021 mmol, 4.36% yield) as an off-white solid. LCMS: (M+H)⁺: 420; Rt: 2.42 min. HRMS: calculated for C₂₆H₁₇N₃O₃ (M+H)⁺: 420.1348; found: 420.1307; Rt: 2.23 min

### Example 40

### 7-Hydroxy-1-(2′-hydroxy-4-biphenylyl)-6-phenyl-1,4-dihydro-5H-pyrrolo[3,2-b]pyridin-5-one

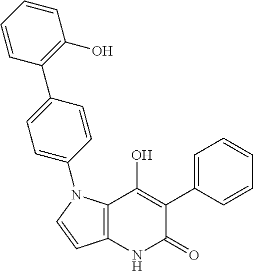

To a solution of 1-(4-bromophenyl)-7-hydroxy-6-phenyl-1,4-dihydro-5H-pyrrolo[3,2-b]pyridin-5-one (Intermediate 18) (200 mg, 0.525 mmol) and (2-hydroxyphenyl)boronic acid (87 mg, 0.630 mmol) in 1,4-dioxane (5 mL) was added a solution of Cs₂CO₃ (513 mg, 1.574 mmol) in water (1 mL). The mixture was purged with nitrogen and Pd(PPh₃)₄ (10 mg, 8.65 μmol) was added. The reaction vessel was sealed and heated in Biotage Initiator at 130° C. for 20 min before being concentrated under reduced pressure. Water was added and the solid was filtered and discarded. The aqueous phase was concentrated under reduced pressure, MeOH was added, and the solution was heated then filtered. The organic phase was concentrated, a little of MeOH was added then product was filtered and dried under reduced pressure to give 7-hydroxy-1-(2′-hydroxy-4-biphenylyl)-6-phenyl-1,4-dihydro-5H-pyrrolo[3,2-b]pyridin-5-one (36 mg, 0.089 mmol, 17.05% yield) as a light brown solid. LCMS: (M+H)⁺: 395; Rt: 2.58 min. HRMS: calculated for C₂₅H₁₉N₂O₃ (M+H)⁺: 395.1396; found: 395.1418; Rt: 2.37 min

### Example 41

### Ethyl 4-(6-cyano-7-hydroxy-5-oxo-4,5-dihydro-1H-pyrrolo[3,2-b]pyridin-1-yl)benzoate

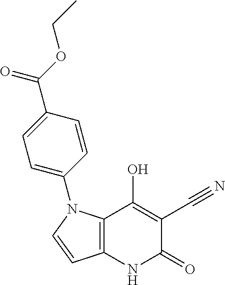

To a solution of ethyl 3-[(cyanoacetyl)amino]-1-{4-[(ethyloxy)carbonyl]phenyl}-1H-pyrrole-2-carboxylate (Intermediate 143) (250 mg, 0.677 mmol) in dry THF (10 mL) was added under nitrogen at RT, sodium hydride (60% suspension in oil, 62.3 mg, 1.557 mmol). The reaction mixture was stirred at 50° C. for 36 hours before water was added. After acidification with 1N HCl, the product was extracted with EtOAc. The organic layer was dried over Na₂SO₄, filtered and evaporated off to give after crystallisation in MeOH, ethyl 4-(6-cyano-7-hydroxy-5-oxo-4,5-dihydro-1H-pyrrolo[3,2-b]pyridin-1-yl)benzoate (210 mg, 0.585 mmol, 86% yield) as white powder. LCMS: (M+H)⁺: 324; Rt: 2.08 min. HRMS: calculated for C₁₇H₁₄N₃O₄(M+H)⁺: 324.0984; found: 324.0964; Rt: 1.95 min.

### Example 42

### 7-Hydroxy-1-[4-(methyloxy)phenyl]-5-oxo-4,5-dihydro-1H-pyrrolo[3,2-b]pyridine-6-carbonitrile

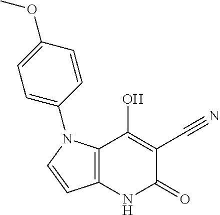

To a solution of ethyl 3-[(cyanoacetyl)amino]-1-[4-(methyloxy)phenyl]-1H-pyrrole-2-carboxylate (Intermediate 56) (180 mg, 0.550 mmol) in THF (2 mL) at RT was added sodium hydride (60% suspension in oil, 33.0 mg, 0.825 mmol) in one portion. After evolution of hydrogen stopped, the reaction mixture was stirred to reflux for 18 h before being cooled and quenched with MeOH. After concentration to dryness, the orange residue was recrystallised in 1N NaOH (with filtration when hot) to give 7-hydroxy-1-[4-(methyloxy)phenyl]-5-oxo-4,5-dihydro-1H-pyrrolo[3,2-b]pyridine-6-carbonitrile (45 mg, 0.160 mmol, 29.1% yield) as a off-white solid. LCMS: (M+H)⁺: 282; Rt: 1.92 min. HRMS: calculated for C₁₅H₁₀N₃O₃ (M−H)⁻: 280.0722; found: 280.0729; Rt: 1.76 min.

### Example 43

### 1-(4-Bromophenyl)-7-hydroxy-1,4-dihydro-5H-pyrrolo[3,2-b]pyridin-5-one

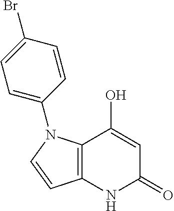

Ethyl 1-(4-bromophenyl)-7-hydroxy-5-oxo-4,5-dihydro-1H-pyrrolo[3,2-b]pyridine-6-carboxylate (Intermediate 6) (31 g, 82.2 mmol) was stirred at 120° C. overnight in a 2N KOH solution (400 mL) before being quenched with 1N HCl. The resulting solid was filtered, washed with water and dried to give the desired product 1-(4-bromophenyl)-7-hydroxy-1,4-dihydro-5H-pyrrolo[3,2-b]pyridin-5-one (25 g, 100% yield). LCMS: (M+H)⁺: 305, 307; Rt: 1.62 min.

Examples 44 to 47 of formula (I), wherein R¹ is CN and R³, R⁴, R⁶ and R⁷ are all H, were prepared by methods analogous to that described for Example 2 from Example 1 using the appropriate boronic acid.

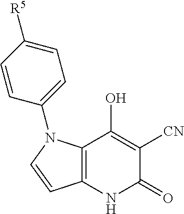

TABLE 16

| Example | Name | R⁵ | Physical data |
|---|---|---|---|
| 44 | 1-(2′-Fluoro-4′-methyl- 4-biphenylyl)-7- hydroxy-5-oxo-4,5- dihydro-1H- pyrrolo[3,2-b]pyridine- 6-carbonitrile | 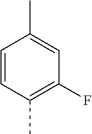 | LCMS: (M + H)⁺: 360; Rt: 2.61 min. HRMS: calculated for C₂₁H₁₃FN₃O₂ (M − H)⁻: 358.0992; found: 358.0996; Rt: 2.34 min |
| 45 | 7-Hydroxy-5-oxo-1-[4′- (trimethylsilyl)-4- biphenylyl]-4,5- dihydro-1H- pyrrolo[3,2-b]pyridine- 6-carbonitrile | 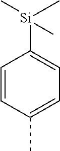 | LCMS: (M + H)⁺: 400; Rt: 3.17 min. HRMS: calculated for C₂₃H₂₀N₃O₂Si (M − H)⁻: 398.1325; found: 398.1332; Rt: 2.74 min |
| 46 | 1-[4-(1,3-Benzodioxol- 5-yl)phenyl]-7- hydroxy-5-oxo-4,5- dihydro-1H- pyrrolo[3,2-b]pyridine- 6-carbonitrile | 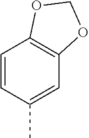 | LCMS: (M + H)⁺: 372; Rt: 2.42 min. HRMS: calculated for C₂₁H₁₂N₃O₄ (M − H)⁻: 370.0828; found: 370.0811; Rt: 2.17 min |
| 47 | 1-(2′,4′-Dimethyl-4- biphenylyl)-7-hydroxy- 5-oxo-4,5-dihydro-1H- pyrrolo[3,2-b]pyridine- 6-carbonitrile | 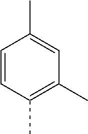 | LCMS: (M + H)⁺: 356; Rt: 2.17 min. HRMS: calculated for C₂₂H₁₆N₃O₂ (M − H)⁻: 354.1243; found: 354.1260; Rt: 2.45 min |

### Example 48

### 7-Hydroxy-5-oxo-1-[4-(3-thienyl)phenyl]-4,5-dihydro-1H-pyrrolo[3,2-b]pyridine-6-carbonitrile

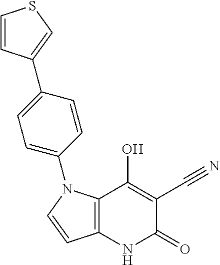

In a 250 mL round bottom flask, 7-hydroxy-5-oxo-1-[4-(4,4,5,5-tetramethyl-1,3,2-dioxaborolan-2-yl)phenyl]-4,5-dihydro-1H-pyrrolo[3,2-b]pyridine-6-carbonitrile (Intermediate 58) (300 mg, 0.795 mmol), 3-bromothiophene (259 mg, 1.591 mmol), Pd(PPh₃)₄ (18.38 mg, 0.016 mmol) and cesium carbonate (777 mg, 2.386 mmol) were mixed in a 1,4-dioxane (10 mL)/water (10.00 mL)/ethanol (1 mL) mixture to give an orange solution. The reaction mixture was stirred at 120° C. for 18 h before being concentrated in vacuo, poured into water and acidified to give a precipitate. Trituration in hot CH₃CN gave 7-hydroxy-5-oxo-1-[4-(3-thienyl)phenyl]-4,5-dihydro-1H-pyrrolo[3,2-b]pyridine-6-carbonitrile (150 mg, 0.450 mmol, 56.6% yield) as a cream powder. LCMS: (M+H)⁺: 334; Rt: 2.63 min. HRMS: calculated for C₁₈H₁₂N₃O₂S (M+H)⁺: 334.0650; found: 334.0671; Rt: 2.15 min

Examples 49 to 55 of formula (I), wherein R¹ is CN and R³, R⁴, R⁶ and R⁷ are all H, were prepared by methods analogous to that described for Example 48 from Intermediate 58 using the appropriate heteroarylbromide.

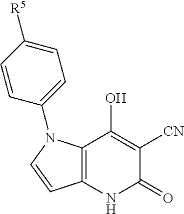

TABLE 17

| Ex- | | | |
|---|---|---|---|
| ample | Name | R⁵ | Physical data |
| 49 | 7-Hydroxy-1-[4-(6- methyl-2- pyridinyl)phenyl]- 5-oxo-4,5-dihydro-1H- pyrrolo[3,2-b]pyridine- 6-carbonitrile | 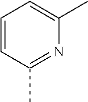 | LCMS: (M + H)⁺: 343; Rt: 2.58 min. HRMS: calculated for C₂₀H₁₅N₄O₂ (M + H)⁺: 343.1195; found: 343.1216; Rt: 2.02 min. |
| 50 | 7-Hydroxy-5-oxo-1-[4- (4-pyridinyl)phenyl]- 4,5-dihydro-1H- pyrrolo[3,2-b]pyridine- 6-carbonitrile | 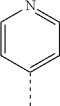 | LCMS: (M + H)⁺: 329; Rt: 2.07 min. HRMS: calculated for C₁₉H₁₃N₄O₂ (M + H)⁺: 329.1038; found: 329.1031; Rt: 1.79 min. |
| 51 | 1-[4-(5-Cyano-2- pyridinyl)phenyl]- 7-hydroxy-5-oxo-4,5- dihydro-1H- pyrrolo[3,2-b]pyridine- 6-carbonitrile | 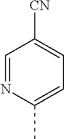 | LCMS: (M + H)⁺: 354; Rt: 2.35 min. HRMS: calculated for C₂₀H₁₂N₅O₂ (M + H)⁺: 354.0991; found: 354.1024; Rt: 1.94 min. |
| 52 | 1-[4-(3,5-Dimethyl-4- isoxazolyl)phenyl]- 7-hydroxy-5-oxo- 4,5-dihydro-1H- pyrrolo[3,2-b]pyridine- 6-carbonitrile | 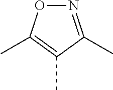 | LCMS: (M + H)⁺: 347; Rt: 2.37 min. HRMS: calculated for C₁₉H₁₅N₄O₃ (M + H)⁺: 347.1144; found: 347.1149; Rt: 1.95 min. |
| 53 | 7-Hydroxy-5-oxo-1-[4- (3-pyridinyl)phenyl]- 4,5-dihydro-1H- pyrrolo[3,2-b]pyridine- 6-carbonitrile | 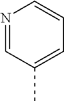 | LCMS: (M + H)⁺: 329; Rt: 2.25 min. HRMS: calculated for C₁₉H₁₃N₄O₂ (M + H)⁺: 329.1039; found: 329.1034; Rt: 1.81 min. |
| 54 | 7-Hydroxy-1-[4-(5- methyl-2- pyridinyl)phenyl]- 5-oxo-4,5-dihydro-1H- pyrrolo[3,2-b]pyridine- 6-carbonitrile | 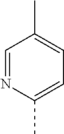 | LCMS: (M + H)⁺: 343; Rt: 2.58 min. HRMS: calculated for C₂₀H₁₅N₄O₂ (M + H)⁺: 343.1195; found: 343.1245; Rt: 2.01 min. |
| 55 | 7-Hydroxy-1-{4-[5- (methyloxy)-2- pyridinyl]phenyl}- 5-oxo-4,5-dihydro-1H- pyrrolo[3,2-b]pyridine- 6-carbonitrile | 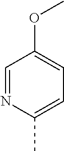 | LCMS: (M + H)⁺: 359; Rt = 2.42 min. HRMS: calculated for C₂₀H₁₅N₄O₃ (M + H)⁺: 359.1144; found: 359.1109; Rt: 1.97 min |

### Example 56

### 7-Hydroxy-5-oxo-1-[4-(1H-pyrrol-2-yl)phenyl]-4,5-dihydro-1H-pyrrolo[3,2-b]pyridine-6-carbonitrile

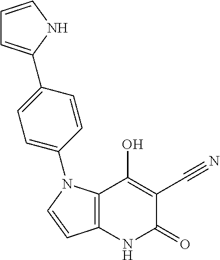

To a mixture of 1-(4-bromophenyl)-7-hydroxy-5-oxo-4,5-dihydro-1H-pyrrolo[3,2-b]pyridine-6-carbonitrile (Example 1) (150 mg, 0.454 mmol) and Pd(PPh₃)₄ (26.3 mg, 0.023 mmol) in a 1,4-dioxane (2.5 mL)/water (1.5 mL) mixture were added Cs₂CO₃ (444 mg, 1.363 mmol) and (1-{[(1,1-dimethylethyl)oxy]carbonyl}-1H-pyrrol-2-yl)boronic acid (128 mg, 0.606 mmol). The reaction mixture was stirred at 150° C. for 15 minutes in a microwave oven Biotage initiator. The mixture was solubilized in DMF and filtered through a SPE guanidine column. The filtrate was then evaporated and purified by chromatography on an Isco Companion RF. The sample was loaded on 25 g Biotage silica (Si) column then the purification was carried out using a DCM/MeOH 100/0 to 40/60 gradient. The appropriate fractions were combined and concentrated in vacuo to give the required product 7-hydroxy-5-oxo-1-[4-(1H-pyrrol-2-yl)phenyl]-4,5-dihydro-1H-pyrrolo[3,2-b]pyridine-6-carbonitrile (30 mg, 16% yield) as a white solid. LCMS: (M+H)⁺: 317; Rt: 2.58 min. HRMS: calculated for C₁₈H₁₁N₄O₂ (M−H)⁻: 315.0882; found: 315.0873; Rt: 2.01 min.

Examples 57 and 58 of formula (I), wherein R¹ is CN and R³, R⁴, R⁶ and R⁷ are all H were prepared by methods analogous to that described for Example 2 from Example 1 using the appropriate boronic acid.

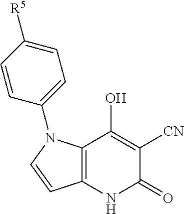

TABLE 18

| Ex- | | | |
|---|---|---|---|
| ample | Name | R⁵ | Physical data |
| 57 | 1-(3′-Fluoro-2′- hydroxy-4-biphenylyl)- 7-hydroxy-5-oxo-4,5- dihydro-1H- pyrrolo[3,2-b]pyridine- 6-carbonitrile | 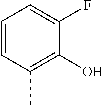 | LCMS: (M + H)⁺: 362; Rt: 5.16 min. HRMS: calculated for C₂₀H₁₃FN₃O₃ (M + H)⁺: 362.0941; found: 362.0961; Rt: 2.10 min. |
| 58 | 7-Hydroxy-1-(2′- hydroxy-3′-methyl-4- biphenylyl)-5-oxo-4,5- dihydro-1H- pyrrolo[3,2-b]pyridine- 6-carbonitrile | 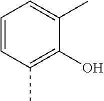 | LCMS: (M + H)⁺: 358; Rt: 5.67 min. HRMS: calculated for C₂₁H₁₆N₃O₃ (M + H)⁺: 358.1191; found: 358.1217; Rt: 2.26 min. |

Example 59 of formula (I), wherein R¹ is CN and R³, R⁴, R⁶ and R⁷ are all H was prepared by methods analogous to that described for Example 48 from Intermediate 58 using the appropriate heteroarylbromide.

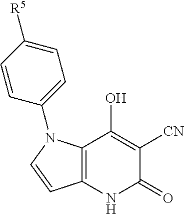

TABLE 19

| Example | Name | R⁵ | Physical data |
|---|---|---|---|
| 59 | 7-Hydroxy-5-oxo-1-[4- (2-thienyl)phenyl]-4,5- dihydro-1H- pyrrolo[3,2-b]pyridine- 6-carbonitrile | 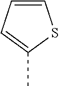 | LCMS: (M + H)⁺: 334; Rt: 2.55 min. HRMS: calculated for C₁₈H₁₂N₃O₂S (M + H)⁺: 334.0650; found: 334.0663; Rt: 2.17 min. |

Examples 60 to 65 of formula (I), wherein R¹ is CN and R³, R⁴, R⁶ and R⁷ are all H, were prepared by methods analogous to that described for Example 2 from Example 1 using the appropriate boronic acid.

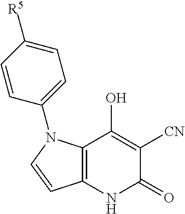

TABLE 20

| Example | Name | R⁵ | Physical data |
|---|---|---|---|
| 60 | 1-(3′-Chloro-2′-hydroxy-4- biphenylyl)-7-hydroxy-5- oxo-4,5-dihydro-1H- pyrrolo[3,2-b]pyridine-6- carbonitrile | 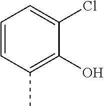 | LCMS: (M + H)⁺: 378; Rt: 5.48 min. HRMS: calculated for C₂₀H₁₃ClN₃O₃ (M + H)⁺: 378.0645: found: 378.0659; Rt: 2.26 min. |
| 61 | 1-(5′-Fluoro-2′-hydroxy-4- biphenylyl)-7-hydroxy-5- oxo-4,5-dihydro-1H- pyrrolo[3,2-b]pyridine-6- carbonitrile | 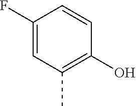 | LCMS: (M + H)⁺: 362; Rt: 5.30 min. HRMS: calculated for C₂₀H₁₁FN₃O₃ (M − H)⁻: 360.0785; found: 360.0772; Rt: 2.13 min. |
| 62 | 1-(2′-Fluoro-6′-hydroxy-4- biphenylyl)-7-hydroxy-5- oxo-4,5-dihydro-1H- pyrrolo[3,2-b]pyridine-6- carbonitrile | 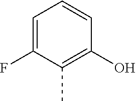 | LCMS: (M + H)⁺: 362; Rt: 5.16 min. HRMS: calculated for C₂₀H₁₁FN₃O₃ (M − H)⁻: 360.0785; found: 360.0778; Rt: 2.12 min. |
| 63 | 1-(4′-Chloro-2′-hydroxy-4- biphenylyl)-7-hydroxy-5- oxo-4,5-dihydro-1H- pyrrolo[3,2-b]pyridine-6- carbonitrile | 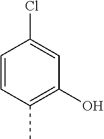 | LCMS: (M + H)⁺: 378; Rt: 5.76 min. HRMS: calculated for C₂₀H₁₃ClN₃O₃ (M + H)⁺: 378.0645; found: 378.0652; Rt: 2.25 min |
| 64 | 7-hydroxy-1-(2′-hydroxy-5′- methyl-4-biphenylyl)-5-oxo- 4,5-dihydro-1H-pyrrolo[3,2- b]pyridine-6-carbonitrile | 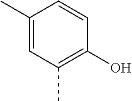 | LCMS: (M + H)⁺: 358; Rt: 5.49 min. HRMS: calculated for C₂₁H₁₄N₃O₃ (M − H)⁻: 356.1035; found: 356.1013; Rt: 2.18 min |
| 65 | 4′-(6-Cyano-7-hydroxy-5- oxo-4,5-dihydro-1H- pyrrolo[3,2-b]pyridin-1-yl)-6- hydroxy-3- biphenylcarboxylic acid | 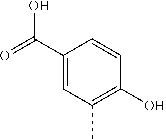 | LCMS: (M + H)⁺: 388; Rt: 2.47 min. HRMS: calculated for C₂₁H₁₂N₃O₅ (M − H)⁻: 386.0777; found: 386.805; Rt: 1.88 min. |

Example 66 of formula (I), wherein R¹ is CN and R³, R⁴ and R⁷ are all H and R⁶ is Cl was prepared by methods analogous to that described for Example 24 from Example 23 using the appropriate boronic acid.

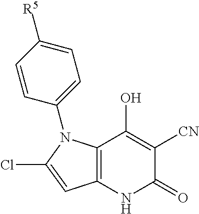

TABLE 21

| Ex- | | | Physical |
|---|---|---|---|
| ample | Name | R⁵ | data |
| 66 | 2-Chloro-1-(5′-fluoro-2′- hydroxy-4-biphenylyl)-7- hydroxy-5-oxo-4,5-dihydro- 1H-pyrrolo[3,2-b]pyridine- 6-carbonitrile | 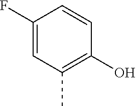 | LCMS: (M + H)⁺: 396; Rt: 6.36 min. |

Examples 67 and 68 of formula (I), wherein R¹ is CN and R³, R⁴, R⁶ and R⁷ are all H, were prepared by methods analogous to that described for Example 2 from Example 1 using the appropriate boronic acid.

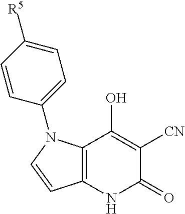

TABLE 22

| Example | Name | R⁵ | Physical data |
|---|---|---|---|
| 67 | 7-Hydroxy-1-[2′-hydroxy-3′- (methyloxy)-5′-(2-propen-1- yl)-4-biphenylyl]-5-oxo-4,5- dihydro-1H-pyrrolo[3,2- b]pyridine-6-carbonitrile | 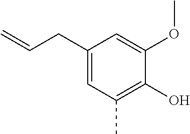 | LCMS: (M + H)⁺: 414; Rt: 6.07 min. |
| 68 | 1-[4-(2,3-Dihydro-1,4- benzodioxin-6-yl)phenyl]-7- hydroxy-5-oxo-4,5-dihydro- 1H-pyrrolo[3,2-b]pyridine-6- carbonitrile | 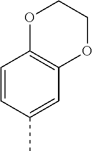 | LCMS: (M + H)⁺: 386; Rt: 2.59 min. HRMS: calculated for C₂₂H₁₄N₃O₄ (M − H)⁻: 384.0984; found: 384.098; Rt: 2.20 min. |

Examples 69 and 70 of formula (I), wherein R¹ is CN and R³, R⁴ and R⁷ are all H and R⁶ is Cl were prepared by methods analogous to that described for Example 24 from Example 23 using the appropriate boronic acid.

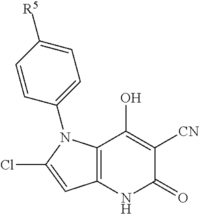

TABLE 23

| Example | Name | R⁵ | Physical data |
|---|---|---|---|
| 69 | 2-Chloro-7-hydroxy-1-(2′- hydroxy-3′-methyl-4- biphenylyl)-5-oxo-4,5- dihydro-1H-pyrrolo[3,2- b]pyridine-6-carbonitrile | 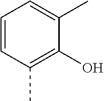 | LCMS: (M + H)⁺: 391; Rt: 5.66 min. HRMS: calculated for C₂₁H₁₅ClN₃O₃ (M + H)⁺: 392.0802; found: 392.0818; Rt: 2.37 min |
| 70 | 4′-(2-Chloro-6-cyano-7- hydroxy-5-oxo-4,5-dihydro- 1H-pyrrolo[3,2-b]pyridin-1- yl)-6-hydroxy-3- biphenylcarboxylic acid | 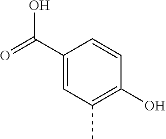 | LCMS: (M + H)⁺: 422; Rt: 3.77 min. HRMS: calculated for C₂₁H₁₃ClN₃O₅ (M + H)⁺: 422.0543; found: 422.0551; Rt: 1.57 min. |

Examples 71 and 72 of formula (I), wherein R¹ is CN and R³, R⁴, R⁶ and R⁷ are all H, were prepared by methods analogous to that described for Example 2 from Example 1 using the appropriate boronic acid.

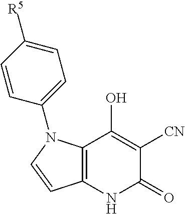

TABLE 24

| Example | Name | R⁵ | Physical data |
|---|---|---|---|
| 71 | 1-(2′-Chloro-6′-hydroxy-4- biphenylyl)-7-hydroxy-5- oxo-4,5-dihydro-1H- pyrrolo[3,2-b]pyridine-6- carbonitrile | 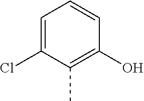 | LCMS: (M + H)⁺: 378; Rt: 5.02 min. HRMS: calculated for C₂₀H₁₁ClN₃O₃ (M − H)⁻: 376.0489; found: 376.0493; Rt: 3.21 min. |
| 72 | 7-Hydroxy-1-{4-[2- (methyloxy)-3- pyridinyl]phenyl}-5-oxo-4,5- dihydro-1H-pyrrolo[3,2- b]pyridine-6-carbonitrile | 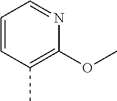 | LCMS: (M + H)⁺: 359; Rt: 4.25 min. HRMS: calculated for C₂₀H₁₃N₄O₃ (M − H)⁻: 357.0988; found: 357.0995; Rt: 2.96 min. |

### Example 73

### 2-Chloro-7-hydroxy-5-oxo-1-[4-(3-thienyl)phenyl]-4,5-dihydro-1H-pyrrolo[3,2-b]pyridine-6-carbonitrile

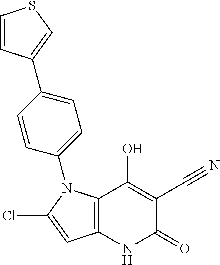

To a solution of ethyl 5-chloro-3-[(cyanoacetyl)amino]-1-[4-(3-thienyl)phenyl]-1H-pyrrole-2-carboxylate (Intermediate 68) (120 mg, 0.290 mmol) in THF (150 mL) was added sodium hydride (60% suspension in oil, 58.0 mg, 1.450 mmol). The reaction mixture was stirred at reflux for 5 hours before MeOH was added to quench excess of NaH. The solvent was evaporated to dryness and the crude mixture poured into water, acidified to pH 5 with 1N HCl solution. The precipitate was filtered off and triturated in hot MeOH to give 2-chloro-7-hydroxy-5-oxo-1-[4-(3-thienyl)phenyl]-4,5-dihydro-1H-pyrrolo[3,2-b]pyridine-6-carbonitrile (60 mg, 0.163 mmol, 56.3% yield) as a cream powder. LCMS: (M+H)⁺: 368; Rt: 5.25 min. ¹H NMR: (DMSO, 300 MHz) δ 8.01 (m, 1H), 7.83 (d, 2H, J=8.4 Hz), 7.67 (m, 2H), 7.43 (d, 2H, J=8.4 Hz), 6.31 (s, 1H). HRMS: calculated for C₁₈H₁₁ClN₃O₂S (M+H)⁺: 368.0260; found: 368.0292; Rt: 2.96 min.

Example 74 of formula (I), wherein R¹ is CN and R³, R⁴, R⁶ and R⁷ are all H, was prepared by a method analogous to that described for Example 2 from Example 1 using the appropriate boronic acid.

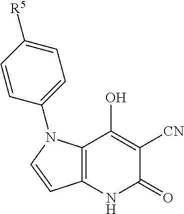

TABLE 25

| Ex- | | | |
|---|---|---|---|
| am- | | | |
| ple | Name | R⁵ | Physical data |
| 74 | 1-(4′-Fluoro-2′- hydroxy- 4-biphenylyl)-7- hydroxy-5-oxo-4,5- dihydro-1H-pyrrolo [3,2-b]pyridine-6- carbonitrile | 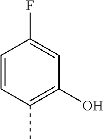 | LCMS: (M + H)⁺: 362; Rt: 4.87 min. HRMS: calculated for C₂₀H₁₃FN₃O₃ (M + H)⁺: 362.0941; found: 362.0905; Rt: 2.12 min. |

### Example 75

### 7-Hydroxy-1-[4-(2-hydroxy-3-pyridinyl)phenyl]-5-oxo-4,5-dihydro-1H-pyrrolo[3,2-b]pyridine-6-carbonitrile

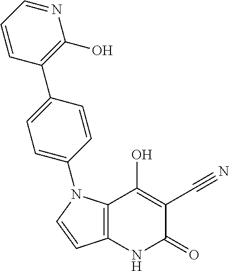

To a suspention of 7-hydroxy-1-{4-[2-(methyloxy)-3-pyridinyl]phenyl}-5-oxo-4,5-dihydro-1H-pyrrolo[3,2-b]pyridine-6-carbonitrile (Example 72) (140 mg, 0.391 mmol) in DCM (30 mL) at 0° C., was added BBr₃ (1.172 mL, 1M in DCM, 1.172 mmol). The reaction was stirred at RT for 24 h before being quenched cautiously with EtOH (30 ml). Water (10 ml) was then added and the mixture was heated to reflux for 48 h. After cooling to RT, the reaction mixture was filtered and washed with acetonitrile. Trituration in diethyle ether and drying gave 7-hydroxy-1-[4-(2-hydroxy-3-pyridinyl)phenyl]-5-oxo-4,5-dihydro-1H-pyrrolo[3,2-b]pyridine-6-carbonitrile (120 mg, 0.349 mmol, 89% yield) as a cream powder. LCMS: (M+H)⁺: 345; Rt: 3.18 min. HRMS: calculated for C₁₉H₁₁N₄O₃ (M−H)⁻: 343.0831; found: 343.0833; Rt: 1.64 min.

Examples 76 and 77 of formula (I), wherein R¹ is CN and R³, R⁴ and R⁷ are all H and R⁶ is Cl, were prepared by methods analogous to that described for Example 24 from Example 23 using the appropriate boronic acid.

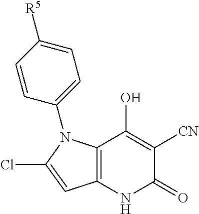

TABLE 26

| Example | Name | R⁵ | Physical data |
|---|---|---|---|
| 76 | 2-Chloro-1-(4′-chloro-2′- hydroxy-4-biphenylyl)-7- hydroxy-5-oxo-4,5-dihydro- 1H-pyrrolo[3,2-b]pyridine-6- carbonitrile | 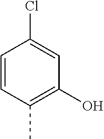 | LCMS: (M + H)⁺: 412, 414; Rt: 5.74 min. HRMS: calculated for C₂₀H₁₂Cl₂N₃O₃ (M + H)⁺: 412.0256; found: 412.0256; Rt: 2.36 min. |
| 77 | 7-Hydroxy-1-{4-[4- (methyloxy)-3- pyridinyl]phenyl}-5-oxo-4,5- dihydro-1H-pyrrolo[3,2- b]pyridine-6-carbonitrile | 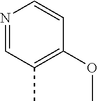 | LCMS: (M + H)⁺: 359; Rt: 3.76 min. |

### Example 78

### 1-[4-(2-Furanyl)phenyl]-7-hydroxy-5-oxo-4,5-dihydro-1H-pyrrolo[3,2-b]pyridine-6-carbonitrile

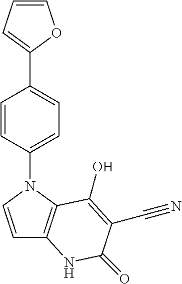

To a solution of ethyl 3-[(cyanoacetyl)amino]-1-[4-(2-furanyl)phenyl]-1H-pyrrole-2-carboxylate (Intermediate 69) (300 mg, 0.826 mmol) in THF (150 mL) was added sodium hydride (60% suspension in oil, 33 mg, 0.826 mmol). The reaction mixture was stirred at reflux for 5 h before MeOH was added to quench excess of NaH. The solvent was evaporated to dryness and the crude mixture poured into water, acidified to pH 5 with 1N HCl. The precipitate was filtered off and triturated in hot CH₃CN to give 1-[4-(2-furanyl)phenyl]-7-hydroxy-5-oxo-4,5-dihydro-1H-pyrrolo[3,2-b]pyridine-6-carbonitrile (250 mg, 0.788 mmol, 95% yield) as a cream powder. LCMS: (M+H)⁺: 318; Rt: 4.46 min.

### Example 79

### 7-Hydroxy-2-methyl-5-oxo-1-[4-(3-thienyl)phenyl]-4,5-dihydro-1H-pyrrolo[3,2-b]pyridine-6-carbonitrile

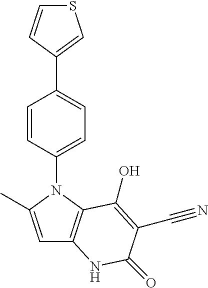

To a solution of ethyl 3-[(cyanoacetyl)amino]-5-methyl-1-[4-(3-thienyl)phenyl]-1H-pyrrole-2-carboxylate (130 mg, 0.330 mmol) (Intermediate 70) in THF (150 mL) was added sodium hydride (60% suspension in oil, 39.6 mg, 0.991 mmol). The reaction mixture was stirred at reflux for 5 h before MeOH was added to quench excess of NaH. The solvent was evaporated to dryness and the crude mixture poured into water, acidified to pH 5 with 1N HCl. The precipitate was filtered off and triturated in hot CH₃CN to give 7-hydroxy-2-methyl-5-oxo-1-[4-(3-thienyl)phenyl]-4,5-dihydro-1H-pyrrolo[3,2-b]pyridine-6-carbonitrile (90 mg, 0.259 mmol, 78% yield) as a cream powder. LCMS: (M+H)⁺: 348; Rt: 4.95 min. HRMS: calculated for C₁₉H₁₂N₃O₂S (M−H)⁻: 346.0650; found: 346.0616; Rt: 2.27 min.

Examples 80 to 82 of formula (I), wherein R¹ is a (4-cyanophenyl) and R³, R⁴, R⁶ and R⁷ are all H, were prepared by methods analogous to that described for Example 39 from Intermediate 16 using the appropriate boronic acid in dioxane/water at 110° C.

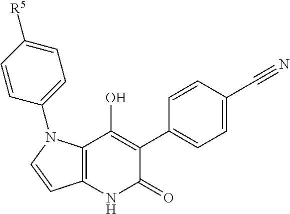

TABLE 27

| Example | Name | R⁵ | Physical data |
|---|---|---|---|
| 80 | 4-[1-(5′-Fluoro-2′-hydroxy-4-bi- phenylyl)-7-hydroxy-5-oxo- 4,5-dihydro-1H-pyrrolo[3,2-b] pyridin-6-yl]benzonitrile | 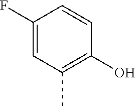 | LCMS: (M + H)⁺: 438; Rt: 3.65 min. |
| 81 | 4-[7-Hydroxy-1-(2′-hydroxy- 5′-methyl-4-biphenylyl)-5-oxo- 4,5-dihydro-1H- pyrrolo[3,2-b]pyridin-6- yl]benzonitrile | 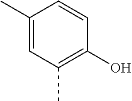 | LCMS: (M + H)⁺: 434; Rt: 3.83 min. |
| 82 | 4-[7-Hydroxy-1-(2′-hydroxy- 3′-methyl-4-biphenylyl)-5- oxo-4,5-dihydro-1H- pyrrolo[3,2-b]pyridin-6- yl]benzonitrile | 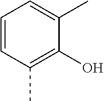 | LCMS: (M + H)⁺: 434; Rt: 3.95 min. |

Examples 83 and 84 of formula (I), wherein R¹ is CN and R³, R⁴ and R⁷ are all H and R⁶ is Cl, were prepared by methods analogous to that described for Example 24 from Example 23 using the appropriate boronic acid.

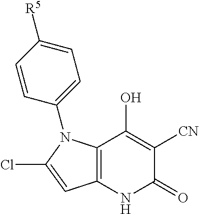

TABLE 28

| Example | Name | R⁵ | Physical data |
|---|---|---|---|
| 83 | 2-Chloro-1-(6′-fluoro-2′-hydroxy- 4-biphenylyl)-7- hydroxy-5-oxo-4,5-dihydro- 1H-pyrrolo[3,2-b]pyridine-6- carbonitrile | 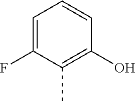 | HRMS: calculated for C₂₀H₁₂ClFN₃O₃ (M + H)⁺: 396.0551; found: 396.0559; Rt: 2.33 min. |
| 84 | 2-Chloro-1-(4′-fluoro-2′- hydroxy-4-biphenylyl)-7- hydroxy-5-oxo-4,5-dihydro- 1H-pyrrolo[3,2-b]pyridine-6- carbonitrile | 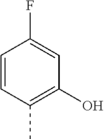 | HRMS: calculated for C₂₀H₁₀ClFN₃O₃ (M − H)⁻: 394.0395; found: 394.0404; Rt: 2.42 min. |

Examples 85 and 86 of formula (I), wherein R¹ is cyano and R³, R⁴, R⁶ and R⁷ are all H, were prepared by methods analogous to that described for Example 2 from Example 1 using the appropriate boronic acid.

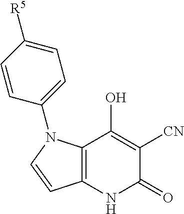

TABLE 29

| Ex- | | | |
|---|---|---|---|
| ample | Name | R⁵ | Physical data |
| 85 | 7-Hydroxy-1-[4-(4-methyl- 2-thienyl)phenyl]-5-oxo- 4,5-dihydro-1H-pyrrolo [3,2-b]pyridine-6- carbonitrile | 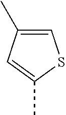 | HRMS: calculated for C₁₉H₁₄N₃O₂S (M + H)⁺: 348.0807; found: 348.0836; Rt: 2.41 min. |
| 86 | 7-Hydroxy-5-oxo-1-[4- (1H-pyrrol-3-yl)phenyl]- 4,5-dihydro-1H-pyrrolo [3,2-b]pyridine-6- carbonitrile | 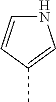 | HRMS: calculated for C₁₈H₁₁N₄O₂ (M − H)⁻: 315.0882; found: 315.0855; Rt: 1.92 min. |

Examples 87 to 92 of formula (I), wherein R¹ is CN and R³, R⁴, R⁶ and R⁷ are all H, were prepared by methods analogous to that described for Example 48 from Intermediate 58 using the appropriate heteroarylbromide.

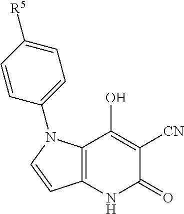

TABLE 30

| Example | Name | R⁵ | Physical data |
|---|---|---|---|
| 87 | 7-Hydroxy-5-oxo-1-[4-(1,3- thiazol-2-yl)phenyl]-4,5- dihydro-1H-pyrrolo[3,2- b]pyridine-6-carbonitrile | 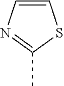 | HRMS: calculated for C₁₇H₉N₄O₂S (M − H)⁻: 333.00446; found: 333.0436; Rt: 1.96 min. |
| 88 | 7-Hydroxy-5-oxo-1-[4-(1,3- thiazol-4-yl)phenyl]-4,5- dihydro-1H-pyrrolo[3,2- b]pyridine-6-carbonitrile | 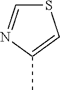 | HRMS: calculated for C₁₇H₉N₄O₂S (M − H)⁻: 333.0446; found: 333.0429; Rt: 1.90 min. |
| 89 | 7-Hydroxy-1-[4-(3-methyl-2- thienyl)phenyl]-5-oxo-4,5- dihydro-1H-pyrrolo[3,2- b]pyridine-6-carbonitrile | 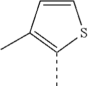 | HRMS: calculated for C₁₉H₁₂N₃O₂S (M − H)⁻: 346.0650; found: 346.0640; Rt: 2.37 min. |
| 90 | 5-[4-(6-Cyano-7-hydroxy-5- oxo-4,5-dihydro-1H- pyrrolo[3,2-b]pyridin-1- yl)phenyl]-2- thiophenesulfonamide | 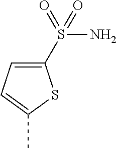 | HRMS: calculated for C₁₈H₁₁N₄O₄S₂ (M − H)⁻: 411.0222; found: 411.0184; Rt: 1.96 min. |
| 91 | 7-Hydroxy-1-[4-(1-methyl- 1H-pyrazol-4-yl)phenyl]-5- oxo-4,5-dihydro-1H- pyrrolo[3,2-b]pyridine-6- carbonitrile | 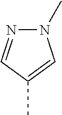 | HRMS: calculated for C₁₈H₁₂N₅O₂ (M − H)⁻: 330.0991; found: 330.0980; Rt: 1.88 min. |
| 92 | 7-Hydroxy-1-[4-(2-methyl- 1,3-thiazol-4-yl)phenyl]-5- oxo-4,5-dihydro-1H- pyrrolo[3,2-b]pyridine-6- carbonitrile | 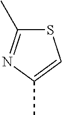 | HRMS: calculated for C₁₈H₁₁N₄O₂S (M − H)⁻: 347.0603; found: 347.061; Rt: 2.07 min. |

### Example 93

### 2-Chloro-1-[2′-fluoro-6′-(methyloxy)-4-biphenylyl]-7-hydroxy-5-oxo-4,5-dihydro-1H-pyrrolo[3,2-b]pyridine-6-carbonitrile

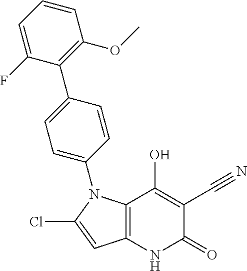

Ethyl 5-chloro-3-[(cyanoacetyl)amino]-1-[2′-fluoro-6′-(methyloxy)-4-biphenylyl]-1H-pyrrole-2-carboxylate (Intermediate 73) (240 mg, 0.526 mmol) was dissolved in DMSO (0.5 mL) and potassium tert-butoxide (1M in THF, 1.053 mL, 1.053 mmol) was added. The reaction mixture was stirred at 80° C. for 2 min and 1N HCl was added. The resulting solid was filtered and purified by chromatography on silica gel eluting with DCM/MeOH 100/0 to 90/10 to give 2-chloro-1-[2′-fluoro-6′-(methyloxy)-4-biphenylyl]-7-hydroxy-5-oxo-4,5-dihydro-1H-pyrrolo[3,2-b]pyridine-6-carbonitrile (90 mg, 0.220 mmol, 41.7% yield) as a beige solid. HRMS: calculated for C₂₁H₁₄ClFN₃O₃ (M+H)⁺: 410.0708; found: 410.0713; Rt: 2.50 min.

### Example 94

### 7-Hydroxy-1-[4-(5-methyl-2-thienyl)phenyl]-5-oxo-4,5-dihydro-1H-pyrrolo[3,2-b]pyridine-6-carbonitrile

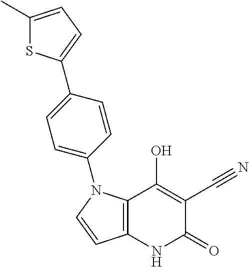

1-(4-Bromophenyl)-7-hydroxy-5-oxo-4,5-dihydro-1H-pyrrolo[3,2-b]pyridine-6-carbonitrile (Example 1) (200 mg, 0.606 mmol), {2′,6′-bis[(1-methylethyl)oxy]-2-biphenylyl}(dicyclohexyl)phosphane (11.31 mg, 0.024 mmol), potassium 2-methyl-5-thiophenetrifluoroborate (247 mg, 1.212 mmol), palladium(II) acetate (2.72 mg, 0.012 mmol) and cesium carbonate (493 mg, 1.515 mmol) are mixed in ethanol (2 mL), DMF (2 mL) and water (1 mL). The reaction vessel was sealed and heated in a microwave reactor to 120° C. for 15 minutes. The reaction mixture was dissolved in DMF and filtered through a SPE guanidine column eluting with DMF/MeOH (1/1). The filtrate was evaporated to dryness and the residue was treated with water and 1N HCl, then filtered. After drying, the resulting solid was triturated in hot EtOH then filtered to obtain 7-hydroxy-1-[4-(5-methyl-2-thienyl)phenyl]-5-oxo-4,5-dihydro-1H-pyrrolo[3,2-b]pyridine-6-carbonitrile (42 mg, 0.121 mmol, 20% yield) as a grey powder. HRMS: calculated for C₁₉H₁₂N₃O₂S (M−H)⁻: 346.0650; found: 346.0616. Rt: 2.44 min.

### Example 95

### 5-[4-(2-Chloro-6-cyano-7-hydroxy-5-oxo-4,5-dihydro-1H-pyrrolo[3,2-b]pyridin-1-yl)phenyl]-2-thiophenesulfonamide

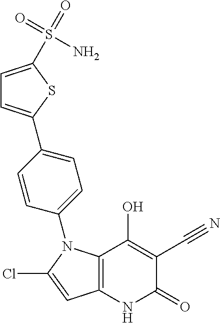

Ethyl 5-chloro-3-[(cyanoacetyl)amino]-1-[4-(4,4,5,5-tetramethyl-1,3,2-dioxaborolan-2-yl)phenyl]-1H-pyrrole-2-carboxylate (Intermediate 76) (150 mg, 0.328 mmol), 5-bromo-2-thiophenesulfonamide (159 mg, 0.655 mmol), cesium carbonate (320 mg, 0.983 mmol) and Pd(PPh₃)₄ (3.79 mg, 3.28 μmol) were mixed in a sealed tube with 1,4-dioxane (4 mL), water (1 mL). The reaction vessel was sealed and heated in a microwave reactor to 120° C. for (3×5 min). The reaction mixture was then filtered and the resulting solid was triturated successively in 1N HCl and CH₃CN to give 5-[4-(2-chloro-6-cyano-7-hydroxy-5-oxo-4,5-dihydro-1H-pyrrolo[3,2-b]pyridin-1-yl)phenyl]-2-thiophenesulfonamide (50 mg, 0.112 mmol, 34.1% yield) as a cream powder. HRMS: calculated for C₁₈H₁₀ClN₄O₄S₂ (M−H)⁻: 444.9832; found: 444.9839. Rt: 2.03 min.

Example 96 of formula (I), wherein R¹ is CN and R³, R⁴ and R⁷ are all H and R⁶ is Cl, was prepared by methods analogous to that described for Example 95 from Intermediate 76 using the appropriate bromo derivative.

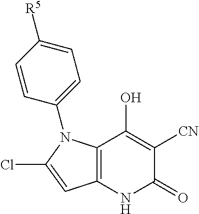

TABLE 31

| Ex- | | | |
|---|---|---|---|
| ample | Name | R⁵ | Physical data |
| 96 | 2-Chloro-1-[4-(5-chloro-2- thienyl)phenyl]-7-hydroxy- 5-oxo-4,5-dihydro-1H- pyrrolo[3,2-b]pyridine-6- carbonitrile | 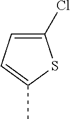 | LCMS: (M + H)⁺: 402; Rt: 2.93 min. |

### Example 97

### 4-[4-(2-Chloro-6-cyano-7-hydroxy-5-oxo-4,5-dihydro-1H-pyrrolo[3,2-b]pyridin-1-yl)phenyl]-2-thiophenecarboxamide

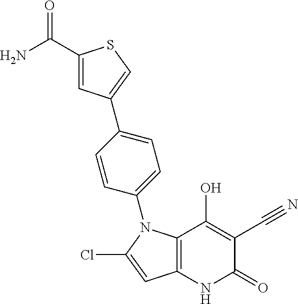

To a solution of ethyl 5-chloro-3-[(cyanoacetyl)amino]-1-[4-(5-cyano-3-thienyl)phenyl]-1H-pyrrole-2-carboxylate (Intermediate 77) (120 mg, 0.273 mmol) in DMSO (600 μL) was added dropwise potassium tert-butoxide (1M in THF, 547 μL, 0.547 mmol). The yellow reaction mixture was stirred at RT for 30 min. Water was added to the mixture before being acidified with 1N HCl to pH 1. The residue was triturated with DCM. The resulting solid was filtered, washed with DCM/MeOH (9/1) to give 4-[4-(2-chloro-6-cyano-7-hydroxy-5-oxo-4,5-dihydro-1H-pyrrolo[3,2-b]pyridine-1-yl)phenyl]-2-thiophenecarboxamide (17 mg, 14% yield) as a beige powder. HRMS: calculated for C₁₉H₁₀ClN₄O₃S (M−H)⁻: 409.0162; found: 409.0136; Rt: 1.91 min.

Examples 98 to 118 of formula (I), wherein R¹ is CN and R³, R⁴ and R⁷ are all H, were prepared by methods analogous to that described for Example 97 from the appropriate Intermediate.

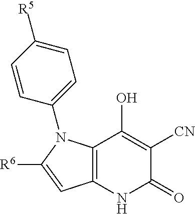

TABLE 32

| | | From | | | |
|---|---|---|---|---|---|
| Example | Name | Int. | R⁵ | R⁶ | Physical data |
| 98 | 1-[4-(1-Benzothien-3- yl)phenyl]-2-chloro-7- hydroxy-5-oxo-4,5- dihydro-1H-pyrrolo[3,2- b]pyridine-6-carbonitrile | 78 | 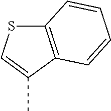 | Cl | HRMS: calculated for C₂₂H₁₁ClN₃O₂S (M − H)⁻: 416.0261; found: 416.0246; Rt: 2.60 min. |
| 99 | 2-Chloro-7-hydroxy-1-{4- [5-(methyloxy)-2- pyridinyl]phenyl}-5-oxo- 4,5-dihydro-1H- pyrrolo[3,2-b]pyridine-6- carbonitrile | 79 | 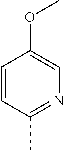 | Cl | LCMS: (M + H)⁺: 393; Rt: 2.40 min. |
| 100 | 2-Chloro-7-hydroxy-5- oxo-1-[4-(1,3-thiazol-4- yl)phenyl]-4,5-dihydro- 1H-pyrrolo[3,2-b]pyridine- 6-carbonitrile | 80 | 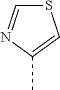 | Cl | LCMS: (M + H)⁺: 369; Rt: 2.26 min. |
| 101 | 2-Chloro-1-[4-(4-cyano-3- thienyl)phenyl]-7- hydroxy-5-oxo-4,5- dihydro-1H-pyrrolo[3,2- b]pyridine-6-carbonitrile | 81 | 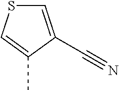 | Cl | LCMS: (M + H)⁺: 393; Rt: 2.46 min. |
| 102 | 4-[4-(2-Chloro-6-cyano-7- hydroxy-5-oxo-4,5- dihydro-1H-pyrrolo[3,2- b]pyridin-1-yl)phenyl]-3- thiophenecarboxamide | 81 | 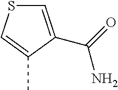 | Cl | LCMS: (M + H)⁺: 411; Rt: 2.16 min. |
| 103 | 2-Chloro-7-hydroxy-5- oxo-1-[4-(3- pyridinyl)phenyl]-4,5- dihydro-1H-pyrrolo[3,2- b]pyridine-6-carbonitrile | 82 | 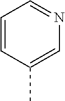 | Cl | LCMS: (M + H)⁺: 363; Rt: 2.21 min. |
| 104 | 2-Chloro-1-[2′-chloro-6′- (methyloxy)-4- biphenylyl]-7-hydroxy-5- oxo-4,5-dihydro-1H- pyrrolo[3,2-b]pyridine-6- carbonitrile | 88 | 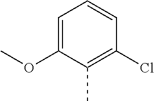 | Cl | LCMS: (M + H)⁺: 426; Rt: 2.78 min. |
| 105 | 2-Chloro-7-hydroxy-1-[4- (3-methyl-2- thienyl)phenyl]-5-oxo-4,5- dihydro-1H-pyrrolo[3,2- b]pyridine-6-carbonitrile | 83 | 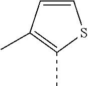 | Cl | LCMS: (M + H)⁺: 382; Rt: 2.70 min. |
| 106 | 2-Chloro-7-hydroxy-1-[4- (1-methyl-1H-pyrazol-4- yl)phenyl]-5-oxo-4,5- dihydro-1H-pyrrolo[3,2- b]pyridine-6-carbonitrile | 84 | 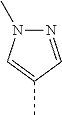 | Cl | LCMS: (M + H)⁺: 366; Rt: 2.12 min. |
| 107 | 2-Chloro-7-hydroxy-1-[4- (2-methyl-1,3-thiazol-4- yl)phenyl]-5-oxo-4,5- dihydro-1H-pyrrolo[3,2- b]pyridine-6-carbonitrile | 85 | 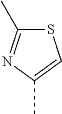 | Cl | LCMS: (M − H)⁻: 383; Rt: 2.42 min. |
| 108 | 2-Chloro-7-hydroxy-1-(3- hydroxy-4-biphenylyl)-5- oxo-4,5-dihydro-1H- pyrrolo[3,2-b]pyridine-6- carbonitrile | 89 | 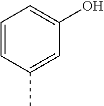 | Cl | LCMS: (M + H)⁺: 378; Rt: 2.31 min. |
| 109 | 1-[4-(6-Amino-2- pyridinyl)phenyl]-2- chloro-7-hydroxy-5-oxo- 4,5-dihydro-1H- pyrrolo[3,2-b]pyridine-6- carbonitrile | 86 | 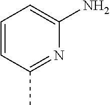 | Cl | LCMS: (M + H)⁺: 378; Rt: 2.16 min. |
| 110 | 2-Chloro-7-hydroxy-1-[3- (hydroxymethyl)-4- biphenylyl]-5-oxo-4,5- dihydro-1H-pyrrolo[3,2- b]pyridine-6-carbonitrile | 87 | 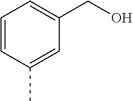 | Cl | LCMS: (M + H)⁺: 392; Rt: 2.23 min. |
| 111 | 7-Hydroxy-5-oxo-1-[4-(3- thienyl)phenyl]-4,5- dihydro-1H-pyrrolo[3,2- b]pyridine-2,6- dicarbonitrile | 74 | 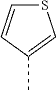 | CN | HRMS: calculated for C₁₉H₁₁N₄O₂S (M + H)⁺: 359.0603; found: 359.0580; Rt: 2.22 min. |
| 112 | 2-Chloro-7-hydroxy-1-[4- (5-methyl-2- thienyl)phenyl]-5-oxo-4,5- dihydro-1H-pyrrolo[3,2- b]pyridine-6-carbonitrile | 71 | 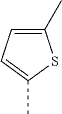 | Cl | HRMS: calculated for C₁₉H₁₆ClN₄O₂S [M + NH₄]⁺: 399.0683; found: 399.0720; Rt: 2.58 min. |
| 113 | 2-Chloro-7-hydroxy-5- oxo-1-[4-(2- thienyl)phenyl]-4,5- dihydro-1H-pyrrolo[3,2- b]pyridine-6-carbonitrile | 72 | 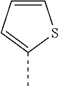 | Cl | LCMS:(M+H)⁺: 368; Rt: 2.59 min. |
| 114 | 2-Chloro-1-(3,-fluoro-2′- hydroxy-4-biphenylyl)-7- hydroxy-5-oxo-4,5- dihydro-1H-pyrrolo[3,2- b]pyridine-6-carbonitrile | 90 | 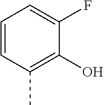 | Cl | LCMS: (M + H):⁺ 396; Rt: 2.53 min. HRMS: calculated for C₂₀H₁₀ClFN₃O₃ (M − H)⁻: 394.0395; found: 394.0432; Rt: 2.40 min. |
| 115 | 2-Chloro-1-(3′-chloro-2′- hydroxy-4-biphenylyl)-7- hydroxy-5-oxo-4,5- dihydro-1H-pyrrolo[3,2- b]pyridine-6-carbonitrile | 91 | 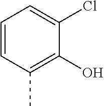 | Cl | LCMS: (M + H)⁺: 412; Rt: 2.71 min. HRMS: calculated for C₂₀H₁₀Cl₂N₃O₃ (M − H)⁻: 410.0099; found: 410.0134; Rt: 2.57 min. |
| 116 | 2-Chloro-1-(2′-fluoro-4′- methyl-4-biphenylyl)-7- hydroxy-5-oxo-4,5- dihydro-1H-pyrrolo[3,2- b]pyridine-6-carbonitrile | 92 | 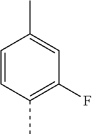 | Cl | LCMS: (M + H)⁺: 394; Rt: 2.84 min. HRMS: calculated for C₂₁H₁₄ClFN₃O₂ (M + H)⁺: 394.0758; found: 394.0737. Rt: 2.52 min. |
| 117 | 2-Chloro-1-[2′-chloro-4′- (methyloxy)-4- biphenylyl]-7-hydroxy-5- oxo-4,5-dihydro-1H- pyrrolo[3,2-b]pyridine-6- carbonitrile | 93 | 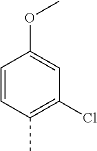 | Cl | LCMS: (M + H)⁺: 426; Rt: 2.80 min. HRMS: calculated for C₂₁H₁₄Cl₂N₃O₃ (M + H)⁺: 426.0412; found: 426.0458. Rt: 2.40 min. |
| 118 | 1-(4-Ethylphenyl)-7- hydroxy-5-oxo-4,5- dihydro-1H-pyrrolo[3,2- b]pyridine-2,6- dicarbonitrile | 75 | Et | CN | LCMS: (M + H)⁺: 305; Rt: 2.23 min. |

### Example 119

### 2-Chloro-1-(2′-chloro-6′-hydroxy-4-biphenylyl)-7-hydroxy-5-oxo-4,5-dihydro-1H-pyrrolo[3,2-b]pyridine-6-carbonitrile

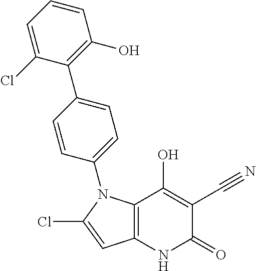

2-Chloro-1-[2′-chloro-6′-(methyloxy)-4-biphenylyl]-7-hydroxy-5-oxo-4,5-dihydro-1H-pyrrolo[3,2-b]pyridine-6-carbonitrile (Example 104) (150 mg, 0.352 mmol) and BBr₃ (1M in DCM, 0.352 mL, 0.352 mmol) were mixed in anhydrous DCM (5 mL). The reaction vessel was sealed and heated in a biotgae initiator to 80° C. for 10 min. Water (2 mL) was added to the reaction mixture. The resulting solid was filtered, washed with iPr₂O and dried. The product was purified by MDAP to give 2-chloro-1-(2′-chloro-6′-hydroxy-4-biphenylyl)-7-hydroxy-5-oxo-4,5-dihydro-1H-pyrrolo[3,2-b]pyridine-6-carbonitrile (12 mg, 8% yield) as a white powder. LCMS: (M+H)⁺: 412; Rt: 2.70 min.

### Example 120

### 2-Chloro-7-hydroxy-1-(2′-hydroxy-5′-methyl-4-biphenylyl)-5-oxo-4,5-dihydro-1H-pyrrolo[3,2-b]pyridine-6-carbonitrile

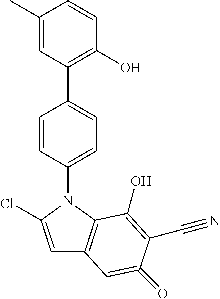

2-Chloro-7-hydroxy-1-[5′-methyl-2′-(methyloxy)-4-biphenylyl]-5-oxo-4,5-dihydro-1H-pyrrolo[3,2-b]pyridine-6-carbonitrile (Intermediate 95) (210 mg, 0.517 mmol) and BBr₃ (1M in DCM, 2.07 mL, 2.07 mmol) were mixed in anhydrous DCM (5 mL). The reaction vessel was sealed and heated in a biotage initiator to 80° C. for 10 min. Water was added and the resulting was filtered, washed with iPr₂O and dried to give the product 2-chloro-7-hydroxy-1-(2′-hydroxy-5′-methyl-4-biphenylyl)-5-oxo-4,5-dihydro-1H-pyrrolo[3,2-b]pyridine-6-carbonitrile (80 mg, 0.194 mmol, 37.5% yield) as a brown powder. LCMS: (M+H)⁺: 392; Rt: 2.65 min. HRMS: calculated for C₂₁H₁₅ClN₃O₃ (M+H)⁺: 392.0802; found: 392.0799; Rt: 2.43 min.

### Example 121

### 2-Chloro-6-(2-fluorophenyl)-7-hydroxy-1-[4-(methyloxy)phenyl]-1,4-dihydro-5H-pyrrolo[3,2-b]pyridin-5-one

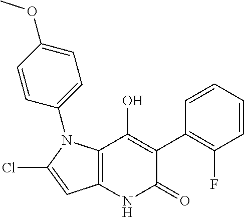

KHMDS (0.5M in toluene, 4 mL, 2.0 mmol) was added dropwise to a solution of ethyl 5-chloro-3-{[(2-fluorophenyl)acetyl]amino}-1-[4-(methyloxy)phenyl]-1H-pyrrole-2-carboxylate (Intermediate 96) (300 mg, 0.696 mmol) in THF (5 mL) at 50° C. under argon. The reaction mixture was stirred for 5 min before being quenched with AcOH and concentrated to dryness. The resulting solid was triturated in hot EtOH, filtered and dried to give 2-chloro-6-(2-fluorophenyl)-7-hydroxy-1-[4-(methyloxy)phenyl]-1,4-dihydro-5H-pyrrolo[3,2-b]pyridin-5-one (56 mg, 0.146 mmol, 20.9% yield) as a white solid. LCMS: (M+H)⁺: 385; Rt: 2.50 min.

Examples 122 to 155 of formula (I), wherein R³ and R⁷ are both H, were prepared by methods analogous to that described for Example 121 using the appropriate intermediate.

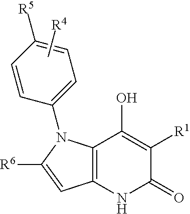

TABLE 33

| | | From | | | | | Physical |
|---|---|---|---|---|---|---|---|
| Example | Name | Int. | R¹ | R⁴ | R⁵ | R⁶ | data |
| 122 | 2-Chloro-6-(3- fluorophenyl)-7-hydroxy- 1-[4-(methyloxy)phenyl]- 1,4-dihydro-5H- pyrrolo[3,2-b]pyridine-5- one | 99 | 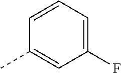 | H | OMe | Cl | LCMS: (M + H)⁺: 385; Rt: 2.60 min. |
| 123 | 2-Chloro-1-(4- ethylphenyl)-7-hydroxy- 6-phenyl-1,4-dihydro- 5H-pyrrolo[3,2- b]pyridine-5-one | 128 | 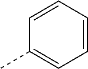 | H | Et | Cl | LCMS: (M + H)⁺: 365; Rt: 2.97 min. |
| 124 | 2-Chloro-6-(4- fluorophenyl)-7-hydroxy- 1-[4-(methyloxy)phenyl]- 1,4-dihydro-5H- pyrrolo[3,2-b]pyridin-5- one | 100 | 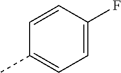 | H | OMe | Cl | LCMS: (M + H)⁺: 385; Rt: 2.66 min |
| 125 | 2-Chloro-7-hydroxy-1- [4-(methyloxy)phenyl]- 6-phenyl-1,4-dihydro- 5H-pyrrolo[3,2-b]pyridin- 5-one | 101 | 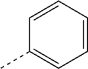 | H | OMe | Cl | LCMS: (M + H)⁺: 367; Rt: 2.65 min |
| 126 | 2-Chloro-1-(4- fluorophenyl)-7-hydroxy- 6-phenyl-1,4-dihydro- 5H-pyrrolo[3,2-b]pyridin- 5-one | 102 | 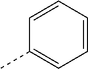 | H | F | Cl | LCMS: (M + H)⁺: 355; Rt: 2.65 min. |
| 127 | 2-Chloro-6-(2- fluorophenyl)-7-hydroxy- 1-[3-(methyloxy)phenyl]- 1,4-dihydro-5H- pyrrolo[3,2-b]pyridin-5- one | 103 | 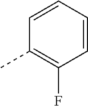 | 3-OMe | H | Cl | LCMS: (M + H⁺): 385; Rt: 2.56 min. |
| 128 | 2-Chloro-6-(3- fluorophenyl)-7-hydroxy- 1-[3-(methyloxy)phenyl]- 1,4-dihydro-5H- pyrrolo[3,2-b]pyridin-5- one | 104 | 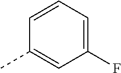 | 3-OMe | H | Cl | LCMS: (M + H)⁺ : 385; Rt: 2.68 min. |
| 129 | 2-Chloro-6-(4- fluorophenyl)-7-hydroxy- 1-[3-(methyloxy)phenyl]- 1,4-dihydro-5H- pyrrolo[3,2-b]pyridin-5- one | 105 | 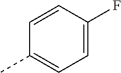 | 3-OMe | H | Cl | LCMS: (M + H)⁺: 385; Rt: 2.77 min. |
| 130 | 2-Chloro-7-hydroxy-1-[3- (methyloxy)phenyl]-6- phenyl-1,4-dihydro-5H- pyrrolo[3,2-b]pyridin-5- one | 106 | 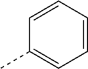 | 3-OMe | H | Cl | LCMS: (M + H)⁺: 367; Rt: 2.63 min. |
| 131 | 2-Chloro-6-(4- fluorophenyl)-7-hydroxy- 1-(4-methylphenyl)-1,4- dihydro-5H-pyrrolo[3,2- b]pyridin-5-one | 107 | 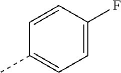 | H | Me | Cl | LCMS: (M + H)⁺: 369; Rt: 2.81 min |
| 132 | 2-Chloro-7-hydroxy-6- phenyl-1-[4- (trifluoromethyl)phenyl]- 1,4-dihydro-5H- pyrrolo[3,2-b]pyridin-5- one | 108 | 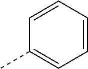 | H | CF₃ | Cl | LCMS: (M + H)⁺: 405; Rt: 2.92 min. |
| 133 | 2-Chloro-7-hydroxy-1- (4-methylphenyl)-6- phenyl-1,4-dihydro-5H- pyrrolo[3,2-b]pyridin-5- one | 109 | 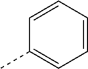 | H | Me | Cl | LCMS: (M + H)⁺: 351; Rt: 2.82 min. |
| 134 | 2-Chloro-6-(2- fluorophenyl)-7-hydroxy- 1-(4-methylphenyl)-1,4- dihydro-5H-pyrrolo[3,2- b]pyridin-5-one | 110 | 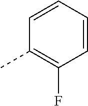 | H | Me | Cl | LCMS: (M + H)⁺: 369; Rt: 2.64 min. |
| 135 | 2-Chloro-6-(3- fluorophenyl)-7-hydroxy- 1-(4-methylphenyl)-1,4- dihydro-5H-pyrrolo[3,2- b]pyridin-5-one | 111 | 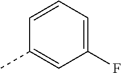 | H | Me | Cl | LCMS: (M + H):⁺ 369; Rt: 2.75 min. |
| 136 | 2-Chloro-6-(2- fluorophenyl)-7-hydroxy- 1-[4- (trifluoromethyl)phenyl]- 1,4-dihydro-5H- pyrrolo[3,2-b]pyridin-5- one | 112 | 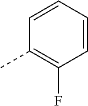 | H | CF₃ | Cl | LCMS: (M + H)⁺: 423; Rt: 2.74 min. |
| 137 | 2-Chloro-6-(3- fluorophenyl)-7-hydroxy- 1-[4- (trifluoromethyl)phenyl]- 1,4-dihydro-5H- pyrrolo[3,2-b]pyridin-5- one | 113 | 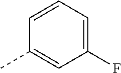 | H | CF₃ | Cl | LCMS: (M + H)⁺: 423; Rt: 2.92 min. |
| 138 | 2-Chloro-6-(2- fluorophenyl)-7-hydroxy- 1-{4- [(trifluoromethyl)oxy] phenyl}-1,4-dihydro-5H- pyrrolo[3,2-b]pyridin-5- one | 114 | 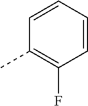 | H | OCF₃ | Cl | LCMS: (M + H)⁺: 439; Rt: 2.88 min. |
| 139 | 2-Chloro-6-[3- (ethyloxy)phenyl]-7- hydroxy-1-(4- methylphenyl)-1,4- dihydro-5H-pyrrolo[3,2- b]pyridin-5-one | 115 | 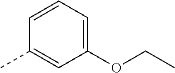 | H | Me | Cl | LCMS: (M + H)⁺: 395; Rt: 3.04 min. |
| 140 | 4-[2-Chloro-7-hydroxy-1- (4-methylphenyl)-5-oxo- 4,5-dihydro-1H- pyrrolo[3,2-b]pyridin-6- yl]benzonitrile | 116 | 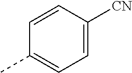 | H | Me | Cl | LCMS: (M − H)⁻: 374; Rt: 2.50 min. |
| 141 | 2-Chloro-1-(3,4- dimethylphenyl)-7- hydroxy-6-phenyl-1,4- dihydro-5H-pyrrolo[3,2- b]pyridin-5-one | 129 | 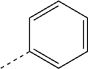 | 3-Me | Me | Cl | LCMS: (M + H)⁺: 365; Rt: 3.09 min. |
| 142 | 2-Chloro-7-hydroxy-1- (4-methylphenyl)-6-(3- nitrophenyl)-1,4-dihydro- 5H-pyrrolo[3,2-b]pyridin- 5-one | 117 | 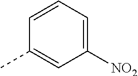 | H | Me | Cl | LCMS: (M + H)⁺: 396; Rt: 2.72 min. |
| 143 | 3-[2-Chloro-7-hydroxy-1- (4-methylphenyl)-5-oxo- 4,5-dihydro-1H- pyrrolo[3,2-b]pyridin-6- yl]benzonitrile | 118 | 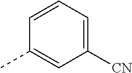 | H | Me | Cl | LCMS: (M + H)⁺: 376; Rt: 2.47 min. |
| 144 | [4-(2-Chloro-7-hydroxy- 5-oxo-6-phenyl-4,5- dihydro-1H-pyrrolo [3,2-b]pyridin-1- yl)phenyl]acetonitrile | 130 | 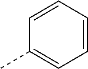 | H | CH₂CN | Cl | LCMS: (M + H)⁺: 376; Rt: 2.31 min. |
| 145 | 6-(3-Bromophenyl)-2- chloro-7-hydroxy-1-(4- methylphenyl)-1,4- dihydro-5H-pyrrolo [3,2-b]pyridin-5-one | 119 | 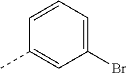 | H | Me | Cl | LCMS: (M + H)⁺: 429, 431; Rt: 2.88 min. |
| 146 | 3-[2-Chloro-7-hydroxy-1- (4-methylphenyl)-5-oxo- 4,5-dihydro-1H- pyrrolo[3,2-b]pyridin-6- yllbenzoic acid | 118 | 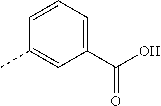 | H | Me | Cl | LCMS: (M + H)⁺: 395; Rt: 2.25 min. |
| 147 | 1-(4-Ethylphenyl)-7- hydroxy-5-oxo-6-phenyl- 4,5-dihydro-1H- pyrrolo[3,2-b]pyridine-2- carbonitrile | 133 | 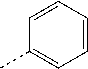 | H | Et | CN | LCMS: (M + H)⁺: 356; Rt: 2.52 min. |
| 148 | 6-(2-Fluorophenyl)-7- hydroxy-1-[4- (methyloxy)phenyl]-1,4- dihydro-5H-pyrrolo[3,2- b]pyridin-5-one | 98 | 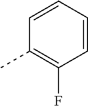 | H | OMe | H | LCMS: (M + H)⁺: 351; Rt: 2.35 min. |
| 149 | 6-(3-Fluorophenyl)-7- hydroxy-1-[4- (methyloxy)phenyl]-1,4- dihydro-5H-pyrrolo[3,2- b]pyridin-5-one | 97 | 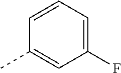 | H | OMe | H | LCMS: (M + H)⁺: 351; Rt: 2.46 min. |
| 150 | 2-Chloro-7-hydroxy-6-[3- (methyloxy)phenyl]-1-[4- (3-thienyl)phenyl]-1,4- dihydro-5H-pyrrolo[3,2- b]pyridin-5-one | 137 | 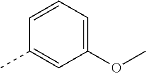 | H | 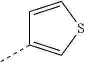 | Cl | HRMS calculated for C₂₄H₁₆ClN₂O₃S (M − H)⁻: 447.0570; found: 447.0545: Rt: 2.81 min |
| 151 | 3-{2-Chloro-7-hydroxy- 5-oxo-1-[4-(3- thienyl)phenyl]-4,5- dihydro-1H-pyrrolo[3,2- b]pyridin-6-yl}benzoic acid | 138 | 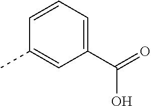 | H | 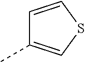 | Cl | HRMS calculated for C₂₄H₁₄ClN₂O₄S (M − H)⁻: 461.0363; found: 461.0371; Rt: 2.38 min |
| 152 | 2-Chloro-7-hydroxy-6- phenyl-1-[4-(3- thienyl)phenyl]-1,4- dihydro-5H-pyrrolo[3,2- b]pyridin-5-one | 120 | 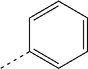 | H | 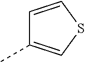 | Cl | LCMS: (M + H)⁺: 419; Rt: 3.14 min. |
| 153 | 2-{2-Chloro-7-hydroxy- 5-oxo-1-[4-(3- thienyl)phenyl]-4,5- dihydro-1H-pyrrolo[3,2- b]pyridin-6-yl}benzoic acid | 139 | 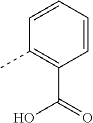 | H | 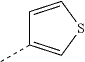 | Cl | HRMS calculated for C₂₄H₁₄ClN₂O₄S (M − H)⁻: 461.0363; found: 461.0343; Rt: 2.54 min. |
| 154 | 4-{2-Chloro-7-hydroxy- 5-oxo-1-[4-(3- thienyl)phenyl]-4,5- dihydro-1H-pyrrolo[3,2- b]pyridin-6- yl}benzonitrile | 121 | 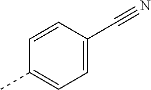 | H | 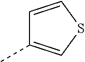 | Cl | HRMS calculated for C₂₄H₁₅ClN₃O₂S (M + H)⁺: 444.0573; found: 444.0573; Rt: 2.52 min. |
| 155 | 3-{2-Chloro-7-hydroxy- 5-oxo-1-[4-(3- thienyl)phenyl]-4,5- dihydro-1H-pyrrolo[3,2- b]pyridin-6- yl}benzonitrile | 122 | 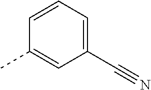 | H | 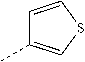 | Cl | HRMS calculated for C₂₄H₁₅ClN₃O₂S (M + H)⁺: 444.0573; found: 444.0578; Rt: 2.56 min. |

### Example 156

### 1-(4-Acetylphenyl)-2-chloro-7-hydroxy-5-oxo-4,5-dihydro-1H-pyrrolo[3,2-b]pyridine-6-carbonitrile

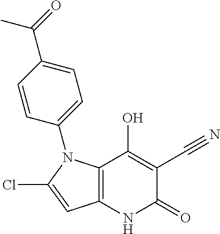

To a solution of ethyl 1-(4-acetylphenyl)-5-chloro-3-[(cyanoacetyl)amino]-1H-pyrrole-2-carboxylate (Intermediate 55) (2.26 g, 6.05 mmol) in DMSO (6 mL) heated to 90° C. was added potassium tert-butoxide (1M in THF, 12.09 mL, 12.09 mmol) and the resulting mixture was stirred at 90° C. for 2 min before being quenched with 1N HCl. The resulting solid was filtered and dried to give 1-(4-acetylphenyl)-2-chloro-7-hydroxy-5-oxo-4,5-dihydro-1H-pyrrolo[3,2-b]pyridine-6-carbonitrile (5 mg, 0.2% yield) as a white solid. HRMS: calculated for C₁₆H₁₄ClN₄O₃ [M+NH₄]: 345.0755; found: 345.0788; Rt: 2.01 min.

### Example 157

### N-[4-(2-Chloro-6-cyano-7-hydroxy-5-oxo-4,5-dihydro-1H-pyrrolo[3,2-b]pyridin-1-yl)phenyl]acetamide

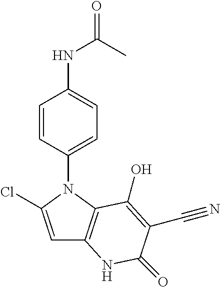

To a solution of 1-(4-aminophenyl)-2-chloro-7-hydroxy-5-oxo-4,5-dihydro-1H-pyrrolo[3,2-b]pyridine-6-carbonitrile (Intermediate 140) (200 mg, 0.665 mmol) in a THF (10 mL)/DMF (2 mL) mixture at 0° C. was added dropwise acetyl chloride (0.047 mL, 0.665 mmol) in 5 min. The reaction mixture was stirred 15 min at 0° C. then 1 h at RT and was concentrated in vacuo, taken up in EtOAc and washed with H₂O, and sat NaCl. The organic layer was dried over anhydrous Na₂SO₄, filtered and concentrated in vacuo. The yellow oily residue was triturated with hot CH₃CN. The resulting solid was filtered, washed with CH₃CN, iPr₂O to give the product (40 mg, 0.117 mmol, n 17% yield) as an off-white powder. LCMS: (M+H)⁺: 343; Rt: 1.89 min.

### Example 158

### 2-Chloro-7-hydroxy-1-(4′-methyl-4-biphenylyl)-6-phenyl-1,4-dihydro-5H-pyrrolo[3,2-b]pyridin-5-one

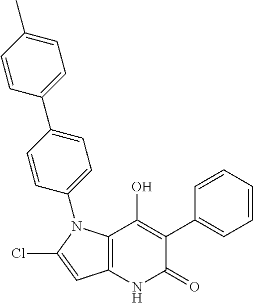

To a solution of ethyl 5-chloro-1-(4′-methyl-4-biphenylyl)-3-[(phenylacetyl)amino]-1H-pyrrole-2-carboxylate (Intermediate 132) (96 mg, 0.203 mmol) in DMSO (2 mL) heated to 90° C. was added potassium tert-butoxide (1M in THF, 0.406 mL, 0.406 mmol) and the resulting mixture was stirred at 90° C. overnight before being quenched with 1N HCl. The resulting solid was filtered, washed with water and dried. The solid was then triturated in hot 1N NaOH and neutralized with concentrated HCl, filtered and dried to give 2-chloro-7-hydroxy-1-(4′-methyl-4-biphenylyl)-6-phenyl-1,4-dihydro-5H-pyrrolo[3,2-b]pyridin-5-one (2 mg, 4.69 μmol, 2% yield) as an off-white solid. LCMS: (M+H)⁺: 427; Rt: 3.42 min.

Example 159 of formula (I), wherein R¹ is phenyl, R³, R⁴ and R⁷ are all H and R⁶ is Cl was prepared by methods analogous to that described for Example 158 using Intermediate 131.

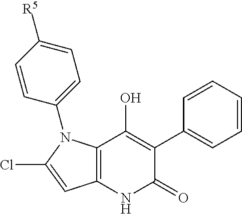

TABLE 34

| Example | Name | R⁵ | Physical data |
|---|---|---|---|
| 159 | 2-Chloro-1-(4- cyclohexylpheny1)-7- hydroxy-6-phenyl-1,4- dihydro-5H- pyrrolo[3,2-b]pyridin- 5-one | 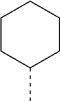 | LCMS: (M + H)⁺: 419; Rt: 3.55 min. HRMS: calculated for C₂₅H₂₄ClN₂O₂ (M + H)⁺: 419.1526; found: 419.1565; Rt: 3.20 min. |

### Example 160

### 4-{2-Chloro-7-hydroxy-5-oxo-1-[4-(3-thienyl)phenyl]-4,5-dihydro-1H-pyrrolo[3,2-b]pyridin-6-yl}benzamide

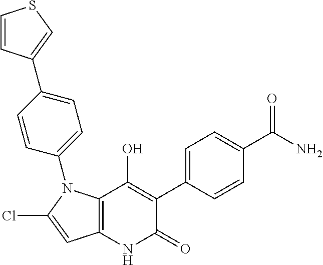

To a solution of 4-{2-chloro-7-hydroxy-5-oxo-1-[4-(3-thienyl)phenyl]-4,5-dihydro-1H-pyrrolo[3,2-b]pyridin-6-yl}benzonitrile (Example 154) (200 mg, 0.451 mmol) in ethanol (30 mL)/water (5 mL) was added KOH (126 mg, 2.253 mmol). The reaction vessel was sealed and heated in a biotage initiator to 100° C. for 1 h. The reaction mixture was diluted with water and the precipitate was filtered, washed successively with water, acetonitrile and iPr₂O to give 4-{2-chloro-7-hydroxy-5-oxo-1-[4-(3-thienyl)phenyl]-4,5-dihydro-1H-pyrrolo[3,2-b]pyridin-6-yl}benzamide (105 mg, 5°% yield) as a beige powder. HRMS: calculated for C₂₄H₁₇ClN₃O₃S (M+H)⁺: 462.0679; found: 462.0713; Rt: 2.41 min.

Examples 161 and 162 of formula (I), wherein R³, R⁴ and R⁷ are all H, were prepared by methods analogous to that described for Example 160.

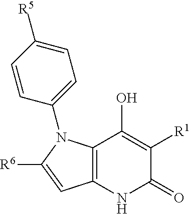

TABLE 35

| | | From | | | | |
|---|---|---|---|---|---|---|
| Example | Name | Example | R¹ | R⁵ | R⁶ | Physical data |
| 161 | 3-{2-Chloro-7-hydroxy- 5-oxo-1-[4-(3- thienyl)phenyl]-4,5- dihydro-1H-pyrrolo[3,2- b]pyridin-6-yl} benzamide | 155 | 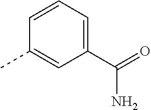 | 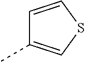 | Cl | HRMS calculated for C₂₄H₁₇ClN₃O₃S (M + H)⁺: 462.0679; found: 462.0642; Rt: 2.41 min. |
| 162 | 4-{2-Chloro-7-hydroxy- 5-oxo-1-[4-(3- thienyl)phenyl]-4,5- dihydro-1H-pyrrolo[3,2- b]pyridin-6-yl}benzoic acid | 154 | 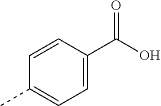 | 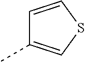 | Cl | HRMS calculated for C₂₄H₁₆ClN₂O₄S (M + H)⁺: 463.0519; found: 463.0475; Rt: 2.30 min. |

### Example 163

### 2-Chloro-7-hydroxy-1-[2′-hydroxy-3′-(methyloxy)-4-biphenylyl]-6-phenyl-1,4-dihydro-5H-pyrrolo[3,2-b]pyridin-5-one

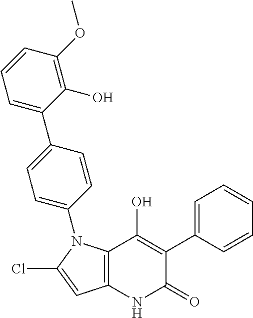

To a solution of ethyl 5-chloro-1-[2′-hydroxy-3′-(methyloxy)-4-biphenylyl]-3-[(phenylacetyl)amino]-1H-pyrrole-2-carboxylate (Intermediate 134) (3.35 g, 6.65 mmol) in THF (110 mL) at RT was added KHMDS (1M in THF, 33.2 mL, 33.2 mmol). The reaction mixture is then stirred at 50° C. for 1 h before being quenched with 1N HCl. The aqueous layer was extracted with DCM, dried and concentrated to dryness. The residue was dissolved in DCM and precipitated with pentane to give 2-chloro-7-hydroxy-1-[2′-hydroxy-3′-(methyloxy)-4-biphenylyl]-6-phenyl-1,4-dihydro-5H-pyrrolo[3,2-b]pyridin-5-one (1.77 g, 58% yield) as a beige solid. ¹H NMR: (CDCl₃, 400 MHz) δ 11.31 (br s, 1H), 9.49 (br s, 1H), 8.77 (br s, 1H), 7.65 (d, J=8.2 Hz, 2H), 7.44 (d, J=8.2 Hz, 2H), 7.32-7.25 (m, 2H), 7.25-7.20 (m, 3H), 7.00-6.86 (m, 3H), 6.26 (s, 1H), 3.87 (s, 3H).

Examples 164 to 168 of formula (I), wherein R³, R⁴ and R⁷ are all H, were prepared by methods analogous to that described for Example 163.

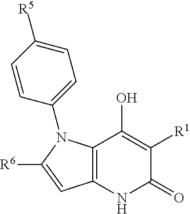

TABLE 36

| | | From | | | | |
|---|---|---|---|---|---|---|
| Example | Name | Int. | R¹ | R⁵ | R⁶ | Physical data |
| 164 | 2-Chloro-6-(2- fluorophenyl)-7- hydroxy-1-[2′-hydroxy- 3′-(methyloxy)-4- biphenylyl]-1,4- dihydro-5H- pyrrolo[3,2-b] pyridin-5-one | 123 | 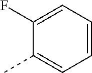 | 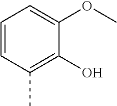 | Cl | HRMS calculated for C₂₆H₁₉ClFN₂O₄ (M + H)⁺: 477.1017; found: 477.1038; Rt: 2.46 min. |
| 165 | 2-Chloro-6-(3- chlorophenyl)-7- hydroxy-1-[2′-hydroxy- 3′-(methyloxy)-4- biphenylyl]-1,4- dihydro-5H- pyrrolo[3,2-b]pyridin-5- one | 124 | 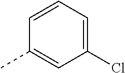 | 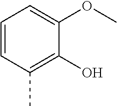 | Cl | HRMS calculated for C₂₆H₁₉Cl₂N₂O₄ (M + H)⁺: 493.0722; found: 493.0764; Rt: 2.59 min. |
| 166 | 3-{2-Chloro-7-hydroxy- 1-[2′-hydroxy-3′- (methyloxy)-4- biphenylyl]-5-oxo-4,5- dihydro-1H- pyrrolo[3,2-b]pyridin-6- yl}benzoic acid | 125 | 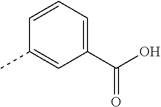 | 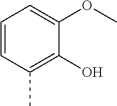 | Cl | HRMS calculated for C₂₇H₂₀ClN₂O₆ (M + H)⁺: 503.1010; found: 503.1007; Rt: 2.22 min. |
| 167 | 4-{2-Chloro-7-hydroxy- 1-[2′-hydroxy-3′- (methyloxy)-4- biphenylyl]-5-oxo-4,5- dihydro-1H- pyrrolo[3,2-b]pyridin-6- yl}benzonitrile | 126 | 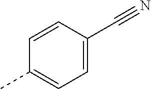 | 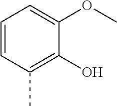 | Cl | HRMS calculated for C₂₇H₁₉ClN₃O₄ (M + H)⁺: 484.1064; found: 484.1095; Rt: 2.43 min. |
| 168 | 2-Chloro-7-hydroxy-1- [2′-hydroxy-3′- (methyloxy)-4- biphenylyl]-6-[3- (methyloxy)phenyl]- 1,4-dihydro-5H- pyrrolo[3,2-b]pyridin-5- one | 127 | 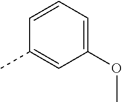 | 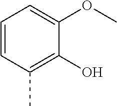 | Cl | ¹H NMR: (CDCl₃, 400 MHz) δ 11.23 (br s, 1H), 9.42 (br s, 1H), 8.74 (br s, 1H), 7.64 (m, 2H), 7.43 (m, 2H), 7.20 (m, 1H), 7.00 (m, 1H), 6.96-6.85 (m, 2H), 6.82-6.75 (m, 3H), 6.25 (s, 1H), 3.87 (s, 3H), 3.72 (s, 3H). |

Example 169 of formula (I), wherein R³, R⁴ and R⁷ are all H, was prepared by methods analogous to that described for Example 160.

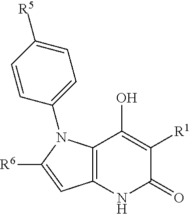

TABLE 37

| | | From | | | | |
|---|---|---|---|---|---|---|
| Example | Name | Example | R¹ | R⁵ | R⁶ | Physical data |
| 169 | 4-{2-Chloro-7- hydroxy-1-[2′-hydroxy- 3′-(methyloxy)-4- biphenylyl[-5-oxo-4,5- dihydro-1H- pyrrolo[3,2-b]pyridin- 6-yl}benzoic acid | 167 | 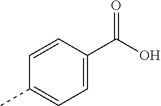 | 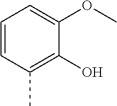 | Cl | HRMS calculated for C₂₇H₂₀ClN₂O₆ (M + H)+: 503.1010; found: 503.1013; Rt: 2.22 min. |

### Biological Assay

### AMPK Enzymatic Assay

Human recombinant AMPK (Invitrogen #PV4673 & #PV4675) is used in a FRET assay format (Z'Lyte—Invitrogen). Assay conditions are as follow: ATP 100 μM, peptide (Invitrogen #PR8650) 2 μM, 1% final DMSO in Z'Lyte kinase buffer. Reaction is initiated by addition of 0.2-0.8 ng of AMPK and incubated for 1-hour @ 30° C. A further 1-hour incubation @ 30° C. with the development reagent (Invitrogen #PR5194) is performed. FRET signal is then measured and converted to “% peptide phosphorylation” according to Z'Lyte given calculation procedure. Evaluation of compounds is carried out using concentration-response curves. Final data are expressed in “% activation” calculating the ratio of “% peptide phosphorylation” between compound-condition and basal-condition. Alternatively pEC200 (−Log(compound concentration leading to a 2-fold AMPK activity increase)) is produced through fitting of the concentration-response curves. All data are means of at least 2 independent experiments.

The compounds of Examples 1 to 34 and 36 to 76 were tested in the assay described above and gave pEC₂₀₀ values of greater than 4.0.

In one aspect, the compounds of the invention give a pEC₂₀₀ value of ≧5.0 when tested in this assay. In a further aspect, the compounds of the invention give a pEC₂₀₀ value of ≧6.0 when tested in this assay. In a further aspect, the compounds of the invention give a pEC₂₀₀ value of ≧7.0 when tested in this assay.

The compounds of Examples 35 and 77 to 169 were tested in the assay described above and gave average pEC₅₀ values of greater than 4.5.

In one aspect, the compounds of the invention give average pEC₅₀ values of ≧5.0 when tested in this assay. In a further aspect, the compounds of the invention give average pEC₅₀ values of ≧6.0 when tested in this assay. In a further aspect, the compounds of the invention give average pEC₅₀ values of ≧7.0 when tested in this assay.

For instance, Example compounds 5 and 72 gave an average pEC₂₀₀ value of 5.8 and 5.6 respectively.

The following compounds were also prepared and when tested by the above described in vitro assay for AMPK activity were found to exhibit an average pEC₂₀₀ value of less than 4 or a PEC₅₀ value of less than 4.5.

| Name | Structure |
|---|---|
| 1-(4′-Fluoro-4- biphenylyl)-7- hydroxy-1,4-dihydro- 5H-pyrrolo[3,2-b] pyridin-5-one | 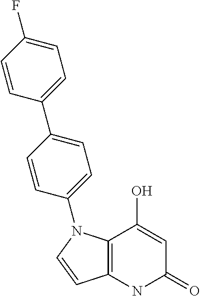 |
| 4′-(7-Hydroxy-5-oxo- 4,5-dihydro-1H- pyrrolo[3,2-b] pyridin-1-yl)-2- biphenylcarbonitrile | 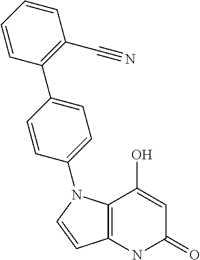 |
| N-[4′-(6-Cyano-7- hydroxy-5-oxo-4,5- dihydro-1H-pyrrolo [3,2-b]pyridin-1- yl)-2-biphenylyl] acetamide | 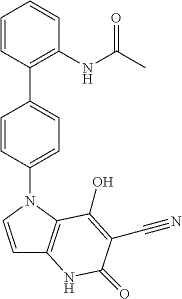 |
| 7-Hydroxy-5-oxo-1- [4-(2-pyridinyl) phenyl]-4,5-dihydro- 1H-pyrrolo[3,2-b] pyridine-6-carbonitrile | 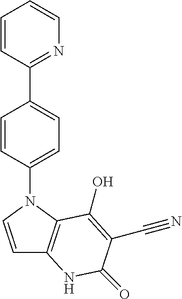 |
| 7-Hydroxy-5-oxo-1- [4-(2-pyrimidinyl) phenyl]-4,5-dihydro- 1H-pyrrolo[3,2-b] pyridine-6-carbonitrile | 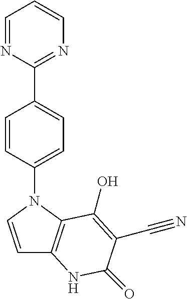 |
| 7-Hydroxy-1-[4-(6- methyl-3-pyridinyl) phenyl]-5-oxo-4,5- dihydro-1H-pyrrolo [3,2-b]pyridine-6- carbonitrile | 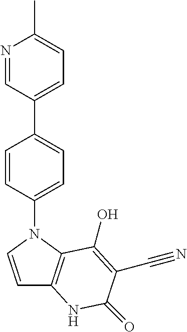 |
| 7-Hydroxy-1-(4′- methyl-4-biphenylyl)- 1,4-dihydro-5H- pyrrolo[3,2-b]pyridin- 5-one | 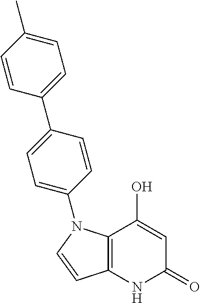 |
| 7-Hydroxy-1-[4- (3-methyl-2- pyridinyl)phenyl]- 5-oxo-4,5-dihydro- 1H-pyrrolo[3,2-b] pyridine-6-carbonitrile | 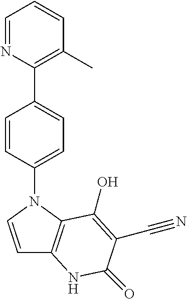 |
| 7-Hydroxy-1-[4- (4-methyl-3-pyridinyl) phenyl]-5-oxo- 4,5-dihydro- 1H-pyrrolo[3,2-b] pyridine-6-carbonitrile | 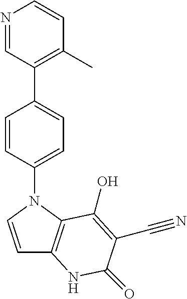 |
| Ethyl 7-hydroxy- 1-(4′-methyl-4- biphenylyl)-5- oxo-4,5-dihydro- 1H-pyrrolo[3,2-b] pyridine-6-carboxylate | 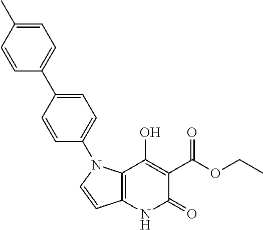 |
| 7-Hydroxy-1-(4′- methyl-4-biphenylyl)- 5-oxo-4,5-dihydro- 1H-pyrrolo[3,2-b] pyridine-6-carboxamide | 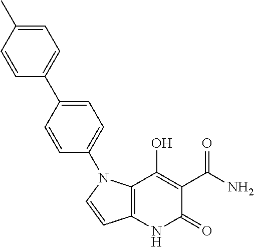 |
| 2-[4-(6-Cyano-7- hydroxy-5-oxo-4,5- dihydro-1H-pyrrolo [3,2-b]pyridin-1-yl) phenyl]-1,3-thiazole- 4-carboxylic acid | 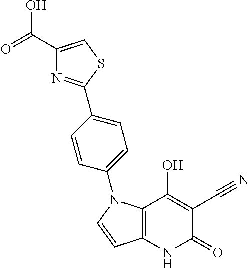 |
| 7-Hydroxy-1-[4- (3-methyl-2-thienyl) phenyl]-5-oxo-4,5- dihydro-1H-pyrrolo [3,2-b]pyridine-6- carbonitrile | 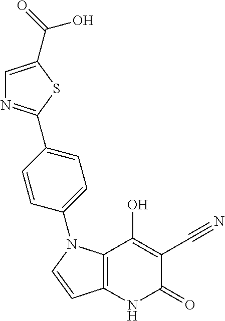 |
| 6-(4-Fluorophenyl)- 7-hydroxy-1-[4- (methyloxy)phenyl]- 1,4-dihydro-5H-pyrrolo [3,2-b]pyridin-5-one | 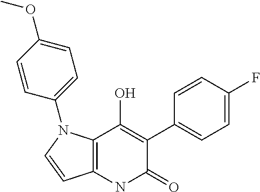 |
| 2-Chloro-1,6-bis (4-fluorophenyl)-7- hydroxy-1,4-dihydro- 5H-pyrrolo[3,2-b] pyridin-5-one | 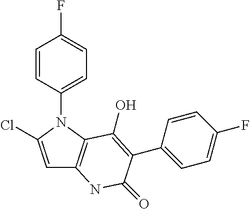 |
| 7-Hydroxy-1-[4- (methyloxy)phenyl]- 6-phenyl-1,4-dihydro- 5H-pyrrolo[3,2-b] pyridin-5-one | 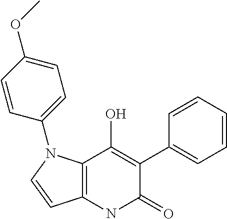 |
| 2-Chloro-6- (4-fluorophenyl)- 7-hydroxy-1-[4- (trifluoromethyl) phenyl]-1,4-dihydro- 5H-pyrrolo[3,2-b] pyridin-5-one | 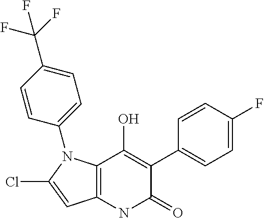 |
| 2-Chloro-6-(4- fluorophenyl)-7- hydroxy-1-{4- [(trifluoromethyl) oxy]phenyl}-1,4- dihydro-5H-pyrrolo [3,2-b]pyridin-5-one | 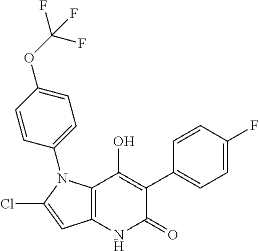 |
| 2-Chloro-6-[2- (ethyloxy)phenyl]- 7-hydroxy-1-(4- methylphenyl)-1,4- dihydro-5H-pyrrolo [3,2-b]pyridin-5-one | 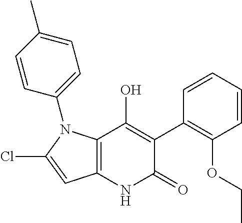 |
| 2-Chloro-6-[4- (ethyloxy)phenyl]- 7-hydroxy-1- (4-methylphenyl)- 1,4-dihydro-5H- pyrrolo[3,2-b] pyridin-5-one | 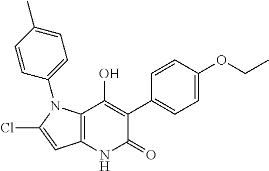 |
| 2-Chloro-6- (3-fluorophenyl)- 1-(4-fluorophenyl)- 7-hydroxy-1,4- dihydro-5H-pyrrolo [3,2-b]pyridin-5-one | 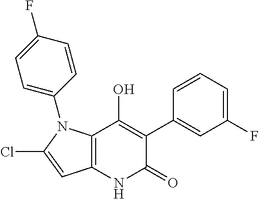 |
| 2-Chloro-7- hydroxy-6-[4- (methyloxy)phenyl]- 1-[4-(3-thienyl) phenyl]-1,4-dihydro- 5H-pyrrolo[3,2-b] pyridin-5-one | 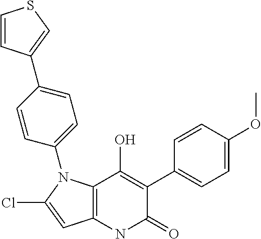 |

1. A compound of formula (I) 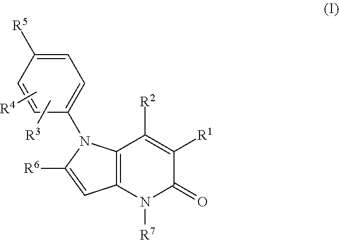
wherein
R¹ represents:
(i) —CN, H or —CO₂C₁₋₃alkyl; or
(ii) -phenyl optionally substituted by one or two groups independently selected from —C₁₋₄alkyl, —OC₁₋₄alkyl, —OH, —C₁₋₄haloalkyl, —NO₂, —CN, —CO₂H, —C(═O)NH₂ or halogen;
R² represents —OH;
R³ and R⁴ each independently represent H, —C₁₋₄alkyl, —OC₁₋₄alkyl or fluoro;
R⁵ represents
(i) H, —C₁₋₄alkyl, —OC₁₋₄alkyl, —CO₂C₁₋₄alkyl, —CN, —C₁₋₄alkylCN, —C₁₋₄haloalkyl, —OC₁₋₄haloalkyl, —C(═O)C₁₋₄alkyl, —XC(═O)C₁₋₄alkyl or halogen; or
(ii) —C₆₋₁₀aryl, -(5-10 membered heteroaryl), -(5-10 membered heterocyclyl) or —C₃₋₈cycloalkyl wherein the —C₆₋₁₀aryl, -(5-10 membered heteroaryl), -(5-10 membered heterocyclyl) or —C₃₋₈cycloalkyl is optionally substituted by one, two or three groups independently selected from —C₁₋₄alkyl, —OC₁₋₄alkyl, —C₂₋₄alkenyl, —OH, —C₁₋₄alkyleneOH, —C₁₋₄haloalkyl, —CN, —CO₂H, —C₁₋₄alkyleneCO₂H, —XC(═O)C₁₋₄alkyl, —Si(C₁₋₃alkyl)₃, —SO₂NR⁹R¹⁰, —C(═O)NR⁸R¹⁰, —NR⁹R¹⁰ or halogen;
X represents O or NR⁸;
R⁶ represents H, —C₁₋₄alkyl, —CN or chloro;
R⁷ represents H or —C₁₋₄alkyl;
R⁸ represents H or —C₁₋₄alkyl; and
R⁹ and R¹⁰ each independently represent H or —C₁₋₄alkyl;
or a salt thereof.

2. A compound of formula (I) or a salt thereof according to claim 1 wherein R¹ represents —CN.

3. A compound of formula (I) or a salt thereof according to claim 1 wherein R¹ represents phenyl optionally substituted by a group independently selected from —CH₃, —OCH₃, —OC₂H₅, —OH, —CF₃, —NO₂, —CN, —CO₂H, —C(═O)NH₂ or halogen.

4. A compound of formula (I) or a salt thereof according to claim 1 wherein R³ and R⁴ each independently represent H.

5. A compound of formula (I) or a salt thereof according to claim 1 wherein R⁵ represents H, —C₁₋₄alkyl, —OC₁₋₄alkyl, —CO₂C₁₋₄alkyl, —CN, —C₁₋₄alkylCN, —C₁₋₄haloalkyl, —OC₁₋₄haloalkyl, —C(═O)C₁₋₄alkyl, —XC(═O)C₁₋₄alkyl or halogen.

6. A compound of formula (I) or a salt thereof according to claim 1 wherein R⁵ represents —C₆₋₁₀aryl, -(5-10 membered heteroaryl), -(5-10 membered heterocyclyl) or —C₃₋₈cycloalkyl wherein the —C₆₋₁₀aryl, -(5-10 membered heteroaryl), -(5-10 membered heterocyclyl) or —C₃₋₈cycloalkyl is optionally substituted by one, two or three groups independently selected from —C₁₋₄alkyl, —OC₁₋₄alkyl, —C₂₋₄alkenyl, —OH, —C₁₋₄alkyleneOH, —C₁₋₄haloalkyl, —CN, —CO₂H, —C₁₋₄alkyleneCO₂H, —XC(═O)C₁₋₄alkyl, —Si(C₁₋₃alkyl)₃, —SO₂NR⁹R¹⁰, —C(═O)NR⁹R¹⁰, —NR⁹R¹⁰ or halogen.

7. A compound of formula (I) or a salt thereof according to claim 1 wherein R⁶ represents H or chloro.

8. A compound of formula (I) or a salt thereof according to claim 1 wherein R⁷ represents H.

9. A compound of formula (I) according to claim 1 selected from the group consisting of
1-(4-Bromophenyl)-7-hydroxy-5-oxo-4,5-dihydro-1H-pyrrolo[3,2-b]pyridine-6-carbonitrile,
7-Hydroxy-1-(2′-hydroxy-4-biphenylyl)-5-oxo-4,5-dihydro-1H-pyrrolo[3,2-b]pyridine-6-carbonitrile,
7-Hydroxy-1-[4′-(methyloxy)-4-biphenylyl]-5-oxo-4,5-dihydro-1H-pyrrolo[3,2-b]pyridine-6-carbonitrile,
7-Hydroxy-1-[2′-(methyloxy)-4-biphenylyl]-5-oxo-4,5-dihydro-1H-pyrrolo[3,2-b]pyridine-6-carbonitrile,
7-Hydroxy-1-[3′-(methyloxy)-4-biphenylyl]-5-oxo-4,5-dihydro-1H-pyrrolo[3,2-b]pyridine-6-carbonitrile,
1-(4′-Fluoro-4-biphenylyl)-7-hydroxy-5-oxo-4,5-dihydro-1H-pyrrolo[3,2-b]pyridine-6-carbonitrile,
1-(2′-Cyano-4-biphenylyl)-7-hydroxy-5-oxo-4,5-dihydro-1H-pyrrolo[3,2-b]pyridine-6-carbonitrile,
1-(3′-Fluoro-4-biphenylyl)-7-hydroxy-5-oxo-4,5-dihydro-1H-pyrrolo[3,2-b]pyridine-6-carbonitrile,
7-Hydroxy-5-oxo-1-phenyl-4,5-dihydro-1H-pyrrolo[3,2-b]pyridine-6-carbonitrile,
7-Hydroxy-1-[2′-hydroxy-3′-(methyloxy)-4-biphenylyl]-5-oxo-4,5-dihydro-1H-pyrrolo[3,2-b]pyridine-6-carbonitrile,
1-(2′-Fluoro-4-biphenylyl)-7-hydroxy-5-oxo-4,5-dihydro-1H-pyrrolo[3,2-b]pyridine-6-carbonitrile,
1-(4′-Chloro-4-biphenylyl)-7-hydroxy-5-oxo-4,5-dihydro-1H-pyrrolo[3,2-b]pyridine-6-carbonitrile,
7-Hydroxy-1-(4′-hydroxy-4-biphenylyl)-5-oxo-4,5-dihydro-1H-pyrrolo[3,2-b]pyridine-6-carbonitrile,
7-Hydroxy-5-oxo-1-[4′-(trifluoromethyl)-4-biphenylyl]-4,5-dihydro-1H-pyrrolo[3,2-b]pyridine-6-carbonitrile,
7-Hydroxy-1-(4′-methyl-4-biphenylyl)-5-oxo-4,5-dihydro-1H-pyrrolo[3,2-b]pyridine-6-carbonitrile,
1-(4′-Cyano-4-biphenylyl)-7-hydroxy-5-oxo-4,5-dihydro-1H-pyrrolo[3,2-b]pyridine-6-carbonitrile,
N-[4′-(6-Cyano-7-hydroxy-5-oxo-4,5-dihydro-1H-pyrrolo[3,2-b]pyridin-1-yl)-4-biphenylyl]acetamide,
1-(2′-Chloro-4-biphenylyl)-7-hydroxy-5-oxo-4,5-dihydro-1H-pyrrolo[3,2-b]pyridine-6-carbonitrile,
7-Hydroxy-1-{4-[6-(methyloxy)-3-pyridinyl]phenyl}-5-oxo-4,5-dihydro-1H-pyrrolo[3,2-b]pyridine-6-carbonitrile,
1-(4′-Fluoro-4-biphenylyl)-7-hydroxy-4-methyl-5-oxo-4,5-dihydro-1H-pyrrolo[3,2-b]pyridine-6-carbonitrile,
7-Hydroxy-1-(2′-hydroxy-4-biphenylyl)-1,4-dihydro-5H-pyrrolo[3,2-b]pyridin-5-one,
1-[4-(6-Fluoro-3-pyridinyl)phenyl]-7-hydroxy-5-oxo-4,5-dihydro-1H-pyrrolo[3,2-b]pyridine-6-carbonitrile,
1-(4-Bromophenyl)-2-chloro-7-hydroxy-5-oxo-4,5-dihydro-1H-pyrrolo[3,2-b]pyridine-6-carbonitrile,
2-Chloro-7-hydroxy-1-[2′-hydroxy-3′-(methyloxy)-4-biphenylyl]-5-oxo-4,5-dihydro-1H-pyrrolo[3,2-b]pyridine-6-carbonitrile,
2-Chloro-1-(2′-fluoro-4-biphenylyl)-7-hydroxy-5-oxo-4,5-dihydro-1H-pyrrolo[3,2-b]pyridine-6-carbonitrile,
2-Chloro-7-hydroxy-1-(2′-hydroxy-4-biphenylyl)-5-oxo-4,5-dihydro-1H-pyrrolo[3,2-b]pyridine-6-carbonitrile,
2-Chloro-7-hydroxy-1-(4′-methyl-4-biphenylyl)-5-oxo-4,5-dihydro-1H-pyrrolo[3,2-b]pyridine-6-carbonitrile,
2-Chloro-1-(4′-fluoro-4-biphenylyl)-7-hydroxy-5-oxo-4,5-dihydro-1H-pyrrolo[3,2-b]pyridine-6-carbonitrile,
2-Chloro-7-hydroxy-1-[4′-(methyloxy)-4-biphenylyl]-5-oxo-4,5-dihydro-1H-pyrrolo[3,2-b]pyridine-6-carbonitrile,
Ethyl 7-hydroxy-1-(2′-hydroxy-4-biphenylyl)-5-oxo-4,5-dihydro-1H-pyrrolo[3,2-b]pyridine-6-carboxylate,
4′-(6-Cyano-7-hydroxy-5-oxo-4,5-dihydro-1H-pyrrolo[3,2-b]pyridin-1-yl)-2-hydroxy-3-biphenylcarboxylic acid,
1-(2′,4′-Difluoro-4-biphenylyl)-7-hydroxy-5-oxo-4,5-dihydro-1H-pyrrolo[3,2-b]pyridine-6-carbonitrile,
7-Hydroxy-5-oxo-1-[2′-(trifluoromethyl)-4-biphenylyl]-4,5-dihydro-1H-pyrrolo[3,2-b]pyridine-6-carbonitrile,
1-(2′,4′-Dichloro-4-biphenylyl)-7-hydroxy-5-oxo-4,5-dihydro-1H-pyrrolo[3,2-b]pyridine-6-carbonitrile,
1-[4-(5-Chloro-2-thienyl)phenyl]-7-hydroxy-5-oxo-4,5-dihydro-1H-pyrrolo[3,2-b]pyridine-6-carbonitrile,
1-(4-Bromophenyl)-6-(4-fluorophenyl)-7-hydroxy-1,4-dihydro-5H-pyrrolo[3,2-b]pyridin-5-one,
6-(4-Fluorophenyl)-7-hydroxy-1-(2′-hydroxy-4-biphenylyl)-1,4-dihydro-5H-pyrrolo[3,2-b]pyridin-5-one,
1-(4-Bromophenyl)-7-hydroxy-6-[4-(methyloxy)phenyl]-1,4-dihydro-5H-pyrrolo[3,2-b]pyridin-5-one,
4-[7-Hydroxy-1-(2′-hydroxy-4-biphenylyl)-5-oxo-4,5-dihydro-1H-pyrrolo[3,2-b]pyridin-6-yl]benzonitrile,
7-Hydroxy-1-(2′-hydroxy-4-biphenylyl)-6-phenyl-1,4-dihydro-5H-pyrrolo[3,2-b]pyridin-5-one,
Ethyl 4-(6-cyano-7-hydroxy-5-oxo-4,5-dihydro-1H-pyrrolo[3,2-b]pyridin-1-yl)benzoate,
7-Hydroxy-1-[4-(methyloxy)phenyl]-5-oxo-4,5-dihydro-1H-pyrrolo[3,2-b]pyridine-6-carbonitrile,
1-(4-Bromophenyl)-7-hydroxy-1,4-dihydro-5H-pyrrolo[3,2-b]pyridin-5-one,
1-(2′-Fluoro-4′-methyl-4-biphenylyl)-7-hydroxy-5-oxo-4,5-dihydro-1H-pyrrolo[3,2-b]pyridine-6-carbonitrile,
7-Hydroxy-5-oxo-1-[4′-(trimethylsilyl)-4-biphenylyl]-4,5-dihydro-1H-pyrrolo[3,2-b]pyridine-6-carbonitrile,
1-[4-(1,3-Benzodioxol-5-yl)phenyl]-7-hydroxy-5-oxo-4,5-dihydro-1H-pyrrolo[3,2-b]pyridine-6-carbonitrile,
1-(2′,4′-Dimethyl-4-biphenylyl)-7-hydroxy-5-oxo-4,5-dihydro-1H-pyrrolo[3,2-b]pyridine-6-carbonitrile,
7-Hydroxy-5-oxo-1-[4-(3-thienyl)phenyl]-4,5-dihydro-1H-pyrrolo[3,2-b]pyridine-6-carbonitrile,
7-Hydroxy-1-[4-(6-methyl-2-pyridinyl)phenyl]-5-oxo-4,5-dihydro-1H-pyrrolo[3,2-b]pyridine-6-carbonitrile,
7-Hydroxy-5-oxo-1-[4-(4-pyridinyl)phenyl]-4,5-dihydro-1H-pyrrolo[3,2-b]pyridine-6-carbonitrile,
1-[4-(5-Cyano-2-pyridinyl)phenyl]-7-hydroxy-5-oxo-4,5-dihydro-1H-pyrrolo[3,2-b]pyridine-6-carbonitrile,
1-[4-(3,5-Dimethyl-4-isoxazolyl)phenyl]-7-hydroxy-5-oxo-4,5-dihydro-1H-pyrrolo[3,2-b]pyridine-6-carbonitrile,
7-Hydroxy-5-oxo-1-[4-(3-pyridinyl)phenyl]-4,5-dihydro-1H-pyrrolo[3,2-b]pyridine-6-carbonitrile,
7-Hydroxy-1-[4-(5-methyl-2-pyridinyl)phenyl]-5-oxo-4,5-dihydro-1H-pyrrolo[3,2-b]pyridine-6-carbonitrile,
7-Hydroxy-1-{4-[5-(methyloxy)-2-pyridinyl]phenyl}-5-oxo-4,5-dihydro-1H-pyrrolo[3,2-b]pyridine-6-carbonitrile,
7-Hydroxy-5-oxo-1-[4-(1H-pyrrol-2-yl)phenyl]-4,5-dihydro-1H-pyrrolo[3,2-b]pyridine-6-carbonitrile,
1-(3′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-3′-methyl-4-biphenylyl)-5-oxo-4,5-dihydro-1H-pyrrolo[3,2-b]pyridine-6-carbonitrile,
7-Hydroxy-5-oxo-1-[4-(2-thienyl)phenyl]-4,5-dihydro-1H-pyrrolo[3,2-b]pyridine-6-carbonitrile,
1-(3′-Chloro-2′-hydroxy-4-biphenylyl)-7-hydroxy-5-oxo-4,5-dihydro-1H-pyrrolo[3,2-b]pyridine-6-carbonitrile,
1-(5′-Fluoro-2′-hydroxy-4-biphenylyl)-7-hydroxy-5-oxo-4,5-dihydro-1H-pyrrolo[3,2-b]pyridine-6-carbonitrile,
1-(2′-Fluoro-6′-hydroxy-4-biphenylyl)-7-hydroxy-5-oxo-4,5-dihydro-1H-pyrrolo[3,2-b]pyridine-6-carbonitrile,
1-(4′-Chloro-2′-hydroxy-4-biphenylyl)-7-hydroxy-5-oxo-4,5-dihydro-1H-pyrrolo[3,2-b]pyridine-6-carbonitrile,
7-Hydroxy-1-(2′-hydroxy-5′-methyl-4-biphenylyl)-5-oxo-4,5-dihydro-1H-pyrrolo[3,2-b]pyridine-6-carbonitrile,
4′-(6-Cyano-7-hydroxy-5-oxo-4,5-dihydro-1H-pyrrolo[3,2-b]pyridine-1-yl)-6-hydroxy-3-biphenylcarboxylic acid,
2-Chloro-1-(5′-fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[2′-hydroxy-3′-(methyloxy)-5′-(2-propen-1-yl)-4-biphenylyl]-5-oxo-4,5-dihydro-1H-pyrrolo[3,2-b]pyridine-6-carbonitrile,
1-[4-(2,3-Dihydro-1,4-benzodioxin-6-yl)phenyl]-7-hydroxy-5-oxo-4,5-dihydro-1H-pyrrolo[3,2-b]pyridine-6-carbonitrile,
2-Chloro-7-hydroxy-1-(2′-hydroxy-3′-methyl-4-biphenylyl)-5-oxo-4,5-dihydro-1H-pyrrolo[3,2-b]pyridine-6-carbonitrile,
4′-(2-Chloro-6-cyano-7-hydroxy-5-oxo-4,5-dihydro-1H-pyrrolo[3,2-b]pyridine-1-yl)-6-hydroxy-3-biphenylcarboxylic acid,
1-(2′-Chloro-6′-hydroxy-4-biphenylyl)-7-hydroxy-5-oxo-4,5-dihydro-1H-pyrrolo[3,2-b]pyridine-6-carbonitrile,
7-Hydroxy-1-{4-[2-(methyloxy)-3-pyridinyl]phenyl}-5-oxo-4,5-dihydro-1H-pyrrolo[3,2-b]pyridine-6-carbonitrile,
2-Chloro-7-hydroxy-5-oxo-1-[4-(3-thienyl)phenyl]-4,5-dihydro-1H-pyrrolo[3,2-b]pyridine-6-carbonitrile,
1-(4′-Fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[4-(2-hydroxy-3-pyridinyl)phenyl]-5-oxo-4,5-dihydro-1H-pyrrolo[3,2-b]pyridine-6-carbonitrile,
2-Chloro-1-(4′-chloro-2′-hydroxy-4-biphenylyl)-7-hydroxy-5-oxo-4,5-dihydro-1H-pyrrolo[3,2-b]pyridine-6-carbonitrile,
7-Hydroxy-1-{4-[4-(methyloxy)-3-pyridinyl]phenyl}-5-oxo-4,5-dihydro-1H-pyrrolo[3,2-b]pyridine-6-carbonitrile,
1-[4-(2-Furanyl)phenyl]-7-hydroxy-5-oxo-4,5-dihydro-1H-pyrrolo[3,2-b]pyridine-6-carbonitrile,
7-Hydroxy-2-methyl-5-oxo-1 [4-(3-thienyl)phenyl]-4,5-dihydro-1H-pyrrolo[3,2-b]pyridine-6-carbonitrile,
4-[1-(5′-Fluoro-2′-hydroxy-4-biphenylyl)-7-hydroxy-5-oxo-4,5-dihydro-1H-pyrrolo[3,2-b]pyridin-6-yl]benzonitrile,
4-[7-Hydroxy-1-(2′-hydroxy-5′-methyl-4-biphenylyl)-5-oxo-4,5-dihydro-1H-pyrrolo[3,2-b]pyridin-6-yl]benzonitrile,
4-[7-Hydroxy-1-(2′-hydroxy-3′-methyl-4-biphenylyl)-5-oxo-4,5-dihydro-1H-pyrrolo[3,2-b]pyridin-6-yl]benzonitrile,
2-Chloro-1-(6′-fluoro-2′-hydroxy-4-biphenylyl)-7-hydroxy-5-oxo-4,5-dihydro-1H-pyrrolo[3,2-b]pyridine-6-carbonitrile,
2-Chloro-1-(4′-fluoro-2′-hydroxy-4-biphenylyl)-7-hydroxy-5-oxo-4,5-dihydro-1H-pyrrolo[3,2-b]pyridine-6-carbonitrile,
7-Hydroxy-1-[4-(4-methyl-2-thienyl)phenyl]-5-oxo-4,5-dihydro-1H-pyrrolo[3,2-b]pyridine-6-carbonitrile,
7-Hydroxy-5-oxo-1-[4-(1H-pyrrol-3-yl)phenyl]-4,5-dihydro-1H-pyrrolo[3,2-b]pyridine-6-carbonitrile,
7-Hydroxy-5-oxo-1-[4-(1,3-thiazol-2-yl)phenyl]-4,5-dihydro-1H-pyrrolo[3,2-b]pyridine-6-carbonitrile,
7-Hydroxy-5-oxo-1-[4-(1,3-thiazol-4-yl)phenyl]-4,5-dihydro-1H-pyrrolo[3,2-b]pyridine-6-carbonitrile,
7-Hydroxy-1-[4-(3-methyl-2-thienyl)phenyl]-5-oxo-4,5-dihydro-1H-pyrrolo[3,2-b]pyridine-6-carbonitrile,
5-[4-(6-Cyano-7-hydroxy-5-oxo-4,5-dihydro-1H-pyrrolo[3,2-b]pyridin-1-yl)phenyl]-2-thiophenesulfonamide,
7-Hydroxy-1-[4-(1-methyl-1H-pyrazol-4-yl)phenyl]-5-oxo-4,5-dihydro-1H-pyrrolo[3,2-b]pyridine-6-carbonitrile,
7-Hydroxy-1-[4-(2-methyl-1,3-thiazol-4-yl)phenyl]-5-oxo-4,5-dihydro-1H-pyrrolo[3,2-b]pyridine-6-carbonitrile,
2-Chloro-1-[2′-fluoro-6′-(methyloxy)-4-biphenylyl]-7-hydroxy-5-oxo-4,5-dihydro-1H-pyrrolo[3,2-b]pyridine-6-carbonitrile,
7-Hydroxy-1-[4-(5-methyl-2-thienyl)phenyl]-5-oxo-4,5-dihydro-1H-pyrrolo[3,2-b]pyridine-6-carbonitrile,
5-[4-(2-Chloro-6-cyano-7-hydroxy-5-oxo-4,5-dihydro-1H-pyrrolo[3,2-b]pyridin-1-yl)phenyl]-2-thiophenesulfonamide,
2-Chloro-1-[4-(5-chloro-2-thienyl)phenyl]-7-hydroxy-5-oxo-4,5-dihydro-1H-pyrrolo[3,2-b]pyridine-6-carbonitrile,
4-[4-(2-Chloro-6-cyano-7-hydroxy-5-oxo-4,5-dihydro-1H-pyrrolo[3,2-b]pyridin-1-yl)phenyl]-2-thiophenecarboxamide,
1-[4-(1-Benzothien-3-yl)phenyl]-2-chloro-7-hydroxy-5-oxo-4,5-dihydro-1H-pyrrolo[3,2-b]pyridine-6-carbonitrile,
2-Chloro-7-hydroxy-1-{4-[5-(methyloxy)-2-pyridinyl]phenyl}-5-oxo-4,5-dihydro-1H-pyrrolo[3,2-b]pyridine-6-carbonitrile,
2-Chloro-7-hydroxy-5-oxo-1-[4-(1,3-thiazol-4-yl)phenyl]-4,5-dihydro-1H-pyrrolo[3,2-b]pyridine-6-carbonitrile,
2-Chloro-1-[4-(4-cyano-3-thienyl)phenyl]-7-hydroxy-5-oxo-4,5-dihydro-1H-pyrrolo[3,2-b]pyridine-6-carbonitrile,
4-[4-(2-Chloro-6-cyano-7-hydroxy-5-oxo-4,5-dihydro-1H-pyrrolo[3,2-b]pyridin-1-yl)phenyl]-3-thiophenecarboxamide,
2-Chloro-7-hydroxy-5-oxo-1-[4-(3-pyridinyl)phenyl]-4,5-dihydro-1H-pyrrolo[3,2-b]pyridine-6-carbonitrile,
2-Chloro-1-[2′-chloro-6′-(methyloxy)-4-biphenylyl]-7-hydroxy-5-oxo-4,5-dihydro-1H-pyrrolo[3,2-b]pyridine-6-carbonitrile,
2-Chloro-7-hydroxy-1-[4-(3-methyl-2-thienyl)phenyl]-5-oxo-4,5-dihydro-1H-pyrrolo[3,2-b]pyridine-6-carbonitrile,
2-Chloro-7-hydroxy-1-[4-(1-methyl-1H-pyrazol-4-yl)phenyl]-5-oxo-4,5-dihydro-1H-pyrrolo[3,2-b]pyridine-6-carbonitrile,
2-Chloro-7-hydroxy-1-[4-(2-methyl-1,3-thiazol-4-yl)phenyl]-5-oxo-4,5-dihydro-1H-pyrrolo[3,2-b]pyridine-6-carbonitrile,
2-Chloro-7-hydroxy-1-(3′-hydroxy-4-biphenylyl)-5-oxo-4,5-dihydro-1H-pyrrolo[3,2-b]pyridine-6-carbonitrile,
1-[4-(6-Amino-2-pyridinyl)phenyl]-2-chloro-7-hydroxy-5-oxo-4,5-dihydro-1H-pyrrolo[3,2-b]pyridine-6-carbonitrile,
2-Chloro-7-hydroxy-1-[3′-(hydroxymethyl)-4-biphenylyl]-5-oxo-4,5-dihydro-1H-pyrrolo[3,2-b]pyridine-6-carbonitrile,
7-Hydroxy-5-oxo-1-[4-(3-thienyl)phenyl]-4,5-dihydro-1H-pyrrolo[3,2-b]pyridine-2,6-dicarbonitrile,
2-Chloro-7-hydroxy-1-[4-(5-methyl-2-thienyl)phenyl]-5-oxo-4,5-dihydro-1H-pyrrolo[3,2-b]pyridine-6-carbonitrile,
2-Chloro-7-hydroxy-5-oxo-1-[4-(2-thienyl)phenyl]-4,5-dihydro-1H-pyrrolo[3,2-b]pyridine-6-carbonitrile,
2-Chloro-1-(3′-fluoro-2′-hydroxy-4-biphenylyl)-7-hydroxy-5-oxo-4,5-dihydro-1H-pyrrolo[3,2-b]pyridine-6-carbonitrile,
2-Chloro-1-(3′-chloro-2′-hydroxy-4-biphenylyl)-7-hydroxy-5-oxo-4,5-dihydro-1H-pyrrolo[3,2-b]pyridine-6-carbonitrile,
2-Chloro-1-(2′-fluoro-4′-methyl-4-biphenylyl)-7-hydroxy-5-oxo-4,5-dihydro-1H-pyrrolo[3,2-b]pyridine-6-carbonitrile,
2-Chloro-1-[2′-chloro-4′-(methyloxy)-4-biphenylyl]-7-hydroxy-5-oxo-4,5-dihydro-1H-pyrrolo[3,2-b]pyridine-6-carbonitrile,
1-(4-Ethylphenyl)-7-hydroxy-5-oxo-4,5-dihydro-1H-pyrrolo[3,2-b]pyridine-2,6-dicarbonitrile,
2-Chloro-1-(2′-chloro-6′-hydroxy-4-biphenylyl)-7-hydroxy-5-oxo-4,5-dihydro-1H-pyrrolo[3,2-b]pyridine-6-carbonitrile,
2-Chloro-7-hydroxy-1-(2′-hydroxy-5′-methyl-4-biphenylyl)-5-oxo-4,5-dihydro-1H-pyrrolo[3,2-b]pyridine-6-carbonitrile,
2-Chloro-6-(2-fluorophenyl)-7-hydroxy-1-[4-(methyloxy)phenyl]-1,4-dihydro-5H-pyrrolo[3,2-b]pyridin-5-one,
2-Chloro-6-(3-fluorophenyl)-7-hydroxy-1-[4-(methyloxy)phenyl]-1,4-dihydro-5H-pyrrolo[3,2-b]pyridine-5-one,
2-Chloro-1-(4-ethylphenyl)-7-hydroxy-6-phenyl-1,4-dihydro-5H-pyrrolo[3,2-b]pyridine-5-one,
2-Chloro-6-(4-fluorophenyl)-7-hydroxy-1-[4-(methyloxy)phenyl]-1,4-dihydro-5H-pyrrolo[3,2-b]pyridin-5-one,
2-Chloro-7-hydroxy-1-[4-(methyloxy)phenyl]-6-phenyl-1,4-dihydro-5H-pyrrolo[3,2-b]pyridin-5-one,
2-Chloro-1-(4-fluorophenyl)-7-hydroxy-6-phenyl-1,4-dihydro-5H-pyrrolo[3,2-b]pyridin-5-one,
2-Chloro-6-(2-fluorophenyl)-7-hydroxy-1-[3-(methyloxy)phenyl]-1,4-dihydro-5H-pyrrolo[3,2-b]pyridin-5-one,
2-Chloro-6-(3-fluorophenyl)-7-hydroxy-1-[3-(methyloxy)phenyl]-1,4-dihydro-5H-pyrrolo[3,2-b]pyridin-5-one,
2-Chloro-6-(4-fluorophenyl)-7-hydroxy-1-[3-(methyloxy)phenyl]-1,4-dihydro-5H-pyrrolo[3,2-b]pyridin-5-one,
2-Chloro-7-hydroxy-1-[3-(methyloxy)phenyl]-6-phenyl-1,4-dihydro-5H-pyrrolo[3,2-b]pyridin-5-one,
2-Chloro-6-(4-fluorophenyl)-7-hydroxy-1-(4-methylphenyl)-1,4-dihydro-5H-pyrrolo[3,2-b]pyridin-5-one,
2-Chloro-7-hydroxy-6-phenyl-1-[4-(trifluoromethyl)phenyl]-1,4-dihydro-5H-pyrrolo[3,2-b]pyridin-5-one,
2-Chloro-7-hydroxy-1-(4-methylphenyl)-6-phenyl-1,4-dihydro-5H-pyrrolo[3,2-b]pyridin-5-one,
2-Chloro-6-(2-fluorophenyl)-7-hydroxy-1-(4-methylphenyl)-1,4-dihydro-5H-pyrrolo[3,2-b]pyridin-5-one,
2-Chloro-6-(3-fluorophenyl)-7-hydroxy-1-(4-methylphenyl)-1,4-dihydro-5H-pyrrolo[3,2-b]pyridin-5-one,
2-Chloro-6-(2-fluorophenyl)-7-hydroxy-1-[4-(trifluoromethyl)phenyl]-1,4-dihydro-5H-pyrrolo[3,2-b]pyridin-5-one,
2-Chloro-6-(3-fluorophenyl)-7-hydroxy-1-[4-(trifluoromethyl)phenyl]-1,4-dihydro-5H-pyrrolo[3,2-b]pyridin-5-one,
2-Chloro-6-(2-fluorophenyl)-7-hydroxy-1-{4-[(trifluoromethyl)oxy]phenyl}-1,4-dihydro-5H-pyrrolo[3,2-b]pyridin-5-one,
2-Chloro-6-[3-(ethyloxy)phenyl]-7-hydroxy-1-(4-methylphenyl)-1,4-dihydro-5H-pyrrolo[3,2-b]pyridin-5-one,
4-[2-Chloro-7-hydroxy-1-(4-methylphenyl)-5-oxo-4,5-dihydro-1H-pyrrolo[3,2-b]pyridin-6-yl]benzonitrile,
2-Chloro-1-(3,4-dimethylphenyl)-7-hydroxy-6-phenyl-1,4-dihydro-5H-pyrrolo[3,2-b]pyridin-5-one,
2-Chloro-7-hydroxy-1-(4-methylphenyl)-6-(3-nitrophenyl)-1,4-dihydro-5H-pyrrolo[3,2-b]pyridin-5-one,
3-[2-Chloro-7-hydroxy-1-(4-methylphenyl)-5-oxo-4,5-dihydro-1H-pyrrolo[3,2-b]pyridin-6-yl]benzonitrile,
[4-(2-Chloro-7-hydroxy-5-oxo-6-phenyl-4,5-dihydro-1H-pyrrolo[3,2-b]pyridin-1-yl)phenyl]acetonitrile,
6-(3-Bromophenyl)-2-chloro-7-hydroxy-1-(4-methylphenyl)-1,4-dihydro-5H-pyrrolo[3,2-b]pyridin-5-one,
3-[2-Chloro-7-hydroxy-1-(4-methylphenyl)-5-oxo-4,5-dihydro-1H-pyrrolo[3,2-b]pyridin-6-yl]benzoic acid,
1-(4-ethylphenyl)-7-hydroxy-5-oxo-6-phenyl-4,5-dihydro-1H-pyrrolo[3,2-b]pyridine-2-carbonitrile,
6-(2-Fluorophenyl)-7-hydroxy-1-[4-(methyloxy)phenyl]-1,4-dihydro-5H-pyrrolo[3,2-b]pyridin-5-one,
6-(3-Fluorophenyl)-7-hydroxy-1-[4-(methyloxy)phenyl]-1,4-dihydro-5H-pyrrolo[3,2-b]pyridin-5-one,
2-Chloro-7-hydroxy-6-[3-(methyloxy)phenyl]-1-[4-(3-thienyl)phenyl]-1,4-dihydro-5H-pyrrolo[3,2-b]pyridin-5-one,
3-{2-Chloro-7-hydroxy-5-oxo-1-[4-(3-thienyl)phenyl]-4,5-dihydro-1H-pyrrolo[3,2-b]pyridin-6-yl}benzoic acid,
2-Chloro-7-hydroxy-6-phenyl-1-[4-(3-thienyl)phenyl]-1,4-dihydro-5H-pyrrolo[3,2-b]pyridin-5-one,
2-{2-Chloro-7-hydroxy-5-oxo-1-[4-(3-thienyl)phenyl]-4,5-dihydro-1H-pyrrolo[3,2-b]pyridin-6-yl}benzoic acid,
4-{2-Chloro-7-hydroxy-5-oxo-1-[4-(3-thienyl)phenyl]-4,5-dihydro-1H-pyrrolo[3,2-b]pyridin-6-yl}benzonitrile,
3-{2-Chloro-7-hydroxy-5-oxo-1-[4-(3-thienyl)phenyl]-4,5-dihydro-1H-pyrrolo[3,2-b]pyridin-6-yl}benzonitrile,
1-(4-Acetylphenyl)-2-chloro-7-hydroxy-5-oxo-4,5-dihydro-1H-pyrrolo[3,2-b]pyridine-6-carbonitrile,
N-[4-(2-Chloro-6-cyano-7-hydroxy-5-oxo-4,5-dihydro-1H-pyrrolo[3,2-b]pyridin-1-yl)phenyl]acetamide,
2-Chloro-7-hydroxy-1-(4′-methyl-4-biphenylyl)-6-phenyl-1,4-dihydro-5H-pyrrolo[3,2-b]pyridin-5-one,
2-Chloro-1-(4-cyclohexylphenyl)-7-hydroxy-6-phenyl-1,4-dihydro-5H-pyrrolo[3,2-b]pyridin-5-one,
4-{2-Chloro-7-hydroxy-5-oxo-1-[4-(3-thienyl)phenyl]-4,5-dihydro-1H-pyrrolo[3,2-b]pyridin-6-yl}benzamide,
3-{2-Chloro-7-hydroxy-5-oxo-1-[4-(3-thienyl)phenyl]-4,5-dihydro-1H-pyrrolo[3,2-b]pyridin-6-yl}benzamide,
4-{2-Chloro-7-hydroxy-5-oxo-1-[4-(3-thienyl)phenyl]-4,5-dihydro-1H-pyrrolo[3,2-b]pyridin-6-yl}benzoic acid,
2-Chloro-7-hydroxy-1-[2′-hydroxy-3′-(methyloxy)-4-biphenylyl]-6-phenyl-1,4-dihydro-5H-pyrrolo[3,2-b]pyridin-5-one,
2-Chloro-6-(2-fluorophenyl)-7-hydroxy-1-[2′-hydroxy-3′-(methyloxy)-4-biphenylyl]-1,4-dihydro-5H-pyrrolo[3,2-b]pyridin-5-one,
2-Chloro-6-(3-chlorophenyl)-7-hydroxy-1-[2′-hydroxy-3′-(methyloxy)-4-biphenylyl]-1,4-dihydro-5H-pyrrolo[3,2-b]pyridin-5-one,
3-{2-Chloro-7-hydroxy-1-[2′-hydroxy-3′-(methyloxy)-4-biphenylyl]-5-oxo-4,5-dihydro-1H-pyrrolo[3,2-b]pyridin-6-yl}benzoic acid,
4-{2-Chloro-7-hydroxy-1-[2′-hydroxy-3′-(methyloxy)-4-biphenylyl]-5-oxo-4,5-dihydro-1H-pyrrolo[3,2-b]pyridin-6-yl}benzonitrile,
2-Chloro-7-hydroxy-1-[2′-hydroxy-3′-(methyloxy)-4-biphenylyl]-6-[3-(methyloxy)phenyl]-1,4-dihydro-5H-pyrrolo[3,2-b]pyridin-5-one, and
4-{2-Chloro-7-hydroxy-1-[2′-hydroxy-3′-(methyloxy)-4-biphenylyl]-5-oxo-4,5-dihydro-1H-pyrrolo[3,2-b]pyridin-6-yl}benzoic acid,
and salts thereof.

10. A compound of formula (I) or a salt thereof according to any claim 1 wherein the salt is a pharmaceutically acceptable salt.

11. A pharmaceutical composition comprising a) a compound of formula (I) or pharmaceutically acceptable salt thereof according to claim 10 and b) at least one pharmaceutically acceptable carrier.

12-16. (canceled)

17. A method of treating a disease or a condition susceptible to amelioration by an AMPK activator comprising administering to a subject a therapeutically effective amount of a compound for formula (I) or a pharmaceutically acceptable salt thereof according to claim 10.

18. A method of treating type 1 diabetes, type 2 diabetes, metabolic syndrome, atherosclerosis, dyslipidaemia, mitochondrial disorders, sarcopenia, obesity, hypertension, cerebral ischemia, cognitive defect Alzheimer's disease, Parkinson's disease, Huntington's disease, schizophrenia, Friedrich's Ataxia, amyotrophic lateral sclerosis, multiple sclerosis, neuroinflammation, inflammatory pain, neuropathic pain, epilepsy, virus infection (HIV, cytomegalovirus and hepatitis C) or cancer comprising administering to a subject a therapeutically effective amount of a compound for formula (I) or a pharmaceutically acceptable salt thereof according to claim 10.
